(19) United States
(12) Patent Application Publication
(10) Pub. No.: US 2008/0051429 A1
(43) Pub. Date: Feb. 28, 2008

(54) USE OF 4-AMINO-PIPERIDINES FOR TREATING SLEEP DISORDERS

(76) Inventors: Daniel van Kammen, San Diego, CA (US); David M. Weiner, San Diego, CA (US); Mark R. Brann, Rye, NH (US); Robert E. Davis, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/737,097

(22) Filed: Apr. 18, 2007

Related U.S. Application Data
(60) Provisional application No. 60/793,741, filed on Apr. 19, 2006.

(51) Int. Cl. A61K 31/445; A61P 43/00

(52) U.S. Cl. 514/315

(57) ABSTRACT

Inverse agonists and antagonists of serotonin receptors are disclosed for use in treating sleep disorders such as insomnia, and specifically sleep maintenance insomnia. The compound increase slow wave sleep, decrease the number of awakenings after sleep onset, and decrease the time awake after sleep onset.

### RELATED APPLICATIONS

This application claims the benefit of U.S. Provisional Patent Application No. 60/793,741, entitled “USE OF 4-AMINO-PIPERIDINES FOR TREATING SLEEP DISORDERS,” filed Apr. 19, 2006, which is hereby incorporated by reference in its entirety, including any drawings.

### BACKGROUND OF THE INVENTION

1. Field of the Invention

The present invention relates to treating sleep disorders using compounds that are selective towards monoamine receptors such as serotonin receptors. Compounds that are useful for treating sleep disorders include 4-amino-piperidines.

2. Description of the Related Art

One common sleep disorder is insomnia. In some patients, the insomnia is sleep maintenance insomnia. Sleep maintenance insomnia (SMI) is the inability to stay asleep or to resume sleep after waking and is a major unmet medical need. Deep, or slow wave, sleep decreases with age, which leads to superficial sleep and difficulty staying asleep. There is also an increased incidence of SMI in medical, neurological and psychiatric conditions. Patients with SMI complain of frequent awakenings and difficulty staying asleep after falling asleep. Patients with these symptoms also frequently report impairments of daytime functioning. Most available sleep agents are sedatives that are ineffective in treating the symptoms of SMI. Thus, there is a need for new drugs to treat insomnia, and specifically, sleep maintenance insomnia.

### SUMMARY OF THE INVENTION

One embodiment described herein includes a method of increasing slow-wave sleep comprising administering N-(1-methylpiperidin-4-yl)-N-(4-fluorophenylmethyl)-N′-(4-(2-methylpropyloxy)phenylmethyl)carbamide to a subject in need of increased slow wave sleep in an amount sufficient to increase slow wave sleep. One embodiment further comprises informing the subject that N-(1-methylpiperidin-4-yl)-N-(4-fluorophenylmethyl)-N′-(4-(2-methylpropyloxy)phenylmethyl)carbamide increases slow wave sleep. In one embodiment, the informing comprises providing printed matter that advises that N-(1-methylpiperidin-4-yl)-N-(4-fluorophenylmethyl)-N′-(4-(2-methylpropyloxy)phenylmethyl)carbamide increases slow wave sleep. In one embodiment, the printed matter is a label.

Another embodiment disclosed herein includes a method of treating insomnia comprising administering N-(1-methylpiperidin-4-yl)-N-(4-fluorophenylmethyl)-N′-(4-(2-methylpropyloxy)phenylmethyl)carbamide to a subject suffering from insomnia in an amount sufficient to ameliorate insomnia. One embodiment further comprises informing the subject that N-(1-methylpiperidin-4-yl)-N-(4-fluorophenylmethyl)-N′-(4-(2-methylpropyloxy)phenylmethyl)carbamide ameliorates insomnia. In one embodiment, the informing comprises providing printed matter that N-(1-methylpiperidin-4-yl)-N-(4-fluorophenylmethyl)-N′-(4-(2-methylpropyloxy)phenylmethyl)carbamide ameliorates insomnia. In one embodiment, the printed matter is a label.

Another embodiment disclosed herein includes a method for decreasing the number of awakenings after sleep onset comprising administering N-(1-methylpiperidin-4-yl)-N-(4-fluorophenylmethyl)-N′-(4-(2-methylpropyloxy)phenylmethyl)carbamide to a subject in need of a decreased number of awakenings after sleep onset in an amount sufficient to decrease the number of awakenings after sleep onset.

Another embodiment disclosed herein includes a method for decreasing the time awake after sleep onset comprising administering N-(1-methylpiperidin-4-yl)-N-(4-fluorophenylmethyl)-N′-(4-(2-methylpropyloxy)phenylmethyl)carbamide to a subject in need of decreased time awake after sleep onset in an amount sufficient to decrease time awake after sleep onset.

Another embodiment disclosed herein includes a method of manufacturing a pharmaceutical composition including obtaining a first dosage form comprising a first amount of N-(1-methylpiperidin-4-yl)-N-(4-fluorophenylmethyl)-N′-(4-(2-methylpropyloxy)phenylmethyl)carbamide, obtaining a second dosage form comprising a second amount of N-(1-methylpiperidin-4-yl)-N-(4-fluorophenylmethyl)-N′-(4-(2-methylpropyloxy)phenylmethyl)carbamide, and packaging together the first dosage form and the second dosage form. In one embodiment, the first dosage form and the second dosage form each comprise an oral dosage form. In one embodiment, the first dosage form comprises a higher dose of N-(1-methylpiperidin-4-yl)-N-(4-fluorophenylmethyl)-N′-(4-(2-methylpropyloxy)phenylmethyl)carbamide than the second dosage form.

In one embodiment of any of the above methods, the amount of N-(1-methylpiperidin-4-yl)-N-(4-fluorophenylmethyl)-N′-(4-(2-methylpropyloxy)phenylmethyl)carbamide is in the range of about 0.5 mg to about 50 mg. In one embodiment, the amount is in the range of about 1 mg to about 40 mg. In one embodiment, the amount is in the range of about 2.5 mg to about 30 mg. In one embodiment, the amount is in the range of about 5 mg to about 20 mg. In one embodiment, the administration does not affect a sleep parameter selected from the group consisting of sleep period time, total sleep time, sleep onset latency, number of stage shifts, total time awake, early morning wake, sleep efficiency index, microarousal index, and a REM sleep parameter. In one embodiment, the REM sleep parameter is selected from the group consisting of REM sleep duration, proportion of REM sleep, REM sleep latency, REM activity and REM density. In one embodiment, the amount results in a steady state blood plasma concentration of N-(1-methylpiperidin-4-yl)-N-(4-fluorophenylmethyl)-N′-(4-(2-methylpropyloxy)phenylmethyl)carbamide in the range of about 2 ng/mL to about 60 ng/mL. In one embodiment, the amount results in a steady state blood plasma concentration in the range of about 4 ng/mL to about 50 ng/mL. In one embodiment, the amount results in a steady state blood plasma concentration of in the range of about 6 ng/mL to about 40 ng/mL. In one embodiment, the amount results in a steady state blood plasma concentration in the range of about 8.5 ng/mL to about 35 ng/mL. In one embodiment, the subject is administered N-(1-methylpiperidin-4-yl)-N-(4-fluorophenylmethyl)-N′-(4-(2-methylpropyloxy)phenylmethyl)carbamide once every day. In one embodiment, the subject is administered a first dosage form at least 24 hours prior to administration of a second dosage form. In one embodiment, the first dosage form comprises a higher dose than the second dosage form. In one embodiment, N-(1-methylpiperidin-4-yl)-N-(4-fluorophenylmethyl)-N′-(4-(2-methylpropyloxy)phenylmethyl)carbamide is administered at a time other than immediately before a sleep period. In one embodiment, it is administered in the morning.

Another embodiment disclosed herein includes a packaged pharmaceutical composition, comprising N-(1-methylpiperidin-4-yl)-N-(4-fluorophenylmethyl)-N′-(4-(2-methylpropyloxy)phenylmethyl)carbamide in a container and instructions for using N-(1-methylpiperidin-4-yl)-N-(4-fluorophenylmethyl)-N′-(4-(2-methylpropyloxy)phenylmethyl)carbamide to treat insomnia.

Another embodiment disclosed herein includes a packaged pharmaceutical composition, comprising N-(1-methylpiperidin-4-yl)-N-(4-fluorophenylmethyl)-N′-(4-(2-methylpropyloxy)phenylmethyl)carbamide in a container and instructions for using N-(1-methylpiperidin-4-yl)-N-(4-fluorophenylmethyl)-N′-(4-(2-methylpropyloxy)phenylmethyl)carbamide to increase slow wave sleep, decrease the number of awakenings after sleep onset or decrease the time awake after sleep onset.

Another embodiment disclosed herein includes a kit comprising a first dosage form of N-(1-methylpiperidin-4-yl)-N-(4-fluorophenylmethyl)-N′-(4-(2-methylpropyloxy)phenylmethyl)carbamide and a second dosage form of N-(1-methylpiperidin-4-yl)-N-(4-fluorophenylmethyl)-N′-(4-(2-methylpropyloxy)phenylmethyl)carbamide. In one embodiment, the first dosage form contains a higher dose of N-(1-methylpiperidin-4-yl)-N-(4-fluorophenylmethyl)-N′-(4-(2-methylpropyloxy)phenylmethyl)carbamide than the second dosage form. In one embodiment, the kit further comprises instructions for taking the first dosage form at least 24 hours before taking the second dosage form. In one embodiment, the instructions are on a label. In one embodiment, the instructions further inform a subject that N-(1-methylpiperidin-4-yl)-N-(4-fluorophenylmethyl)-N′-(4-(2-methylpropyloxy)phenylmethyl)carbamide increases slow wave sleep. In one embodiment, the instructions further inform a subject that N-(1-methylpiperidin-4-yl)-N-(4-fluorophenylmethyl)-N′-(4-(2-methylpropyloxy)phenylmethyl)carbamide ameliorates insomnia. In one embodiment, the instructions further inform a subject that N-(1-methylpiperidin-4-yl)-N-(4-fluorophenylmethyl)-N′-(4-(2-methylpropyloxy)phenylmethyl)carbamide decreases the number of awakenings after sleep onset. In one embodiment, the instructions further inform a subject that N-(1-methylpiperidin-4-yl)-N-(4-fluorophenylmethyl)-N′-(4-(2-methylpropyloxy)phenylmethyl)carbamide decreases the time awake after sleep onset.

### BRIEF DESCRIPTION OF THE DRAWINGS

FIG. 1 is a graph depicting the change in slow wave sleep duration from baseline for placebo and various doses of N-(1-methylpiperidin-4-yl)-N-(4-fluorophenylmethyl)-N′-(4-(2-methylpropyloxy)phenylmethyl)carbamide.

FIG. 2 is a graph depicting the correlation between slow wave sleep duration and blood plasma level of N-(1-methylpiperidin-4-yl)-N-(4-fluorophenylmethyl)-N′-(4-(2-methylpropyloxy)phenylmethyl)carbamide.

FIG. 3 is a graph depicting the change in number of awakenings from baseline for placebo and various doses of N-(1-methylpiperidin-4-yl)-N-(4-fluorophenylmethyl)-N′-(4-(2-methylpropyloxy)phenylmethyl)carbamide.

### DETAILED DESCRIPTION OF THE PREFERRED EMBODIMENT

The present application relates to the use of compounds that are inverse agonists or antagonists of a serotonin receptor to treat a sleep disorder. In some embodiments, the compounds are inverse agonists or antagonists of a 5-HT2A receptor. In some embodiments, the compounds are selective inverse agonists or antagonists. In some embodiments, the sleep disorder is insomnia. In some embodiments, the insomnia is sleep maintenance insomnia. In some embodiments, the sleep maintenance insomnia is caused by other disorders. For example, in some embodiments, the sleep maintenance insomnia is comorbid with a psychiatric disorder including, but not limited to, schizophrenia, affective disorders including MDD, bipolar 2 depression, bipolar 1 mania, rapid cyclers, dysthymia, PTSD (e.g., nightmares or arousals), alcoholism, substance abuse, drug withdrawal, and anxiety. In some embodiments, the compounds described herein may be administered in combination with an SSRI or NASR to decrease insomnia, increase efficacy, decrease sexual side effects and akathisia, provide a faster response, and limit treatment resistance. In other embodiments, the sleep maintenance insomnia is caused by a neurological disorder, including but not limited to Parkinson's disease, multisystems atrophy, migraines, multiple sclerosis, Huntington's Chorea, “Sun-downing” in DAT and other dementia's, and epilepsy. Other disorders that may cause sleep maintenance insomnia that can be treated using the compounds described herein include, but are not limited to, rheumatoid and osteo-arthritis and other chronic disorders with pain, fibro-myalgia, and female menopause.

In some embodiments, the compounds described herein are administered to a patient to increase slow wave sleep in the patient for any purpose. In some embodiments, the compounds decrease the number of awakenings after sleep onset and the time awake after sleep onset.

In some embodiments, the compounds described herein are administered to a patient to treat other sleep related disorders including periodic limb movement syndrome or obstructive sleep apnea.

In some embodiments, the compounds achieve the desired effect on slow wave sleep, number of awakenings after sleep onset, and the time awake after sleep onset without affecting other sleep parameters. In some embodiments, sleep parameters unaffected include sleep period time, total sleep time, sleep onset latency, number of sleep stage shifts, total time awake, early morning wake, sleep efficiency index, microarousal index, and REM sleep parameters (e.g., REM sleep duration, proportion of REM sleep, REM sleep latency, REM activity, and REM density).

In some embodiments, the compounds are administered at a dose between about 0.5 mg and about 50 mg, between about 1 mg and about 40 mg, between about 2.5 mg and about 30 mg, or between about 1 mg and about 20 mg. In some embodiments, the dosage and administration regiman are sufficient to achieve a steady state blood plasma concentration of between about 2 ng/ml and about 60 ng/ml, between about 4 ng/ml and about 50 ng/ml, between about 6 ng/ml and about 40 ng/ml, or between about 8.5 ng/ml and about 35 ng/ml.

In some embodiments, the dosage is varied over the dosage regimen. For example, in one embodiment, the first administration or series of administrations have a higher dosage than subsequent administrations. In some embodiments, the compound administered has a relatively high half-life such that after an initial dosage sufficient to raise the blood plasma concentration to a desired level, subsequent dosages sufficient to maintain a desired steady state blood plasma concentration can be lower. In some embodiments, pharmaceutical compositions comprising multiple doses are packaged together.

In some embodiments, a sleep-inducing agent is administered in combination with the compounds described herein. The sleep-inducing agent may be administered to induce onset of sleep in the patient while the inverse agonist or antagonist of a serotonin receptor may be administered to maintain sleep in the patient. For example, in some embodiments, the compounds described herein may be administered to maintain slow wave sleep in the patient. Non-limiting examples of suitable sleep-inducing agents include AMBIEN®, indiplon, LUNESTA®, and melatonin.

By administration in “combination,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered intravenously. In one advantageous embodiment, the pharmacokinetics of the two or more agents are substantially the same.

In some embodiments, the compounds described herein are long acting (e.g., have a long half-life), allowing them to be given relatively infrequently and at times other than immediately prior to a desired sleep period. For example, in some embodiments, the compounds have a half-life long enough so that that they don't need to be administered more often than once a day. In various embodiments, the compound may be administered once every 1, 2, 3, 4, or 5 days and still provide sleep maintenance properties during each sleep period. In various embodiments, the half-life of the compound is from about 10 hours to about 100 hours, from about 20 hours to about 60 hours, or from about 35 hours to about 55 hours. In some embodiments, the compound is not administered immediately prior to a sleep period. For example, the compound may be administered in the morning or afternoon. In one embodiment, the compound is administered in the morning. In some embodiments, the long acting properties of the compounds eliminate or reduce any withdrawal effects experienced by patients as either a co-administered sleep-inducing agent or the inverse agonist or antagonist of a serotonin receptor wears off. For example, many short acting sleep agents cause a patient to wake in the middle of their sleep period and experience a withdrawal effect. In some embodiments, patients taking a compound described herein experience no such withdrawal effect.

### DEFINITIONS

For the purpose of the current disclosure, the following definitions shall in their entireties be used to define technical terms, and shall also, in their entireties, be used to define the scope of the composition of matter for which protection is sought in the claims. The term “constitutive activity” is defined as the basal activity of a receptor which is independent of the presence of an agonist. Constitutive activity of a receptor may be measured using a number of different methods, including cellular (e.g., membrane) preparations (see, e.g., Barr &. Manning, J. Biol. Chem. 272:32979-87 (1997)), purified reconstituted receptors with or without the associated G-protein in phospholipid vesicles (Cerione et al., Biochemistry 23:4519-25 (1984)), and functional cellular assays (U.S. Pat. No. 6,358,698).

The term “agonist” is defined as a compound that increases the activity of a receptor when it contacts the receptor.

The term “antagonist” is defined as a compound that competes with an agonist or inverse agonist for binding to a receptor, thereby blocking the action of an agonist or inverse agonist on the receptor. However, an antagonist (also known as a “neutral” antagonist) has no effect on constitutive receptor activity.

The term “inverse agonist” is defined as a compound that decreases the basal activity of a receptor (i.e., signaling mediated by the receptor). Such compounds are also known as negative antagonists. An inverse agonist is a ligand for a receptor that causes the receptor to adopt an inactive state relative to a basal state occurring in the absence of any ligand. Thus, while an antagonist can inhibit the activity of an agonist, an inverse agonist is a ligand that can alter the conformation of the receptor in the absence of an agonist. The concept of an inverse agonist has been explored by Bond et al. in Nature 374:272 (1995). More specifically, Bond et al. have proposed that unliganded β₂-adrenoceptor exists in an equilibrium between an inactive conformation and a spontaneously active conformation. Agonists are proposed to stabilize the receptor in an active conformation. Conversely, inverse agonists are believed to stabilize an inactive receptor conformation. Thus, while an antagonist manifests its activity by virtue of inhibiting an agonist, an inverse agonist can additionally manifest its activity in the absence of an agonist by inhibiting the spontaneous conversion of an unliganded receptor to an active conformation.

The term “5-HT2A receptor” is defined as a receptor, having an activity corresponding to the activity of the human serotonin receptor subtype, which was characterized through molecular cloning and pharmacology as detailed in Saltzman et al., Biochem. Biophys. Res. Comm. 181:1469-78; and Julius et al., Proc. Natl. Acad. Sci. USA 87:928-932.

The term “subject” refers to an animal, preferably a mammal, most preferably a human, who is the object of treatment, observation or experiment.

The term “selective” is defined as a property of a compound whereby an amount of the compound sufficient to effect a desired response from a particular receptor type, subtype, class or subclass causes a substantially smaller or no effect upon the activity other receptor types.

The terms “selectivity” or “selective,” in relation to an inverse agonist, are understood as a property of a compound of the invention whereby an amount of compound that effectively inversely agonizes the 5-HT2A receptor, and thereby decreases its activity, causes little or no inverse agonistic or antagonistic activity at other, related or unrelated, receptors. In particular, certain compounds of the invention have been found not to interact strongly with other serotonin receptors (5-HT 1A, 1B, 1D, 1E, 1F, 2B, 2C, 4A, 6, and 7) at concentrations where the signaling of the 5-HT2A receptor is strongly or completely inhibited. Preferably, the compounds of the invention are also selective with respect to other monoamine-binding receptors, such as the dopaminergic, histaminergic, adrenergic and muscarinic receptors. Compounds that are highly selective for 5-HT2A receptors may have a beneficial effect in the treatment of psychosis, schizophrenia or similar neuropsychiatric disorders, while avoiding adverse effects associated with drugs hitherto suggested for this purpose.

The EC₅₀ for an agonist is intended to denote the concentration of a compound needed to achieve 50% of a maximal response seen in R-SAT. For inverse agonists, EC₅₀ is intended to denote the concentration of a compound needed to achieve 50% inhibition of an R-SAT response from basal, no compound, levels.

In the present context the term “aryl” is intended to mean a carbocyclic aromatic ring or ring system. Moreover, the term “aryl” includes fused ring systems wherein at least two aryl rings, or at least one aryl and at least one C₃₋₈-cycloalkyl share at least one chemical bond. Some examples of “aryl” rings include optionally substituted phenyl, naphthalenyl, phenanthrenyl, anthracenyl, tetralinyl, fluorenyl, indenyl, and indanyl. The term “aryl” relates to aromatic, preferably benzenoid groups, connected via one of the ring-forming carbon atoms, and optionally carrying one or more substituents selected from heterocyclyl, heteroaryl, halo, hydroxy, amino, cyano, nitro, alkylamido, acyl, C₁₋₆ alkoxy, C₁₋₆ alkyl, C₁₋₆ hydroxyalkyl, C₁₋₆ aminoalkyl, C₁₋₆ alkylamino, alkylsulfenyl, alkylsulfinyl, alkylsulfonyl, sulfamoyl, or trifluoromethyl. The aryl group may be substituted at the para and/or meta positions. Representative examples of aryl groups include, but are not limited to, phenyl, 3-halophenyl, 4-halophenyl, 3-hydroxyphenyl, 4-hydroxyphenyl, 3-aminophenyl, 4-aminophenyl, 3-methylphenyl, 4-methylphenyl, 3-methoxyphenyl, 4-methoxyphenyl, 4-trifluoromethoxyphenyl 3-cyanophenyl, 4-cyanophenyl, dimethylphenyl, naphthyl, hydroxynaphthyl, hydroxymethylphenyl, trifluoromethylphenyl, alkoxyphenyl, 4-morpholin-4-ylphenyl, 4-pyrrolidin-1-ylphenyl, 4-pyrazolylphenyl, 4-triazolylphenyl, and 4-(2-oxopyrrolidin-1-yl)phenyl.

In the present context, the term “heteroaryl” is intended to mean a heterocyclic aromatic group where one or more carbon atoms in an aromatic ring have been replaced with one or more heteroatoms selected from the group comprising nitrogen, sulfur, phosphorous, and oxygen.

Furthermore, in the present context, the term “heteroaryl” comprises fused ring systems wherein at least one aryl ring and at least one heteroaryl ring, at least two heteroaryl rings, at least one heteroaryl ring and at least one heterocyclyl ring, or at least one heteroaryl ring and at least one C₃₋₈-cycloalkyl ring share at least one chemical bond.

The term “heteroaryl” is understood to relate to aromatic, C₃₋₈ cyclic groups further containing one oxygen or sulfur atom or up to four nitrogen atoms, or a combination of one oxygen or sulfur atom with up to two nitrogen atoms, and their substituted as well as benzo- and pyrido-fused derivatives, preferably connected via one of the ring-forming carbon atoms. Heteroaryl groups may carry one or more substituents, selected from halo, hydroxy, amino, cyano, nitro, alkylamido, acyl, C₁₋₆-alkoxy, C₁₋₆-alkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl. In some embodiments, heteroaryl groups may be five- and six-membered aromatic heterocyclic systems carrying 0, 1, or 2 substituents, which may be the same as or different from one another, selected from the list above. Representative examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quionoline, isoquinoline, pyridazine, pyrimidine, purine and pyrazine, which are all preferred, as well as furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline. In some embodiments, the substituents are halo, hydroxy, cyano, O—C₁₋₆-alkyl, C₁₋₆-alkyl, hydroxy-C₁₋₆-alkyl, amino-C₁₋₆-alkyl.

In the present context, the term “alkyl”, “C₁₋₆-alkyl” are intended to mean a linear or branched saturated hydrocarbon chain wherein the longest chain has from one to six carbon atoms, such as methyl, ethyl, n-propyl, isopropyl, n-butyl, isobutyl, sec-butyl, tert-butyl, pentyl, isopentyl, neopentyl, and hexyl. An alkyl chain may be optionally substituted.

“Lower alkyl groups” are C₁₋₆ cyclic, straight-chained or branched aliphatic substituent groups connected via a carbon atom. Examples include methyl, ethyl, propyl, butyl, methylbutyl, cyclopropyl, cyclohexyl, iso-propyl, tert-butyl.

In the present context, the term “C₂₋₈-alkenyl” is intended to mean a linear or branched hydrocarbon group having from two to eight carbon atoms and containing one or more double bonds. Some examples of C₂₋₈-alkenyl groups include allyl, homo-allyl, vinyl, crotyl, butenyl, pentenyl, hexenyl, heptenyl and octenyl. Some examples of C₂₋₈-alkenyl groups with more than one double bond include butadienyl, pentadienyl, hexadienyl, heptadienyl, heptatrienyl and octatrienyl groups as well as branched forms of these. The position of the unsaturation (the double bond) may be at any position along the carbon chain.

In the present context the term “C₂₋₈-alkynyl” is intended to mean a linear or branched hydrocarbon group containing from two to eight carbon atoms and containing one or more triple bonds. Some examples of C₂₋₈-alkynyl groups include ethynyl, propynyl, butynyl, pentynyl, hexynyl, heptynyl and octynyl groups as well as branched forms of these. The position of unsaturation (the triple bond) may be at any position along the carbon chain. More than one bond may be unsaturated such that the “C₂₋₈-alkynyl” is a di-yne or enedi-yne as is known to the person skilled in the art.

In the present context, the term “C₃₋₈-cycloalkyl” is intended to cover three-, four-, five-, six-, seven-, and eight-membered rings comprising carbon atoms only. A C₃₋₈-cycloalkyl may optionally contain one or more unsaturated bonds situated in such a way, however, that an aromatic π-electron system does not arise.

Some examples of preferred “C₃₋₈-cycloalkyl” are the carbocycles cyclopropane, cyclobutane, cyclopentane, cyclopentene, cyclopentadiene, cyclohexane, cyclohexene, 1,3-cyclohexadiene, 1,4-cyclohexadiene, cycloheptane, cycloheptene.

“Cyclic organyl groups” are aliphatic, alicyclic groups in which carbon atoms form a ring. In preferred embodiments containing three, four, five, six or seven carbon atoms, the ring, as a substituent, is connected either directly via one of the ring atoms or via one or more appended carbon atoms. Particular examples of such groups include cyclopropyl, cyclobutyl, cyclopentyl, cyclohexyl, cycloheptyl, cyclopropylmethyl, cyclobutylmethyl, cyclopentylmethyl, cyclohexylmethyl, cyclohexylethyl groups, and the like.

“Straight-chained acyclic organyl groups” are substituent groups consisting of a linear arrangement of carbon atoms, where accordingly each carbon atom binds a maximum of two other carbon atoms, connected through single, double, or triple bonds. The straight-chained organyl groups may contain none, one, or several multiple bonds, and are, for example, commonly referred to as alkyl, alkenyl or alkynyl, or alkadienyl groups, respectively. Examples of straight-chained organyl groups include methyl, ethyl, propyl, butyl, pentyl, hexyl, propenyl, butenyl, pentadienyl, propargyl, butynyl.

“Branched acyclic organyl groups” are substituent groups consisting of a branched arrangement of carbon atoms, where accordingly one or more carbon atoms may bind more than two other carbon atoms, connected through single, double, or triple bonds. The branched organyl groups may contain none, one, or several multiple bonds. Examples of branched organyl groups include iso-propyl, iso-butyl, tert-butyl, methylbutyl, methylbutenyl, methylbutynyl.

The term “C₁₋₆ alkoxy” and “lower alkoxy groups” are understood as C₁₋₆ cyclic or acyclic organyl groups connected, as substituents, via an oxygen atom. Examples of lower alkoxy groups include methoxy, ethoxy, iso-propoxy, butoxy, tert-butoxy, cyclopropyl, cyclobutyl, cyclopropylmethyl, and cyclobutylmethyl.

The term “C₁₋₆ alkylamino” and “lower alkylamino groups” are understood as lower alkyl groups connected, as substituents, via a nitrogen atom, which may carry one or two lower alkyl groups. Particular examples include methylamino, dimethylamino, iso-propylamino. Optionally, lower aminoalkyl groups may consist of 4-6 membered nitrogen-containing rings, such as pyrrolidino.

The term “C₁₋₆ aminoalkyl” and “lower aminoalkyl groups” are understood as lower alkyl groups carrying, as a substituent, an additional amino group. Examples include aminomethyl and aminoethyl.

The term “C₁₋₆-hydroxyalkyl” and “lower hydroxyalkyl groups” are understood as lower alkyl groups carrying, as a substituent, an additional hydroxy group. Examples include hydroxymethyl, hydroxyethyl, 2-hydroxy-2-propyl, hydroxypentyl.

The term “heterocyclyl” is intended to mean three-, four-, five-, six-, seven-, and eight-membered rings wherein carbon atoms together with from 1 to 3 heteroatoms constitute the ring. A heterocyclyl may optionally contain one or more unsaturated bonds situated in such a way, however, that an aromatic π-electron system does not arise. The heteroatoms are independently selected from oxygen, sulfur, and nitrogen.

A heterocyclyl may further contain one or more carbonyl or thiocarbonyl functionalities, so as to make the definition include oxo-systems and thio-systems such as lactams, lactones, cyclic imides, cyclic thioimides, cyclic carbamates, and the like.

Heterocyclyl rings may optionally also be fused to aryl rings, such that the definition includes bicyclic structures. Preferred such fused heterocyclyl groups share one bond with an optionally substituted benzene ring. Examples of benzo-fused heterocyclyl groups include, but are not limited to, benzimidazolidinone, tetrahydroquinoline, and methylenedioxybenzene ring structures.

Some examples of “heterocyclyls” include, but are not limited to, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Binding to the heterocycle may be at the position of a heteroatom or via a carbon atom of the heterocycle, or, for benzo-fused derivatives, via a carbon of the benzenoid ring.

The term “(heterocyclyl)C₁₋₆-alkyl” is understood as heterocyclyl groups connected, as substituents, via a lower alkylene, each as defined herein. The heterocyclyl groups of (heterocyclyl)C₁₋₆-alkyl groups may be substituted or unsubstituted.

The terms “(aryl)C₁₋₆-alkyl” and “aralkyl” are intended to mean an aryl group connected, as a substituent, via a lower alkylene, each as defined herein. The aryl groups of (aryl)C₁₋₆-alkyl may be substituted or unsubstituted. Examples include benzyl, substituted benzyl, 2-phenylethyl, 3-phenylpropyl, and naphthylalkyl.

The terms “(heteroaryl)C₁₋₆-alkyl” and “heteroaralkyl” are understood as heteroaryl groups connected, as substituents, via a lower alkylene, each as defined herein. The heteroaryl groups of heteroaralkyl groups may be substituted or unsubstituted. Examples include 2-thienylmethyl, 3-thienylmethyl, furylmethyl, thienylethyl, pyrrolylalkyl, pyridylalkyl, isoxazolylalkyl, imidazolylalkyl, and their substituted as well as benzo-fused analogs.

The terms “(cycloalkyl)C₁₋₆-alkyl” is intended to mean a cycloalkyl groups connected, as substituents, via a lower alkylene, each as defined herein.

When used herein, the term “O—C₁₋₆-alkyl” is intended to mean C₁₋₆-alkyloxy, or alkoxy, such as methoxy, ethoxy, n-propoxy, isopropoxy, n-butoxy, isobutoxy, sec-butoxy, tert-butoxy, pentyloxy, isopentyloxy, neopentyloxy and hexyloxy. Further, the definition of “O—C₁₋₆-alkyl” is intended to cover cyclic alkoxy groups having a maximum of six carbon atoms. Illustrative non-limiting examples of cyclic alkoxy groups include cyclobutyloxy, cyclopropylmethyloxy, cyclohexyloxy, and the like.

In the present context the term “lower alkylene” means a bivalent hydrocarbon tether, containing from one to six carbon atoms. Additionally, “lower alkylene” tethers may optionally contain one or more substituents selected from C₁₋₆ alkyl, halogen, hydroxyl, and amino. Non-limiting examples of “lower alkylene” groups are methylene, ethylene, propylene, tetramethylene, hexamethylene.

“Vinylene groups” are ethene-1,2-diyl groups (—CHCH—) having (E) or (Z) configuration.

“Acyl groups” are hydrogen or lower alkyl groups connected, as substituents, via a carbonyl group. Examples include formyl, acetyl, propanoyl.

The terms “haloalkyl”, “hydroxyalkyl” and “aminoalkyl” are intended to cover C₁₋₆-alkyl groups, defined above, carrying at least one halogen, hydroxy group, or amino group, respectively.

The term “halogen” includes fluorine, chlorine, bromine and iodine.

In the present context, i.e. in connection with the terms “C₁₋₆-alkyl”, “aryl”, “heteroaryl”, “heterocyclyl”, “C₃₋₈-cycloalkyl”, “(aryl)C₁₋₁₆-alkyl”, “(heteroaryl)C₁₋₆-alkyl”, “(heterocyclyl)C₁₋₆-alkyl”, “(cycloalkyl)C₁₋₆alkyl”, “O—C₁₋₆-alkyl”, “C₂₋₈-alkenyl”, and “C₂₋₈-alkynyl”, the term “optionally substituted” is intended to mean that the group in question may be substituted one or several times, such as 1 to 5 times, or 1 to 3 times, or 1 to 2 times, with one or more groups selected from C₁₋₆-alkyl, C₁₋₆-alkoxy, oxo (which may be represented in the tautomeric enol form), carboxyl, amino, hydroxy (which when present in an enol system may be represented in the tautomeric keto form), nitro, alkylsulfonyl, alkylsulfenyl, alkylsulfinyl, C₁₋₆-alkoxycarbonyl, C₁₋₆-alkylcarbonyl, formyl, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, C₁₋₆-alkylhydroxyimino, cyano, guanidino, carbamido, C₁₋₆-alkanoyloxy, C₁₋₆-alkylsulphonyloxy, dihalogen-C₁₋₆-alkyl, trihalogen-C₁₋₆-alkyl, heterocyclyl, heteroaryl, and halo. In general, the above substituents may be susceptible to further optional substitution.

The term “salts” is intended to mean pharmaceutically acceptable acid addition salts obtainable by treating the base form of a functional group, such as an amine, with appropriate acids such as inorganic acids, for example hydrohalic acids; typically hydrochloric, hydrobromic, hydrofluoric, or hydroiodic acid; sulfuric acid; nitric acid; phosphoric acid and the like; or organic acids, for example acetic, propionic, hydroacetic, 2-hydroxypropanoic acid, 2-oxopropanoic acid, ethandioic, propanedioic, butanedioic, (Z)-2-butenedioic, (E)-butenedioic, 2-hydroxybutanedioic, 2,3-dihydroxybutanedioic, 2-hydroxy-1,2,3-propanetricarboxylic, methanesulfonic, ethanesulfonic, benzenesulfonic, 4-methylbenzenesulfonic acid, cyclohexanesulfamic, 2-hydroxybenzoic, 4-amino-2-hydroxybenzoic, and other acids known to the skilled practitioner.

The present invention includes within its scope prodrugs of the compounds of this invention. In general, such prodrugs are inactive derivatives of the compounds of this invention that are readily convertible in vivo into the required compound. Conventional procedures for the selection and preparation of suitable prodrug derivatives are described, for example, in Design of Prodrugs, (ed. H. Bundgaard, Elsevier, 1985). Metabolites of these compounds include active species that are produced upon introduction of compounds of this invention into the biological milieu.

Where the compounds according to the invention have at least one chiral center, they may exist as a racemate or as enantiomers. It should be noted that all such isomers and mixtures thereof are included in the scope of the present invention. Furthermore, some of the crystalline forms for compounds of the present invention may exist as polymorphs and as such are intended to be included in the present invention. In addition, some of the compounds of the present invention may form solvates with water (i.e., hydrates) or common organic solvents. Such solvates are also included in the scope of this invention.

Where the processes for the preparation of the compounds according to the invention give rise to mixtures of stereoisomers, such isomers may be separated by conventional techniques such as preparative chiral chromatography. The compounds may be prepared in racemic form or individual enantiomers may be prepared by stereoselective synthesis or by resolution. The compounds may be resolved into their component enantiomers by standard techniques, such as the formation of diastereomeric pairs by salt formation with an optically active acid, such as (−)-di-p-toluoyl-d-tartaric acid and/or (+)-di-p-toluoyl-1-tartaric acid, followed by fractional crystallization and regeneration of the free base. The compounds may also be resolved using a chiral auxiliary by formation of diastereomeric derivatives such as esters, amides or ketals followed by chromatographic separation and removal of the chiral auxiliary.

Compounds

One embodiment includes the use of N-(1-methylpiperidin-4-yl)-N-(4-fluorophenylmethyl)-N′-(4-(2-methylpropyloxy)phenylmethyl)carbamide, a 5-HT2A inverse agonist which has the structure:

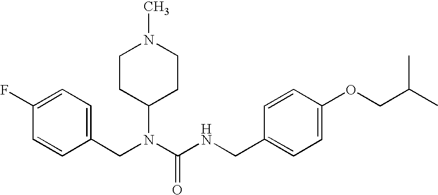

Other compounds that are selective serotonin inverse agonists or antagonists are described in U.S. Pat. Nos. 6,756,393 and 6,815,458 entitled “AZACYCLIC COMPOUNDS”, filed Apr. 7, 2003 and Mar. 6, 2001 respectively; U.S. Pat. No. 6,911,452 entitled “SPIROAZACYCLIC COMPOUNDS AS MONOAMINE RECEPTOR MODULATORS,” filed Dec. 23, 2002; U.S. patent application Ser. Nos. 10/802,970, filed Mar. 16, 2004, 11/417,790, filed May 3, 2006, and 11/417,782, filed May 3, 2006, each entitled “AZACYCLIC COMPOUNDS”; U.S. patent application Ser. Nos. 10/601,070, filed Jun. 20, 2003 and 11/299,566, filed Dec. 12, 2005, 11/417,866, filed May 3, 2006, and 11/418,353, filed May 3, 2006, each entitled “N-SUBSTITUTED PIPERDINE DERIVATIVES AS SEROTONIN RECEPTOR AGENTS; U.S. patent application Ser. Nos. 11/154,08, filed Jun. 16, 2005, 11/418,322, filed May 3, 2006, and 11/417,439, filed May 3, 2006, each entitled “SPIROAZACYCLIC COMPOUNDS AS MONOAMINE RECEPTOR MODULATORS,”; U.S. patent application Ser. Nos. 10/759,561 filed Jan. 15, 2004, 11/416,527, filed May 3, 2006, and 11/416,855, filed May 3, 2006, each entitled “SELECTIVE SEROTONIN 2A/2C RECEPTOR INVERSE AGONISTS AS THERAPEUTICS FOR NEURODEGENERATIVE DISEASES”; and U.S. patent application Ser. Nos. 11/235,558, filed Sep. 26, 2005, 11/235,381, filed Sep. 26, 2005, 11/418,341, filed May 3, 2006 and 11/417,447, filed May 3, 2006, each entitled “SYNTHESIS OF N-(4-FLUOROBENZYL)-N-(1-METHYLPIPERIDIN-4-YL)-N′-(4-(2-METHYLPROPYLOXY)PHENYLMETHYL)CARBAMIDE AND ITS TARTRATE SALT AND CRYSTALLINE FORMS”; U.S. Patent Application No. 60/187,289 entitled “AZACYCLIC COMPOUNDS” filed Mar. 6, 2000; U.S. Patent Application No. 60/344,750 entitled “SPIROAZCYCLIC COMPOUNDS AS MONOAMINE RECEPTOR MODULATORS” filed Dec. 28, 2001; U.S. Patent Application No. 60/391,269 entitled “N-[HETEROCYCLYLALKY]PIPERDINE DERIVATIVES AS SEROTONIN RECEPTOR AGENTS” filed Jun. 24, 2002; U.S. Patent Application No. 60/441,406 entitled “SELECTIVE SEROTONIN 2A/2C RECEPTOR INVERSE AGONISTS AS THERAPEUTICS FOR NEURODEGENERATIVE DISEASES” filed Jan. 16, 2003; U.S. Patent Application No. 60/614,014 entitled “SYNTHESIS OF 1-(4-FLUOROBENZYL)-3-(4-ISOBUTOXYBENZYL)-1-(1-METHYLPIPERIDIN-4-YL)UREA, ITS SALTS, AND POLYMORPHS” filed Sep. 27, 2004; the disclosures of which are hereby incorporated by reference in their entirety.

One embodiment includes the use of compounds of the general Formula (I): 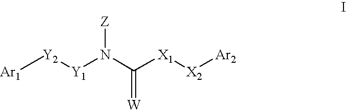
wherein Z is

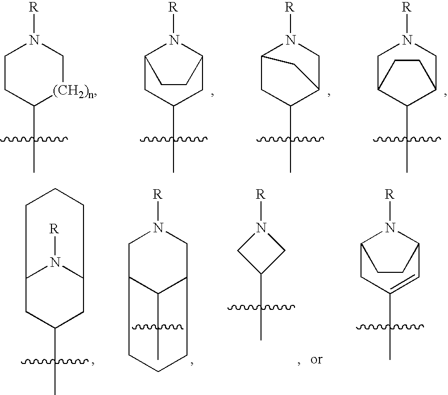

R is a hydrogen, a cyclic or straight-chained or branched acyclic organyl group, a lower hydroxyalkyl group, a lower aminoalkyl group, or an aralkyl or heteroaralkyl group; and n can be 0, 1, or 2.

X₁ is methylene, vinylene, or an NH or N(lower alkyl) group; and X₂ is methylene, or, when X₁ is methylene or vinylene, X₂ is methylene or a bond; or when X₁ is methylene, X₂ is O, S, NH, or N(lower alkyl) or a bond.

Y₁ is methylene and Y₂ is methylene, vinylene, ethylene, propylene, or a bond; or Y₁ is a bond and Y₂ is vinylene; or Y₁ is ethylene and Y₂ is O, S, NH, or N(lower alkyl).

Ar₁ and Ar₂ each independently is unsubstituted or substituted aryl or heteroaryl groups.

W is oxygen or sulfur.

In some embodiments of the compounds of Formula (I), Y₁ is methylene and Y₂ is a bond, methylene, ethylene, or vinylene; or Y₁ is ethylene and Y₂ is O or S; and X₁ is methylene and X₂ is a bond, methylene, O, or S; or X₁ is NH or N(lower alkyl) and X₂ is methylene.

In another embodiment, Z is 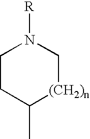
and W is oxygen.

In certain embodiments, Ar₁ and Ar₂ independently are mono- or disubstituted phenyl groups.

In other embodiments, the R of Formula (I) is a hydrogen, a lower alkyl group, a cyclic organyl group, or a substituted or unsubstituted aralkyl or heteroaralkyl group; n is 1; Y₁ is methylene, and Y₂ is a bond, methylene, ethylene, or vinylene; X₁ is methylene and X₂ is a bond, or X₁ is NH or N(lower alkyl) and X₂ is methylene; and Ar₁ and Ar₂ are phenyl groups, independently p-substituted with groups selected from lower alkyl, lower alkoxy and halogen.

In another embodiment, compounds of Formula (Ia) are used:

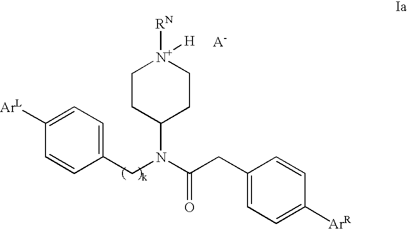

wherein R^{N} is hydrogen, lower alkyl, aralkyl, or heteroaralkyl; Ar^{L} is selected from lower alkyl, lower alkoxy and halogen; Ar^{R} is selected from lower alkyl, lower alkoxy and halogen; k is 1 or 2; and A⁻ is a suitable anion.

It will be understood that “Formula I” as used herein refers to compounds of Formulae (I) and (Ia).

Suitable embodiments of the compounds of Formula I may be selected from the group consisting of:
N-((4-methylphenyl)methyl)-N-(piperidin-4-yl)-N′-phenylmethylcarbamide;
N-((4-methylphenyl)methyl)-N-(1-(2-methylpropyl)piperidin-4-yl)-N′-phenylmethylcarbamide;
N-(1-(cyclohexylmethyl)piperidin-4-yl)-N-((4-methylphenyl)methyl)-N′-phenylmethylcarbamide;
N-((4-methylphenyl)methyl)-N-(piperidin-4-yl)-4-methoxyphenylacetamide;
N-(1-(3,3-dimethylbutyl)piperidin-4-yl)-N-((4-methylphenyl)methyl)-4-methoxyphenylacetamide;
N-(1-(cyclohexylmethyl)piperidin-4-yl)-N-((4-methylphenyl)methyl)-4-methoxyphenylacetamide;
N-((4-methylphenyl)methyl)-N-(1-(2-methylpropyl)piperidin-4-yl)-4-methoxyphenylacetamide;
N-(3-phenylpropyl)-N-(piperidin-4-yl)-4-methoxyphenylacetamide;
N-(2-phenylethyl)-N-(piperidin-4-yl)-4-methoxyphenylacetamide;
N-((2-methoxyphenyl)methyl)-N-(piperidin-4-yl)-4-methoxyphenylacetamide;
N-((2-chlorophenyl)methyl)-N-(piperidin-4-yl)-4-methoxyphenylacetamide;
N-((3,4-di-methoxyphenyl)methyl)-N-(piperidin-4-yl)-4-methoxyphenylacetamide;
N-((4-fluorophenyl)methyl)-N-(piperidin-4-yl)-4-methoxyphenylacetamide;
N-((2,4-di-chlorophenyl)methyl)-N-(piperidin-4-yl)-4-methoxyphenylacetamide;
N-((3-methylphenyl)methyl)-N-(piperidin-4-yl)-4-methoxyphenylacetamide;
N-((3-bromophenyl)methyl)-N-(piperidin-4-yl)-4-methoxyphenylacetamide;
N-((4-methylphenyl)methyl)-N-(1-piperidin-4-yl)-phenylacetamide;
N-((4-methylphenyl)methyl)-N-(1-piperidin-4-yl)-3-phenylpropionamide;
N-((4-methylphenyl)methyl)-N-(1-piperidin-4-yl)-(phenylthio)acetamide;
N-((4-methylphenyl)methyl)-N-(1-piperidin-4-yl)-phenoxyacetamide;
N-((4-methylphenyl)methyl)-N-(1-piperidin-4-yl)-(4-chlorophenoxy)acetamide;
N-((4-methylphenyl)methyl)-N-(1-piperidin-4-yl)-3-methoxyphenylacetamide;
N-((4-methylphenyl)methyl)-N-(1-piperidin-4-yl)-4-fluorophenylacetamide;
N-((4-methylphenyl)methyl)-N-(1-piperidin-4-yl)-2,5-di-methoxyphenylacetamide;
N-((4-methylphenyl)methyl)-N-(1-piperidin-4-yl)-4-chlorophenylacetamide;
2-(4-methoxyphenyl)-N-(4-methylbenzyl)-N-(piperidin-4-yl)acetamide;
2-(4-methoxyphenyl)-N-(4-methylbenzyl)-N-(1-methylpiperidin-4-yl)acetamide;
2-(4-methoxyphenyl)-N-(4-methylbenzyl)-N-(1-ethylpiperidin-4-yl)acetamide;
2-(4-methoxyphenyl)-N-(4-chlorbenzyl)-N-(1-ethylpiperidin-4-yl)acetamide.
2-(4-methoxyphenyl)-N-(4-methylbenzyl)-N-(1-isopropylpiperidin-4-yl)acetamide;
2-(4-methoxyphenyl)-N-(4-chlorobenzyl)-N-(piperidin-4-yl)acetamide;
2-(4-methoxyphenyl)-N-(4-chlorbenzyl)-N-(1-cyclopentylpiperidin-4-yl)acetamide;
2-(4-methoxyphenyl)-N-(4-chlorbenzyl)-N-(1-isopropylpiperidin-4-yl)acetamide;
2-(phenyl)-N-(4-trifluoromethylbenzyl)-N-(1-methylpiperidin-4-yl)acetamide;
2-(4-fluorophenyl)-N-(4-trifluoromethylbenzyl)-N-(1-methylpiperidin-4-yl)acetamide;
2-(4-Methoxyphenyl)-N-(4-trifluoromethylbenzyl)-N-(1-methylpiperidin-4-yl)acetamide;
2-(4-Trifluoromethylphenyl)-N-(4-trifluoromethylbenzyl)-N-(1-methylpiperidin-4-yl)acetamide;
2-(4-Fluorophenyl)-N-(4-fluorobenzyl)-N-(1-methylpiperidin-4-yl)acetamide;
2-(4-Methoxyphenyl)-N-(4-fluorobenzyl)-N-(1-methylpiperidin-4-yl)acetamide;
2-(phenyl)-N-(4-fluorobenzyl)-N-(1-methylpiperidin-4-yl)acetamide;
2-(4-Trifluoromethylphenyl)-N-(4-fluorobenzyl)-N-(1-methylpiperidin-4-yl)acetamide;
2-(4-trifluoromethylphenyl)-N-[4-(methoxycarbonyl)benzyl]-N-(1-methylpiperidin-4-yl)acetamide;
2-(4-Methoxyphenyl)-N-[4-(methoxycarbonyl)benzyl]-N-(1-methylpiperidin-4-yl)acetamide;
2-(4-trifluoromethylphenyl)-N-[4-(methoxycarbonyl)benzyl]-N-(1-methylpiperidin-4-yl)acetamide;
2-Phenyl-N-[4-(methoxycarbonyl)benzyl]-N-(1-methylpiperidin-4-yl)acetamide;
2-(4-Chlorophenyl)-N-[4-(methoxycarbonyl)benzyl]-N-(1-methylpiperidin-4-yl)acetamide;
2-(4-Methoxyphenyl)-N-[4-(methoxycarbonyl)benzyl]-N-(1-methylpiperidin-4-yl)acetamide;
2-(4-methoxyphenyl)-N-(2-4(fluorophenyl)ethyl)-N-(1-methylpiperidin-4-yl)acetamide;
2-(4-methoxyphenyl)-N-[2-(2,5-dimethoxyphenyl)ethyl]-N-(1-methyl piperidin-4-yl)acetamide;
2-(4-methoxyphenyl)-N-[2-(2,4-dichlorophenyl)ethyl]-N-(1-methylpiperidin-4-yl)acetamide;
2-(4-methoxyphenyl)-N-[2-(3-chlorophenyl)ethyl]-N-(1-methylpiperidin-4-yl)acetamide;
2-(4-methoxyphenyl)-N-[2-(4-methoxyphenyl)ethyl]-N-(1-methylpiperidin-4-yl)acetamide;
2-(4-methoxyphenyl)-N-[2-(3-fluorophenyl)ethyl]-N-(1-methylpiperidin-4-yl)acetamide;
2-(4-ethoxyphenyl)-N-[2-(4-fluorophenethyl]-N-(1-methylpiperidin-4-yl)acetamide;
2-(4-ethoxyphenyl)-N-(4-fluorobenzyl)-N-(1-methylpiperidin-4-yl)acetamide;
2-(4-Chlorophenyl)-N-(4-methylbenzyl)-N-(1-isopropylpiperidin-4-yl)-acetamide;
2-(4-Chlorophenyl)-N-(4-methylbenzyl)-N-(1-ethylpiperidin-4-yl)-acetamide;
2-Phenyl-N-(4-methylbenzyl)-N-(1-methylpiperidin-4-yl)-acetamide;
2-(4-Chlorophenyl)-N-(4-methylbenzyl)-N-(1-cyclopentylpiperidin-4-yl)-acetamide;
2-(4-Fluorophenyl)-N-(4-methylbenzyl)-N-(1-methylpiperidin-4-yl)-acetamide;
2-(4-Chlorophenyl)-N-(4-methylbenzyl)-N-(1-(2-hydroxyethyl)-piperidin-4-yl)-acetamide;
2-(4-Chlorophenyl)-N-(4-methylbenzyl)-N-(1-cyclobutylpiperidin-4-yl)-acetamide;
2-(4-Methoxyphenyl)-N-(4-methylbenzyl)-N-(1-cyclobutylpiperidin-4-yl)-acetamide;
N-(4-Methylbenzyl)-N-(1-methylpiperidin-4-yl)-N′-benzyl-carbamide;
2-Phenyl-N-(4-methoxybenzyl)-N-(1-methylpiperidin-4-yl)-acetamide;
2-(4-Trifluoromethylphenyl)-N-(4-methoxybenzyl)-N-(1-methylpiperidin-4-yl)-acetamide;
2-(4-Fluorophenyl)-N-(4-methoxybenzyl)-N-(1-methylpiperidin-4-yl)-acetamide;
2-(4-Methoxyphenyl)-N-(4-methoxybenzyl)-N-(1-methylpiperidin-4-yl)-acetamide;
2-(4-Methylphenyl)-N-(4-chlorobenzyl)-N-(1-methylpiperidin-4-yl)-acetamide;
2-(4-Hydroxyphenyl)-N-(4-methylbenzyl)-N-(1-methylpiperidin-4-yl)-acetamide;
2-(4-Methoxyphenyl)-2,2-ethylene-N-(4-methylbenzyl)-N-(1-methylpiperidin-4-yl)acetamide;
2-(4-Methoxyphenyl)-N-alpha-methylbenzyl-N-(1-methylpiperidin-4-yl)acetamide;
N-Phenethyl-N-(4-methylbenzyl)-N-(1-methylpiperidin-4-yl)-amine;
N-(4-Methylbenzyl)-N-(1-methylpiperidin-4-yl)-N′-(4-methoxybenzyl)-carbamide;
2-(3,4-dimethoxyphenyl)-N-(4-methylbenzyl)-N-(1-methylpiperidin-4-yl)acetamide;
2-(3,4-Methylenedioxyphenyl)-N-(4-methylbenzyl)-N-(1-methylpiperidin-4-yl)acetamide;
2-(4-Methoxyphenyl)-N-(4-methylbenzyl)-N-(1-t-butylpiperidin-4-yl)-acetamide;
N-(4-Methylbenzyl)-N-(1-t-butylpiperidin-4-yl)-N′-(4-methoxybenzyl)-carbamide;
2-(4-Ethoxyphenyl)-N-(4-methylbenzyl)-N-(1-methylpiperidin-4-yl)acetamide;
2-(4-Butoxyphenyl)-N-(4-methylbenzyl)-N-(1-methylpiperidin-4-yl)acetamide;
2-(4-i-Propoxyphenyl)-N-(4-methylbenzyl)-N-(1-methylpiperidin-4-yl)acetamide;
N-(4-Fluorobenzyl)-N-(piperidin-4-yl)-2-(4-isobutoxyphenyl)acetamide;
N-(4-Fluorobenzyl)-N′-(4-isopropoxybenzyl)-N-piperidin-4-yl-carbamide, oxalate;
N-(1-Benzyloxycarbonylpiperidin-4-yl)-N-(4-fluorobenzyl)-N′-(4-isopropoxybenzyl)carbamide;
2-(4-methoxyphenyl)-N-(3-phenyl-1-propyl)-N-(1-methylpiperidin-4-yl)acetamide;
2-(4-methoxyphenyl)-N-[2-(4-methylphenyl)ethyl]-N-(1-methylpiperidin-4-yl)acetamide;
2-(4-methoxyphenyl)-N-[2-(4-nitrophenyl)ethyl]-N-(1-methylpiperidin-4-yl)acetamide;
2-(4-methoxyphenyl)-N-(4-methylbenzyl)-N-(1-cyclopentylpiperidin-4-yl)acetamide;
N-((4-(hydroxymethyl)phenyl)methyl)-N-(1-methylpiperidin-4-yl)-2-(4-methoxyphenyl)acetamide;
N-((4-methylphenyl)methyl)-N-(1-methylpiperidin-4-yl)-2-(3-hydroxyl-4-methoxyphenyl)acetamide;
N-((4-methylphenyl)methyl)-N-(1-methylpiperidin-4-yl)-2-(3,4-dihydroxyphenyl)acetamide;
N-((3-hydroxy-4-methylphenyl)methyl)-N-(1-methylpiperidin-4-yl)-2-(4-methoxyphenyl)acetamide;
N-((4-methylphenyl)methyl)-N-(1-methylpiperidin-4-yl)-2-(4-bromophenyl)acetamide;
N-((4-methylphenyl)methyl)-N-(1-methylpiperidin-4-yl)-2-(4-iodophenyl)acetamide;
N-((4-methylphenyl)methyl)-N-(1-methylpiperidin-4-yl)-2-(4-(2-propyl)phenyl)acetamide;
N-((4-methylphenyl)methyl)-N-(1-methylpiperidin-4-yl)-2-(4-trifluoromethoxyphenyl)acetamide;
N-((4-methylphenyl)methyl)-N-(1-methylpiperidin-4-yl)-2-(4-methylthiophenyl)acetamide;
N-((4-methylphenyl)methyl)-N-(1-methylpiperidin-4-yl)-2-(4-(N,N′-dimethylamino)phenyl)acetamide;
N-((4-methylphenyl)methyl)-N-(1-methylpiperidin-4-yl)-2-(4-nitrophenyl)acetamide
N-((4-methylphenyl)methyl)-N-(1-methylpiperidin-4-yl)-2-(4-methoxy-3-methylphenyl)acetamide;
N-((4-methylphenyl)methyl)-N-(1-methylpiperidin-4-yl)-2-(4-pyridyl)acetamide;
N-((4-methylphenyl)methyl)-N-(1-methylpiperidin-4-yl)-2-(4-methylphenyl)acetamide;
2-(4-methoxyphenyl)-N-(phenylethyl)-N-(1-methylpiperidin-4-yl)acetamide;
N-((4-methylphenyl)methyl)-N-(1-(phenylmethyl)piperidin-4-yl)-N′-phenylmethylcarbamide;
N-((4-methylphenyl)methyl)-N-(1-(phenylmethyl)piperidin-4-yl)-N′-phenylmethylcarbamide;
N-((4-methylphenyl)methyl)-N-(1-(phenylmethyl)piperidin-4-yl)-4-methoxyphenylacetamide;
N-((4-methylphenyl)methyl)-N-(1-(phenylmethyl)piperidin-4-yl)-4-methoxyphenylacetamide;
2-(4-methoxyphenyl)-N-(4-methylbenzyl)-N-(1-cyclohexylmethylpiperidin-4-yl)acetamide;
N-((4-methylphenyl)methyl)-N-(1-(phenylmethyl)piperidin-4-yl)-4-methoxyphenylthioacetamide;
2-(4-Methoxyphenyl)-N-(4-methylbenzyl)-N-[1-(2-methylthiazol-4-ylmethyl)piperidin-4-yl]acetamide;
2-(4-Methoxyphenyl)-N-[2-(2-thienyl)ethyl]-N-(1-methylpiperidin-4-yl)acetamide;
2-(4-Methoxyphenyl)-N-(2-thienylmethyl)-N-(1-methylpiperidin-4-yl)acetamide;
2-(4-Methoxyphenyl)-N-(furfuryl)-N-(1-methylpiperidin-4-yl)acetamide;
2(2-thienyl)-N-(4-methylphenylmethyl)-N-(1-methylpiperidin-4-yl)acetamide;
N-[1-((S)-3,5-Dihydroxypentyl)piperidine-4-yl]-N-(4-fluorobenzyl)-2-(4-isobutoxyphenyl)acetamide, tartrate; and
N-[1-((R)-3,5-Dihydroxypentyl)piperidine-4-yl]-N-(4-fluorobenzyl)-2-(4-isobutoxyphenyl)acetamide, tartrate.

In some embodiments, the compound of Formula I is selected from the group consisting of:
N-(1-((2-bromophenyl)methyl)piperidin-4-yl)-N-((4-methylphenyl)methyl)-N′-phenylmethylcarbamide;
N-(1-((4-hydroxy-3-methoxyphenyl)methyl)piperidin-4-yl)-N-((4-methylphenyl)methyl)-N′-phenylmethylcarbamide;
N-(1-((5-ethylthien-2-yl)methyl)piperidin-4-yl)-N-((4-methylphenyl)methyl)-N′-phenylmethylcarbamide;
N-(1-(imidazol-2-ylmethyl)piperidin-4-yl)-N-((4-methylphenyl)methyl)-N′-phenylmethylcarbamide;
N-(1-((4-fluorophenyl)methyl)piperidin-4-yl)-N-((4-methylphenyl)methyl)-N′-phenylmethylcarbamide;
N-((4-methylphenyl)methyl)-N-(1-(phenylmethyl)piperidin-4-yl)-N′-phenylmethylcarbamide;
N-((4-methylphenyl)methyl)-N-(1-(phenylmethyl)piperidin-4-yl)-4-methoxyphenylacetamide;
N-((4-methylphenyl)methyl)-N-(1-((4-methylphenyl)methyl)piperidin-4-yl)-4-methoxyphenylacetamide;
N-(1-((4-hydroxyphenyl)methyl)piperidin-4-yl)-N-((4-methylphenyl)methyl)-4-methoxyphenylacetamide;
N-(1-((2-hydroxyphenyl)methyl)piperidin-4-yl)-N-((4-methylphenyl)methyl)-4-methoxyphenylacetamide;
N-(1-(phenylmethyl)piperidin-4-yl)-N-(3-phenyl-2-propen-1-yl)-4-methoxyphenylacetamide;
N-((Methylphenyl)methyl)-N-(1-(phenylmethyl)piperidin-4-yl)-4-methoxyphenylthioacetamide; and
2-(4-Methoxyphenyl)-N-(4-methylbenzyl)-N-[1-(2-methylthiazol-4-ylmethyl)piperidin-4-yl]acetamide.

In some embodiments, the compound of Formula I is selected from the group consisting of:
N-((4-methylphenyl)methyl)-N-(1-(phenylmethyl)pyrrolidin-3-yl)-N′-phenylmethylcarbamide; and
N-((4-methylphenyl)methyl)-N-(1-(phenylmethyl)pyrrolidin-3-yl)-4-methoxyphenylacetamide.

In one embodiment, the compound of Formula I is 2-(4-methoxyphenyl)-N-(4-methylbenzyl)-N-(8-methyl-8-aza-bicyclo[3.2.1]oct-3-yl)-acetamide.

In some embodiments, the compound of Formula I is selected from the group consisting of:
N-(1-(1-methylethyl)piperidin-4-yl)-N-((4-methylphenyl)methyl)-4-methoxyphenylacetamide;
N-(1-(2,2-dimethylethyl)piperidin-4-yl)-N-((4-methylphenyl)methyl)-4-methoxyphenylacetamide;
N-(1-pentylpiperidin-4-yl)-N-((4-methylphenyl)methyl)-4-methoxyphenylacetamide;
N-(1-hexylpiperidin-4-yl)-N-((4-methylphenyl)methyl)-4-methoxyphenylacetamide;
N-(1-cyclohexylpiperidin-4-yl)-N-((4-methylphenyl)methyl)-4-methoxyphenylacetamide;
N-(1-cyclopentylpiperidin-4-yl)-N-((4-methylphenyl)methyl)-4-methoxyphenylacetamide;
N-(1-cyclobutylpiperidin-4-yl)-N-((4-methylphenyl)methyl)-4-methoxyphenylacetamide;
N-(cyclopropylpiperidin-4-yl)-N-((4-methylphenyl)methyl)-4-methoxyphenylacetamide;
N-(1-(cyclopentylmethyl)piperidin-4-yl)-N-((4-methylphenyl)methyl)-4-methoxyphenylacetamide;
N-(1-(cyclobutylmethyl)piperidin-4-yl)-N-((4-methylphenyl)methyl)-4-methoxyphenylacetamide;
N-(1-(cyclopropylmethyl)piperidin-4-yl)-N-((4-methylphenyl)methyl)-4-methoxyphenylacetamide;
N-(1-(2-hydroxyethyl)piperidin-4-yl)-N-((4-methylphenyl)methyl)-4-methoxyphenylacetamide;
N-(1-(3-hydroxyethyl)piperidin-4-yl)-N-((4-methylphenyl)methyl)-4-methoxyphenylacetamide;
N-((4-methylphenyl)methyl)-N-(1-methylipieridin-4-yl)-4-methoxyphenylacetamide;
N-(1-ethylpiperidin-4yl)-N-((4-methylphenyl)methyl)-4-methoxyphenylacetamide;
N-((4-methylphenyl)methyl)-N-(1-propylpiperidin-4-yl)-4-methoxyphenylacetamide;
N-(1-butylpiperidin-4-yl)-N-((4-methylphenyl)methyl)-4-methoxyphenylacetamide;
2-Phenyl-N-[4-(methoxycarbonyl)benzyl]-N-(1-methylpiperidin-4-yl)acetamide;
2-(4-Chlorophenyl)-N-[4-(methoxycarbonyl)benzyl]-N-(1-methylpiperidin-4-yl)acetamide;
2-(4-methoxyphenyl)-N-(4-methylbenzyl)-N-[1-(4-chloromethyl-2-thiazolylmethyl)piperidin-4-yl]acetamide;
2-(4-methoxyphenyl)-N-(4-methylbenzyl)-N-{1-[2-(2-hydroxyethoxy)ethyl]piperidin-4-yl}acetamide;
2-(4-methoxyphenyl)-N-(4-methylbenzyl)-N-[1-((2-chloro-5-thienyl)methyl)piperidin-4-yl]acetamide;
2-(4-methoxyphenyl)-N-(4-methylbenzyl)-N-{1-[2-(3-indolyl)ethyl]piperidin-4-yl}acetamide;
2-(4-methoxyphenyl)-N-(4-methylbenzyl)-N-{1-[3-(1,2,4-triazol-1-yl)propyl]piperidin-4-yl}acetamide;
2-(4-methoxyphenyl)-N-(4-methylbenzyl)-N-[1-(5-benzofurazanyl methyl)piperidin-4-yl]acetamide;
2-(4-methoxyphenyl)-N-(4-methylbenzyl)-N-[1-(5-chlorobenzo[b]thien-3-ylmethyl)piperidin-4-yl]acetamide;
2-(4-methoxyphenyl)-N-(4-methylbenzyl)-N-[1-(5-phenyl-1,2,4-oxadiazol-3-ylmethyl)piperidin-4-yl]acetamide;
2-(4-t-Butoxyphenyl)-N-(4-methylbenzyl)-N-(1-methylpiperidin-4-yl)acetamide;
2-(4-Butoxyphenyl)-N-(4-fluorobenzyl)-N-(1-methylpiperidin-4-yl)acetamide;
2-(4-Propoxyphenyl)-N-(4-fluorobenzyl)-N-(1-methylpiperidin-4-yl)acetamide;
2-(4-i-Propoxyphenyl)-N-(4-fluorobenzyl)-N-(1-methylpiperidin-4-yl)acetamide; and
2-(4-t-Butoxyphenyl)-N-(4-fluorobenzyl)-N-(1-methylpiperidin-4-yl)acetamide.

In some embodiments, the compound of Formula I is selected from the group consisting of:
N-(4-Methylbenzyl)-N-(1-methylpiperidin-4-yl)-N′-phenyl-carbamide;
N-Phenethyl-N-(1-methylpiperidin-4-yl)-N′-benzyl-carbamide;
N-Phenethyl-N-(1-methylpiperidin-4-yl)-N′-phenyl-carbamide;
N-(3-Phenylpropyl)-N-(1-methylpiperidin-4-yl)-N′-benzyl-carbamide;
N-(3-Phenylpropyl)-N-(1-methylpiperidin-4-yl)-N′-phenyl-carbamide;
2-Phenyl-2-ethyl-N-(4-methylbenzyl)-N-(1-methylpiperidin-4-yl)acetamide;
N-(4-Methylbenzyl)-N-(1-methylpiperidin-4-yl)-N′-phenethyl-carbamide; and
N-Phenethyl-N-(1-methylpiperidin-4-yl)-N′-phenethyl-carbamide.

In one embodiment, the compound of Formula I is 2-(4-Methoxyphenyl)-N-(4-methylbenzyl)-N-(8-methyl-8-aza-bicyclo[3.2.1]octen-3-yl)-acetamide.

Other embodiments includes use of compounds of the general Formula (II): 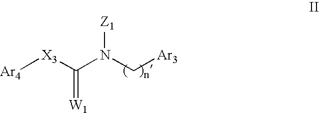
or a pharmaceutically acceptable salt, amide, ester, or prodrug thereof, where:

Z₁ is

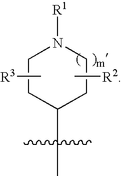

R¹ is selected from the group consisting of optionally substituted heterocyclyl, and optionally substituted (heterocyclyl)C₁₋₆-alkyl.

R² and R³ are independently selected from the group consisting of hydrogen, C₁₋₆-alkyl and halogen or such that R² together with R³ forms a ring. For example, R² and R³ together can form a 3-, 4-, 5-, 6-, or 7-membered ring system with the atoms of the piperidine ring.

m′ is selected from the group consisting of 0, 1, and 2.

n′ is selected from the group consisting of 1, 2, and 3;

Ar₃ is an optionally substituted aryl or heteroaryl. For example, the aryl or heteroaryl can be optionally substituted with a substituent selected from the group consisting of C₁₋₆-alkyl, C₁₋₆-alkoxy, carboxyl, amino, hydroxy, thiol, nitro, cyano, guanidino, carbamido and halogen.

W₁ is selected from the group consisting of O and S.

X₃ is selected from the group consisting of optionally substituted methylene, optionally substituted ethylene, optionally substituted propylene, optionally substituted vinylene, and CH₂N(R^{N1}), wherein R^{N1} is selected from hydrogen and C₁₋₆-alkyl.

Ar₄ is an optionally substituted aryl or heteroaryl.

While not being bound by any particular theory, the presence of a heterocyclic substituent at the nitrogen of the piperidine ring of these compounds is believed to improve the bioavailability of the compounds in comparison to related compounds.

In some embodiments, the heterocyclyl or (heterocyclyl)C₁₋₆-alkyl of R¹ may be optionally substituted. The substituent may be selected from halogen, hydroxy, alkyl, alkoxy, and amino. In some embodiments, the substituent may be on the alkyl chain or the ring system. In further embodiments the substituent is on the ring system.

In certain embodiments, the heterocyclyl ring in R¹ may be selected from the group consisting of tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. In some embodiments, the heterocyclyl ring is selected from 1,3-dioxane, 1,3-dioxolane, and tetrahydropyran.

The azacyclic ring may be a 5, 6, or 7-membered ring as reflected in that m′ may be selected from 0, 1 and 2. In certain embodiments, however, the azacyclic ring is a 6-membered ring, wherein m′ is 1.

The azacyclic ring, further to being substituted at the nitrogen position, may be substituted with R² and R³. R² and R³ may be independently selected from the group consisting of hydrogen, C₁₋₆-alkyl, and halogen, or such that R² together with R³ forms a ring. That is to say that R² and R³ may be biradicals which combine to form a 3-, 4-, 5-, 6-, or 7-membered ring system with the atoms of the azacyclic ring.

In some embodiments, the azacyclic ring system is selected from 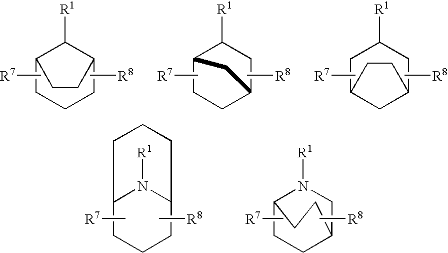
wherein R⁷ and R⁸ are independently selected from the group consisting of hydrogen, halogen, hydroxyl, and C₁₋₆ alkyl. In certain embodiments R⁷ and R⁸ are hydrogen.

In other embodiments, R² and R³ are hydrogen.

In some embodiments, R¹ is an optionally substituted (heterocyclyl)C₁₋₆-alkyl. In certain of these embodiments, R¹ is an optionally substituted (heterocyclyl)methyl, an optionally substituted (heterocyclyl)ethyl, or an optionally substituted (heterocyclyl)propyl. In other embodiments, R¹ is an optionally substituted (heterocyclyl)ethyl.

Ar₃ is linked to a central nitrogen atom via a short aliphatic chain 1, 2, or 3 carbon atoms in length. In certain embodiments, n′ is 1, resulting in a methylene spacer between the central nitrogen atom and Ar₃. Ar₃ may be an optionally substituted aryl or heteroaryl. In some embodiments, Ar₃ is an optionally substituted aryl. In some embodiments, the central nitrogen atom is linked to an optionally substituted benzyl group.

In certain embodiments Ar₃ is an optionally substituted aryl, which may be a 4-substituted aryl. The 4-substituent of the 4-substituted aryl may be any substituent known to the person skilled in the art, such as a C₁₋₆-alkyl, C₁₋₆-alkoxy, carboxyl, amino, hydroxy, thiol, nitro, cyano, guanidino, carbamido and halogen. In some embodiments, the halogen is fluoro, while in other embodiments, the halogen is chloro.

In other embodiments, Ar₃ is selected from the group consisting of alkyl-substituted phenyl, alkoxy-substituted phenyl, halogen-substituted phenyl, hydroxy-substituted phenyl and amino-substituted phenyl. In some embodiments, the substituent may be present 0 to 5 times, or 0 to 4 times, or 0 to 3 times, such as 0, 1, 2, or 3 times. In certain embodiments, the substituent is present 1 to 2 times. In some embodiments, Ar₃ is a 4-substituted aryl selected from the group consisting of 4-halophenyl and 4-alkylphenyl. In some embodiments, the phenyl group is 4-fluorophenyl.

In other embodiments, Ar₃ is an optionally substituted heteroaryl. The heteroaryl may be substituted with substituents known to the person skilled in the art, such as a C₁₋₆-alkyl, C₁₋₆-alkoxy, carboxyl, amino, hydroxy, thiol, nitro, cyano, guanidino, carbamido and halogen.

Further to being linked to both the azacyclic ring and to Ar₃ via a short aliphatic chain, the central nitrogen is linked to Ar₄ via a 2 to 4 carbon spacer unit. This spacer unit comprises a carbonyl or thiocarbonyl function wherein W₁ is selected from the group consisting of oxygen and sulfur. In some embodiments W₁ is oxygen.

In certain embodiments, X₃ may be selected from the group consisting of optionally substituted methylene, optionally substituted ethylene, optionally substituted propylene, optionally substituted vinylene, and CH₂N(R^{N1}). Thus X₃ may extend the spacer unit by 1 to 3 atoms between the central nitrogen and Ar₄ and render the central nitrogen part of an amide or carbamide. In some embodiments, X₃ is selected from the group consisting of optionally substituted methylene, optionally substituted ethylene, and CH₂N(R^{N1}). In some embodiments, X₃ is an optionally substituted methylene, or CH₂N(R^{N1}), wherein R^{N1} may be hydrogen.

In certain embodiments, Ar₄ may be an optionally substituted aryl or heteroaryl. In certain embodiments, Ar₄ is an optionally substituted aryl. In some embodiments, Ar₄ is a 4-substituted aryl.

In a further embodiment, Ar₄ may be selected from the group consisting of alkoxy-substituted phenyl, halogen-substituted phenyl, hydroxy-substituted phenyl, amino-substituted phenyl, and heterocyclyl-substituted phenyl.

In certain embodiments, Ar₄ is a 4-substituted aryl wherein the substituent is selected from the group consisting of alkyl, alkoxy, halogen, hydroxy, amino, alkylamino, heterocyclyl, and heteroaryl. In some embodiments, the substituent on Ar₄ is selected from chloro, fluoro, hydroxy, methoxy, ethoxy, propoxy, isopropoxy, n-butoxy, sec-butoxy, tert-butoxy, trifluoromethoxy, N-morpholinyl, N-pyrrolidinyl, N-pyrazolyl, N-triazolyl and 2-oxopyrrolidinyl.

Suitable embodiments of the compounds of Formula II can be selected from the group consisting of:
N-{1-[2-(1,3-Dioxolan-2-yl)ethyl]piperidin-4-yl}-N-(4-fluorobenzyl)-N′-(4-isobutoxybenzyl)carbamide, hydrochloride;
N-{1-[2-(1,3-Dioxan-2-yl)ethyl]piperidin-4-yl}-N-(4-fluorobenzyl)-2-[4-(2-hydroxy-2-methylpropoxy)phenyl]acetamide, tartrate;
N-{1-[3-(3,5-Dimethylpiperidin-1-yl)propyl]piperidin-4-yl}-N-(4-fluorobenzyl)-2-(4-isobutoxyphenyl)acetamide, dihydrochloride;
1-[3-(4-{(4-Fluorobenzyl)-[2-(4-isobutoxyphenyl)acetyl]amino}piperidin-1-yl)propyl]piperidine-4-carboxylic acid methyl ester, dihydrochloride;
N-(4-Fluorobenzyl)-2-(4-isobutoxyphenyl)-N-{1-[2-(1-methylpyrrolidin-2-yl)ethyl]piperidin-4-yl}acetamide, dioxalate;
N-{1-[3-(2,6-Dimethylmorpholin-4-yl)propyl]piperidin-4-yl}-N-(4-fluorobenzyl)-2-(4-isobutoxyphenyl)acetamide, dioxalate;
N-(4-Fluorobenzyl)-N-{1-[3-(3-hydroxypiperidin-1-yl)propyl]piperidin-4-yl}-2-(4-isobutoxyphenyl)acetamide, dioxalate;
N-(4-Fluorobenzyl)-2-(4-isobutoxyphenyl)-N-{1-[3-(2-methylpiperidin-1-yl)propyl]piperidin-4-yl}acetamide, dioxalate;
N-(4-Fluorobenzyl)-2-(4-isobutoxyphenyl)-N-[1-(3-pyrrolidin-1-yl-propyl)piperidin-4-yl]acetamide, dioxalate;
N-{1-[3-(2,5-Dimethylpyrrolidin-1-yl)propyl]piperidin-4-yl}-N-(4-fluorobenzyl)-2-(4-isobutoxyphenyl)acetamide, dioxalate;
N-(4-Fluorobenzyl)-N-{1-[3-(3-hydroxymethylpiperidin-1-yl)propyl]piperidin-4-yl}-2-(4-isobutoxyphenyl)acetamide, dioxalate;
N-(4-Fluorobenzyl)-2-(4-isobutoxyphenyl)-N-{1-[3-(4-(S)-isopropyl-2-oxo-oxazolidin-3-yl)propyl]piperidin-4-yl}acetamide, oxalate;
N-[2-(4-Fluorophenyl)ethyl]-2-(4-isobutoxyphenyl)-N-{1-[3-(4-(S)-isopropyl-2-oxo-oxazolidin-3-yl)propyl]piperidin-4-yl}acetamide, oxalate;
N-[2-(4-Fluorophenyl)ethyl]-N-{1-[3-(4-(S)-isopropyl-2-oxo-oxazolidin-3-yl)propyl]piperidin-4-yl}-2-(4-propoxyphenyl)acetamide, oxalate;
N-(4-Fluorobenzyl)-N-{1-[3-(4-(S)-isopropyl-2-oxo-oxazolidin-3-yl)propyl]piperidin-4-yl}-2-(4-propoxyphenyl)acetamide, oxalate;
N-{1-[2-(1,3-Dioxan-2-yl)ethyl]piperidin-4-yl}-N-(4-fluorobenzyl)-2-(4-isobutoxyphenyl)acetamide, oxalate;
N-{1-[2-(1,3-Dioxan-2-yl)ethyl]piperidin-4-yl}-N-[2-(4-fluorophenyl)ethyl]-2-(4-isobutoxyphenyl)acetamide, oxalate;
N-{1-[2-(1,3-Dioxan-2-yl)ethyl]piperidin-4-yl}-N-[2-(4-fluorophenyl)ethyl]-2-(4-propoxyphenyl)acetamide, oxalate;
N-{1-[2-(1,3-Dioxan-2-yl)ethyl]piperidin-4-yl}-N-(4-fluorobenzyl)-2-(4-propoxyphenyl)acetamide, tartrate;
N-{1-[2-(1,3-Dioxan-2-yl)ethyl]piperidin-4-yl}-N-(4-fluorobenzyl)-N′-(4-isobutoxybenzyl)carbamide, tartrate;
N-{1-[2-(1,3-Dioxan-2-yl)ethyl]piperidin-4-yl}-N-(4-fluorobenzyl)-2-(4-fluorophenyl)acetamide, tartrate;
N-{1-[2-(1,3-Dioxan-2-yl)ethyl]piperidin-4-yl}-N-(4-fluorobenzyl)-2-p-tolylacetamide, tartrate;
2-Benzofuran-5-yl-N-{1-[2-(1,3-dioxan-2-yl)ethyl]piperidin-4-yl}-N-(4-fluorobenzyl)acetamide, tartrate;
2-(2,3-Dihydrobenzofuran-5-yl)-N-{1-[2-(1,3-dioxan-2-yl)ethyl]piperidin-4-yl}-N-(4-fluorobenzyl)acetamide, tartrate;
N-{1-[2-(2,2-Dimethyl-1,3-dioxolan-4-yl)ethyl]piperidin-4-yl}-N-(4-fluorobenzyl)-2-(4-isobutoxyphenyl)acetamide, tartrate;
N-{1-[2-(1,3-Dioxan-4-yl)ethyl]piperidin-4-yl}-N-(4-fluorobenzyl)-2-(4-isobutoxyphenyl)acetamide, tartrate;
N-{1-[2-(1,3-Dioxan-4-yl)ethyl]piperidin-4-yl}-N-(4-fluorobenzyl)-2-(4-trifluoromethylphenyl)acetamide, tartrate;
2-(4-Cyanophenyl)-N-{1-[2-(1,3-dioxan-4-yl)ethyl]piperidin-4-yl}-N-(4-fluorobenzyl)acetamide, tartrate;
N-(4-Fluorobenzyl)-2-(4-isobutoxyphenyl)-N-{1-[2-(2-oxo-imidazolidin-1-yl)ethyl]piperidin-4-yl}acetamide, hydrochloride;
2-(4-Methoxyphenyl)-N-(4-methylbenzyl)-N-{1-[2-(2-oxo-imidazolidin-1-yl)ethyl]piperidin-4-yl}acetamide, hydrochloride;
N-(4-Fluorobenzyl)-2-(4-isopropoxyphenyl)-N-{1-[2-(2-oxo-imidazolidin-1-yl)ethyl]piperidin-4-yl}acetamide, hydrochloride;
N-(4-Fluorobenzyl)-2-(4-isopropoxyphenyl)-N-{1-[3-(3-methyl-2-oxo-2,3-dihydro-benzoimidazol-1-yl)propyl]piperidin-4-yl}acetamide; hydrochloride;
N-{1-[2-(2,4-Dioxo-1,4-dihydro-2H-quinazolin-3-yl)ethyl]piperidin-4-yl}-2-(4-methoxyphenyl)-N-(4-methylbenzyl)acetamide, hydrochloride;
2-(4-Methoxyphenyl)-N-(4-methylbenzyl)-N-{1-[3-(2-oxo-2,3-dihydrobenzoimidazol-1-yl)propyl]piperidin-4-yl}-acetamide, hydrochloride;
N-(4-Fluorobenzyl)-2-(4-isopropoxyphenyl)-N-{1-[4-(2-oxo-2,3-dihydrobenzoimidazol-1-yl)butyl]piperidin-4-yl}acetamide, hydrochloride;
N-{1-[2-(2,4-Dioxo-1,4-dihydro-2H-quinazolin-3-yl)ethyl]piperidin-4-yl}-N-(4-fluorobenzyl)-2-(4-isopropoxyphenyl)acetamide, hydrochloride;
N-{1-[2-(1,3-Dioxolan-2-yl)ethyl]piperidin-4-yl}-N-(4-fluorobenzyl)-N′-(4-isopropoxy-benzyl)carbamide, oxalate;
N-{1-[2-(1,3-Dioxolan-2-yl)ethyl]piperidin-4-yl]-2-(4-methoxyphenyl)-N-(4-methylbenzyl)acetamide, hydrochloride;
N-{1-[2-(1,3-Dioxolan-2-yl)ethyl]piperidin-4-yl}-N-(4-fluorobenzyl)-2-(4-isobutoxyphenyl)acetamide, hydrochloride;
N-{1-[2-(1,3-Dioxolan-2-yl)ethyl]piperidin-4-yl}-2-(4-isopropoxyphenyl)-N-(4-methylbenzyl)acetamide, hydrochloride;
N-{1-[2-(1,3-Dioxolan-2-yl)ethyl]piperidin-4-yl}-N-(4-fluorobenzyl)-2-(4-propoxyphenyl)acetamide, tartrate;
N-(4-Fluorobenzyl)-N′-(4-isopropoxybenzyl)-N-{1-[2-((S)-4-methyl-1,3-dioxolane-2-yl)ethyl]piperidin-4-yl}carbamide, oxalate;
N-(4-Fluorobenzyl)-N′-(4-isopropoxybenzyl)-N-[1-(3-morpholin-4-yl-propyl)piperidin-4-yl]carbamide, oxalate;
2-(4-Methoxyphenyl)-N-(4-methylbenzyl)-N-[1-(2-morpholin-4-ylethyl)piperidin-4-yl]acetamide, dihydrochloride;
2-(4-Methoxyphenyl)-N-(4-methylbenzyl)-N-[1-(3-morpholin-4-ylpropyl)piperidin-4-yl]acetamide, dihydrochloride;
N-(4-Fluorobenzyl)-2-(4-isobutoxyphenyl)-N-[1-(3-morpholin-4-ylpropyl)piperidin-4-yl]acetamide, dihydrochloride;
N-(4-Fluorobenzyl)-2-(4-isopropoxyphenyl)-N-[1-(3-morpholin-4-yl-propyl)piperidin-4-yl]acetamide, dihydrochloride;
N-(4-Fluorobenzyl)-N′-(4-isopropoxybenzyl)-N-[1-(3-piperidin-1-yl-propyl)piperidin-4-yl]carbamide, oxalate;
N-(4-Fluorobenzyl)-N′-(4-isopropoxybenzyl)-N-[1-(3-((S)-4-isopropyl-2-oxazolidinon-1-yl-propyl)piperidin-4-yl]carbamide, tartrate;
N-(4-Fluorobenzyl)-N′-(4-isopropoxybenzyl)-N-{1-[2-(2,5,5-trimethyl-1,3-dioxan-2-yl)ethyl]}piperidin-4-yl]carbamide, oxalate;
N-{1-[3-(1,3-Dioxolan-2-yl)propyl]piperidin-4-yl}-N-(4-fluorobenzyl)-N′-(4-isopropoxybenzyl)carbamide, oxalate;
N-[1-(2,2-Dimethyl-1,3-dioxan-5-yl)piperidin-4-yl]-N-(4-fluorobenzyl)-N′-(4-isopropoxybenzyl)carbamide, oxalate;
N-(4-Fluorobenzyl)-N′-(4-isopropoxybenzyl)-N-{[2-(1-methylpyrrolidin-2-yl)ethyl]-piperidin-4-yl}carbamide, oxalate;
N-[1-(2,2-Dimethyl-1,3-dioxan-5-yl)piperidin-4-yl]-N-(4-fluorobenzyl)-2-(4-isobutoxyphenyl)acetamide, oxalate;
N-[1-(1,3-Dioxan-5-yl)-piperidin-4-yl)-N-(4-fluorobenzyl)-2-(4-isobutoxyphenyl)acetamide, tartrate;
N-[1-(2,2-Dimethyl-1,3-dioxan-5-yl)piperidin-4-yl]-N-(4-fluorobenzyl)-2-(4-fluorophenyl)acetamide, tartrate;
N-{1-[2-(1,3-Dioxan-4-yl)ethyl]piperidin-4-yl}-N-(4-fluorobenzyl)-2-(4-fluorophenyl)acetamide, tartrate:
N-{1-[2-(1,3-Dioxan-4-yl)ethyl]piperidin-4-yl}-N-(4-fluorobenzyl)-2-(4-trifluoromethoxyphenyl)acetamide, tartrate:
N-{1-[2-(1,3-Dioxan-4-yl)ethyl]piperidin-4-yl}-N-(4-fluorobenzyl)-2-(4-propoxyphenyl)acetamide, tartrate;
N-(4-Fluorobenzyl)-2-(4-isobutoxyphenyl)-N-[1-(tetrahydropyran-4-yl)piperidin-4-yl]acetamide, tartrate;
N-(4-Fluorobenzyl)-2-(4-isobutoxyphenyl)-N-[1-(tetrahydropyran-4-ylmethyl)piperidin-4-yl]acetamide, tartrate;
N-(4-Fluorobenzyl)-2-(4-isobutoxyphenyl)-N-{1-[2-(tetrahydropyran-4-yl)ethyl]piperidin-4-yl]acetamide, tartrate;
N-(4-Fluorobenzyl)-2-(4-fluorophenyl)-N-[1-(tetrahydropyran-4-yl)piperidin-4-yl]acetamide, tartrate;
N-{1-[2-((4S)-1,3-Dioxane-4-yl)ethyl]piperidine-4-yl}-N-(4-fluorobenzyl)-2-(4-isobutoxyphenyl)acetamide, tartrate;
2-(4-Benzyloxyphenyl)-N-{1-[2-(1,3-dioxan-2-yl)ethyl]piperidin-4-yl}-N-(4-fluorobenzyl)acetamide, tartrate;
N-{1-[2-(1,3-Dioxan-2-yl)ethyl]piperidin-4-yl}-N-(4-fluorobenzyl)-2-(4-hydroxyphenyl)-acetamide, tartrate;
N-{1-[2-(1,3-Dioxan-2-yl)ethyl]piperidin-4-yl}-N-(4-fluorobenzyl)-2-(4-methoxyphenyl)-acetamide, tartrate;
N-{1-[2-(1,3-Dioxan-2-yl)ethyl]piperidin-4-yl}-N-(4-fluorobenzyl)-2-(4-isopropylphenyl)-acetamide, tartrate;
N-{1-[2-(1,3-Dioxan-2-yl)ethyl]piperidin-4-yl}-N-(4-fluorobenzyl)-2-(4-trifluoromethoxy-phenyl)acetamide, tartrate;
N-{1-[2-(1,3-Dioxan-2-yl)ethyl]piperidin-4-yl}-N-(4-fluorobenzyl)-2-(4-ethoxyphenyl)-acetamide, oxalate;
N-{1-[2-(1,3-Dioxan-2-yl)ethyl]piperidin-4-yl}-N-(4-fluorobenzyl)-2-(4-isopropoxyphenyl)-acetamide, oxalate;
N-{1-[2-(1,3-Dioxan-2-yl)ethyl]piperidin-4-yl}-N-(4-fluorobenzyl)-2-phenylacetamide, oxalate;
N-{1-[2-(1,3-Dioxan-2-yl)ethyl]piperidin-4-yl}-N-(4-fluorobenzyl)-2-[4-(2-fluoroethoxy)-phenyl]acetamide, oxalate;
N-{1-[2-(5,5-Dimethyl-1,3dioxan-2-yl)ethyl]piperidin-4-yl}-N-(4-fluorobenzyl)-2-(4-isobutoxyphenyl)acetamide, oxalate;
N-(4-Fluorobenzyl)-2-(4-isobutoxyphenyl)-N-{1-[2-((R)-4-methyl-1,3-dioxan-2-yl)ethyl]-piperidin-4-yl}acetamide, oxalate;
N-(4-Fluorobenzyl)-2-(4-isobutoxyphenyl)-N-{1-[2-((S)-4-methyl-1,3-dioxolan-2-yl)ethyl]-piperidin-4-yl}acetamide, oxalate;
N-{1-[2-(4,6-Dimethyl-1,3-dioxan-2-yl)ethyl]piperidin-4-yl}-N-(4-fluorobenzyl)-2-(4-isobutoxyphenyl)acetamide, oxalate;
N-(4-Fluorobenzyl)-N-{1-[2-((S)-4-methyl-1,3-dioxolan-2-yl)ethyl]piperidin-4-yl}-2-(4-trifluoromethoxyphenyl)acetamide, oxalate;
N-(4-Fluorobenzyl)-2-(4-isopropylphenyl)-N-{1-[2-((S)-4-methyl-1,3-dioxolan-2-yl)ethyl]-piperidin-4-yl}acetamide, oxalate;
N-(4-Fluorobenzyl)-N-{1-[2-((R)-4-methyl-1,3-dioxan-2-yl)ethyl]piperidin-4-yl}-2-(4-trifluoromethoxyphenyl)acetamide, oxalate;
N-(4-Fluorobenzyl)-2-(4-isobutoxyphenyl)-N-{1-[2-(2,5,5-trimethyl-1,3-dioxan-2-yl)ethyl]piperidin-4-yl}acetamide, oxalate;
N-(4-Fluorobenzyl)-2-(4-isobutoxyphenyl)-N-{1-[2-(2-methyl-1,3-dioxolan-2-yl)ethyl]-piperidin-4-yl}acetamide, oxalate;
N-(4-Fluorobenzyl)-2-(4-isobutoxyphenyl)-N-{1-[3-(1,3-dioxolan-2-yl)propyl]piperidin-4-yl}acetamide, tartrate;
N-(4-Fluorobenzyl)-2-(4-isobutoxyphenyl)-N-{1-(3-piperidin-1-yl-propyl)piperidin-4-yl}-acetamide, dihydrochloride;
N-(4-Fluorobenzyl)-2-(4-isobutoxyphenyl)-N-{1-[2-(tetrahydropyran-2-yloxy)ethyl]-piperidin-4-yl}acetamide, oxalate;
N-(4-Fluorobenzyl)-2-(4-isobutoxyphenyl)-N-{1-[3-(2-oxo-piperidin-1-yl)propyl]piperidin-4-yl}acetamide;
N-(4-Fluorobenzyl)-2-(4-isobutoxyphenyl)-N-{1-[3-(2-oxo-pyrrolidin-1-yl)propyl]piperidin-4-yl}acetamide, hydrochloride;
N-(4-Fluorobenzyl)-2-(4-isobutoxyphenyl)-N-{1-[3-((R)-4-isopropyl-2-oxo-oxazolidin-3-yl)propyl]piperidin-4-yl}acetamide, oxalate;
N-(4-Fluorobenzyl)-2-(4-isobutoxyphenyl)-N-{1-[3-(2-oxo-oxazolidin-3-yl)propyl]piperidin-4-yl}acetamide, oxalate;
N-(4-Fluorobenzyl)-2-(4-isobutoxyphenyl)-N-{1-[3-((S)-4-methyl-2-oxo-oxazolidin-3-yl)propyl]piperidin-4-yl}acetamide, tartrate;
N-(4-Fluorobenzyl)-2-(4-isobutoxyphenyl)-N-{1-[3-((S)-4-ethyl-2-oxo-oxazolidin-3-yl)-propyl]piperidin-4-yl}acetamide, oxalate;
N-(4-Fluorobenzyl)-2-(4-isobutoxyphenyl)-N-{1-[2-(1,3-oxothiolan-2-yl)ethyl]piperidin-4-yl}acetamide, L-tartrate;
2-(4-Bromophenyl)-N-{1-[2-(1,3-dioxan-2-yl)ethyl)piperidin-4-yl}-N-(4-fluorobenzyl)-acetamide, L-tartrate;
N-{1-[2-(1,3-Dioxan-2-yl)ethyl)piperidin-4-yl}-N-(4-fluorobenzyl)-2-(4-isobutylamino-phenyl)acetamide, L-tartrate;
N-{1-[2-(1,3-Dioxan-2-yl)ethyl)piperidin-4-yl}-N-(4-fluorobenzyl)-2-(4-propylamino-phenyl)acetamide, L-tartrate;
N-{1-[2-(1,3-Dioxan-2-yl)ethyl)piperidin-4-yl}-N-(4-fluorobenzyl)-2-(4-(1-nitropropyl)-phenyl)acetamide, L-tartrate;
N-{1-[2-(1,3-Dioxan-2-yl)ethyl)piperidin-4-yl}-N-(4-fluorobenzyl)-2-[4-(2-oxopyrrolidin-1-yl)phenyl)acetamide, L-tartrate;
N-{1-[2-(1,3-Dioxan-2-yl)ethyl)piperidin-4-yl}-N-(4-fluorobenzyl)-2-(4-isobutylsulfanyl-phenyl)acetamide, L-tartrate;
N-{1-[2-(1,3-Dioxan-2-yl)ethyl)piperidin-4-yl}-N-(4-fluorobenzyl)-2-(4-iodophenyl)-acetamide, L-tartrate;
2-(4-Acetophenyl)-N-{1-[2-(1,3-dioxan-2-yl)ethyl)piperidin-4-yl}-N-(4-fluorobenzyl)-acetamide, L-tartrate;
2-[4-(1-Hydroxyiminoethyl)phenyl]-N-{1-[2-(1,3-dioxan-2-yl)ethyl)piperidin-4-yl}-N-(4-fluorobenzyl)acetamide, L-tartrate;
N-{1-[2-(1,3-Dioxan-2-yl)ethyl)piperidin-4-yl}-N-(4-fluorobenzyl)-2-(4-morpholin-4-yl-phenyl)acetamide, L-tartrate;
N-{1-[2-(1,3-Dioxan-2-yl)ethyl)piperidin-4-yl}-N-(4-fluorobenzyl)-2-(4-pyrazol-1-yl-phenyl)acetamide, L-tartrate;
N-{1-[2-(1,3-Dioxan-2-yl)-1-methylethyl]piperidin-4-yl}-N-(4-fluorobenzyl)-2-(4-iso-butoxyphenyl)-acetamide, L-tartrate;
N-{1-[2-(1,3-Dioxan-4-yl)ethyl)piperidin-4-yl}-N-(4-fluorobenzyl)-2-(4-pyrazol-1-yl-phenyl)acetamide, L-tartrate;
N-{1-[2-((4R)-1,3-Dioxane-4-yl)ethyl]piperidine-4-yl}-N-(4-fluorobenzyl)-2-(4-isobutoxyphenyl)acetamide, tartrate;
N-{1-[2-(1,3-Dioxan-2-yl)ethyl]piperidin-4-yl}-N-(4-fluorobenzyl)-2-[4-(1,2,4-triazol-4-yl)phenyl]acetamide, L-tartrate;
2-(4-methoxyphenyl)-N-(4-methylbenzyl)-N-{1-[2-(1,3-dioxolan-2-yl)ethyl]piperidin 4-yl}acetamide;
2-(4-methoxyphenyl)-N-(4-methylbenzyl)-N-{1-[3(1,3-dihydro-2H-benzimidazol-2-on-1-yl)propyl]piperidin-4-yl}acetamide;
2-(4-methoxyphenyl)-N-(4-methylbenzyl)-N-[1-(2-(imidazolidinon-1-yl)ethyl)piperidin-4-yl]acetamide;
2-(4-methoxyphenyl)-N-(4-methylbenzyl)-N-{1-[2-(2,4(1H,3H) quinazolinedion-3-yl)ethyl]piperidin-4-yl}acetamide;
2-(4-methoxyphenyl)-N-(4-methylbenzyl)-N-{1-[2-(1,3-dioxolan-2-yl)ethyl]piperidin-4-yl}acetamide;
2-(4-methoxyphenyl)-N-(4-methylbenzyl)-N-[1-(2-(imidazolidinon-1-yl)ethyl)piperidin-4-yl]acetamide; and
2-(4-methoxyphenyl)-N-(4-methylbenzyl)-N-{1-[2-(2,4(1H,3H)quinazolinedion-3-yl)ethyl]piperidin-4-yl}acetamide.

Still another embodiment disclosed herein includes use of compounds of the general Formula III: 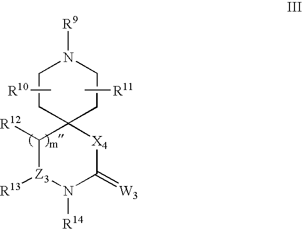
or a pharmaceutically acceptable salt, amide, ester, or prodrug thereof, wherein:

X₄ is selected from the group consisting of CH₂, CH₂CH₂, CH₂O, OCH₂, O, CH₂S, SCH₂, S, CH₂N(R^{N2}), N(R^{N2})CH₂ and N(R^{N2}); wherein R^{N2} is selected from hydrogen and C₁₋₆ alkyl.

W₂ is selected from the group consisting of O and S.

Z₃ is absent or selected from the group consisting of CH and N.

R⁹ is hydrogen, or an optionally substituted substituent selected from the group consisting of C₁₋₆ alkyl, C₂₋₈ alkenyl, C₂₋₈ alkynyl, C₃₋₈ cycloalkyl, aryl, heteroaryl, aryl(C₁₋₆ alkyl), heteroaryl(C₁₋₆ alkyl), heterocyclyl(C₁₋₆ alkyl), C₃₋₈ cycloalkyl(C₁₋₆ alkyl), hydroxy(C₁₋₆ alkyl), amino(C₁₋₆ alkyl), and halo(C₁₋₆ alkyl).

m″ is selected from the group consisting of 0 and 1.

R¹², R¹³, and R¹⁴ are independently hydrogen, or an optionally substituted substituent selected from the group consisting of C₁₋₆ alkyl, aryl(C₁₋₆ alkyl), heteroaryl(C₁₋₆ alkyl), heterocyclyl(C₁₋₆ alkyl), hydroxy(C₁₋₆ alkyl), amino(C₁₋₆ alkyl), halo(C₁₋₆ alkyl), C₃₋₆ cycloalkyl, aryl, and heteroaryl, wherein at least two of R¹², R¹³, and R¹⁴ are independently selected from the group consisting of aryl(C₁₋₆ alkyl), heteroaryl(C₁₋₆ alkyl), and heterocyclyl(C₁₋₆ alkyl).

R¹⁰ and R¹¹ are independently selected from the group consisting of hydrogen, halogen, hydroxy, and optionally substituted C₁₋₆ alkyl or selected such that R¹⁰ and R¹¹ together form a ring system such that 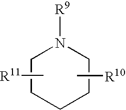
is selected from the group consisting of

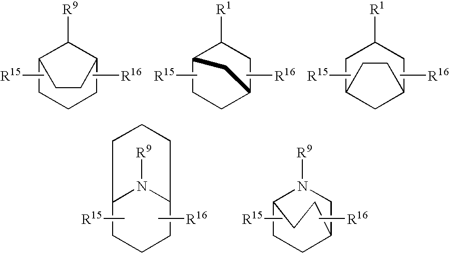

wherein R¹⁵ and R¹⁶ are independently selected from the group consisting of hydrogen, halogen, hydroxy, and C₁₋₆ alkyl.

Within this embodiment, at least two of R¹², R¹³, and R¹⁴ may be independently selected from the group consisting of 4-monosubstituted-aryl(C₁₋₆ alkyl), and 4-monosubstituted-heteroaryl(C₁₋₆ alkyl).

Typically, at least one of the at least two of R¹², R¹³, and R¹⁴ independently selected from the group consisting of aryl(C₁₋₆ alkyl) and heteroaryl(C₁₋₆ alkyl) is selected from the group consisting of fluoro-substituted-aryl(C₁₋₆ alkyl), and fluoro-substituted-heteroaryl(C₁₋₆ alkyl). Also typically, the other of the at least two of R¹², R¹³, and R¹⁴ independently selected from the group consisting of aryl(C₁₋₆ alkyl) and heteroaryl(C₁₋₆ alkyl) is selected from the group consisting of (O—C₁₋₆ alkyl)-substituted-aryl(C₁₋₆ alkyl), and (O—C₁₋₆ alkyl)-substituted-heteroaryl(C₁₋₆ alkyl).

In some embodiments, at least one of R¹², R¹³, and R¹⁴ is selected from the group consisting of fluoro-substituted-aryl(C₁₋₆ alkyl), and fluoro-substituted-heteroaryl(C₁₋₆ alkyl).

Typically, at least two of R¹², R¹³, and R¹⁴ are independently selected from the group consisting of aryl(C₁₋₆ alkyl), heteroaryl(C₁₋₆ alkyl), and heterocyclyl(C₁₋₆ alkyl) are each substituted 1, 2, or 3 times, with a substituent selected from the group consisting of halogen and optionally substituted O—C₁₋₆-alkyl. In one aspect, the halogen is fluorine. In one embodiment, the ring system of one of the at least two of R¹², R¹³, and R¹⁴ independently selected from the group consisting of aryl(C₁₋₆ alkyl), heteroaryl(C₁₋₆ alkyl), heterocyclyl(C₁₋₆ alkyl) is substituted 1 to 3 times, such as 1, 2, or 3 times with an optionally substituted O—C₁₋₆-alkyl, such as a fluorinated O—C₁₋₆-alkyl.

In yet another embodiment, at least two of R¹², R¹³, and R¹⁴ are optionally substituted aryl(C₁₋₆ alkyl). In a preferred embodiment, at least two of R¹², R¹³, and R¹⁴ are optionally substituted benzyl.

As stated, at least two of R¹², R¹³, and R¹⁴ are independently selected from the group consisting of aryl(C₁₋₆ alkyl), heteroaryl(C₁₋₆ alkyl), heterocyclyl(C₁₋₆ alkyl). Typically, the C₁₋₆ alkyl of the aryl(C₁₋₆ alkyl), heteroaryl(C₁₋₆ alkyl), heterocyclyl(C₁₋₆ alkyl) is C₁₋₄ alkyl, such as methylene (C₁ alkyl), ethylene (C₂ alkyl), or propylene (C₃ alkyl), or butylene (C₄ alkyl), more typically a C₁ alkyl or C₂ alkyl, most typically a C₁ alkyl. In a suitable embodiment, the C₁₋₆ alkyl of the aryl(C₁₋₆ alkyl), heteroaryl(C₁₋₆ alkyl), heterocyclyl(C₁₋₆ alkyl) may be substituted so as to form a branched hydrocarbon.

In a combination of embodiments, at least two of R¹², R¹³, and R¹⁴ are an optionally substituted benzyl. One of R¹², R¹³, and R¹⁴ may be a 4-halo-benzyl group and one may be a 4-alkoxy-benzyl group. The 4-halo-benzyl group is typically a 4-fluoro-benzyl. The 4-alkoxy-benzyl group is typically a C₂₋₅ alkoxybenzyl or optionally fluorinated 4-methoxy-benzyl group such as a fluoromethoxy-benzyl, difluoromethoxy-benzyl, trifluoromethoxy-benzyl group, and 2,2,2-trifluorethoxy-benzyl.

As stated, the compounds of the invention may be selected from the group consisting of (i) 1-oxa-4,9-diaza-spiro[5.5]undecan-3-one; (ii) 1-oxa-3,8-diaza-spiro[4.5]decan-2-one; (iii) 1,3,8-triaza-spiro[4.5]decan-2-one; (iv) 1,2,9-triaza-spiro[5.5]undecan-3-one; (v) 1,2,8-triaza-spiro[4.5]decan-3-one; (vi) 1,2,8-triaza-spiro[4.5]decan-3-one (vii) 1,2,4,8-tetraaza-spiro[4.5]decan-3-one; (viii) 2,4,9-triaza-spiro[5.5]undecan-3-one; (ix) 2,8-diaza-spiro[4.5]decan-3-one (x) 2-oxa-4,9-diaza-spiro[5.5]undecan-3-one; (xi) 1-thia-3,8-diaza-spiro[4.5]decan-2-one; and (xii) 1-oxa-3,9-diaza-spiro[5.5]undecan-2-one.

Suitable embodiments of the compounds of Formula III may be selected from the group consisting of:
4-(4-Fluorobenzyl)-3-(4-methoxybenzyl)-8-methyl-1-oxa-3,8-diaza-spiro[4.5]decan-2-one;
3-(4-Ethoxybenzyl)-4-(4-fluorobenzyl)-8-methyl-1-oxa-3,8-diaza-spiro[4.5]decan-2-one;
4-(4-Fluorobenzyl)-8-methyl-3-(4-propoxybenzyl)-1-oxa-3,8-diaza-spiro[4.5]decan-2-one;
3-(4-Cyclopropylmethoxybenzyl)-4-(4-fluorobenzyl)-8-methyl-1-oxa-3,8-diaza-spiro[4.5]decan-2-one;
4-(4-Fluorobenzyl)-3-(4-isopropoxybenzyl)-8-methyl-1-oxa-3,8-diaza-spiro[4.5]decan-2-one;
3-(4-Butoxybenzyl)-4-(4-fluorobenzyl)-8-methyl-1-oxa-3,8-diaza-spiro[4.5]decan-2-one;
4-(4-Fluorobenzyl)-3-(4-isobutoxybenzyl)-8-methyl-1-oxa-3,8-diaza-spiro[4.5]decan-2-one;
3-(4-Difluoromethoxybenzyl)-4-(4-fluorobenzyl)-8-methyl-1-oxa-3,8-diaza-spiro[4.5]decan-2-one;
4-(4-Fluorobenzyl)-8-methyl-3-(4-trifluoromethoxybenzyl)-1-oxa-3,8-diaza-spiro[4.5]decan-2-one;
4-(4-Fluorobenzyl)-8-methyl-3-(4-pentoxybenzyl)-1-oxa-3,8-diaza-spiro[4.5]decan-2-one;
8-Ethyl-4-(4-fluorobenzyl)-3-(4-isobutoxybenzyl)-1-oxa-3,8-diaza-spiro[4.5]decan-2-one;
4-(4-Fluorobenzyl)-3-(4-isobutoxybenzyl)-8-isopropyl-1-oxa-3,8-diaza-spiro[4.5]decan-2-one;
8-Cyclopropylmethyl-4-(4-fluorobenzyl)-3-(4-isobutoxybenzyl)-1-oxa-3,8-diaza-spiro[4.5]decan-2-one;
8-Cyclohexylmethyl-4-(4-fluorobenzyl)-3-(4-isobutoxybenzyl)-1-oxa-3,8-diaza-spiro[4.5]decan-2-one;
8-Cyclopentyl-4-(4-fluorobenzyl)-3-(4-isobutoxybenzyl)-1-oxa-3,8-diaza-spiro[4.5]decan-2-one;
4-(4-Fluorobenzyl)-3-(4-isobutoxybenzyl)-8-(3-morpholin-4-yl-propyl)-1-oxa-3,8-diaza-spiro[4.5]decan-2-one;
8-(2-[1,3]Dioxolan-2-yl-ethyl)-4-(4-fluorobenzyl)-3-(4-isobutoxybenzyl)-1-oxa-3,8-diaza-spiro[4.5]decan-2-one;
4-(4-Fluorobenzyl)-3-(4-isobutoxybenzyl)-8-[2-(2-oxo-imidazolidin-1-yl)-ethyl]-1-oxa-3,8-diaza-spiro[4.5]decan-2-one;
4-(4-Fluorobenzyl)-3-(4-isobutoxybenzyl)-8-[3-(2-oxo-2,3-dihydro-benzoimidazol-1-yl)-propyl]-1-oxa-3,8-diaza-spiro[4.5]decan-2-one;
4-(4-Fluorobenzyl)-3-(4-isobutoxybenzyl)-8-(2-methyl-thiazol-4-yl-methyl)-1-oxa-3,8-diaza-spiro[4.5]decan-2-one;
4-(4-Chlorobenzyl)-3-(4-isobutoxybenzyl)-8-methyl-1-oxa-3,8-diaza-spiro[4.5]decan-2-one;
8-Ethyl-4-(4-chlorobenzyl)-3-(4-isobutoxybenzyl)-1-oxa-3,8-diaza-spiro[4.5]decan-2-one;
4-(4-Chlorobenzyl)-3-(4-isobutoxybenzyl)-8-isopropyl-1-oxa-3,8-diaza-spiro[4.5]decan-2-one;
8-Cyclopropylmethyl-4-(4-chlorobenzyl)-3-(4-isobutoxybenzyl)-1-oxa-3,8-diaza-spiro[4.5]decan-2-one;
8-Cyclohexylmethyl-4-(4-chlorobenzyl)-3-(4-isobutoxybenzyl)-1-oxa-3,8-diaza-spiro[4.5]decan-2-one;
8-(2-[1,3]Dioxolan-2-yl-ethyl)-4-(4-chlorobenzyl)-3-(4-isobutoxybenzyl)-1-oxa-3,8-diaza-spiro[4.5]decan-2-one;
4-(4-Chlorobenzyl)-3-(4-isobutoxybenzyl)-8-[2-(2-oxo-imidazolidin-1-yl)-ethyl]-1-oxa-3,8-diaza-spiro[4.5]decan-2-one;
3-(4-Difluoromethoxybenzyl)-4-(4-fluorobenzyl)-8-methyl-1-oxa-3,8-diaza-spiro[4.5]decan-2-one;
3-(4-Difluoromethoxybenzyl)-8-ethyl-4-(4-fluorobenzyl)-1-oxa-3,8-diaza-spiro[4.5]decan-2-one;
3-(4-Difluoromethoxybenzyl)-4-(4-fluorobenzyl)-8-isopropyl-1-oxa-3,8-diaza-spiro[4.5]decan-2-one;
8-Cyclopropylmethyl-3-(4-difluoromethoxybenzyl)-4-(4-fluorobenzyl)-1-oxa-3,8-diaza-spiro[4.5]decan-2-one;
8-Cyclohexylmethyl-3-(4-difluoromethoxybenzyl)-4-(4-fluorobenzyl)-1-oxa-3,8-diaza-spiro[4.5]decan-2-one;
3-(4-Difluoromethoxybenzyl)-8-(2-[1,3]dioxolan-2-yl-ethyl)-4-(4-fluorobenzyl)-1-oxa-3,8-diaza-spiro[4.5]decan-2-one;
3-(4-Difluoromethoxybenzyl)-4-(4-fluorobenzyl)-8-[2-(2-oxo-imidazolidin-1-yl)-ethyl]-1-oxa-3,8-diaza-spiro[4.5]decan-2-one;
8-Ethyl-4-(4-fluorobenzyl)-3-(4-trifluoromethoxybenzyl)-1-oxa-3,8-diaza-spiro[4.5]decan-2-one;
4-(4-Fluorobenzyl)-8-isopropyl-3-(4-trifluoromethoxybenzyl)-1-oxa-3,8-diaza-spiro[4.5]decan-2-one;
8-Cyclopropylmethyl-4-(4-fluorobenzyl)-3-(4-trifluoromethoxybenzyl)-1-oxa-3,8-diaza-spiro[4.5]decan-2-one;
8-Cyclohexylmethyl-4-(4-fluorobenzyl)-3-(4-trifluoromethoxybenzyl)-1-oxa-3,8-diaza-spiro[4.5]decan-2-one;
8-Cyclopentyl-4-(4-fluorobenzyl)-3-(4-trifluoromethoxybenzyl)-1-oxa-3,8-diaza-spiro[4.5]decan-2-one;
8-(2-[1,3]Dioxolan-2-yl-ethyl)-4-(4-fluorobenzyl)-3-(4-trifluoromethoxybenzyl)-1-oxa-3,8-diaza-spiro[4.5]decan-2-one;
4-(4-Fluorobenzyl)-8-[2-(2-oxo-imidazolidin-1-yl)-ethyl]-3-(4-trifluoromethoxy benzyl)-1-oxa-3,8-diaza-spiro[4.5]decan-2-one;
8-Ethyl-4-(4-fluorobenzyl)-3-(4-propoxybenzyl)-1-oxa-3,8-diaza-spiro[4.5]decan-2-one;
4-(4-Fluorobenzyl)-8-isopropyl-3-(4-propoxybenzyl)-1-oxa-3,8-diaza-spiro[4.5]decan-2-one;
8-Cyclopropylmethyl-4-(4-fluorobenzyl)-3-(4-propoxybenzyl)-1-oxa-3,8-diaza-spiro[4.5]decan-2-one;
8-Cyclohexylmethyl-4-(4-fluorobenzyl)-3-(4-propoxybenzyl)-1-oxa-3,8-diaza-spiro[4.5]decan-2-one;
8-Cyclopentyl-4-(4-fluorobenzyl)-3-(4-propoxybenzyl)-1-oxa-3,8-diaza-spiro[4.5]decan-2-one;
8-(2-[1,3]Dioxolan-2-yl-ethyl)-4-(4-fluorobenzyl)-3-(4-propoxybenzyl)-1-oxa-3,8-diaza-spiro[4.5]decan-2-one;
4-(4-Fluorobenzyl)-8-[2-(2-oxo-imidazolidin-1-yl)-ethyl]-3-(4-propoxybenzyl)-1-oxa-3,8-diaza-spiro[4.5]decan-2-one;
3-(4-Cyclopropylmethoxybenzyl)-8-ethyl-4-(4-fluorobenzyl)-1-oxa-3,8-diaza-spiro[4.5]decan-2-one;
3-(4-Cyclopropylmethoxybenzyl)-4-(4-fluorobenzyl)-8-isopropyl-1-oxa-3,8-diaza-spiro[4.5]decan-2-one;
3-(4-Cyclopropylmethoxybenzyl)-8-cyclopropylmethyl-4-(4-fluorobenzyl)-1-oxa-3,8-diaza-spiro[4.5]decan-2-one;
3-(4-Cyclopropylmethoxybenzyl)-8-(2-[1,3]dioxolan-2-yl-ethyl)-4-(4-fluorobenzyl)-1-oxa-3,8-diaza-spiro[4.5]decan-2-one;
3-(4-Cyclopropylmethoxybenzyl)-4-(4-fluorobenzyl)-8-[2-(2-oxo-imidazolidin-1-yl)-ethyl]-1-oxa-3,8-diaza-spiro[4.5]decan-2-one;
8-(2-[1.3]-Dioxan-2-yl-ethyl)-4-(4-fluorobenzyl)-3-(4-isobutoxybenzyl)-1-oxa-3,8-diaza-spiro[4.5]decane-2-one;
4-(4-Fluorobenzyl)-3-(4-isobutoxybenzyl)-8-{3-[(S)-4-isopropyl-2-oxo-oxazolidin-3-yl]-propyl}-1-oxa-3,8-diaza-spiro[4.5]decane-2-one;
3-(4-Methoxybenzyl)-8-methyl-4-(4-methylbenzyl)-1-oxa-3,8-diaza-spiro[4.5]decan-2-one;
8-Methyl-4-(4-methylbenzyl)-3-(4-trifluoromethoxybenzyl)-1-oxa-3,8-diaza-spiro[4.5]decan-2-one;
1-(4-Fluorobenzyl)-2-(4-methoxybenzyl)-8-methyl-1,2,8-triaza-spiro[4.5]decan-3-one;
2-(4-Ethoxybenzyl)-1-(4-fluorobenzyl)-8-methyl-1,2,8-triaza-spiro[4.5]decan-3-one;
1-(4-Fluorobenzyl)-8-methyl-2-(4-propoxybenzyl)-1,2,8-triaza-spiro[4.5]decan-3-one;
1-(4-Fluorobenzyl)-2-(4-isopropoxybenzyl)-8-methyl-1,2,8-triaza-spiro[4.5]decan-3-one;
2-(4-Butoxybenzyl)-1-(4-fluorobenzyl)-8-methyl-1,2,8-triaza-spiro[4.5]decan-3-one;
2-(4-Cyclopropylmethoxybenzyl)-1-(4-fluorobenzyl)-8-methyl-1,2,8-triaza-spiro[4.5]decan-3-one;
1-(4-Fluorobenzyl)-2-(4-isobutoxybenzyl)-8-methyl-1,2,8-triaza-spiro[4.5]decan-3-one;
2-(4-Difluoromethoxybenzyl)-1-(4-fluorobenzyl)-8-methyl-1,2,8-triaza-spiro[4.5]decan-3-one;
1-(4-Fluorobenzyl)-8-methyl-2-(4-trifluoromethoxybenzyl)-1,2,8-triaza-spiro[4.5]decan-3-one;
1-(4-Fluorobenzyl)-8-methyl-2-(4-pentoxybenzyl)-1,2,8-triaza-spiro[4.5]decan-3-one;
1-(4-Chlorobenzyl)-2-(4-ethoxybenzyl)-8-methyl-1,2,8-triaza-spiro[4.5]decan-3-one;
1-(4-Chlorobenzyl)-8-methyl-2-(4-propoxybenzyl)-1,2,8-triaza-spiro[4.5]decan-3-one;
1-(4-Chlorobenzyl)-2-(4-isobutoxybenzyl)-8-methyl-1,2,8-triaza-spiro[4.5]decan-3-one;
1-(4-Chlorobenzyl)-2-(4-cyclopropylmethoxybenzyl)-8-methyl-1,2,8-triaza-spiro[4.5]decan-3-one;
1-(4-Chlorobenzyl)-2-(4-difluoromethoxybenzyl)-8-methyl-1,2,8-triaza-spiro[4.5]decan-3-one;
1-(4-Ethylbenzyl)-2-(4-ethoxybenzyl)-8-methyl-1,2,8-triaza-spiro[4.5]decan-3-one;
1-(4-Ethylbenzyl)-2-(4-isopropoxybenzyl)-8-methyl-1,2,8-triaza-spiro[4.5]decan-3-one;
1-(4-Ethylbenzyl)-2-(4-isobutoxybenzyl)-8-methyl-1,2,8-triaza-spiro[4.5]decan-3-one;
1-(4-Ethylbenzyl)-2-(4-cyclopropylmethoxybenzyl)-8-methyl-1,2,8-triaza-spiro[4.5]decan-3-one;
1-(4-Ethylbenzyl)-8-methyl-2-(4-trifluoromethoxybenzyl)-1,2,8-triaza-spiro[4.5]decan-3-one;
2-(4-Difluoromethoxybenzyl)-1-(4-fluorobenzyl)-8-ethyl-1,2,8-triaza-spiro[4.5]decan-3-one;
2-(4-Difluoromethoxybenzyl)-1-(4-fluorobenzyl)-8-isopropyl-1,2,8-triaza-spiro[4.5]decan-3-one;
2-(4-Difluoromethoxybenzyl)-1-(4-fluorobenzyl)-8-cyclopropylmethyl-1,2,8-triaza-spiro[4.5]decan-3-one;
2-(4-Difluoromethoxybenzyl)-1-(4-fluorobenzyl)-8-(2-[1,3]dioxolan-2-yl-ethyl)-1,2,8-triaza-spiro[4.5]decan-3-one;
8-Ethyl-1-(4-fluorobenzyl)-2-(4-isobutoxybenzyl)-1,2,8-triaza-spiro[4.5]decan-3-one;
1-(4-Fluorobenzyl)-2-(4-isobutoxybenzyl)-8-isopropyl-1,2,8-triaza-spiro[4.5]decan-3-one;
8-Cyclopropylmethyl-1-(4-fluorobenzyl)-2-(4-isobutoxybenzyl)-1,2,8-triaza-spiro[4.5]decan-3-one;
8-(2-[1,3]dioxolan-2-yl-ethyl)-1-(4-fluorobenzyl)-2-(4-isobutoxybenzyl)-1,2,8-triaza-spiro[4.5]decan-3-one;
4-(4-Ethoxybenzyl)-5-(4-fluorobenzyl)-9-methyl-1-oxa-4,9-diaza-spiro[5.5]undecan-3-one;
5-(4-Fluorobenzyl)-9-methyl-4-(4-propoxybenzyl)-1-oxa-4,9-diaza-spiro[5.5]undecan-3-one;
5-(4-fluorobenzyl)-4-(4-isobutoxybenzyl)-9-methyl-1-oxa-4,9-diaza-spiro[5.5]undecan-3-one;
5-(4-fluorobenzyl)-9-methyl-4-(4-trifluoromethoxybenzyl)-1-oxa-4,9-diaza-spiro[5.5]undecan-3-one;
5-(4-Chlorobenzyl)-4-(4-isobutoxybenzyl)-9-methyl-1-oxa-4,9-diaza-spiro[5.5]undecan-3-one;
5-(4-Chlorobenzyl)-4-(4-cyclopropylmethoxybenzyl)-9-methyl-1-oxa-4,9-diaza-spiro[5.5]undecan-3-one;
9-Ethyl-5-(4-fluorobenzyl)-4-(4-propoxybenzyl)-1-oxa-4,9-diaza-spiro[5.5]undecan-3-one;
1-(4-Fluorobenzyl)-2-(4-ethoxybenzyl)-9-methyl-1,2,9-triaza-spiro[5.5]undecan-3-one;
1-(4-Fluorobenzyl)-2-(4-cyclopropylmethoxybenzyl)-9-methyl-1,2,9-triaza-spiro[5.5]undecan-3-one;
1-(4-Fluorobenzyl)-2-(4-isobutoxybenzyl)-9-methyl-1,2,9-triaza-spiro[5.5]undecan-3-one;
1-(4-Fluorobenzyl)-2-(4-propoxybenzyl)-9-methyl-1,2,9-triaza-spiro[5.5]undecan-3-one; 1-(4-Ethylbenzyl)-2-(4-isobutoxybenzyl)-9-methyl-1,2,9-triaza-spiro[5.5]undecan-3-one;
1-(4-Fluorobenzyl)-2-(4-cyclopropylmethoxybenzyl)-9-ethyl-1,2,9-triaza-spiro[5.5]undecan-3-one;
2-(4-Ethoxybenzyl)-1-(4-fluorobenzyl)-8-methyl-1,2,4,8-tetraaza-spiro[4.5]decan-3-one;
1-(4-Fluorobenzyl)-2-(4-isobutoxybenzyl)-8-methyl-1,2,4,8-tetraaza-spiro[4.5]decan-3-one;
2-(4-Difluoromethoxybenzyl)-1-(4-fluorobenzyl)-8-methyl-2,8-diaza-spiro[4.5]decan-3-one;
1-(4-Fluorobenzyl)-2-(4-isobutoxybenzyl)-8-methyl-2,8-diaza-spiro[4.5]decan-3-one;
2-(4-Cyclopropylmethoxybenzyl)-1-(4-fluorobenzyl)-8-methyl-2,8-diaza-spiro[4.5]decan-3-one;
8-Ethyl-1-(4-fluorobenzyl)-2-(4-isobutoxybenzyl)-2,8-diaza-spiro[4.5]decan-3-one;
8-(2-[1,3]Dioxolan-2-yl-ethyl)-1-(4-fluorobenzyl)-2-(4-isobutoxybenzyl)-2,8-diaza-spiro[4.5]decan-3-one;
3-(4-Difluoromethoxybenzyl)-4-(4-fluorobenzyl)-8-methyl-1,3,8-triaza-spiro[4.5]decan-2-one;
4-(4-Fluorobenzyl)-3-(4-isobutoxybenzyl)-8-methyl-1,3,8-triaza-spiro[4.5]decan-2-one;
3-(4-Cyclopropylmethoxybenzyl)-4-(4-fluorobenzyl)-8-methyl-1,3,8-triaza-spiro[4.5]decan-2-one;
8-Ethyl-4-(4-fluorobenzyl)-3-(4-isobutoxybenzyl)-1,3,8-triaza-spiro[4.5]decan-2-one;
8-(2-[1,3]Dioxolan-2-yl-ethyl)-4-(4-fluorobenzyl)-3-(4-isobutoxybenzyl)-1,3,8-triaza-spiro[4.5]decan-2-one;
1-(4-Fluorobenzyl)-2-(4-ethoxybenzyl)-9-methyl-2,4,9-triaza-spiro[5.5]undecan-3-one;
1-(4-Fluorobenzyl)-2-(4-cyclopropylmethoxybenzyl)-9-methyl-2,4,9-triaza-spiro[5.5]undecan-3-one;
1-(4-Fluorobenzyl)-2-(4-isobutoxybenzyl)-9-methyl-2,4,9-triaza-spiro[5.5]undecan-3-one;
1-(4-Fluorobenzyl)-2-(4-trifluoromethoxybenzyl)-9-methyl-2,4,9-triaza-spiro[5.5]undecan-3-one;
1-(4-Fluorobenzyl)-2-(4-isobutoxybenzyl)-9-ethyl-2,4,9-triaza-spiro[5.5]undecan-3-one;
4-(4-Ethoxybenzyl)-5-(4-fluorobenzyl)-9-methyl-1-oxa-3,9-diaza-spiro[5.5]undecan-2-one;
3-(4-Ethoxybenzyl)-5-(4-fluorobenzyl)-9-methyl-1-oxa-3,9-diaza-spiro[5.5]undecan-2-one;
3-(4-Ethoxybenzyl)-4-(4-fluorobenzyl)-9-methyl-1-oxa-3,9-diaza-spiro[5.5]undecan-2-one;
5-(4-Fluorobenzyl)-4-(4-propoxybenzyl)-9-methyl-1-oxa-3,9-diaza-spiro[5.5]undecan-2-one;
5-(4-Fluorobenzyl)-3-(4-propoxybenzyl)-9-methyl-1-oxa-3,9-diaza-spiro[5.5]undecan-2-one;
4-(4-Fluorobenzyl)-3-(4-propoxybenzyl)-9-methyl-1-oxa-3,9-diaza-spiro[5.5]undecan-2-one;
5-(4-Fluorobenzyl)-4-(4-isobutoxybenzyl)-9-methyl-1-oxa-3,9-diaza-spiro[5.5]undecan-2-one;
4-(4-Fluorobenzyl)-3-(4-isobutoxybenzyl)-9-methyl-1-oxa-3,9-diaza-spiro[5.5]undecan-2-one;
4-(4-Ethoxybenzyl)-5-(4-fluorobenzyl)-9-methyl-2-oxa-4,9-diaza-spiro[5.5]undecan-3-one;
5-(4-Fluorobenzyl)-4-(4-methoxybenzyl)-9-methyl-2-oxa-4,9-diaza-spiro[5.5]undecan-3-one;
5-(4-Fluorobenzyl)-4-(4-propoxybenzyl)-9-methyl-2-oxa-4,9-diaza-spiro[5.5]undecan-3-one;
5-(4-Fluorobenzyl)-4-(4-isobutoxybenzyl)-9-methyl-2-oxa-4,9-diaza-spiro[5.5]undecan-3-one;
4-(4-Ethoxybenzyl)-5-(4-fluorobenzyl)-9-methyl-2-oxa-4,9-diaza-spiro[5.5]undecan-3-one;
3-(4-Ethoxybenzyl)-4-(4-fluorobenzyl)-8-methyl-1-thia-3,8-diaza-spiro[4.5]decan-2-one;
4-(4-Fluorobenzyl)-3-(4-methoxybenzyl)-8-methyl-1-thia-3,8-diaza-spiro[4.5]decan-2-one;
4-(4-Fluorobenzyl)-3-(4-propoxybenzyl)-8-methyl-1-thia-3,8-diaza-spiro[4.5]decan-2-one; and
4-(4-Fluorobenzyl)-3-(4-isobutoxybenzyl)-8-methyl-1-thia-3,8-diaza-spiro[4.5]decan-2-one.

Other embodiments include use of compounds selected from the group consisting of:
2-(4-methoxyphenyl)-N-(4-methylbenzyl)-N-{1-[3-(1,3 dihydro-2H-benzimidazol-2-on-1-yl)propyl]piperidin-4-yl}acetamide;
1-(4-methoxyphenyl)-N-(4-methylbenzyl)-N-(1-methylpiperidin-4-yl)cyclopropane carboxamide;
2-(4-Methoxyphenyl)-(1-phenylethyl)-N-(1-methylpiperidin-4-yl)acetamide;
2-(4-Methoxyphenyl)-N-(1-indanyl)-N-(1-methylpiperidin-4-yl)acetamide; and
2-(3,4-Methylenedioxyphenyl)-N-(4-methylbenzyl)-N-(1-methylpiperidin-4-yl)acetamide.

The compounds of Formula I, II, and III exhibit activity at monoamine receptors, specifically serotonin receptors. Certain compounds share the common property of acting as inverse agonists at the 5-HT2A receptor. Thus, experiments performed on cells transiently expressing the human phenotype of the receptor have shown that the compounds of general Formula I, II, and III attenuate the signaling of such receptors in the absence of additional ligands acting upon the receptor. The compounds have thus been found to possess intrinsic activity at this receptor and are able to attenuate the basal, non-agonist-stimulated, constitutive signaling responses that the 5-HT2A receptor displays. The observation that the compounds of general Formula I, II, and III are inverse agonists also indicates that these compounds have the ability to antagonize the activation of 5-HT2A receptors that is mediated by endogenous agonists or exogenous synthetic agonist ligands.

Therefore, some embodiments include use of compounds of Formula I, II, III and the salts and stereoisomers thereof, including compounds that show a relatively high degree of selectivity towards the 5-HT2A subtype of serotonin receptors relative to other subtypes of the serotonin (5-HT) family of receptors as well as to other receptors, most particularly the monoaminergic G-protein coupled receptors, such as dopamine receptors. In one embodiment, these compounds act as inverse agonists and/or antagonists at the 5-HT2A subtype of serotonin receptors.

Methods of Preparation

The compounds of Formulae I and II may in general be prepared by routes such as those summarized below, or by modification of these methods. Ways of modifying the methodology include, among others, temperature, solvent, reagents etc, and will be obvious to those skilled in the art.

For instance, compounds of the Formula C may be synthesized from the corresponding ketone A by reductive amination utilizing any primary amine. The reaction is conveniently carried out by stirring the reactants in an inert solvent such as methanol or ethanol containing acetic acid. As reducing agent NaBH₄, NaCNBH₃, BH₃.pyridine or any related reagent may be used including solid-supported reagents. The reaction is typically carried out at room temperature. The ketone A, as exemplified by the piperidone, may be chosen from a list of compounds corresponding to the Z and Z₁-groups listed in Formulae I and II. The ketones can either be obtained commercially or synthesized by methodology disclosed in Lowe et al. J. Med. Chem. 37: 2831-40 (1994); Carroll et al. J. Med. Chem. 35:2184-91 (1992); or Rubiralta et al. Piperidine—Structure, Preparation, Reactivity and Synthetic Applications of Piperidine and its Derivatives. (Studies in Organic Chemistry 43, Elsevier, Amsterdam, 1991). The protecting group P includes groups such as those described in T. W. Greene and P. G. M. Wuts, Protective Groups in Organic Chemistry, 3. Ed. John Wiley & Sons, 1999, and they should be chosen in such a way, that they are stable to the reaction conditions applied and readily removed at a convenient stage using methodology known from the art. Typical protecting groups are N-Boc, N-Cbz, N-Bn.

Alternatively, the amine C can be synthesized from the primary amine B by reductive amination with any aldehyde. The reaction is conveniently carried out by stirring the reactants in an inert solvent such as methanol or ethanol containing acetic acid. As reducing agent NaBH₄, NaCNBH₃, BH₃.pyridine or any related reagent may be used including solid-supported reagents. The reaction is typically carried out at room temperature. The primary amine B, as exemplified by the 4-aminopiperidine, may be chosen from a list of compounds corresponding to the Z and Z₁-groups listed in Formulae I and II. The amines can either be obtained commercially or synthesized from the corresponding ketones. The protecting group P may be chosen as stated above.

Alternatively, the amine C can be synthesized from the primary amine B by alkylation with any alkylating agent (R-L₁). The leaving group L₁ is suitably a halogen atom, e.g., bromine or iodine, or a sulfonate, e.g. tosylate or mesylate, or another leaving group favoring the reaction. The reaction is conveniently carried out by stirring the reagents under basic conditions in an inert solvent, e.g., diisopropylethylamine in acetonitrile, or K₂CO₃ in N,N-dimethylformamide. The reaction is typically carried out at temperatures between room temperature and 80° C. The primary amine B, as exemplified by the 4-aminopiperidine, may be chosen from a list of compounds corresponding to the Z and Z1-groups listed in Formulae I and II. The amines can either be obtained commercially or synthesized from the corresponding ketones. The protecting group P may be chosen as stated above.

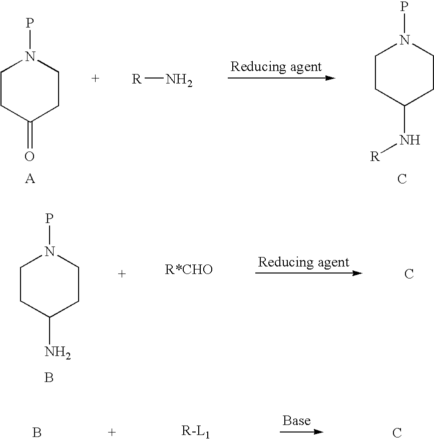

Wherein R and R* are defined in agreement with Formulae I and II, and P represents a suitable protecting group, and L₁ represents a suitable leaving group.

The secondary amine C may be acylated using any isocyanate or isothiocyanate (Q₁-N═C═W or Q₁-N═C═W₁) to give the corresponding ureas or thioureas D. The reaction is typically carried out by stirring the reactants, using an excess of isocyanate or isothiocyanate in an inert solvent, e.g., dichloromethane at a temperature between 0° C. and room temperature and under dry conditions. The amine C may also be acylated using any carboxylic acid halide (Q₂COX), e.g., chloride, or carboxylic anhydride ((Q₂C═O)₂O) to give amides of the general structure E. The reaction is typically carried out using an excess of the acylating agent and a suitable base, e.g., triethylamine or diisopropylethylamine in an inert solvent, e.g., dichloromethane, at a temperature between 0° C. and room temperature and under dry conditions. As an alternative to the carboxylic acid halides and carboxylic acid anhydrides, the amine C may be acylated using a carboxylic acid (Q₂COOH) and a suitable coupling reagent e.g. PyBroP, DCC or EDCI. The reaction is typically carried out using an excess of the acylating agent and the coupling reagent in an inert solvent, e.g., dichloromethane at a temperature between 0° C. and room temperature and under dry conditions. The compounds of the general structure (E) may be converted into the corresponding thioamides using methodology disclosed in Varma et al., Org. Lett. 1: 697-700 (1999); Cherkasov et al. Tetrahedron 41:2567 (1985); or Scheibye et al, Bull. Soc. Chim. Belg. 87:229 (1978).

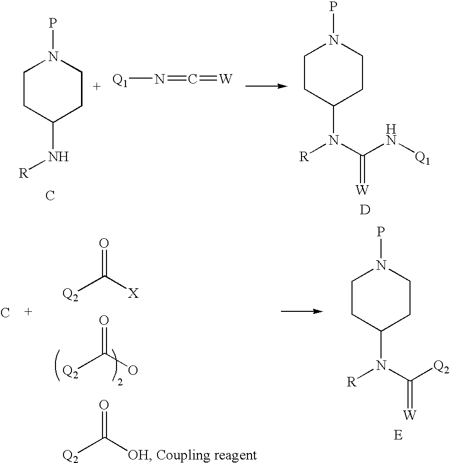

Wherein R, Q₁, Q₂, W and W₁ are defined in agreement with Formulae I and II, P represents a suitable protecting group, and X represents a halide.

The substituent T on the ring nitrogen in compounds F or G can be introduced by a two step procedure. First, the protecting group on the urea D or the amide E is removed using well-known methods. For example, the N-Boc group is removed by treating the protected compound with 4 M HCl in dioxane or trifluoroacetic acid in dichloromethane. Second, the secondary amines obtained from D and E can be alkylated by reductive amination using any aldehyde (T*-CHO) or ketone (T=O). The reaction is conveniently carried out by stirring the reactants in an inert solvent such as methanol or ethanol. As a reducing agent, solid-supported borohydride, NaBH₄, NaCNBH₃, BH₃.pyridine, H₂/Pd—C or any related reagent may be used, including solid-supported reagents. The reaction is typically carried out at room temperature.

Alternatively, the compounds F and G can be synthesized from the secondary amine obtained from D or E as described above by alkylation with any alkylating agent (T-L₁). The leaving group L₁ is suitably a halogen atom, e.g., bromine or iodine, or a sulfonate, e.g., tosylate or mesylate, or another leaving group favoring the reaction. The reaction is conveniently carried out by stirring the reagents under basic conditions in an inert solvent, for example diisopropylethylamine in acetonitrile, or K₂CO₃ in N,N-dimethylformamide. The reaction is typically carried out at temperatures between room temperature and 80° C.

Alternatively, the T-group can be introduced in the first step of the synthetic sequence leading to the compounds in accordance with the present invention by N-alkylation of compound H with any alkylating agent (T-L₁). The leaving group L₁ is suitably a halogen atom, e.g., bromine or iodine, or a sulfonate, e.g., tosylate or mesylate, or another leaving group favoring the reaction. The reaction is conveniently carried out by stirring the reagent under basic conditions in an inert solvent, e.g., diisopropylethylamine in acetonitrile, or K₂CO₃ in N,N-dimethylformamide. The reaction is typically carried out at temperatures between room temperature and 80° C. Alternatively the T-group can be introduced in the first step by reductive amination using any aldehyde (T*-CHO) or ketone (T=O) and a suitably protected compound H′, exemplified by 4-piperidone ethylene ketal. The reaction is conveniently carried out by stirring the reactants in an inert solvent such as methanol or ethanol. As a reducing agent, solid-supported borohydride, NaBH₄, NaCNBH₃, BH₃.pyridine, H₂/Pd—C or any related reagent may be used, including solid-supported reagents. The reaction is typically carried out at room temperature, but less reactive carbonyl compounds may require higher temperatures and/or the pre-formation of the corresponding imine under water removal before addition of the reducing agent. Removal of the protecting group gives the desired compound J. The secondary amine H and H′, as exemplified by 4-piperidone and its protected derivative, may be chosen from a list of compounds corresponding to the Z and Z1-groups listed in Formulae I and II. The amines can either be obtained commercially or synthesized from methodology disclosed in Lowe et al., J. Med. Chem. 37:2831-40 (1994); and Carroll et al., J. Med. Chem. 35:2184-91 (1992).

Alternatively, compounds of the general structure J may be synthesized starting from K using the method disclosed in: Kuehne et al., J. Org. Chem. 56:2701 (1991); and Kuehne et al., J. Org. Chem. (1991), 56:513.

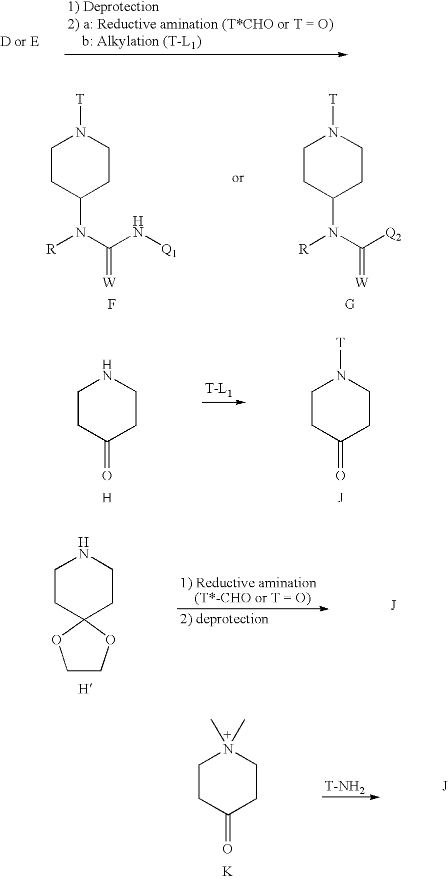

Wherein R, Q₁, Q₂, W, and T are defined in agreement with Formulae I and II, and L₁ is a suitable leaving group.

Heterocyclylalkyl alkylating agents such as T-L₁ may be commercially available or are typically obtained by alkylation of a heterocycle with a bifunctional alkyl-linker, as shown below. The leaving groups L₁ and L₂ are suitably a halogen atom, e.g., chlorine, bromine or iodine, or a sulfonate, e.g., tosylate or mesylate, or another leaving group favoring the reaction. The reaction is conveniently carried out by stirring the reagent under basic conditions in an inert solvent, e.g., diisopropylethylamine in acetonitrile, or K₂CO₃ in N,N-dimethylformamide. The reaction is typically carried out at temperatures between room temperature and 80° C. The alkylating agent hence obtained can be either reacted in situ in the next step with the secondary amine (i.e. deprotected D/E, or H) or isolated from the reaction mixture before its further use. Heterocyclylalkyl alcohols such as T*-CH₂OH or T-OH may also be converted into suitable alkylating agents T-L₁ by transforming the hydroxyl into a leaving group, e.g. by tosylation, mesylation or halogenation. Alternatively, T*-CH₂OH or T-OH may be oxidized to the corresponding aldehydes or ketones T*-CHO or T=O with, for example, pyridinium chlorochromate, CrO₃—H₂SO₄, or via the Swern or Dess-Martin procedures, to be used in a reductive amination step with the secondary amines as described above.

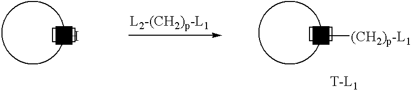

Wherein Y, p and T are defined in agreement with Formulae I and II, and L₁ and L₂ are suitable leaving groups.

The building blocks incorporating the aromatic groups Ar₁, Ar₂, Ar₃ and Ar₄ may either be obtained commercially or synthesized from methodology disclosed in the literature. The introduction of substituents on Ar₁, Ar₂, Ar₃ and/or Ar₄ may be performed from a suitable precursor at any appropriate stage of the preparation of the compounds.

For instance, compounds containing an alkoxy substituents may be typically prepared by Williamson ether synthesis from the corresponding hydroxyaryl derivatives.

Structures bearing an amine substituent on Ar₁, Ar₂, Ar₃ and/or Ar₄ may be obtained from a suitable halo- or pseudohalo precursor (e.g. Br, I-, Cl-, triflate-, nonaflate-, tosylate-substituted aryl derivatives) by metal-catalyzed amination chemistries, such as Pd— or Ni— (Hartwig, Angew. Chem. Int. Ed., 1998, 37, 2046-2067; Yang & Buchwald, J. Organometallic Chem., 1999, 576, 125-146; Hartwig in Modern Amination Methods; Ricci, Ed.; Wiley-VCH: Weinheim, Germany, 2000) or Cu-catalyzed (Buchwald et al, Org. Lett., 2002, 4, 581-584; Kwong & Buchwald, Org. Lett., 2003, 5, 793-796). Alternatively, these compounds can be obtained from aniline-based precursors either by alkylation (Hickinbottom, J. Chem. Soc. 1930, 992), or by reductive amination (Emerson & Walters, J. Am. Chem. Soc., 1938, 60, 2023; Milovic et al, Synthesis, 1991, 11, 1043-1045), or by dehydrative alkylation (Rice & Kohn, J. Am. Chem. Soc., 1955, 77, 4052; Brown & Reid, J. Am. Chem. Soc., 1924, 46, 1838). Additionally, compounds of this type may also be synthesized from corresponding boronic acids by Cu-catalyzed coupling (Antilla & Buchwald, Org. Lett., 2001, 3, 2077-2079).

The structures bearing an amide substituent on Ar₁, Ar₂, Ar₃ and/or Ar₄ may be obtained from a suitable halo- or pseudohalo precursor either by Pd catalyzed (Yin & Buchwald, J. Am. Chem. Soc., 2002, 124, 6043-6048) or by Cu catalyzed (Buchwald et al, J. Am. Chem. Soc., 2002, 124, 7421-7428) amidation chemistries. Alternatively, these compounds may also be obtained from the corresponding aniline precursors either by acylation (Wolf, Liebigs Ann. Chem., 1952, 576, 35; Yasukara et al, J. Chem. Soc. Perkin Trans. 1, 2000, 17, 2901-2902; Nigam & Weedon, J. Chem. Soc., 1957, 2000) or by formylation (Hirst & Cohen, J. Chem. Soc., 1895, 67, 830; Olah & Kuhn, Chem. Ber. 1956, 89, 2211; Guthrie et al, Can. J. Chem., 1993, 71, 2109-2122).

Compounds that carry an alkylsulfanyl substituent on Ar₁, Ar₂, Ar₃ and/or Ar₄ be obtained from a suitable halo- or pseudohalo precursor by Pd catalyzed (Li, J. Org. Chem., 2002, 67, 3643-3650), or Cu catalyzed (Kwong & Buchwald, Org. Lett., 2002, 4, 3517-3520) thioetherification chemistry. Alternatively, these compounds may be prepared by alkylation of corresponding benzenethiol precursors (Vogel, J. Chem. Soc., 1948, 1809; Landini & Rocca, Synthesis, 1974, 565-566; Bun-Hoi et al, J. Org. Chem., 1951, 16, 988). Alternatively, alkylarylsulfanyls may be obtained by irradiation of benzenethiols and alkenes (Screttas & Micha-Screttas, J. Org. Chem., 1978, 43, 1064-1071).

Compounds that bear an acyl group on Ar₁, Ar₂, Ar₃ and/or Ar₄ may be prepared from corresponding aryl iodides by Pd catalyzed (Cacchi et al, Org. Lett, 2003, 5, 289-293) acylation chemistry. Alternatively, they may be obtained from the corresponding benzenes by Friedel-Crafts chemistry (Read, J. Am. Chem. Soc., 1922, 44, 1746-1755), or by addition of aryl-Grignard reagents to nitriles (Whitmore et al, J. Am. Chem. Soc., 1947, 69, 235-237) or to acyl chlorides (Whitmore & Lester, J. Am. Chem. Soc., 1942, 64, 1247), or by either Pd-catalyzed (Gooβen & Ghosh, Angew. Chem. Int. Ed. Engl., 2001, 40, 3458-3460) or Rh-catalyzed acylation of arylboronic acids.

Compounds that bear an N-containing aromatic heterocycle on Ar₁, Ar₂, Ar₃ and/or Ar₄ can be obtained either by metal-catalyzed cross-couplings (Buchwald et al, Org. Lett., 2002, 2, 1403-1406; Buchwald et al, J. Am. Chem. Soc., 2001, 123, 7727-7729; Buchwald et al, J. Am. Chem. Soc., 2002, 124, 11684-11688). Alternatively, they may be accessed from suitable precursors such as aryl hydrazines, aryl amines or aryl nitriles according to literature procedures (e.g. Alvisi, Gazz. Chem. Ital., 1892, 22, 159; Finar, Godfrey, J. Chem. Soc., 1954, 2293; Muri et al, Synth. Commun., 1998, 28, 1299-1321; Artico et al, Europ. J. Med. Chem. Chim. Ther., 1992, 27, 219-228; Biagi et al., Farmaco Ed. Sci. 1988, 43, 597-612; Stefancich et al, Farmaco Ed. Sci., 1984, 39, 752-764).

The compounds of Formula III may in general be prepared by routes such as those summarized below, or by modification of these methods. Cyclization of the appropriate intermediates may be generally achieved with phosgene or its analogues such as CDI, with chloroacetylchloride or equivalents thereof or by treatment with carbondisulfide and subsequent oxidation.

Introduction of the desired piperidine substituent (R⁹) can generally be achieved, after N-deprotection if required, by alkylation or by reductive amination.

Compounds of Formula III in which m″=1, Z₃=CH and X₄═O or OCH₂ may be synthesized from suitably protected 4-piperidone as described by Bassus et al. (Eur. J. Med. Chem.—Chim. Ther. 9:416-423 (1974)). Alkylation of the carbamate nitrogen and introduction of the desired piperidine N-substituent leads to the 3,5-disubstituted spirocycle. 3, 4 or 4,5-disubstituted derivatives may be prepared as described by Fisera et al. (Monatsh. Chem. 125:909-919 (1994)), via an appropriately substituted isoxazoline, followed by reductive cleavage. Cyclisation of the resulting γ-aminoalcohols gives the desired spirocycles. Alternatively, 4,5-disubstituted or 3,4,5-trisubstituted spirocycles may be prepared from an appropriate β-ketoester by reaction with a halide to introduce the 5-substituent, reductive amination (with a primary amine for the 3-substituent), treatment with allyl magnesium bromide, cyclisation as described above, oxidative cleavage of the double bonds (e.g. by ozonolysis) and formation of the piperidine ring by reductive amination.

Compounds of Formula III in which m″=1, Z₃=N and X₄=O or OCH₂ may be prepared by a Strecker synthesis involving an appropriate aldehyde and e.g. tert-butyl carbazate. The nitrile may be transformed into an ester, which is reacted with allyl magnesium bromide, followed by oxidative cleavage of the two olefins, formation of the piperidine by reductive amination. Alkylation, deprotection of the hydrazide and cyclisation gives the desired spirocycles. Depending on the desired substituents and their positions, these steps may be inversed and additional protection steps of functional groups may be required. Alternatively, the compounds may be obtained from an appropriate β-ketoester by reaction with a bis(2-chloroethyl)amine derivative to form the piperidine ring. Reductive amination, saponification and Curtius rearrangement lead to the cyclic urea derivative.

Compounds of Formula III in which m″=1, Z₃=N and X₄=N(R) may be prepared adapting methods described by Bhatia et al. (J. Med. Chem. 39:3938-3950 (1996)) starting from the suitably protected Strecker-product 4-amino-4-cyano-piperidine.

Compounds of Formula III in which m″=0, Z₃=CH and X₄=CH₂CH₂ may be synthesized by Michael addition of a nitrile-derivative to suitably protected 4-methoxycarbonyl-methylenepiperidine, reduction of the nitrile-group to the amine, followed by lactam ring formation, alkylation of the resulting amide and introduction of the desired piperidine substituent.

Compounds of Formula III in which m″=0, Z₃=CH and X₄=CH₂O may be prepared from the appropriate β-ketoester by reaction with a suitably protected bis(2-chloroethyl)amine. Reductive amination, followed by reduction of the nitrile to the alcohol followed by cyclisation and introduction of the piperidine substituent by alkylation or reductive amination leads to the desired spirocycle.

Compounds of Formula III in which m″=0, Z₃=CH and X₄=CH₂N may be prepared from the appropriate β-ketonitrile by reaction with a suitably protected bis(2-chloroethyl)amine. Reductive amination, followed by reduction of the nitrile to the amine followed by cyclisation and introduction of the piperidine substituent by alkylation or reductive amination leads to the desired spirocycle.

Compounds of Formula III in which m″=0, Z₃=CH and X₄═OCH₂ may be prepared as described in herein.

Compounds of Formula III in which m″=0, Z₃=N and X₄=CH₂CH₂ may be prepared from protected 8-aza-1-oxa-spiro[4.5]decan-2-one (Mullen et al. J. Med. Chem. 43:4045 (2000)) by reaction with hydrazine, then S-ethyltrifluorothioacetate, followed by a Mitsunobu reaction according to Meng et al. (Tetrahedron 47:6251 (1991)). The appropriate substituents in 1,2- and 9-positions are introduced by alkylation and/or reductive amination.

Compounds of Formula III in which m″=0, Z₃=N and X₄=CH₂O may be prepared by a Strecker synthesis from suitably protected 4-piperidone and a carbazate (or an appropriate hydrazine). Transformation of the resulting nitrile into the alcohol, cyclisation using methods described above, alkylation (after basic hydrolysis of the exocyclic carbazate function if present) and introduction of the piperidine N-substituent leads to the desired compound.

Compounds of Formula III in which m″=0, Z₃=N and X₄=CH₂N may be prepared using the same strategy as for the preparation of 1,2,9-triaza-4-oxa-spiro[5.5]undecan-3-one, except that the nitrile is reduced to the corresponding amine.

Compounds of Formula III in which m″=0, Z₃=CH and X₄=CH₂ may be synthesized by Michael addition of a nitro-derivative to suitably protected 4-methoxycarbonyl-methylenepiperidine, reduction of the nitro-group to the amine, followed by lactam ring formation, alkylation of the resulting amide and introduction of the desired piperidine substituent.

Compounds of Formula III in which m″=0, Z₃=CH and X₄=O may be prepared as described in herein. Alternatively, a nitroaldol reaction may be used to obtain the desired intermediate 1,2-aminoalcohol after reduction of the nitro-group, followed by cyclisation. Alternatively, the compounds may be prepared by epoxidation of an appropriate olefin, obtained by Wittig or Horner-Wadsworth-Emmons reactions from suitably protected 4-piperidone. Epoxide opening with ammonia or a primary amine, followed by cyclisation with a phosgene equivalent, alkylation of the carbamates if required and introduction of the desired piperidine substituent by alkylation or reductive amination leads to the target compound. Besides the preparation of enantiopure compound as described in herein, an enantioselective modification may include an asymmetric epoxidation method of the olefin as described in the literature, e.g. Jacobsen or others. Alternatively, Sharpless asymmetric dihydroxylation method may be used followed by epoxide ring formation. Alternatively, suitably protected 4-methoxycarbonyl-methylenepiperidine may be reduced to the allyl alcohol which is subjected to Sharpless asymmetric epoxidation according to literature procedure. Epoxide opening with a metallorganic reagent and oxidation of the resulting primary alcohol leads to the β-hydroxy carboxylic acid, which is converted into the desired spirocyclic enantiomer as described in herein. Alternatively, suitably protected 4-methoxycarbonyl-methylenepiperidine may be converted to an enantiomerically pure epoxide by Jacobsen epoxidation, followed by ring opening with ammonia or an appropriate amine, reaction with a metallorganic reagent with the ester group, reduction, cyclisation, alkylation if required and introduction of the piperidine substituent. Alternatively, the stereocenter may be introduced by using an appropriate α-amino acid ester as the chiral template. Reaction with allyl magnesium bromide, cyclisation, oxidative cleavage of the allyl groups, formation of the piperidine ring by reductive amination and final alkylation of the carbamate leads to the desired enantiomerically pure derivative.

Compounds of Formula III in which m″=0, Z₃=CH and X₄=N or NCH₂ may be prepared by a Strecker synthesis involving suitably protected 4-piperidone and an appropriate primary amine. Reaction of the resulting nitrile with a Grignard reagent gives the ketone, which is then subjected to a reductive amination. Cyclisation of the resulting diamine (after deprotection step if necessary), with phosgene or an equivalent thereof leads to the cyclic urea. Alkylation or reductive amination steps may be used to introduce N-piperidine substituents. In a similar way, the 6-membered analogue (X₄=NCH₂) may be obtained by using chloroacetyl chloride or a similar reagent for cyclisation (additional use of protecting groups may be necessary).

Compounds of Formula III in which m″=0, Z₃=N and X₄=CH₂ may be prepared as outlined herein.

Compounds of Formula III in which m″=0, Z₃=N and X₄=N(R) may be obtained according to literature methods (Gstach et al. Synthesis 803-808 (1990)) by treatment of a suitably protected 4-piperidone by reaction with a hydrazine derivative and reaction of the intermediate hydrazone with potassium cyanate. Alkylation and/or reductive amination after deprotection may be used to introduce the desired substituents.

Alternatively, compounds of Formula III in which X₄=S or SCH₂ or CH₂S may be obtained as described by routes described for the equivalent compounds bearing X₄=O, OCH₂ or CH₂O by transforming, prior to cyclisation, the appropriate hydroxyl group into a thiol using well known literature procedures (e.g. treatment with acetyl chloride and substitution with benzylthiol and conversion to the free mercaptane). Similarly, the hydroxyl group may be converted into the corresponding amines, which constitutes an alternative way to access some compounds of Formula III in which X₄=N(R), N(R)CH₂, CH₂(R).

Compounds of Formula III in which W₂=S may be prepared from the corresponding compounds in which W₂=O by treatment with e.g. the Lawesson reagent or bis(tricyclohexyltin)sulfide and BCl₃ or other sulfur-transferring reagents.

In general, during any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and T. W. Greene and P. G. M. Wuts, Protective Groups in Organic Chemistry, 3. Ed. John Wiley & Sons, 1999, and they should be chosen in such a way, that they are stable to the reaction conditions applied and readily removed at a convenient stage using methodology known from the art. Typical protecting groups are N-Boc, N-Cbz, N-Bn.

Pharmaceutical Compositions

Other embodiments includes the use of pharmaceutical compositions comprising a compound as described above, and a physiologically acceptable carrier, diluent, or excipient, or a combination thereof.

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, intramuscular, intraocular, intranasal, intravenous, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical compositions will generally be tailored to the specific intended route of administration.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990, which is hereby incorporated by reference in its entirety.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, intraocular injections or as an aerosol inhalant.

Alternately, one may administer the compound in a local rather than systemic manner, for example, via injection of the compound directly into the area of pain or inflammation, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes.

Pharmaceutical compositions for use in accordance with the present disclosure thus may be formulated in conventional manner using one or more physiologically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., as disclosed in Remington's Pharmaceutical Sciences, cited above.

For injection, the agents disclosed herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increases the solubility of the compounds to allow for the preparation of highly, concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds disclosed herein is a co-solvent system comprising benzyl alcohol, a nonpolar surfactant, a water-miscible organic polymer, and an aqueous phase. A common co-solvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of Polysorbate 80™; the fraction size of polyethylene glycol may be varied; and other biocompatible polymers may replace polyethylene glycol, e.g., polyvinyl pyrrolidone. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations disclosed herein may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acids or base forms.

Pharmaceutical compositions suitable for use in the methods disclosed herein include compositions where the active ingredients are contained in an amount effective to achieve its intended purpose. More specifically, a “therapeutically effective amount” means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions disclosed herein can be chosen by the individual physician in view of the patient's condition. (See e.g., Fingl et al. 1975, in “The Pharmacological Basis of Therapeutics”, Chapter 1, which is hereby incorporated by reference in its entirety). Typically, the dose range of the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Where no human dosage is established, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions disclosed herein or a pharmaceutically acceptable salt thereof calculated as the free base, the composition being administered 1 to 4 times per day. Alternatively the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. In some embodiments, the compounds will be administered for a period of continuous therapy, for example for a week or more, or for months or years.

Dosage amount and interval may be adjusted individually to provide plasma levels of the active moiety, which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen, which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

### EXAMPLES

The invention is disclosed in further detail in the following examples that are not in any way intended to limit the scope of the invention as claimed.

Chemical Synthesis

General procedures. ¹H NMR spectra were recorded at 400 MHz on a Varian Mercury-VX400 MHz spectrometer. ¹³C NMR spectra were measured at 100 MHz with proton decoupling at ambient temperature. Chemical shifts are given in δ-values [ppm] referenced to the residual solvent peak chloroform (CDCl₃) at 7.26 and methanol (CD₃OD) at 3.31 ppm. Unless otherwise stated, the NMR spectra of the compounds are described for their free amine form. Due to the presence of rotamers, two sets of signals are generally observed and rotamer ratios are reported. Where the corresponding signals for each of the two rotamers could unmistakably be identified, they are reported together [e.g. 4.66-4.58 and 3.76-3.68 (2m, 1H)].

Materials and solvents were of the highest grade available from commercial sources and used without further purification. Acidic ion-exchange solid phase extraction (SPE) cartridges were MEGA BE-SCX from Varian.

General LC-MS Procedure for Examples 1-41:

All spectra were obtained using an HP1100 LC/MSD-instrument. A setup with a binary pump, autosampler, column oven, diode array detecter, and electrospray ionization interface was used. A reversed phase column (C18 Luna 3 mm particle size, 7.5 cm×4.6 mm ID) with a guard cartridge system was used. The column was maintained at a temperature of 30° C. The mobile phase was acetonitrile/8 mM aqueous ammonium acetate. A 15 minute gradient program was used, starting at 70% acetonitrile, over 12 minutes to 95% acetonitrile, over 1 minute back to 70% acetonitrile, where it stayed for 2 minutes. The flow rate was 0.6 ml/min. The tᵣ values reported in the specific examples below were obtained using this procedure.

### Example 1

### N-((4-Methylphenyl)methyl)-N-(piperidin-4-yl)-N′-phenylmethylcarbamide (26HCH65)

To a solution of commercially available tert-butyl 4-oxo-1-piperidine carboxylate (1.75 g, 8.8 mmol) and 4-methylbenzylamine (970 mg, 8.0 mmol) in methanol (7 ml) was added acetic acid in methanol (1 M, 6.7 ml) followed by NaCNBH₃ in methanol (0.3 M, 30 ml). The resulting solution was stirred at room temperature. After 20 h, water (5 ml) was added, and the mixture was stirred for 1 h before it was concentrated. Flash chromatography in dichloromethane:methanol 10:1 gave tert-butyl 4-(4-methylphenyl)methyl)amino-piperidine carboxylate. Yield: 2.4 g, 98%. To a solution of tert-butyl 4-(4-methylphenyl)methyl)amino-piperidine carboxylate (800 mg, 2.63 mmol) in dry dichloromethane (20 ml) was added benzylisocyanate (0.65 ml, 5.26 mmol). The solution was stirred at room temperature. After 48 h, an excess of 2-dimethylaminoethylamine was added. The mixture was stirred for another 24 h, before it was concentrated. The resulting solid was redissolved in dichloromethane (20 ml), sequentially washed with HCl (0.2 N, 3×30 ml), and water (20 ml), dried (Na₂SO₄), filtered and concentrated. Flash chromatography in dichloromethane:methanol 10:1 gave N-((4-methylphenyl)methyl)-N-(1-(tert-butyloxycarbonyl)piperidin-4-yl)-N′-phenylmethylcarbamide (760 mg, 66%), which was dissolved in diethyl ether (5 ml). HCl (4 M) in dioxane (3 ml) was added, and the solution was stirred at room temperature for 60 min, then concentrated. The resulting oil was redissolved in a mixture of dichloromethane and diethyl ether (4:1). The organic layer was extracted with HCl (0.2 M, 3×20 ml). The combined aqueous layers were treated with NaOH (0.2 M) until basic (pH>8), then extracted with dichloromethane (3×20 ml). The combined organic layers were dried (Na₂SO₄), filtered, and concentrated to give the title compound. Yield: 406 mg, 70%; ¹³C-NMR (CDCl₃): δ 21.3, 31.6, 45.0, 45.9, 46.4, 53.0, 126.3, 127.2, 127.4, 128.6, 129.8, 135.3, 137.4, 139.7, 158.5.

### Example 2

### N-((4-Methylphenyl)methyl)-N-(1-(2-methylpropyl)piperidin-4-yl)-N′-phenylmethylcarbamide (26HCH66-02)

The product from example 1 above (20 mg, 0.06 mmol)was dissolved in abs. ethanol (2 ml). 2-Methylpropionaldehyde (0.08 ml, 0.6 mmol)was added followed by solid-supported borohydride (150 mg, 2.5 mmol/g resin; Aldrich 32,864-2). The mixture was shaken at room temperature. After 48 h, the resin was filtered off and acetic anhydride (0.02 ml, 0.2 mmol)was added to the organic solution. After 24 h, the mixture was concentrated and redissolved in methanol (2 ml). The solution was added on to a column carrying strongly acidic cation exchange resin (0.3 mmol/g resin), which was washed with methanol (3×6 ml), and eluted with 10% NH₃ in methanol, and concentrated to give the title compound. IR: 1640, 1185, 1110 cm⁻¹; LC-MS: (M+H)⁺ 394.2, tᵣ 5.60 min.

### Example 3

### N-(1-((2-Bromophenyl)methyl)piperidin-4-yl)-N-((4-methylphenyl)methyl)-N′-phenylmethylcarbamide (26HCH66-03)

The product from example 1 above (20 mg, 0.06 mmol) was dissolved in abs. ethanol (2 ml). 2-Bromobenzaldehyde (0.07 ml, 0.6 mmol) was added followed by solid-supported borohydride (150 mg, 2.5 mmol/g resin; Aldrich 32,864-2). The mixture was shaken at room temperature. After 48 h, the resin was filtered off and acetic anhydride (0.02 ml, 0.2 mmol) was added to the organic solution. After 24 h, the mixture was concentrated and redissolved in methanol (2 ml). The solution was added on to a column carrying strongly acidic cation exchange resin (0:3 mmol/g resin), which was washed with methanol (3×6 ml), and eluted with 10% NH₃ in methanol, and concentrated to give the title compound. IR: 1635, 1180, 1110 cm⁻¹; LC-MS: (M+H)⁺ 506.1, tᵣ 8.37 min.

### Example 4

### N-(1-((4-Hydroxy-3-methoxyphenyl)methyl)piperidin-4-yl)-N-((4-methylphenyl)methyl)-N′-phenylmethylcarbamide (26HCH66-04)

The product from example 1 above (20 mg, 0.06 mmol) was dissolved in abs. ethanol (2 ml). 4-Hydroxy-3-methoxybenzaldehyde (91 mg, 0.6 mmol) was added followed by solid-supported borohydride (150 mg, 2.5 mmol/g resin; Aldrich 32,864-2). The mixture was shaken at room temperature. After 48 h, the resin was filtered off and acetic anhydride (0.02 ml, 0.2 mmol) was added to the organic solution. After 24 h, the mixture was concentrated and redissolved in methanol (2 ml). The solution was added on to a column carrying strongly acidic cation exchange resin (0.3 mmol/g resin), which was washed with methanol (3×6 ml), and eluted with 10% NH₃ in methanol, and concentrated to give the title compound. ¹³C-NMR (CD₃OD, selected): δ 19.9, 55.4, 126.5, 127.0, 128.1, 129.0, 140.3, 148.0, 148.1, 158.8.

### Example 5

### N-(1-((5-Ethylthien-2-yl)methyl)piperidin-4-yl)-N-((4-methylphenyl)methyl)-N′-phenylmethylcarbamide (26HCH66-05)

The product from example 1 above (20 mg, 0.06 mmol) was dissolved in abs. ethanol (2 ml). 5-Ethyl-2-thiophencarboxaldehyde (84 mg, 0.6 mmol) was added followed by solid-supported borohydride (150 mg, 2.5 mmol/g resin; Aldrich 32,864-2). The mixture was shaken at room temperature. After 48 h, the resin was filtered off and acetic anhydride (0.02 ml, 0.2 mmol) was added to the organic solution. After 24 h, the mixture was concentrated and redissolved in methanol (2 ml). The solution was added on to a column carrying strongly acidic cation exchange resin (0.3 mmol/g resin), which was washed with methanol (3×6 ml), and eluted with 10% NH₃ in methanol, and concentrated to give the title compound. IR: 1640, 1185, 1110, 805, 700, 620 cm⁻¹; LC-MS: (M+H)⁺ 462.3, tᵣ 7.52 min.

### Example 6

### N-(1-(Imidazol-2-ylmethyl)piperidin-4-yl)-N-((4-methylphenyl)methyl)-N′-phenylmethylcarbamide (26HCH66-06)

The product from example 1 above (20 mg, 0.06 mmol) was dissolved in abs. ethanol (2 ml). Imidazole-2-carboxaldehyde (58 mg, 0.6 mmol) was added followed by solid-supported borohydride (150 mg, 2.5 mmol/g resin; Aldrich 32,864-2). The mixture was shaken at room temperature. After 48 h, the resin was filtered off and acetic anhydride (0.02 ml, 0.2 mmol) was added to the organic solution. After 24 h, the mixture was concentrated and redissolved in methanol (2 ml). The solution was added on to a column carrying strongly acidic cation exchange resin (0.3 mmol/g resin), which was washed with methanol (3×6 ml), and eluted with 10% NH₃ in methanol, and concentrated to give the title compound. IR: 1620, 1190, 1100, 805, 700, 620 cm⁻¹; LC-MS: (M+H)⁺ 418.2, tᵣ 2.05 min.

### Example 7

### N-(1-(Cyclohexylmethyl)piperidin-4-yl)-N-((4-methylphenyl)methyl)-N′-phenylmethylcarbamide (26HCH66-09)

The product from example 1 above (20 mg, 0.06 mmol) was dissolved in abs. ethanol (2 ml). Cyclohexanecarboxaldehyde (67 mg, 0.6 mmol) was added followed by solid-supported borohydride (150 mg, 2.5 mmol/g resin; Aldrich 32,864-2). The mixture was shaken at room temperature. After 48 h, the resin was filtered off and acetic anhydride (0.02 ml, 0.2 mmol) was added to the organic solution. After 24 h, the mixture was concentrated and redissolved in methanol (2 ml). The solution was added on to a column carrying strongly acidic cation exchange resin (0.3 mmol/g resin), which was washed with methanol (3×6 ml), and eluted with 10% NH₃ in methanol, and concentrated to give the title compound. IR: 1635, 1175, 1100, 805, 695, 620 cm⁻¹; LC-MS: (M+H)⁺ 434.4, tᵣ 7.44 min.

### Example 8

### N-(1-((4-Fluorophenyl)methyl)piperidin-4-yl)-N-((4-methylphenyl)methyl)-N′-phenylmethylcarbamide (26HCH66-10)

The product from example 1 above (20 mg, 0.06 mmol) was dissolved in abs. ethanol (2 ml). 4-Fluorobenzaldehyde (0.08 ml, 0.6 mmol) was added followed by solid-supported borohydride (150 mg, 2.5 mmol/g resin; Aldrich 32,864-2). The mixture was shaken at room temperature. After 48 h, the resin was filtered off and acetic anhydride (0.02 ml, 0.2 mmol) was added to the organic solution. After 24 h, the mixture was concentrated and redissolved in methanol (2 ml). The solution was added on to a column carrying strongly acidic cation exchange resin (0.3 mmol/g resin), which was washed with methanol (3×6 ml), and eluted with 10% NH₃ in methanol, and concentrated to give the title compound. IR: 1640, 1175, 1110, 805, 700, 620 cm⁻¹; LC-MS: (M+H)⁺ 446.3, tᵣ 5.62 min.

### Example 9

### N-((4-Methylphenyl)methyl)-N-(1-(phenylmethyl)piperidin-4-yl)-N′-phenylmethylcarbamide hydrochloride (26HCH16D)

To a solution of 1-benzyl-4-piperidone (1.74 g, 9.2 mmol) and 4-methylbenzylamine (0.97 g, 8 mmol) in methanol (30 ml) was added sodium borohydride (525 mg) in small portions over 30 min. The reaction mixture was stirred at room temperature. After 16 h, the mixture was concentrated. Water (30 ml) was added, and the mixture was extracted with dichloromethane (2×20 ml). The combined organic layers were dried (Na₂SO₄), filtered, and concentrated to give 4-((4-methylphenyl)methyl)amino-1-phenylmethylpiperidine. The crude product was used without further purification.

4-((4-Methylphenyl)methyl)amino-1-phenylmethylpiperidine (800 mg, 2.7 mmol) was dissolved in dry dichloromethane (30 ml). Benzylisocyanate (543 mg, 4.1 mmol) was added. The reaction mixture was stirred at room temperature. After 16 h, water (10 ml) was added followed by NaOH (6 N, 2 ml). After additional 30 minutes of stirring the white crystals were filtered off. The organic layer was isolated and dried (Na₂SO₄), filtered, and concentrated. Flash chromatography in dichloromethane/methanol 10/1 left N-((4-Methylphenyl)methyl)-N-(1-(phenylmethyl)piperidin-4-yl)-N′-phenylmethylcarbamide Yield: 820 mg, 71%; A sample was concentrated with HCl (4 M in dioxane)followed by recrystallization from dichloromethane/diethyl ether leaving the title compound. ¹H-NMR (CDCl₃): δ 1.87 (brd, 2H), 2.30 (s, 3H), 2.59 (dq, 2H), 2.76 (br q, 2H), 3.44 (br d, 2H), 4.09 (d, 2H), 4.30 (d, 2H), 4.40 (s, 2H), 4.64-4.76 (m, 2H), 6.98-7.64 (Aromatic protons, 14H); ¹³C-NMR (CDCl₃): δ 21.2, 26.7, 45.0, 46.0, 49.7, 52.2, 61.0, 126.2, 127.26, 126.31, 128.2, 128.6, 129.6, 129.9, 130.4, 131.6, 134.4, 137.6, 139.3, 158.5; ¹³C-NMR (CD₃OD, rotamers): δ 19.8, 26.4, 27.8, 40.3, 44.3, 51.6, 51.9, 54.5, 60.5, 110.0, 112.1, 114.0, 114.2, 117.5, 125.9, 126.2, 126.7, 126.8, 128.9, 129.1, 129.2, 129.4, 129.7, 130.1, 131.2, 134.5, 137.4, 159.1, 173.8, 175.0; Mp. 109-112° C.; Elemental analysis: Found C. 70.06; H, 7.62; N, 8.60; calcd for monohydrate: C, 69.76; H, 7.53; N, 8.72.

### Example 10

### N-((4-Methylphenyl)methyl)-N-(1-(phenylmethyl)piperidin-4-yl)-N′-phenylmethylcarbamide oxalate (34JJ59oxal)

N-((4-Methylphenyl)methyl)-N-(1-(phenylmethyl)piperidin-4-yl)-N′-phenylmethylcarbamide was prepared as described in example 9 above. A sample was precipitated as the oxalate and recrystallized from ethyl acetate to give the title compound.

¹³C-NMR (CDCl₃): δ 21.2, 27.0, 45.0, 45.9, 49.9, 52.1, 60.6, 126.1, 127.3, 127.4, 128.5, 128.7, 129.6, 130.0, 130.4, 131.2, 134.3, 137.7, 139.3, 158.4, 163.4; Mp. 180-182° C.; Elemental analysis: Found C. 69.54; H, 6.73; N, 7.96; calcd for monooxalate: C, 69.61; H, 6.82; N, 8.12.

### Example 11

### N-((4-Methylphenyl)methyl)-N-(1-(phenylmethyl)piperidin-4-yl)-4-methoxyphenylacetamide hydrochloride (26HCH17)

To a solution of 1-benzyl-4-piperidone (1.74 g, 9.2 mmol) and 4-methylbenzylamine (0.97 g, 8 mmol) in methanol (30 ml) was added sodium borohydride (525 mg) in small portions over 30 min. The reaction mixture was stirred at room temperature. After 16 h, the mixture was concentrated. Water (30 ml) was added, and the mixture was extracted with dichloromethane (2×20 ml). The combined organic layers were dried (Na₂SO₄), filtered, and concentrated to give 4-((4-methylphenyl)methyl)amino-1-phenylmethylpiperidine. The crude product was used without further purification.

To a solution of 4-((4-Methylphenyl)methyl)amino-1-phenylmethylpiperidine (800 mg, 2.7 mmol) in dry dichloromethane (30 ml) was added diisopropylethylamine (1.5 ml)followed by 4-methoxyphenylacetyl chloride (997 mg, 5.4 mmol). The reaction mixture was stirred at room temperature. After 16 h, the reaction mixture was concentrated, redissolved in diethyl ether, and extracted with HCl (0.6 N). The aqueous layer was isolated, treated with NaOH (1 N) until basic, and extracted with dichloromethane (20 ml). The organic layer was isolated and dried (Na₂SO₄), filtered, and concentrated, and redissolved in diethyl ether. The hydrochloride was formed by addition of HCl (4 M in dioxane), and recrystallized from diethyl ether to give the title compound. Yield: 600 mg, 50%; ¹H-NMR (CDCl₃): δ 1.75 (d, 2H), 2.32 (s, 3H), 2.50 (q, 2H), 2.70 (q, 2H), 3.38 (d, 2H), 3.54 (s, 2H), 3.78 (s, 3H), 4.06 (d, 2H), 4.54 (s, 2H), 4.82 (m, 1H), 6.78-7.60 (aromatic protons, 13H); ¹³C-NMR (CDCl₃): δ 21.0, 26.0, 40.3, 46.3, 49.0, 51.8, 55.3, 60.8, 114.2, 125.6, 126.6, 127.9, 129.4, 129.60, 129.62, 130.3, 131.4, 134.8, 137.1, 158.7, 172.9; Mp. 197-200° C.; Elemental analysis: Found C. 71.29; H, 7.25; N, 5.73; calcd for hydrate: C, 71.37; H, 7.43; N, 5.74.

### Example 12

### N-((4-Methylphenyl)methyl)-N-(1-(phenylmethyl)piperidin-4-yl)-4-methoxyphenylacetamide oxalate (34JJ61oxal)

N-((4-Methylphenyl)methyl)-N-(1-(phenylmethyl)piperidin-4-yl)-4-methoxyphenylacetamide was prepared as described in example 11 above. A sample was precipitated as the oxalate and recrystallized from tetrahydrofuran to give the title compound. ¹³C-NMR (CDCl₃): δ 21.2, 26.4, 40.6, 52.0, 55.5, 114.4, 125.9, 126.7, 128.4, 129.6, 129.8, 129.9, 130.4, 131.2, 134.6, 137.6, 158.9, 163.3, 172.9; Mp. 171-173° C.; Elemental analysis: Found C. 69.56; H, 6.74; N, 5.16; calcd for monooxalate: C, 69.48; H, 6.61; N, 5.40.

### Example 13

### N-((4-Methylphenyl)methyl)-N-(piperidin-4-yl)-4-methoxyphenylacetamide (26HCH71B)

To a solution of commercially available tert-butyl 4-oxo-1-piperidine carboxylate (1.75 g, 8.8 mmol) and 4-methylbenzylamine (970 mg, 8.0 mmol) in methanol (7 ml) was added acetic acid in methanol (1 M, 6.7 ml) followed by NaCNBH₃ in methanol (0.3 M, 30 ml). The resulting solution was stirred at room temperature. After 20 h, water (5 ml) was added, and the mixture was stirred for 1 h before it was concentrated. Flash chromatography in dichloromethane:methanol 10:1 gave tert-butyl 4-(4-methylphenyl)methyl)amino-piperidine carboxylate. Yield: 2.4 g, 98%. To a solution of tert-butyl 4-(4-methylphenyl)methyl)amino-piperidine carboxylate (862 mg, 2.83 mmol) in dry dichloromethane (10 ml) was added diisopropylethylamine (1.1 ml, 6.5 mmol) followed by 4-methoxyphenylacetyl chloride (0.66 ml, 4.3 mmol). The reaction mixture was stirred at room temperature. After 48 h, water (5 ml) was added, and the mixture was stirred for additional 2 h before extracted with NaOH (0.2 N, 2×15 ml), HCl (0.2 N, 2×15 ml), and water (15 ml). The organic layer was dried (Na₂SO₄) and concentrated to give N-((4-methylphenyl)methyl)-N-(1-(tert-butyloxycarbonyl)piperidin-4-yl)-4-methoxyphenylacetamide. The crude product was used without any further purification. N-((4-Methylphenyl)methyl)-N-(1-(tert-butyloxycarbonyl)piperidin-4-yl)-4-methoxyphenylacetamide was dissolved in ether (2 ml) and HCl (3 ml, 4 M in dioxane) was added. The reaction mixture was stirred at room temperature. After 2 h, water (5 ml) was added, and the mixture was extracted with HCl (0.1 N, 3×30 ml). The combined aqueous layers were treated with NaOH (0.2 N) until basic (pH>8). The aqueous layer was extracted with diethyl ether (2×20 ml). The combined organic layers were dried (Na₂SO₄) and concentrated, before dissolved in methanol (2 ml). The solution was added on to a column carrying strongly acidic cation exchange resin (0.3 mmol/g resin), which was washed with methanol (3×6 ml), and eluted with 10% NH₃ in methanol, and concentrated. Additional flash chromatography in dichloromethane/methanol 1/1.fwdarw.methanol containing 2% NH₃ gave the title compound. Yield: 466 mg, 47%; ¹³C-NMR (CD₃OD, rotamers): δ 19.9, 27.8, 29.7, 40.2, 40.3, 44.4, 44.45, 44.50, 52.4, 54.5, 55.5, 114.0, 114.1, 126.0, 126.7, 126.9, 127.3, 128.7, 129.3, 129.6, 129.7, 135.1, 136.1, 136.2, 137.1, 159.0, 159.1, 173.1, 173.7.

### Example 14

### N-(1-(3,3-Dimethylbutyl)piperidin-4-yl)-N-((4-methylphenyl)methyl)-4-methoxyphenylacetamide (26HCH79-5)

The product from example 13 above (20 mg, 0.06 mmol) was dissolved in abs. ethanol (2 ml). 3,3-Dimethylbutyraldehyde (0.143 ml, 1.1 mmol) was added followed by solid-supported borohydride (150 mg, 2.5 mmol/g resin; Aldrich 32,864-2). The mixture was shaken at room temperature. After 48 h, the resin was filtered off and acetic anhydride (0.02 ml, 0.2 mmol) was added to the organic solution. After 24 h, the mixture was concentrated and redissolved in methanol (2 ml). The solution was added on to a column carrying strongly acidic cation exchange resin (0.3 mmol/g resin), which was washed with methanol (3×6 ml), and eluted with 10% NH₃ in methanol, and concentrated to give the title compound. Yield: 26 mg; ¹³C-NMR (CD₃OD, rotamers): δ 19.9, 27.4, 28.4, 28.8, 29.2, 29.3, 38.3, 38.4, 40.2, 40.3, 44.3, 52.0, 52.3, 52.4, 53.9, 54.6, 54.9, 114.0, 114.1, 126.0, 126.8, 127.0, 127.3, 128.8, 129.4, 129.8, 129.9, 135.0, 136.1, 136.3, 137.1, 158.96, 159.05, 173.2, 173.8.

### Example 15

### N-(1-(Cyclohexylmethyl)piperidin-4-yl)-N-((4-methylphenyl)methyl)-4-methoxy phenylacetamide (26HCH79-6)

The product from example 13 above (20 mg, 0.06 mmol) was dissolved in abs. ethanol (2 ml). Cyclohexanecarboxaldehyde (0.138 ml, 1.1 mmol) was added followed by solid-supported borohydride (150 mg, 2.5 mmol/g resin; Aldrich 32,864-2). The mixture was shaken at room temperature. After 48 h, the resin was filtered off and acetic anhydride (0.02 ml, 0.2 mmol) was added to the organic solution. After 24 h, the mixture was concentrated and redissolved in methanol (2 ml). The solution was added on to a column carrying strongly acidic cation exchange resin (0.3 mmol/g resin), which was washed with methanol (3×6 ml), and eluted with 10% NH₃ in methanol, and concentrated to give the title compound. Yield: 17 mg; HRMS (FAB⁺, NBA) (M+H)⁺ 449.3163, C₂₉H₄₁N₂O₂ requires 449.3168; LC-MS: (M+H)⁺ 449.2, tᵣ 7.92 min.

### Example 16

### N-((4-Methylphenyl)methyl)-N-(1-(2-methylpropyl)piperidin-4-yl)-4-methoxyphenylacetamide (26HCH79-7)

The product from example 13 above (20 mg, 0.06 mmol) was dissolved in abs. ethanol (2 ml). 2-Methylpropionaldehyde (0.104 ml, 1.1 mmol) was added followed by solid-supported borohydride (150 mg, 2.5 mmol/g resin; Aldrich 32,864-2). The mixture was shaken at room temperature. After 48 h, the resin was filtered off and acetic anhydride (0.02 ml, 0.2 mmol) was added to the organic solution. After 24 h, the mixture was concentrated and redissolved in methanol (2 ml). The solution was added on to a column carrying strongly acidic cation exchange resin (0.3 mmol/g resin), which was washed with methanol (3×6 ml), and eluted with 10% NH₃ in methanol, and concentrated to give the title compound. Yield: 19 mg; HRMS (FAB⁺, NBA) (M+H)⁺ 09.2858, C₂₆H₃₇N₂O₂ requires 409.2855; LC-MS: (M+H)⁺ 409.2, tᵣ 5.97 min.

### Example 17

### N-((4-Methylphenyl)methyl)-N-(1-((4-methylphenyl)methyl)piperidin-4-yl)-4-methoxyphenylacetamide (26HCH79-8)

The product from example 13 above (20 mg, 0.06 mmol) was dissolved in abs. ethanol (2 ml). 4-Methylbenzaldehyde (0.134 ml, 1.1 mmol) was added followed by solid-supported borohydride (150 mg, 2.5 mmol/g resin; Aldrich 32,864-2). The mixture was shaken at room temperature. After 48 h, the resin was filtered off and acetic anhydride (0.02 ml, 0.2 mmol) was added to the organic solution. After 24 h, the mixture was concentrated and redissolved in methanol (2 ml). The solution was added on to a column carrying strongly acidic cation exchange resin (0.3 mmol/g resin), which was washed with methanol (3×6 ml), and eluted with 10% NH₃ in methanol, and concentrated to give the title compound. Yield: 22 mg; HRMS (FAB⁺, NBA) (M+H)⁺ 457.2853, C₃₀H₃₇N₂O₂ requires 457.2855; LC-MS: (M+H)⁺ 457.2, tᵣ 6.97 min.

### Example 18

### N-(1-((4-Hydroxyphenyl)methyl)piperidin-4-yl)-N-((4-methylphenyl)methyl)-4-methoxyphenylacetamide (26HCH79-9)

The product from example 13 above (20 mg, 0.06 mmol) was dissolved in abs. ethanol (2 ml). 4-Hydroxybenzaldehyde (139 mg, 1.1 mmol) was added followed by solid-supported borohydride (150 mg, 2.5 mmol/g resin; Aldrich 32,864-2). The mixture was shaken at room temperature. After 48 h, the resin was filtered off and acetic anhydride (0.02 ml, 0.2 mmol) was added to the organic solution. After 24 h, the mixture was concentrated and redissolved in methanol (2 ml). The solution was added on to a column carrying strongly acidic cation exchange resin (0.3 mmol/g resin), which was washed with methanol (3×6 ml), and eluted with 10% NH₃ in methanol, and concentrated to give the title compound. Yield: 19 mg; HRMS (FAB⁺, NBA) (M+H)⁺ 459.2655, C₂₉H₃₅N₂O₃ requires 459.2648; LC-MS: (M+H)⁺ 459.2, tᵣ 2.84 min.

### Example 19

### N-(1-((2-Hydroxyphenyl)methyl)piperidin-4-yl)-N-((4-methylphenyl)methyl)-4-methoxyphenylacetamide (26HCH79-10)

The product from example 13 above (20 mg, 0.06 mmol) was dissolved in abs. ethanol (2 ml). 2-Hydroxybenzaldehyde (0.122 ml, 1.1 mmol) was added followed by solid-supported borohydride (150 mg, 2.5 mmol/g resin; Aldrich 32,864-2). The mixture was shaken at room temperature. After 48 h, the resin was filtered off and acetic anhydride (0.02 ml, 0.2 mmol) was added to the organic solution. After 24 h, the mixture was concentrated and redissolved in methanol (2 ml). The solution was added on to a column carrying strongly acidic cation exchange resin (0.3 mmol/g resin), which was washed with methanol (3×6 ml), and eluted with 10% NH₃ in methanol, and concentrated to give the title compound. Yield: 16 mg; HRMS (FAB⁺, NBA) (M+H)⁺ 459.2633, C₂₉H₃₅N₂O₃ requires 459.2648; LC-MS: (M+H)⁺ 459.2, tᵣ 5.81 min.

### Example 20

### N-(3-Phenylpropyl)-N-(piperidin-4-yl)-4-methoxyphenylacetamide (26HCH80-1)

To a solution of commercially available tert-butyl 4-oxo-1-piperidine carboxylate (400 mg, 2 mmol) in methanol (1 ml) and 3-phenylpropylamine (0.143 ml, 1 mmol) in methanol (1 ml) was added acetic acid in methanol (1 M, 1.34 ml) followed by NaCNBH₃ in methanol (0.3 M, 4.4 ml). The resulting solution was stirred at room temperature. After 24 h, water (2 ml) was added, and the mixture was stirred for 1 h, before it was concentrated. The resulting oil was redissolved in diethyl ether (20 ml), extracted with HCl (0.1 N, 1×15 ml). The aqueous layer was washed with diethyl ether (10 ml) and treated with 0.2 N NaOH until basic (pH>8), before extracted with dichloromethane (20 ml). The organic layer was dried (Na₂SO₄), filtered, and concentrated to give tert-butyl 4-(3-phenylpropyl)amino-piperidine carboxylate. Yield: 110 mg. To a solution of tert-butyl 4-(3-phenylpropyl)amino-piperidine carboxylate (50 mg, 0.16 mmol) in dichloromethane (6 ml) was added diisopropylethylamine (0.070 ml, 0.4 mmol) followed by 4-methoxyphenylacetyl chloride (0.055 ml, 0.35 mmol). The reaction mixture was stirred at room temperature. After 18 h, water (2 ml) was added. The mixture was stirred for another 2 h. The mixture was sequentially washed with HCl (0.2 N, 2×15 ml), NaOH (0.2 N, 2×15 ml), and water (10 ml), dried (Na₂SO₄), filtered and concentrated to give N-(3-phenylpropyl)-N-(1-(tert-butyloxycarbonyl)piperidin-4-yl)-4-methoxyphenylacetamide. The crude product was used without any further purification. N-(3-Phenylpropyl)-N-(1-(tert-butyloxycarbonyl)piperidin-4-yl)-4-methoxyphenylacetamide was dissolved in diethyl ether (2 ml) and HCl (1 ml, 4 M in dioxane) was added. The reaction mixture was stirred at room temperature. After 2.5 h, NaOH (1 ml, 6 N) was added followed by dichloromethane (10 ml). The mixture was extracted with water (2×15 ml), dried (Na₂SO₄), filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 61 mg; ¹³C-NMR (CD₃OD, rotamers): δ 27.8, 29.4, 30.8, 32.3, 32.7, 33.3, 40.2, 40.5, 42.0, 44.5, 44.6, 44.9, 52.7, 54.56, 54.57, 54.9, 114.0, 114.1, 125.7, 126.1, 127.0, 127.4, 128.2, 128.3, 128.5, 129.47, 129.55, 141.2, 141.8, 158.9, 159.0, 172.5, 172.7.

### Example 21

### N-(2-Phenylethyl)-N-(piperidin-4-yl)-4-methoxyphenylmethylacetamide (26HCH80-2)

To a solution of commercially available tert-butyl 4-oxo-1-piperidine carboxylate (400 mg, 2 mmol) in methanol (1 ml) and 2-phenylethylamine (0.143 ml, 1 mmol) in methanol (1 ml) was added acetic acid in methanol (1 M, 1.34 ml) followed by NaCNBH₃ in methanol (0.3 M, 4.4 ml). The resulting solution was stirred at room temperature. After 24 h, water (2 ml) was added, and the mixture was stirred for 1 h, before it was concentrated. The resulting oil was redissolved in diethyl ether (20 ml), extracted with HCl (0.1 N, 1×15 ml). The aqueous layer was washed with diethyl ether (10 ml) and treated with 0.2 N NaOH until basic (pH>8), before extracted with dichloromethane (20 ml). The organic layer was dried (Na₂SO₄), filtered, and concentrated to give tert-butyl 4-(2-phenylethyl)amino-piperidine carboxylate. Yield: 221 mg. To a solution of tert-butyl 4-(2-phenylethyl)amino-piperidine carboxylate (50 mg, 0.16 mmol) in dichloromethane (6 ml) was added diisopropylethylamine (0.070 ml, 0.4 mmol) followed by 4-methoxyphenylacetyl chloride (0.055 ml, 0.35 mmol). The reaction mixture was stirred at room temperature. After 18 h, water (2 ml) was added. The mixture was stirred for another 2 h. The mixture was sequentially washed with HCl (0.2 N, 2×15 ml), NaOH (0.2 N, 2×15 ml), and water (10 ml), dried (Na₂SO₄), filtered and concentrated to give N-(2-phenylethyl)-N-(1-(tert-butyloxycarbonyl)piperidin-4-yl)-4-methoxyphenylacetamide. The crude product was used without any further purification. N-(2-Phenylethyl)-N-(1-(tert-butyloxycarbonyl)piperidin-4-yl)-4-methoxyphenylacetamide was dissolved in diethyl ether (2 ml) and HCl (1 ml, 4 M in dioxane) was added. The reaction mixture was stirred at room temperature. After 2.5 h, NaOH (1 ml, 6 N) was added followed by dichloromethane (10 ml). The mixture was extracted with water (2×15 ml), dried (Na₂SO₄), filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 52 mg; ¹³C-NMR (CD₃OD, rotamers): δ 27.1, 28.5, 34.9, 36.6, 40.2, 40.4, 44.1, 44.2, 44.4, 53.3, 54.2, 54.6, 114.0, 114.1, 126.2, 126.6, 127.2, 127.4, 128.3, 128.6, 128.79, 128.82, 129.7, 138.5, 139.5, 158.96, 159.0, 172.7, 173.1

### Example 22

### N-((2-Methoxyphenyl)methyl)-N-(piperidin-4-yl)-4-methoxyphenylmethylacetamide (26HCH80-4)

To a solution of commercially available tert-butyl 4-oxo-1-piperidine carboxylate (400 mg, 2 mmol) in methanol (1 ml) and 2-methoxybenzylamine (0.130 ml, 1 mmol) in methanol (1 ml) was added acetic acid in methanol (1 M, 1.34 ml) followed by NaCNBH₃ in methanol (0.3 M, 4.4 ml). The resulting solution was stirred at room temperature. After 24 h, water (2 ml) was added, and the mixture was stirred for 1 h, before it was concentrated. The resulting oil was redissolved in diethyl ether (20 ml), extracted with HCl (0.1 N, 1×15 ml). The aqueous layer was washed with diethyl ether (10 ml) and treated with 0.2 N NaOH until basic (pH>8), before extracted with dichloromethane (20 ml). The organic layer was dried (Na₂SO₄), filtered, and concentrated to give tert-butyl 4-((2-methoxyphenyl)methyl)amino-piperidine carboxylate. Yield: 211 mg. To a solution of tert-butyl 4-((2-methoxyphenyl)methyl)amino-piperidine carboxylate (50 mg, 0.16 mmol) in dichloromethane (6 ml) was added diisopropylethylamine (0.070 ml, 0.4 mmol) followed by 4-methoxyphenylacetyl chloride (0.055 ml, 0.35 mmol). The reaction mixture was stirred at room temperature. After 18 h, water (2 ml) was added. The mixture was stirred for another 2 h. The mixture was sequentially washed with HCl (0.2 N, 2×15 ml), NaOH (0.2 N, 2×15 ml), and water (10 ml), dried (Na₂SO₄), filtered and concentrated to give N-((2-methoxyphenyl)methyl)-N-(1-(tert-butyloxycarbonyl)piperidin-4-yl)-4-methoxyphenylacetamide. The crude product was used without any further purification. N-((2-Methoxyphenyl)methyl)-N-(1-(tert-butyloxycarbonyl)piperidin-4-yl)-4-methoxyphenylacetamide was dissolved in diethyl ether (2 ml) and HCl (1 ml, 4 M in dioxane) was added. The reaction mixture was stirred at room temperature. After 2.5 h, NaOH (1 ml, 6 N) was added followed by dichloromethane (10 ml). The mixture was extracted with water (2×15 ml), dried (Na₂SO₄), filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 40 mg; ¹³C-NMR (CD₃OD, rotamers): δ 26.1, 27.4, 40.0, 40.1, 43.5, 43.9, 51.5, 53.4, 54.5, 54.58, 54.63, 54.78, 54.83, 110.1, 110.5, 113.76, 113.78, 113.84, 114.0, 114.1, 120.1, 120.5, 125.4, 126.0, 126.5, 126.7, 127.1, 127.3, 127.7, 128.8, 129.8, 130.0, 130.08, 130.14, 156.5, 157.0, 159.0, 159.1, 173.2, 173.8.

### Example 23

### N-((2-Chlorophenyl)methyl)-N-(piperidin-4-yl)-4-methoxyphenylacetamide (26HCH80-5)

To a solution of commercially available tert-butyl 4-oxo-1-piperidine carboxylate (400 mg, 2 mmol) in methanol (1 ml) and 2-chlorobenzylamine (0.121 ml, 1 mmol) in methanol (1 ml) was added acetic acid in methanol (1 M, 1.34 ml) followed by NaCNBH₃ in methanol (0.3 M, 4.4 ml). The resulting solution was stirred at room temperature. After 24 h, water (2 ml) was added, and the mixture was stirred for 1 h before it was concentrated. The resulting oil was redissolved in diethyl ether (20 ml), extracted with HCl (0.1 N, 1×15 ml). The aqueous layer was washed with diethyl ether (10 ml) and treated with 0.2 N NaOH until basic (pH>8), before extracted with dichloromethane (20 ml). The organic layer was dried (Na₂SO₄), filtered, and concentrated to give tert-butyl 4-((2-chlorophenyl)methyl)amino-piperidine carboxylate. Yield: 137 mg. To a solution of tert-butyl 4-((2-chlorophenyl)methyl)amino-piperidine carboxylate (50 mg, 0.15 mmol) in dichloromethane (6 ml) was added diisopropylethylamine (0.070 ml, 0.4 mmol) followed by 4-methoxyphenylacetyl chloride (0.055 ml, 0.35 mmol). The reaction mixture was stirred at room temperature. After 18 h, water (2 ml) was added. The mixture was stirred for another 2 h. The mixture was sequentially washed with HCl (0.2 N, 2×15 ml), NaOH (0.2 N, 2×15 ml), and water (10 ml), dried (Na₂SO₄), filtered and concentrated to give N-((2-chlorophenyl)methyl)-N-(1-(tert-butyloxycarbonyl)piperidin-4-yl)-4-methoxyphenylacetamide. The crude product was used without any further purification. N-((2-Chlorophenyl)methyl)-N-(1-(tert-butyloxycarbonyl)piperidin-4-yl)-4-methoxyphenylacetamide was dissolved in diethyl ether (2 ml) and HCl (1 ml, 4 M in dioxane) was added. The reaction mixture was stirred at room temperature. After 2.5 h, NaOH (1 ml, 6 N) was added followed by dichloromethane (10 ml). The mixture was extracted with water (2×15 ml), dried (Na₂SO₄), filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 45 mg; ¹³C-NMR (CD₃OD, rotamers): δ 25.8, 26.9, 40.0, 40.1, 42.9, 43.4, 43.7, 46.0, 51.1, 53.0, 54.6, 113.77, 113.84, 114.0, 114.1, 126.6, 126.8, 127.08, 127.13, 127.3, 127.4, 128.1, 129.0, 129.2, 129.8, 130.0, 130.2, 131.9, 132.2, 135.0, 135.3, 159.1, 173.4, 173.8.

### Example 24

### N-((3,4-Di-methoxyphenyl)methyl)-N-(piperidin-4-yl)-4-methoxyphenylacetamide (26HCH80-6)

To a solution of commercially available tert-butyl 4-oxo-1-piperidine carboxylate (400 mg, 2 mmol) in methanol (1 ml) and 3,4-di-methoxybenzylamine (0.151 ml, 1 mmol) in methanol (1 ml) was added acetic acid in methanol (1 M, 1.34 ml) followed by NaCNBH₃ in methanol (0.3 M, 4.4 ml). The resulting solution was stirred at room temperature. After 24 h, water (2 ml) was added, and the mixture was stirred for 1 h, before it was concentrated. The resulting oil was redissolved in diethyl ether (20 ml), extracted with HCl (0.1 N, 1×15 ml). The aqueous layer was washed with diethyl ether (10 ml) and treated with 0.2 N NaOH until basic (pH>8), before extracted with dichloromethane (20 ml). The organic layer was dried (Na₂SO₄), filtered, and concentrated to give tert-butyl 4-((3,4-di-methoxyphenyl)methyl)amino-piperidine carboxylate. Yield: 162 mg. To a solution of tert-butyl 4-((3,4-di-methoxyphenyl)methyl)amino-piperidine carboxylate (50 mg, 0.14 mmol) in dichloromethane (6 ml) was added diisopropylethylamine (0.070 ml, 0.4 mmol) followed by 4-methoxyphenylacetyl chloride (0.055 ml, 0.35 mmol). The reaction mixture was stirred at room temperature. After 18 h, water (2 ml) was added. The mixture was stirred for another 2 h. The mixture was sequentially washed with HCl (0.2 N, 2×15 ml), NaOH (0.2 N, 2×15 ml), and water (10 ml), dried (Na₂SO₄), filtered and concentrated to give N-((3,4-di-methoxyphenyl)methyl)-N-(1-(tert-butyloxycarbonyl)piperidin-4-yl)-4-methoxyphenylacetamide. The crude product was used without any further purification. N-((3,4-Di-methoxyphenyl)methyl)-N-(1-(tert-butyloxycarbonyl)piperidin-4-yl)-4-methoxyphenylacetamide was dissolved in diethyl ether (2 ml) and HCl (1 ml, 4 M in dioxane) was added. The reaction mixture was stirred at room temperature. After 2.5 h, NaOH (1 ml, 6 N) was added followed by dichloromethane (10 ml). The mixture was extracted with water (2×15 ml), dried (Na₂SO₄), filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 54 mg; ¹³C-NMR (CD₃OD, rotamers): δ 25.9, 27.3, 40.0, 40.1, 43.5, 43.8, 44.1, 51.4, 53.5, 54.6, 55.4, 110.2, 111.0, 111.9, 112.2, 114.0, 114.2, 118.6, 119.4, 127.1, 127.4, 129.9, 130.0, 130.5, 132.1, 148.2, 148.7, 149.2, 149.7, 158.98, 159.05, 173.3, 173.6.

### Example 25

### N-((4-Fluorophenyl)methyl)-N-(piperidin-4-yl)-4-methoxyphenylacetamide (26HCH80-7)

To a solution of commercially available tert-butyl 4-oxo-1-piperidine carboxylate (400 mg, 2 mmol) in methanol (1 ml) and 4-fluorobenzylamine (0.114 ml, 1 mmol) in methanol (1 ml) was added acetic acid in methanol (1 M, 1.34 ml) followed by NaCNBH₃ in methanol (0.3 M, 4.4 ml). The resulting solution was stirred at room temperature. After 24 h, water (2 ml) was added, and the mixture was stirred for 1 h, before it was concentrated. The resulting oil was redissolved in diethyl ether (20 ml), extracted with HCl (0.1 N, 1×15 ml). The aqueous layer was washed with diethyl ether (10 ml) and treated with 0.2 N NaOH until basic (pH>8), before extracted with dichloromethane (20 ml). The organic layer was dried (Na₂SO₄), filtered, and concentrated to give tert-butyl 4-((4-fluorophenyl)methyl)amino-piperidine carboxylate. Yield: 130 mg. To a solution of tert-butyl 4-((4-fluorophenyl)methyl)amino-piperidine carboxylate (50 mg, 0.16 mmol) in dichloromethane (6 ml) was added diisopropylethylamine (0.070 ml, 0.4 mmol) followed by 4-methoxyphenylacetyl chloride (0.055 ml, 0.35 mmol). The reaction mixture was stirred at room temperature. After 18 h, water (2 ml) was added. The mixture was stirred for another 2 h. The mixture was sequentially washed with HCl (0.2 N, 2×15 ml), NaOH (0.2 N, 2×15 ml), and water (10 ml), dried (Na₂SO₄), filtered and concentrated to give N-((4-fluorophenyl)methyl)-N-(1-(tert-butyloxycarbonyl)piperidin-4-yl)-4-methoxyphenylacetamide. The crude product was used without any further purification. N-((4-Fluorophenyl)methyl)-N-(1-(tert-butyloxycarbonyl)piperidin-4-yl)-4-methoxyphenylacetamide was dissolved in diethyl ether (2 ml) and HCl (1 ml, 4 M in dioxane) was added. The reaction mixture was stirred at room temperature. After 2.5 h, NaOH (1 ml, 6 N) was added followed by dichloromethane (10 ml). The mixture was extracted with water (2×15 ml), dried (Na₂SO₄), filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 45 mg; ¹³C-NMR (CD₃OD, rotamers): δ 26.1, 27.5, 40.1, 43.6, 43.8, 44.0, 51.6, 53.6, 54.6, 113.77, 113.84, 114.0, 114.1, 114.7, 114.9, 115.3, 115.6, 126.8, 127.2, 128.1, 128.6, 128.7, 129.8, 130.0, 130.1, 130.6, 131.0, 133.8, 159.1, 173.3, 173.6.

### Example 26

### N-((2,4-Di-chlorophenyl)methyl)-N-(piperidin-4-yl)-4-methoxyphenylacetamide (26HCH80-8)

To a solution of commercially available tert-butyl 4-oxo-1-piperidine carboxylate (400 mg, 2 mmol) in methanol (1 ml) and 2,4-di-chlorobenzylamine (0.135 ml, 1 mmol) in methanol (1 ml) was added acetic acid in methanol (1 M, 1.34 ml) followed by NaCNBH₃ in methanol (0.3 M, 4.4 ml). The resulting solution was stirred at room temperature. After 24 h, water (2 ml) was added, and the mixture was stirred for 1 h, before it was concentrated. The resulting oil was redissolved in diethyl ether (20 ml), extracted with HCl (0.1 N, 1×15 ml). The aqueous layer was washed with diethyl ether (10 ml) and treated with 0.2 N NaOH until basic (pH>8), before extracted with dichloromethane (20 ml). The organic layer was dried (Na₂SO₄), filtered, and concentrated to give tert-butyl 4-((2,4-di-chlorophenyl)methyl)amino-piperidine carboxylate. Yield: 97 mg. To a solution of tert-butyl 4-((2,4-di-chlorophenyl)methyl)amino-piperidine carboxylate (50 mg, 0.14 mmol) in dichloromethane (6 ml) was added diisopropylethylamine (0.070 ml, 0.4 mmol) followed by 4-methoxyphenylacetyl chloride (0.055 ml, 0.35 mmol). The reaction mixture was stirred at room temperature. After 18 h, water (2 ml) was added. The mixture was stirred for another 2 h. The mixture was sequentially washed with HCl (0.2 N, 2×15 ml), NaOH (0.2 N, 2×15 ml), and water (10 ml), dried (Na₂SO₄), filtered and concentrated to give N-((2,4-di-chlorophenyl)methyl)-N-(1-(tert-butyloxycarbonyl)piperidin-4-yl)-4-methoxyphenylacetamide. The crude product was used without any further purification. N-((2,4-Di-chlorophenyl)methyl)-N-(1-(tert-butyloxycarbonyl)piperidin-4-yl)-4-methoxyphenylacetamide was dissolved in diethyl ether (2 ml) and HCl (1 ml, 4 M in dioxane) was added. The reaction mixture was stirred at room temperature. After 2.5 h, NaOH (1 ml, 6 N) was added followed by dichloromethane (10 ml). The mixture was extracted with water (2×15 ml), dried (Na₂SO₄), filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 39 mg; ¹³C-NMR (CD₃OD, rotamers): δ 25.7, 26.8, 40.0, 42.6, 43.3, 43.7, 51.2, 53.0, 54.5, 54.6, 113.8, 113.8, 114.0, 114.1, 127.0, 128.4, 128.8, 129.8, 130.0, 130.1, 131.0, 132.7, 132.9, 134.5, 159.1, 173.4, 173.6.

### Example 27

### N-((3-Methylphenyl)methyl)-N-(piperidin-4-yl)-4-methoxyphenylacetamide (26HCH80-9)

To a solution of commercially available tert-butyl 4-oxo-1-piperidine carboxylate (400 mg, 2 mmol) in methanol (1 ml) and 3-methylbenzylamine (0.125 ml, 1 mmol) in methanol (1 ml) was added acetic acid in methanol (1 M, 1.34 ml) followed by NaCNBH₃ in methanol (0.3 M, 4.4 ml). The resulting solution was stirred at room temperature. After 24 h, water (2 ml) was added, and the mixture was stirred for 1 h, before it was concentrated. The resulting oil was redissolved in diethyl ether (20 ml), extracted with HCl (0.1 N, 1×15 ml). The aqueous layer was washed with diethyl ether (10 ml) and treated with 0.2 N NaOH until basic (pH>8), before extracted with dichloromethane (20 ml). The organic layer was dried (Na₂SO₄), filtered, and concentrated to give tert-butyl 4-((3-methylphenyl)methyl)amino-piperidine carboxylate. Yield: 136 mg. To a solution of tert-butyl 4-((3-methylphenyl)methyl)amino-piperidine carboxylate (50 mg, 0.16 mmol) in dichloromethane (6 ml) was added diisopropylethylamine (0.070 ml, 0.4 mmol) followed by 4-methoxyphenylacetyl chloride (0.055 ml, 0.35 mmol). The reaction mixture was stirred at room temperature. After 18 h, water (2 ml) was added. The mixture was stirred for another 2 h. The mixture was sequentially washed with HCl (0.2 N, 2×15 ml), NaOH (0.2 N, 2×15 ml), and water (10 ml), dried (Na₂SO₄), filtered and concentrated to give N-((3-methylphenyl)methyl)-N-(1-(tert-butyloxycarbonyl)piperidin-4-yl)-4-methoxyphenylacetamide. The crude product was used without any further purification. N-((3-Methylphenyl)methyl)-N-(1-(tert-butyloxycarbonyl)piperidin-4-yl)-4-methoxyphenylacetamide was dissolved in diethyl ether (2 ml) and HCl (1 ml, 4 M in dioxane) was added. The reaction mixture was stirred at room temperature. After 2.5 h, NaOH (1 ml, 6 N) was added followed by dichloromethane (10 ml). The mixture was extracted with water (2×15 ml), dried (Na₂SO₄), filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 48 mg; ¹³C-NMR (CD₃OD, rotamers): δ 20.4, 26.8, 28.3, 40.2, 43.9, 44.1, 44.5, 51.8, 54.2, 54.57, 54.61, 114.0, 114.1, 123.2, 123.7, 126.7, 127.0, 127.1, 127.3, 128.0, 128.1, 128.7, 129.8, 129.9, 137.9, 138.6, 138.9, 159.0, 159.1, 173.1, 173.7.

### Example 28

### N-((3-Bromophenyl)methyl)-N-(piperidin-4-yl)-4-methoxyphenylacetamide (26HCH80-10)

To a solution of commercially available tert-butyl 4-oxo-1-piperidine carboxylate (400 mg, 2 mmol) in methanol (1 ml) and 3-bromobenzylamine hydrobromide (222 mg, 1 mmol) in methanol (1 ml) was added acetic acid in methanol (1 M, 1.34 ml) followed by NaCNBH₃ in methanol (0.3 M, 4.4 ml). The resulting solution was stirred at room temperature. After 24 h, water (2 ml) was added, and the mixture was stirred for 1 h, before it was concentrated. The resulting oil was redissolved in diethyl ether (20 ml), extracted with HCl (0.1 N, 1×15 ml). The aqueous layer was washed with diethyl ether (10 ml) and treated with 0.2 N NaOH until basic (pH>8), before extracted with dichloromethane (20 ml). The organic layer was dried (Na₂SO₄), filtered, and concentrated to give tert-butyl 4-((3-bromophenyl)methyl)amino-piperidine carboxylate. Yield: 142 mg. To a solution of tert-butyl 4-((3-bromophenyl)methyl)amino-piperidine carboxylate (50 mg, 0.14 mmol) in dichloromethane (6 ml) was added diisopropylethylamine (0.070 ml, 0.4 mmol) followed by 4-methoxyphenylacetyl chloride (0.055 ml, 0.35 mmol). The reaction mixture was stirred at room temperature. After 18 h, water (2 ml) was added. The mixture was stirred for another 2 h. The mixture was sequentially washed with HCl (0.2 N, 2×15 ml), NaOH (0.2 N, 2×15 ml), and water (10 ml), dried (Na₂SO₄), filtered and concentrated to give N-((3-bromophenyl)methyl)-N-(1-(tert-butyloxycarbonyl)piperidin-4-yl)-4-methoxyphenylacetamide. The crude product was used without any further purification. N-((3-Bromophenyl)methyl)-N-(1-(tert-butyloxycarbonyl)piperidin-4-yl)-4-methoxyphenylacetamide was dissolved in diethyl ether (2 ml) and HCl (1 ml, 4 M in dioxane) was added. The reaction mixture was stirred at room temperature. After 2.5 h, NaOH (1 ml, 6 N) was added followed by dichloromethane (10 ml). The mixture was extracted with water (2×15 ml), dried (Na₂SO₄), filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 49 mg; ¹³C-NMR (CD₃OD, rotamers): δ 26.6, 28.2, 40.2, 43.9, 44.0, 51.8, 54.1, 54.6, 113.76, 113.84, 114.1, 114.2, 122.2, 125.0, 125.5, 126.7, 127.1, 129.2, 129.5, 129.7, 129.8, 129.9, 130.0, 130.5, 130.6, 140.8, 141.8, 159.1, 173.3, 173.7.

### Example 29

### N-(1-(Phenylmethyl)piperidin-4-yl)-N-(3-phenyl-2-propen-1-yl)-4-methoxyphenylacetamide (26HCH76B)

To a solution of 4-amino-N-benzylpiperidine (200 mg, 1.05 mmol) in methanol (2 ml) was added trans-cinnamaldehyde (211 mg, 1.6 mmol), followed by Acetic acid in methanol (1 M, 1.4 ml) and sodiumcyanoborohydride in methanol (0.3 M, 4.4 ml). The reaction mixture was stirred at room temperature. After 48 h, water (2 ml) was added. The mixture was stirred for another 2 h before concentrated and redissolved in diethyl ether (20 ml). The organic layer was extracted with HCl (0.1 N, 2×10 ml). The combined aqueous layers were treated with NaOH (0.2 N) until basic (pH>8). The mixture was extracted with dichloromethane (2×10 ml). The combined organic layers were dried (Na₂SO₄) and concentrated. The crude product, which was used without any further purification, was dissolved in dichloromethane (5 ml). Diisopropylethylamine (284 mg, 2.1 eq.) was added, followed by 4-methoxyphenylacetyl chloride (387 mg, 2.0 eq). The reaction mixture was stirred at room temperature. After 18 h, water (2 ml) was added. After additional 2 h dichloromethane (10 ml) was added. The mixture was extracted with NaOH (0.2 N, 3×15 ml), and water (15 ml). The organic layer was dried (Na₂SO₄) and concentrated. The crude product was redissolved in methanol (2 ml) and added on to a column carrying strongly acidic cation exchange resin (0.3 mmol/g resin), which was washed with methanol (3×6 ml), and eluted with 10% NH₃ in methanol, and concentrated to give the title compound. ¹³C-NMR (CDCl₃): δ 28.5, 38.1, 46.6, 47.4, 50.9, 54.7, 62.9, 113.7, 125.5, 126.4, 126.6, 127.4, 127.9, 128.5, 128.6, 129.6, 130.0, 135.2, 135.3, 138.0, 158.2, 173.2.

### Example 30

### N-((4-Methylphenyl)methyl)-N-(1-piperidin-4-yl)-phenylacetamide (26HCH78-1)

To a solution of commercially available tert-butyl 4-oxo-1-piperidine carboxylate (1.75 g, 8.8 mmol) and 4-methylbenzylamine (970 mg, 8.0 mmol) in methanol (7 ml) was added acetic acid in methanol (1 M, 6.7 ml) followed by NaCNBH₃ in methanol (0.3 M, 30 ml). The resulting solution was stirred at room temperature. After 20 h, water (5 ml) was added, and the mixture was stirred for 1 h, before it was concentrated. Flash chromatography in dichloromethane:methanol 10:1 gave tert-butyl 4-(4-methylphenyl)methyl)amino-piperidine carboxylate. Yield: 2.4 g, 98%. To a solution of tert-butyl 4-(4-methylphenyl)methyl)amino-piperidine carboxylate (80 mg, 0.26 mmol) in dichloromethane (1.8 ml) was added diisopropylethylamine (0.11 ml, 2.4 eq.) followed by phenylacetyl chloride (81 mg, 0.53 mmol). The reaction mixture was stirred at room temperature. After 20 h, water (1 ml) was added. The mixture was stirred for another 2 h, before diethyl ether (20 ml) was added. The mixture was sequentially extracted with HCl (0.2 N, 2×15 ml), NaOH (0.2 N, 2×15 ml), and H₂O (10 ml), dried (Na₂SO₄), filtered and concentrated. The crude material was dissolved in diethyl ether (2 ml) and HCl (4 M in dioxane, 1 ml). The reaction mixture was stirred at room temperature. After 2 h, NaOH (6 N, 1 ml) was added followed by dichloromethane (10 ml). The mixture was extracted with water (2×10 ml), dried (Na₂SO₄), and filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 38 mg; ¹³C-NMR (CD₃OD, rotamers): δ 19.9, 26.9, 28.4, 41.0, 41.1, 44.0, 44.1, 44.4, 51.9, 54.4, 126.1, 126.7, 126.8, 126.9, 128.5, 128.7, 128.78, 128.81, 128.9, 129.4, 129.5, 134.9, 135.2, 135.6, 136.0, 136.3, 137.2, 172.8, 173.3.

### Example 31

### N-((4-Methylphenyl)methyl)-N-(1-piperidin-4-yl)-3-phenylpropionamide (26HCH78-2)

To a solution of commercially available tert-butyl 4-oxo-1-piperidine carboxylate (1.75 g, 8.8 mmol) and 4-methylbenzylamine (970 mg, 8.0 mmol) in methanol (7 ml) was added acetic acid in methanol (1 M, 6.7 ml) followed by NaCNBH₃ in methanol (0.3 M, 30 ml). The resulting solution was stirred at room temperature. After 20 h, water (5 ml) was added, and the mixture was stirred for 1 h, before it was concentrated. Flash chromatography in dichloromethane:methanol 10:1 gave tert-butyl 4-(4-methylphenyl)methyl)amino-piperidine carboxylate. Yield: 2.4 g, 98%. To a solution of tert-butyl 4-(4-methylphenyl)methyl)amino-piperidine carboxylate (80 mg, 0.26 mmol) in dichloromethane (1.8 ml) was added diisopropylethylamine (0.11 ml, 2.4 eq.) followed by 3-phenylpropionyl chloride (0.078 ml, 0.53 mmol). The reaction mixture was stirred at room temperature. After 20 h, water (1 ml) was added. The mixture was stirred for another 2 h, before diethyl ether (20 ml) was added. The mixture was sequentially extracted with HCl (0.2 N. 2×15 ml), NaOH (0.2 N, 2×15 ml), and H₂O (10 ml), dried (Na₂SO₄), filtered and concentrated. The crude material was dissolved in diethyl ether (2 ml) and HCl (4 M in dioxane, 1 ml). The reaction mixture was stirred at room temperature. After 2 h, NaOH (6 N, 1 ml) was added followed by dichloromethane (10 ml). The mixture was extracted with water (2×10 ml), dried (Na₂SO₄), and filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 43 mg; ¹³C-NMR (CD₃OD, rotamers): δ 19.9, 27.4, 29.0, 31.4, 31.7, 34.7, 35.7, 44.2, 44.3, 51.6, 54.2, 125.9, 126.07, 126.15, 126.8, 128.3, 128.4, 128.7, 128.8, 129.3, 135.1, 136.1, 136.2, 137.0, 141.1, 141.2, 173.9, 174.4.

### Example 32

### N-((4-Methylphenyl)methyl)-N-(1-piperidin-4-yl)-(phenylthio)acetamide (26HCH78-3)

To a solution of commercially available tert-butyl 4-oxo-1-piperidine carboxylate (1.75 g, 8.8 mmol) and 4-methylbenzylamine (970 mg, 8.0 mmol) in methanol (7 ml) was added acetic acid in methanol (1 M, 6.7 ml) followed by NaCNBH₃ in methanol (0.3 M, 30 ml). The resulting solution was stirred at room temperature. After 20 h, water (5 ml) was added, and the mixture was stirred for 1 h, before it was concentrated. Flash chromatography in dichloromethane:methanol 10:1 gave tert-butyl 4-(4-methylphenyl)methyl)amino-piperidine carboxylate. Yield: 2.4 g, 98%. To a solution of tert-butyl 4-(4-methylphenyl)methyl)amino-piperidine carboxylate (80 mg, 0.26 mmol) in dichloromethane (1.8 ml) was added diisopropylethylamine (0.11 ml, 2.4 eq.) followed by (phenylthio)acetyl chloride (0.078 ml, 0.53 mmol). The reaction mixture was stirred at room temperature. After 20 h, water (1 ml) was added. The mixture was stirred for another 2 h, before diethyl ether (20 ml) was added. The mixture was sequentially extracted with HCl (0.2 N, 2×15 ml), NaOH (0.2 N, 2×15 ml), and H₂O (10 ml), dried (Na₂SO₄), filtered and concentrated. The crude material was dissolved in diethyl ether (2 ml) and HCl (4 M in dioxane, 1 ml). The reaction mixture was stirred at room temperature. After 2 h, NaOH (6 N, 1 ml) was added followed by dichloromethane (10 ml). The mixture was extracted with water (2×10 ml), dried (Na₂SO₄), and filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 18 mg; HRMS (FAB⁺, NBA) (M+H)⁺ 355.1841, C₂₁H₂₇N₂OS requires 355.1844; LC-MS: (M+H)⁺ 355.1, tᵣ 2.62 min.

### Example 33

### N-((4-Methylphenyl)methyl)-N-(1-piperidin-4-yl)-phenoxyacetamide (26HCH78-4)

To a solution of commercially available tert-butyl 4-oxo-1-piperidine carboxylate (1.75 g, 8.8 mmol) and 4-methylbenzylamine (970 mg, 8.0 mmol) in methanol (7 ml) was added acetic acid in methanol (1 M, 6.7 ml) followed by NaCNBH₃ in methanol (0.3 M, 30 ml). The resulting solution was stirred at room temperature. After 20 h, water (5 ml) was added, and the mixture was stirred for 1 h, before it was concentrated. Flash chromatography in dichloromethane:methanol 10:1 gave tert-butyl 4-(4-methylphenyl)methyl)amino-piperidine carboxylate. Yield: 2.4 g, 98%. To a solution of tert-butyl 4-(4-methylphenyl)methyl)amino-piperidine carboxylate (80 mg, 0.26 mmol) in dichloromethane (1.8 ml) was added diisopropylethylamine (0.11 ml, 2.4 eq.) followed by phenoxyacetyl chloride (0.073 ml, 0.53 mmol). The reaction mixture was stirred at room temperature. After 20 h, water (1 ml) was added. The mixture was stirred for another 2 h, before diethyl ether (20 ml) was added. The mixture was sequentially extracted with HCl (0.2 N, 2×15 ml), NaOH (0.2 N, 2×15 ml), and H₂O (10 ml), dried (Na₂SO₄), filtered and concentrated. The crude material was dissolved in diethyl ether (2 ml) and HCl (4 M in dioxane, 1 ml). The reaction mixture was stirred at room temperature. After 2 h, NaOH (6 N, 1 ml) was added followed by dichloromethane (10 ml). The mixture was extracted with water (2×10 ml), dried (Na₂SO₄), and filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 24 mg; ¹³C-NMR (CD₃OD, rotamers): δ 19.9, 25.8, 27.4, 43.5, 43.7, 44.4, 51.9, 52.3, 66.9, 114.7, 114.8, 116.7, 117.0, 121.4, 123.6, 126.3, 126.8, 128.4, 128.9, 129.3, 129.5, 129.6, 131.0, 134.4, 136.1, 137.4, 158.3, 169.8, 170.1.

### Example 34

### N-((4-Methylphenyl)methyl)-N-(1-piperidin-4-yl)-(4-chlorophenoxy)acetamide (26HCH78-5)

To a solution of commercially available tert-butyl 4-oxo-1-piperidine carboxylate (1.75 g, 8.8 mmol) and 4-methylbenzylamine (970 mg, 8.0 mmol) in methanol (7 ml) was added acetic acid in methanol (1 M, 6.7 ml) followed by NaCNBH₃ in methanol (0.3 M, 30 ml). The resulting solution was stirred at room temperature. After 20 h, water (5 ml) was added, and the mixture was stirred for 1 h, before it was concentrated. Flash chromatography in dichloromethane:methanol 10:1 gave tert-butyl 4-(4-methylphenyl)methyl)amino-piperidine carboxylate. Yield: 2.4 g, 98%. To a solution of tert-butyl 4-(4-methylphenyl)methyl)amino-piperidine carboxylate (80 mg, 0.26 mmol) in dichloromethane (1.8 ml) was added diisopropylethylamine (0.11 ml, 2.4 eq.) followed by 4-chlorophenoxyacetyl chloride (0.082 ml, 0.53 mmol). The reaction mixture was stirred at room temperature. After 20 h, water (1 ml) was added. The mixture was stirred for another 2 h, before diethyl ether (20 ml) was added. The mixture was sequentially extracted with HCl (0.2 N, 2×15 ml), NaOH (0.2 N, 2×15 ml), and H₂O (10 ml), dried (Na₂SO₄), filtered and concentrated. The crude material was dissolved in diethyl ether (2 ml) and HCl (4 M in dioxane, 1 ml). The reaction mixture was stirred at room temperature. After 2 h, NaOH (6 N, 1 ml) was added followed by dichloromethane (10 ml). The mixture was extracted with water (2×10 ml), dried (Na₂SO₄), and filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 21 mg; ¹³C-NMR (CD₃OD, rotamers): δ 19.9, 26.2, 27.8, 43.6, 43.9, 44.4, 52.2, 52.5, 67.0, 116.2, 116.4, 126.2, 126.3, 126.8, 128.6, 128.9, 129.1, 129.3, 129.5, 131.0, 134.4, 135.6, 136.4, 137.5, 157.1, 169.4, 169.7.

### Example 35

### N-((4-Methylphenyl)methyl)-N-(1-piperidin-4-yl)-3-methoxyphenylacetamide (26HCH78-6)

To a solution of commercially available tert-butyl 4-oxo-1-piperidine carboxylate (1.75 g, 8.8 mmol) and 4-methylbenzylamine (970 mg, 8.0 mmol) in methanol (7 ml) was added acetic acid in methanol (1 M, 6.7 ml) followed by NaCNBH₃ in methanol (0.3 M, 30 ml). The resulting solution was stirred at room temperature. After 20 h, water (5 ml) was added, and the mixture was stirred for 1 h, before it was concentrated. Flash chromatography in dichloromethane:methanol 10:1 gave tert-butyl 4-(4-methylphenyl)methyl)amino-piperidine carboxylate. Yield: 2.4 g, 98%. To a solution of tert-butyl 4-(4-methylphenyl)methyl)amino-piperidine carboxylate (80 mg, 0.26 mmol) in dichloromethane (1.8 ml) was added diisopropylethylamine (0.11 ml, 2.4 eq.) followed by 3-methoxyphenylacetyl chloride (97 mg, 0.53 mmol). The reaction mixture was stirred at room temperature. After 20 h, water (1 ml) was added. The mixture was stirred for another 2 h, before diethyl ether (20 ml) was added. The mixture was sequentially extracted with HCl (0.2 N, 2×15 ml), NaOH (0.2 N, 2×15 ml), and H₂O (10 ml), dried (Na₂SO₄), filtered and concentrated. The crude material was dissolved in diethyl ether (2 ml) and HCl (4 M in dioxane, 1 ml). The reaction mixture was stirred at room temperature. After 2 h, NaOH (6 N, 1 ml) was added followed by dichloromethane (10 ml). The mixture was extracted with water (2×10 ml), dried (Na₂SO₄), and filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 26 mg; ¹³C-NMR (CD₃OD, rotamers): δ 19.9, 26.3, 27.7, 41.0, 43.7, 43.9, 44.4, 51.5, 53.8, 54.5, 54.6, 112.2, 112.6, 114.3, 114.5, 121.0, 121.2, 126.1, 126.8, 128.8, 129.4, 129.5, 129.8, 134.8, 136.0, 136.3, 136.5, 136.9, 137.2, 160.2, 160.3, 172.8, 173.2.

### Example 36

### N-((4-Methylphenyl)methyl)-N-(1-piperidin-4-yl)-4-fluorophenylacetamide (26HCH78-7)

To a solution of commercially available tert-butyl 4-oxo-1-piperidine carboxylate (1.75 g, 8.8 mmol) and 4-methylbenzylamine (970 mg, 8.0 mmol) in methanol (7 ml) was added acetic acid in methanol (1 M, 6.7 ml) followed by NaCNBH₃ in methanol (0.3 M, 30 ml). The resulting solution was stirred at room temperature. After 20 h, water (5 ml) was added, and the mixture was stirred for 1 h, before it was concentrated. Flash chromatography in dichloromethane:methanol 10:1 gave tert-butyl 4-(4-methylphenyl)methyl)amino-piperidine carboxylate. Yield: 2.4 g, 98%. To a solution of tert-butyl 4-(4-methylphenyl)methyl)amino-piperidine carboxylate (80 mg, 0.26 mmol) in dichloromethane (1.8 ml) was added diisopropylethylamine (0.11 ml, 2.4 eq.) followed by 4-fluorophenylacetyl chloride (0.072 ml, 0.53 mmol). The reaction mixture was stirred at room temperature. After 20 h, water (1 ml) was added. The mixture was stirred for another 2 h, before diethyl ether (20 ml) was added. The mixture was sequentially extracted with HCl (0.2 N, 2×15 ml), NaOH (0.2 N, 2×15 ml), and H₂O (10 ml), dried (Na₂SO₄), filtered and concentrated. The crude material was dissolved in diethyl ether (2 ml) and HCl (4 M in dioxane, 1 ml). The reaction mixture was stirred at room temperature. After 2 h, NaOH (6 N, 1 ml) was added followed by dichloromethane (10 ml). The mixture was extracted with water (2×10 ml), dried (Na₂SO₄), and filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 26 mg; ¹³C-NMR (CD₃OD, rotamers): δ 19.9, 26.1, 27.4, 39.7, 39.9, 43.5, 43.8, 44.4, 51.3, 53.4, 114.9, 115.1, 115.3, 126.1, 126.7, 128.5, 128.8, 129.4, 130.7, 130.8, 130.9, 131.0, 131.2, 131.6, 134.8, 136.0, 136.3, 137.2, 160.9, 163.3, 172.7, 173.2.

### Example 37

### N-((4-Methylphenyl)methyl)-N-(1-piperidin-4-yl)-2,5-dimethoxyphenylacetamide (26HCH78-8)

To a solution of commercially available tert-butyl 4-oxo-1-piperidine carboxylate (1.75 g, 8.8 mmol) and 4-methylbenzylamine (970 mg, 8.0 mmol) in methanol (7 ml) was added acetic acid in methanol (1 M, 6.7 ml) followed by NaCNBH₃ in methanol (0.3 M, 30 ml). The resulting solution was stirred at room temperature. After 20 h, water (5 ml) was added, and the mixture was stirred for 1 h, before it was concentrated. Flash chromatography in dichloromethane:methanol 10:1 gave tert-butyl 4-(4-methylphenyl)methyl)amino-piperidine carboxylate. Yield: 2.4 g, 98%. To a solution of tert-butyl 4-(4-methylphenyl)methyl)amino-piperidine carboxylate (80 mg, 0.26 mmol) in dichloromethane (1.8 ml) was added diisopropylethylamine (0.11 ml, 2.4 eq.) followed by 2,5-di-methoxyphenylacetyl chloride (0.092 ml, 0.53 mmol). The reaction mixture was stirred at room temperature. After 20 h, water (1 ml) was added. The mixture was stirred for another 2 h, before diethyl ether (20 ml) was added. The mixture was sequentially extracted with HCl (0.2 N, 2×15 ml), NaOH (0.2 N, 2×15 ml), and H₂O (10 ml), dried (Na₂SO₄), filtered and concentrated. The crude material was dissolved in diethyl ether (2 ml) and HCl (4 M in dioxane, 1 ml). The reaction mixture was stirred at room temperature. After 2 h, NaOH (6 N, 1 ml) was added followed by dichloromethane (10 ml). The mixture was extracted with water (2×10 ml), dried (Na₂SO₄), and filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. 36 mg; ¹³C-NMR (CD₃OD, rotamers): δ 20.0, 26.5, 28.2, 35.1, 35.7, 44.0, 44.4, 51.6, 53.8, 54.99, 55.03, 55.2, 55.5, 111.4, 111.7, 112.4, 112.9, 116.6, 116.9, 124.98, 125.02, 126.1, 126.7, 128.8, 129.3, 135.0, 136.1, 136.3, 137.0, 151.3, 151.7, 153.9, 154.0, 173.1, 173.5.

### Example 38

### N-((4-Methylphenyl)methyl)-N-(1-piperidin-4-yl)-4-chlorophenylacetamide (26HCH78-9)

To a solution of commercially available tert-butyl 4-oxo-1-piperidine carboxylate (1.75 g, 8.8 mmol) and 4-methylbenzylamine (970 mg, 8.0 mmol) in methanol (7 ml) was added acetic acid in methanol (1 M, 6.7 ml) followed by NaCNBH₃ in methanol (0.3 M, 30 ml). The resulting solution was stirred at room temperature. After 20 h, water (5 ml) was added, and the mixture was stirred for 1 h, before it was concentrated. Flash chromatography in dichloromethane:methanol 10:1 gave tert-butyl 4-(4-methylphenyl)methyl)amino-piperidine carboxylate. Yield: 2.4 g, 98%. To a solution of tert-butyl 4-(4-methylphenyl)methyl)amino-piperidine carboxylate (80 mg, 0.26 mmol) in dichloromethane (1.8 ml) was added diisopropylethylamine (0.11 ml, 2.4 eq.) followed by 4-chlorophenylacetyl chloride (99 mg, 0.53 mmol). The reaction mixture was stirred at room temperature. After 20 h, water (1 ml) was added. The mixture was stirred for another 2 h, before diethyl ether (20 ml) was added. The mixture was sequentially extracted with HCl (0.2 N, 2×15 ml), NaOH (0.2 N, 2×15 ml), and H₂O (10 ml), dried (Na₂SO₄), filtered and concentrated. The crude material was dissolved in diethyl ether (2 ml) and HCl (4 M in dioxane, 1 ml). The reaction mixture was stirred at room temperature. After 2 h, NaOH (6 N, 1 ml) was added followed by dichloromethane (10 ml). The mixture was extracted with water (2×10 ml), dried (Na₂SO₄), and filtered to give a clear solution. The solution was added on to a column carrying strongly acidic cation exchange resin (0.3 mmol/g resin), which was washed with methanol (3×6 ml), and eluted with 10% NH₃ in methanol, and concentrated to give the title compound. Yield: 22 mg; ¹³C-NMR (CD₃OD, rotamers): δ 19.9, 26.3, 27.7, 39.9, 40.0, 43.6, 43.9, 44.4, 51.5, 53.6, 126.1, 126.7, 128.2, 128.4, 128.6, 128.9, 129.4, 129.6, 130.7, 130.9, 131.2, 131.6, 132.5, 132.7, 133.9, 134.1, 134.4, 134.8, 135.9, 136.3, 137.2, 172.4, 172.9.

### Example 39

### N-((4-Methylphenyl)methyl)-N-(1-(phenylmethyl)pyrrolidin-3-yl)-N′-phenylmethylcarbamide (26HCH50)

To a solution of 3-amino-1-phenylmethylpyrrolidine (353 mg, 2 mmol) and 4-methylbenzaldehyde (361 mg, 3 mmol) in methanol (20 ml) was added acetic acid in methanol (2 M, 6.7 ml) followed by NaCNBH₃ in methanol (0.3 M, 3 ml). The mixture was stirred at room temperature. After 24 h, water (5 ml) was added. The mixture was stirred for another hour before concentrated. Flash chromatography in dichloromethane/methanol 10/1 gave N-((4-methylphenyl)methyl)amino-1-phenylmethylpyrrolidine.

N-((4-Methylphenyl)methyl)amino-1-phenylmethylpyrrolidine (35 mg, 0.125 mmol) was dissolved in dichloromethane (1.5 ml), and benzylisocyanate (0.09 ml, 0.3 mmol) was added. The reaction mixture was stirred at room temperature. After 48 h, the crude reaction mixture was added on to a column carrying strongly acidic cation exchange resin, which was washed with methanol (3×6 ml), and eluted with 10% NH₃ in methanol, and concentrated to give the title compound. Yield: 48 mg, 92%; ¹³C-NMR (CD₃OD): δ 20.0, 29.7, 44.2, 51.3, 53.4, 56.4, 57.8, 58.7, 126.8, 127.1, 127.3, 127.6, 128.3, 128.4, 128.9, 129.1, 135.9, 136.8, 140.3, 158.5.

### Example 40

### N-((4-Methylphenyl)methyl)-N-(1-(phenylmethyl)pyrrolidin-3-yl)-4-methoxyphenylacetamide (26HCH52)

To a solution of 3-amino-1-phenylmethylpyrrolidine (353 mg, 2 mmol) and 4-methylbenzaldehyde (361 mg, 3 mmol) in methanol (20 ml) was added acetic acid in methanol (2 M, 6.7 ml) followed by NaCNBH₃ in methanol (0.3 M, 3 ml). The mixture was stirred at room temperature. After 24 h, water (5 ml) was added. The mixture was stirred for another hour before concentrated. Flash chromatography in dichloromethane/methanol 10/1 gave N-((4-methylphenyl)methyl)amino-1-phenylmethylpyrrolidine.

To a solution of N-((4-Methylphenyl)methyl)amino-1-phenylmethylpyrrolidine (35 mg, 0.125 mmol), diisopropylethylamine (0.14 ml) in dichloromethane (1.5 ml) was added 4-methoxyphenylacetyl chloride (0.1 ml, 0.5 mmol). The reaction mixture was stirred at room temperature. After 48 h, the crude reaction mixture was concentrated and redissolved in methanol. The solution was added on to a column carrying strongly acidic cation exchange resin, which was washed with methanol (3×6 ml), and eluted with 10% NH₃ in methanol, and concentrated. Flash chromatography in dichloromethane/methanol 10/1 gave the title compound. Yield: 20 mg, 38%; ¹³C-NMR (CD₃OD): δ 21.3, 30.2, 40.8, 47.8, 53.6, 53.9, 55.5, 57.5, 60.2, 114.4, 125.7, 127.0, 127.1, 127.3, 127.4, 128.4, 128.5, 128.7, 128.9, 129.2, 129.8, 130.0, 135.9, 137.0, 158.6.

### Example 41

### N-((4-Methylphenyl)methyl)-N-(1-(phenylmethyl)piperidin-4-yl)-4-methoxyphenylthioacetamide (RO)

A mixture of N-((4-Methylphenyl)methyl)-N-(1-(phenylmethyl)piperidin-4-yl)-(4-methoxyphenylmethyl)acetamide (20 mg, 0.045 mmol) and Lawesson's reagent (25 mg, 0.062 mmol), was taken in a glass vial and mixed thoroughly with magnetic stirbars. The glass vial was then irradiated in a microwave oven (900 W, Whirlpool M401)for 8 min. Upon completion of the reaction, the yellow-colored material was transferred to an ion-exchange column with the aid of methanol (2 ml). The ion-exchange column was subsequently washed with CH₂Cl₂ (2 ml) and methanol (2 ml) and the product was thereafter eluted from the ion-exchange column (10% NH₃ in methanol, 2 ml)to give N-((4-Methylphenyl)methyl)-N-(1-(phenylmethyl)piperidin-4-yl)-4-methoxyphenylmethyl thioacetamide (20 mg, 97%) as a white solid; LC-MS: (N+H)⁺ 459, tᵣ 9.60 min; TLC(CH₂Cl₂/methanol 20:1) R^{f}=0.38.

Pharmalogical Data

### Example 42

### Receptor Selection and Amplification (R-SAT) Assays

The functional receptor assay, Receptor Selection and Amplification Technology (R-SAT), was used (with minor modifications from that previously described U.S. Pat. No. 5,707,798) to screen compounds for efficacy at the 5-HT2A receptor. Briefly, NIH3T3 cells were grown in 96 well tissue culture plates to 70-80% confluence. Cells were transfected for 12-16 hours with plasmid DNAs using superfect (Qiagen Inc.) as per manufacture's protocols. R-SAT's were generally performed with 50 ng/well of receptor and 20 ng/well of Beta-galactosidase plasmid DNA. All receptor and G-protein constructs used were in the pSI mammalian expression vector (Promega Inc) as described in U.S. Pat. No. 5,707,798. The 5HT2A receptor gene was amplified by nested PCR from brain cDNA using the oligodeoxynucleotides based on the published sequence (see Saltzman et. al. Biochem. Biophys. Res. Comm. 181:1469-78 (1991)). Large-scale transfections, cells were transfected for 12-16 hours, then trypsinized and frozen in DMSO. Frozen cells were later thawed, plated at 10,000-40,000 cells per well of a 96 well plate that contained drug. With both methods, cells were then grown in a humidified atmosphere with 5% ambient CO₂ for five days. Media was then removed from the plates and marker gene activity was measured by the addition of the beta-galactosidase substrate ONPG (in PBS with 5% NP-40). The resulting colorimetric reaction was measured in a spectrophotometric plate reader (Titertek Inc.)at 420 nM. All data were analyzed using the computer program XLFit (IDBSm). Efficacy is the percent maximal repression compared to repression by a control compound (ritanserin in the case of 5HT2A). pIC50 is the negative of the log(IC50), where IC50 is the calculated concentration in Molar that produces 50% maximal repression. The results obtained for six compounds of the invention are presented in Table 1.

TABLE 1
Efficacy of Compounds at the 5-HT2A Receptor Efficacy.

| | Efficacy | Efficacy | pIC50 | pIC50 |
|---|---|---|---|---|
| Compound | (average) | (stdev) | (average) | (stdev) |
| 26HCH52 | 98 | 5.0 | 7.31 | 0.16 |
| 26HCH66-03 | 76 | 13.3 | 7.42 | 0.01 |
| 26HCH66-05 | 109 | 3.0 | 7.55 | 0.15 |
| 26HCH80-2 | 89 | 4.6 | 7.78 | 0.17 |
| 26HCH80-7 | 87 | 3.7 | 7.70 | 0.26 |
| 26HCH80-10 | 91 | 4.9 | 7.21 | 0.05 |

### Example 43

### In Vitro Efficacy of 26HCH17 as an Inverse Agonist at the 5-HT2A Receptor

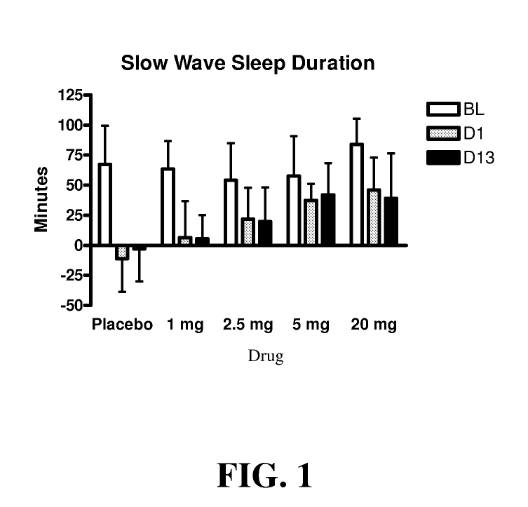

The graph shown in FIG. 1 represents the data obtained from a dose response analysis of 26HCH17 and ritanserin as 5-HT2A receptor inverse agonists. Briefly, the 5-HT2A receptor, and the alpha subunit of the guanine nucleotide binding protein Gq were transiently transfected into NIH3T3 cells and assayed using the functional receptor assay, Receptor Selection and Amplification Technology (R-SAT) essentially as disclosed in U.S. Pat. No. 5,707,798. Each compound was screened at seven serially diluted concentrations in triplicate. Data were analyzed using least squares fit analysis with GraphPad Prism (San Diego, Calif.), and are reported normalized to percent response.

### Example 44

### Selectivity Profile of Inverse Agonist 26HCH16D

R-SAT assays (as described in Example 42) were carried out with cells transfected with receptors (listed below) to determine the receptor selectivity profile for compound 26HCH16D. 5HT2A inverse agonist data (IC50 nM; % efficacy) were derived from detailed dose response curves (7 points in triplicate). All other data (initial concentration at which at least 30% efficacy observed; actual efficacy figure) derived from the 4 dose profiling protocol in which compounds were tested at 4 doses in duplicate. nr=activity less than 30% at all doses tested (3, 30, 300, 3000 nM), therefore EC50/IC50 greater than 3000 nM). The results are presented in Table 2.

TABLE 2
Profile of 5-HT2A Inverse Agonist 26HCH16D.

| Receptor | | Efficacy |
|---|---|---|
| 5HT2A (human) | Agonist | nr |
| | Inverse Agonist | 0.9 nM; 79% |
| 5HT2B (human) | Agonist | nr |
| | Antagonist | 3000 nM; 60% |
| 5HT2C (human) | Agonist | nr |
| | Inverse Agonist | 3000 nM; 79% |
| 5HT1A (human) | Agonist | nr |
| | Antagonist | |
| 5HT1A (rat) | Antagonist | nr |
| 5HT1E (human) | Agonist | nr |
| D2 (human) | Agonist | nr |
| | Antagonist | 3000 nM; 73% |
| H1 (human) | Agonist | nr |
| | Antagonist | 3000 nM; 30% |
| alpha1a/D (rat) | Agonist | nr |
| | Antagonist | nr |
| alpha1b/B (hamster) | Agonist | nr |
| | Antagonis | nr |
| alpha1c/A (human) | Agonist | nr |
| | Antagonist | 3000 nM; 46% |
| alpha2A (human) | Agonist | nr |
| | Antagonist | nr |
| alpha2B (human) | Agonist | nr |
| | Antagonist | nr |
| alpha2C (human) | Agonist | nr |
| | Antagonist | nr |
| m1 (human) | Agonist | nr |
| | Antagonist | nr |

As indicated above, 26HCH16D is a highly selective 5-HT2A inverse agonist.

General LC-MS Procedure for Working Examples ELHO1-46, MBT01-14 and AKU01-38

In the following examples, HPLC/MS analyses were performed using either of two general methods (Method A or Method B). The tᵣ values reported below were obtained using one of these procedures, as indicated in the specific examples.

The methods were as follows:

Method A: Agilent HP1100 HPLC/MSD.

G1312A Binary pump, G1313A Autosampler, G1316A Column compartment, G1315A Diode array detector (190-450 nm), 1946A MSD, electrospray ionization.

Chromatography: 8 mM ammoniumacetate in water/acetonitrile.

Gradient start at 70% org. up to 100% org. over 12 min, down to 70% org. over 0.5 min, held for 3.5 min. Total runtime 16 min. Flowrate 1 ml/min

Column, Phenomenex Luna C18 (2) 3 um 75×4.6 mm.

MS Parameters: Drying gas, 10 l/min. Nebulizer pressure, 40 psig. Gas temp, 350 C. VCap, 4000.

Method B: Waters/Micromass HPLC/MS

600 LC-pump, 2700 Sample manager, 2487 Dual absorbance detector (channel A-205 nm, channel B-235 nm), Micromass ZMD-mass-spectrometer, electrospray ionization.

Chromatography: 0.15% TFA in water/acetonitrile.

Gradient start at 30% org. up to 100% org. over 10 min, held for 3 min. down to 30% org. over 0.5 min, held for 4.5 min. Total run time 18 min. Flowrate, 1 ml/min.

Column, Symmetry C18, 5 μm, 4.6×50 mm. or 10 mM ammoniumacetate in water/acetonitrile.

Gradient start at 30% org. for 2.5 min, up to 100% org. over 10 min, held for 9 min, down to 30% org. over 0.5 min, held for 5 min. Total run time 27 min. Flowrate, 1 ml/min.

Column: Phenomenex Synergi C12, 4 μm, 4.6×50 mm.

MS Parameters: Desolvation Gas, 404 l/H. Capillary, 5.3 kV. Cone, 36V. Extractor, 3V. Source Block Temp, 130 C. Desolvation Temp, 250 C.

### Example 45

### 2-(4-methoxyphenyl)-N-(4-methylbenzyl)-N-(piperidin-4-yl)acetamide (50ELH87)

Reaction Step 1: N-trifluoroacetyl-4-piperidone (50ELH84)

4-Piperidone hydrochloride monohydrate (4.0 g, 26 mmol, 1.0 eq) was dissolved in 130 ml of dichloromethane. After addition of triethylamine (8.66 g, 3.3 eq) the reaction mixture was stirred for 10 min. The mixture was cooled on an ice-bath (0° C.). Trifluoroacetic anhydride (12.0 g, 2.2 eq) was added dropwise under stirring. After 2 hours the reaction was quenched by addition of distilled water. The aqueous phase was extracted twice with dichloromethane. The combined organic layers were collected and dried with sodium sulfate. Concentration afforded N-trifluoroacetyl-4-piperidone.

Reaction Step 2: 4-(4-Methylbenzylamino)-1-(trifluoroacetyl)piperidin (50ELH85)

Methanol (150 ml) was added to an Erlenmeyer flask and acetic acid was added under stirring until pH 5.4-Methylbenzylamine (3.14 g, 25.9 mmol) and N-trifluoroacetyl-4-piperidone(from reaction step 1) (5.065 g, 25.9 mmol) were added to a 250 ml round-bottomed flask and dissolved in the methanol/acetic acid (150 ml) solution previously made. The reaction mixture was stirred for 5 min and NaCNBH₃ (2.46 g, 38.9 mmol) was added slowly under stirring. After 20 hours the reaction was concentrated and transferred to a separatory funnel containing dichloromethane and distilled water. The aqueous phase was made basic by addition of Na₂CO₃. The aqueous phase was extracted twice with dichloromethane. The combined organic layers were collected and dried with Na₂SO₄. Concentration afforded, 4-(4-methylbenzylamine)-1-(trifluoroacetyl)piperidine. UV/MS 60/53 (M⁺ 301), tᵣ (A, MS) 3.267.

Reaction Step 3: 2-(4-Methoxyphenyl)-N-(4-methylbenzyl)-N-(1-trifluoroacetylpiperidin-4-yl)acetamide (50ELH86)

The product from reaction step 2 (7.8 g, 25.9 mmol) was dissolved in 100 ml of dichloromethane and stirred while 4-methoxyphenylacetyl chloride (4.8 g, 25.9 mmol) was added. After 4 hours, heptane was added whereupon the product precipitated as the hydrochloride salt. The solvent was removed by evaporation. The crude material was purified by flash chromatography EtOAc/Heptane (1:2) Yield (overall: Reaction steps 1+2+3) 3.912 g (34%), UV/MS 91/58 (M⁺ 449), tᵣ (A, MS) 4.319. ¹H-NMR (400 MHz, CDCl₃) δ 6.80-7.15 (Ar, 4H), 4.64 (brt, 1H), 4.4 (s, 2H), 3.95 (d, 2H), 3.72 (s, 3H), 3.50 (s, 2H), 3.09 (t, 2H), 2.7 (t, 2H), 2.32 (s, 3H), 1.75 (btr, 2H). ¹³C-NMR δ 172.5; 158.8; 137.4; 134.9; 129.9; 129.9; 129.8; 127.1; 125.8; 114.3; 55.4; 52.2; 47.3; 45.3; 43.4. 40.6; 30.1; 29.2; 21.2.

Reaction Step 4: 2-(4-Methoxyphenyl)-N-(4-methylbenzyl)-N-(piperidin-4-yl)Acetamide (50ELH87)

The product from reaction step 3 (3.9 g, 8.7 mmol) was dissolved in methanol (12 ml). In a 250 ml round bottom flask a saturated solution of potassium carbonate in methanol was prepared. To this solution, the 2-(4-methoxyphenyl)-N-(4-methylbenzyl)-N-(N-trifluoroacetpiperidin-4-yl)acetamide solution was added under stirring. After 4 hours, the solution was concentrated and the remaining solid taken upon base and dichloromethane. The combined organic layers were dried with sodium sulfate and concentrated. UV/MS 91/72 (M⁺ 353), tᵣ (A, MS) 2.210.

The corresponding hydrochloride salt was also prepared, by dissolving the free base in dichloromethane (1 ml) and HCl (1 eq. 2 M HCl in ether) was added with stirring. The salt was precipitated by addition of the dichloromethane solution into heptane. Concentration on the rotary evaporator returned the product is white crystals.

### Example 46

### 2-(4-Methoxyphenyl)-N-(4-methylbenzyl)-N-(1-methylpiperidin-4-yl)acetamide (50ELH27)

Reaction Step 1: 4-(4-Methylbenzylamino)-1-methylpiperidine (50ELH25)

Methanol (50 ml) was added to an Erlenmeyer flask and acetic acid was added under stirring until pH 5. Methylbenzylamine (1.0 g, 8.8 mmol) and 1-Methyl-4-piperidone (1.1 g, 8.8 mmol) were added to a 100 ml round-bottomed flask and dissolved in the methanol/acetic acid (40 ml) solution previously made. The reaction mixture was stirred for 5 min and NaCNBH₃ (0.83 g, 13.2 mmol) was added slowly under stirring. After 20 hours the reaction was concentrated and transferred to a separatory funnel containing dichloromethane and distilled water. The aqueous phase was made basic by addition of Na₂CO₃. The aqueous phase was extracted twice with dichloromethane. The combined organic layers were collected and dried with Na₂SO₄. Concentration afforded the title compound. Yield (crude): 98%. UV/MS 89/88 (M⁺ 353), tᵣ (A, MS) 3.982.

Reaction Step 2: 2-(4-Methoxyphenyl)-N-(4-methylbenzyl)-N-(1-methylpiperidin-4-yl)acetamide (50ELH27)

The product from reaction step 1 (1.9 g, 8.7 mmol) was dissolved in 40 ml of dichloromethane and stirred while 4-methoxyphenylacetylchloride (1.606 g, 8.7 mmol) was added. After 4 hours, heptane was added whereupon the product precipitated as the hydrochloride salt. The solvent was removed by evaporation. The crude material was purified by flash chromatography first eluting with 10% MeOH in CH₂Cl₂ and thereafter eluting with 0-20% MeOH in CH₂Cl₂ and 5% NEt₃. Yield (overall: Reaction steps 1+2): 77%. UV/MS: 100/100 (M⁺ 367), tᵣ (A, MS) 4.359, R^{f} 0.15 (2% MeOH in CH₂Cl₂). ¹H-NMR (400 MHz, CDCl₃) δ 12.6 (s, 1H), 7.16 (d, J=7.0 Hz, 2H), 7.10 (d, J=7.0 Hz, 2H), 7.04 (d, J=8.0 Hz, 2H), 6.82 (d, J=8.0 Hz, 2H), 4.87 (tt, J=11.0, 4.0 Hz, 1H), 4.53 ppm (s, 2H), 3.78 (s, 3H), 3.55 (s, 2H), 3.42 (brd, J=11.0 Hz, 2H), 2.80 (brq, J=11.0 Hz, 2H), 2.7 (d, J=4.0 Hz, 3H), 2.42 (dq, J=13.0, 3.0 Hz, 2H), 2.34 (s, 3H), 1.78 (brd, J=13.0 Hz, 2H). ¹³C-NMR δ 173.1; 158.9; 137.4; 134.8; 129.9; 126.7; 125.8; 114.4; 76.9; 55.5; 54.6; 48.8; 43.7; 40.5; 26.4; 21.2

### Example 47

### 2-(4-Methoxyphenyl)-N-(4-methylbenzyl)-N-(1-cyclohexylmethylpiperidin-4-yl)acetamide (42ELH45)

50ELH87 (the hydrochloride salt) (0.5 g, 1.29 mmol, 1.0 eq) was dissolved in ethanol (100 ml). Cyclohexanecarboxaldehyde (2.5 g, 20 eq.) was added followed by addition of sodium borohydride (0.084 g, 2.0 eq.). The reaction was stirred for 36 h and acetic acid (3 ml) was added. The reaction was stirred for additionally 2 h and extracted with sodium hydrogen carbonate (3 times) and dichloromethane. The organic layers were dried with sodium sulfate and concentrated. The product was purified by flash chromatography (1-10% MeOH in CH₂Cl₂). The resulting product was dissolved in ether (20 ml) and MeOH (added dropwise until dissolved) and HCl (1 eq. 2 M HCl in ether) was added under stirring. The hydrochloride salt of 2-(4-methoxyphenyl)-N-(4-methylbenzyl)-N-(1-cyclohexylmethylpiperidin-4-yl)acetamide precipitated and the white crystals were filtered. Yield 80 mg (16%), UV/MS 100/100 (M⁺ 449), rₜ (A, MS) 7.105, mp 133-135° C., R^{f} 0.25 (2% MeOH/CH₂Cl₂). ¹H-NMR (400 MHz, CDCl₃) δ 11.9 (brs, 1H), 7.12 (q, 4H), 7.02 (d, 2H), 6.80 (d, 2H), 4.87 (m, 1H), 4.58 (s, 2H), 3.77 (s, 3H), 3.55 (s, 2H), 3.48 (m, 2H), 2.70 (m, 4H), 2.31 (s, 3H), 1.91 (d, 2H), 1.75 (m, 3H), 1.64 (d, 1H), 1.22 (d, 2H), 1.13 (tt, 2H), 1.02 (brq, 2H). ¹³C-NMR δ 173.1; 158.8; 137.2; 135.1; 129.9; 129.8; 126.8; 125.8; 114.4; 64.1; 55.5; 53.4; 49.2; 46.5; 40.4; 33.9; 25.9; 25.8; 25.7; 21.2.

### Example 48

### 2-(4-Methoxyphenyl)-N-(4-methylbenzyl)-N-(1-ethylpiperidin-4-yl)acetamide (42ELH80)

50ELH87 (0.25 g, 0.71 mmol, 1.0 eq) was dissolved in acetonitrile (15 ml) and ethyl bromide (0.232 g, 3.0 eq.) was added under stirring. After 2 min Hunings base (0.084 g, 10.0 eq.) was added. After 36 hours, the solution was extracted with sodium hydrogen carbonate solution and dichloromethane (3 times). The organic layers were dried with sodium sulfate and concentrated yielding a yellow oil. The product was purified by flash chromatography (2% MeOH in CH₂Cl₂). The resulting product was dissolved in dichloromethane (1 ml) and HCl (1 eq. 2 M HCl in ether) was added under stirring. The salt was precipitated by addition of the dichloromethane solution into heptane. Concentration on the rotary evaporator gave the product as white crystals. Yield 170 mg (63%), UV/MS 98/95 (M⁺ 381), mp 153-155° C., rₜ (A, MS) 3.033, R^{f} 0.35 (3% MeOH/CH₂Cl₂). ¹H-NMR (400 MHz, CDCl₃) δ 12.2 (s, 1H), 7.15 (d, 2H), 7.12 (d, 2H), 7.08 (d, 2H), 6.82 (d, 2H), 4.89 (m, 1H), 4.58 (s, 2H), 3.79 (s, 3H), 3.58 (s, 2H), 3.50 (d, 2H), 2.90 (m, 1H), 2.7 (brq, 2H), 2.45 (m, 2H), 2.34 (s, 3H), 1.80 (d, 2H), 1.44 (t, 3H). ¹³C-NMR δ 173.1; 158.9; 137.3; 134.9; 129.9; 125.8; 114.4; 55.5; 52.3; 52.0; 49.2; 46.5; 40.5; 26.2; 21.2; 9.5.

### Example 49

### 2-(4-Methoxyphenyl)-N-(4-chlorbenzyl)-N-(1-ethylpiperidin-4-yl)acetamide (42ELH85)

This compound was prepared similarly to 50ELH27

Reaction-Step 1: (42ELH84)

Starting materials: 1-Methyl-4-piperidone (0.5 g, 4.4 mmol, 1.0 eq.), 4-chlorobenzylamine (0.626 g, 1.0 eq.), sodium cyanoborohydride (0.279 g, 1.5 eq.).

Reaction-Step 2: (42ELH85)

Starting materials: 42ELH84, 4-methoxyphenylacetylchloride (0.774 g, 1.0 eq.).

The procedure was analogous to 50ELH27, but the product was purified by ion-exchange chromatography followed by HPLC. The hydrochloride salt was made by dissolving the free base in dichloromethane (1 ml) and HCl (1 eq. 2 M HCl in ether) was added under stirring. The salt was precipitated by addition of the dichloromethane solution into heptane followed by concentration on the rotary evaporator.

Product White crystals. UV/MS 98/97 (M⁺ 387), rₜ (A, MS) 2.953. ¹H-NMR (400 MHz, CDCl₃) δ 12.6 (s, 1H), 7.35 (d, 2H), 7.18 (d, 2H), 7.05 (d, 2H), 6.82 (d, 2H), 4.89 (m, 1H), 4.55 (s, 2H), 3.80 (s, 3H), 3.55 (s, 2H), 3.45 (brs, 2H), 2.80 (brs, 2H), 2.72 (s, 3H), 2.25 (brs, 3H), 1.80 (brs, 2H). ¹³C-NMR δ 173.0; 158.9; 136.5; 133.6; 129.8; 129.4; 127.3; 126.3; 114.5; 55.5; 54.6; 48.7; 46.3; 43.7; 40.5; 26.3.

### Example 50

### 2-(4-Methoxyphenyl)-N-(4-methylbenzyl)-N-(1-isopropylpiperidin-4-yl)acetamide (42ELH79)

Procedure as 42ELH80

Starting materials: 50ELH87 (0.25 g, 0.71 mmol, 1.0 eq.), isopropylbromide (0.262 g, 3.0 eq.).

Product: Yield 130 mg (46%), UV/MS 100/100 (M⁺ 395), rₜ (A, MS) 3.360. ¹H-NMR (400 MHz, CDCl₃) δ 12.0 (s, 1H), 7.15 (d, 2H), 7.10 (d, 2H), 7.05 (d, 2H), 6.82 (d, 2H), 4.87 (m, 1H), 4.60 (s, 2H), 3.79 (s, 3H), 3.57 (s, 2H), 3.38 (brd, 3H), 2.79 (q, 2H), 2.63 (q, 2H), 2.34 (s, 3H), 1.80 (d, 2H), 1.39 (d, 6H). ¹³C-NMR δ 173.1; 158.9; 137.3; 135.1; 129.8; 126.8; 125.8; 114.4; 57.9; 49.4; 48.2; 46.5; 40.5; 25.9; 21.2; 16.9.

### Example 51

### 2-(4-Methoxyphenyl)-N-(4-chlorobenzyl)-N-(piperidin-4-yl)acetamide (42ELH89) (As Starting Material in Other Reactions, Used Unpurified)

Procedure as 50ELH27.

Reaction Step 1: N-Trifluoroacetyl-4-piperidone (42ELH86)

Starting materials: 4-Piperidone hydrochloride monohydrate (2.0 g, 13 mmol, 1.0 eq), trifluoroacetic anhydride (6.0, 2.2 eq.). TLC showed full conversion.

Product: R^{f} 0.9 (10% MeOH/CH₂Cl₂).

Reaction Step 2: 4-(4-Chlorobenzoylamino)-1-(trifluoroacetyl)piperidin (42ELH87)

Starting materials: 42ELH86 (2.5 g, 12.8 mmol, 1.0 eq.), 4-Chlorobenzylamine (1.8 g, 1.0 eq.)

Reaction Step 3: 2-(4-Methoxyphenyl)-N-(4-chlorobenzyl)-N-(1-trifluoroacetylpiperidin-4-yl)acetamide (42ELH88)

Starting materials: 42ELH87 (4.0 g, 12.5 mmol, 1.0 eq.), 4-methoxyphenylacetylchloride (2.31 g, 1.0 eq.)

Reaction Step 4: 2-(4-Methoxyphenyl)-N-(4-chlorobenzyl)-N-(piperidin-4-yl)acetamide (42ELH89)

Product: Yield: 2 g (57%), UV/MS 80/82 (M⁺ 373), Rf 0.2 (50% EtOAc/Heptane).

### Example 52

### 2-(4-Methoxyphenyl)-N-(4-chlorbenzyl)-N-(1-cyclopentylpiperidin-4-yl)acetamide (42ELH91)

Procedure as 42ELH80, but the product was purified by HPLC. The acidic eluent was made basic with sodium carbonate and extracted with dichloromethane (3 times). The combined organic layers were collected and dried with sodium sulfate and concentrated. The remaining product was dissolved in 1 ml of dichloromethane and HCl (1 eq. 2 M HCl in ether) was added under stirring. This solution was added drop-wise to a large excess of n-heptane to make the hydrochloride precipitate. The solvent was evaporated off to form white crystals of 2-(4-methoxyphenyl)-N-(4-chlorbenzyl)-N-(1-cyclopentylpiperidin-4-yl)acetamide, hydrochloride.

Starting materials: 42ELH89 (0.25 g, 0.67 mmol, 1.0 eq.), cyclopentyl bromide (0.3, 3.0 eq.)

Product: Yield. 211.2 mg (76%). Purification by ion-exchange: UV/MS 90/98. Purification by HPLC UV/MS 100/100 (M⁺ 441), R^{f} 0.2 (3% MeOH/CH₂Cl₂), rₜ (A, MS) 4.067. ¹H-NMR (400 MHz, CDCl₃) δ12.2 (brs, 1H), 7.32 (d, 2H), 7.17 (d, 2H), 7.04 (d, 2H), 6.82 (d, 2H), 4.90 (brt, 1H), 4.58 (s, 2H), 3.79 (s, 3H), 3.58 (brd, 2H), 3.54 (s, 2H), 3.14 (brq, 2H), 2.58 (brq, 2H), 2.04 (m, 4H), 1.89 (m, 4H), 1.75 (brd, 2H). ¹³C-NMR δ 173.0; 158.9; 133.5; 129.8; 129.3; 127.3; 126.4; 114.5; 68.4; 55.5; 51.9; 49.1; 46.2; 40.5; 28.5; 26.0; 23.8.

### Example 53

### 2-(4-Methoxyphenyl)-N-(4-chlorbenzyl)-N-(1-isopropylpiperidin-4-yl)acetamide (42ELH90)

42ELH89 (0.25 g, 0.67 mmol, 1.0 eq) was transferred to a 4 ml vial and dissolved in acetonitrile (2 ml). Isopropyl bromide (0.25 g, 3.0 eq.) was added along with Hunings base (0.87 g, 10.0 eq.). The vial was sealed and shaken for 4 days at 60° C. The reaction mixture was transferred to a separatory funnel with distilled water and CH₂Cl₂. The aqueous phase was made basic with sodium hydrogen carbonate and extracted with dichloromethane (3 times). The organic layers were collected and dried with sodium sulfate and concentrated, this resulted in a yellow oil. The product was purified by flash chromatography (3% MeOH in CH₂Cl₂). The resulting product was dissolved in dichloromethane (1 ml) and HCl (1 eq. 2 M HCl in ether) was added under stirring. The salt precipitated by addition of the dichloromethane solution into heptane. Concentration on the rotary evaporator returned the product as white crystals. Yield 101.2 mg (63%), UV/MS 94/96 (M⁺ 415), R^{f} 0.25 (3% MeOH/CH₂Cl₂).

¹H-NMR (400 MHz, CDCl₃) δ 12.05 (brs, 1H), 7.36 (d, 2H), 7.18 (d, 2H), 7.04 (d, 2H), 6.82 (d, 2H), 4.88 (m, 1H), 4.60 (s, 2H), 3.79 (s, 3H), 3.55 (d, 2H), 3.36 (d, 3H), 2.80 (brq, 2H), 2.65 (brq, 2H), 1.76 (brd, 2H), 1.39 (d, 6H). ¹³C-NMR δ 173.0; 159.0; 137.0; 136.0; 129.7; 129.3; 127.4; 126.4; 114.5; 57.9; 55.5; 49.2; 48.2; 46.2; 40.5; 25.8; 16.9.

### Example 54

### 2-(Phenyl)-N-(4-trifluoromethylbenzyl)-N-(1-methylpiperidin-4-yl)acetamide (50ELH14b)

Procedure as for 50ELH27. Purification was done by HPLC. The hydrochloride salt was made by dissolving the free-base in dichloromethane (1 ml) and HCl (1 eq. 2 M HCl in ether) was added under stirring. The salt was precipitated by addition of the dichloromethane solution into heptane followed by concentration.

Reaction-Step 1: 4-(4-Trifluoromethylbenzylamino)-1-methylpiperidin (50ELH2).

Starting materials: 1-Methyl-4-piperidone (1.13 g, 10.0 mmol, 1.0 eq.), 4-trifluoromethylbenzylamine (1.75 g, 1.0 eq.).

Product: UV/MS 80/92 (M+273).

Reaction-Step 2: 2-(Phenyl)-N-(4-trifluoromethylbenzyl)-N-(1-methylpiperidin-4-yl)acetamide (50ELH14b).

Starting materials: 50ELH2 (0.12 g, 0.44 mmol, 1.0 eq.), phenylacetylchloride (0.068 g, 1.0 eq.).

Product: UV/MS 100/97 M⁺ 390), rₜ (A, MS) 3.797, R^{f} 0.3 (5% MeOH/CH₂Cl₂). ¹H-NMR (400 MHz, CDCl₃, rotamers 54/46) δ 7.52 (d, 2H), 7.42 (d, 2H), 7.12 7.30 (m, 4H), 4.63 and 3.74 (2m, 1H), 4.38 (brs, 2H), 3.80 and 3.50 (2s, 3H), 3.31 and 2.78 (2d, 2H), 2.33 and 2.18 (2s, 2H), 2.24 and 1.65-1.90 (t and m, 4H), 1.60 and 1.22 (2d, 2H), 1. ¹³C-NMR δ 172.3; 171.8; 143.9; 135.1; 134.8; 129.1; 129.0; 128.9; 128.7; 127.4; 127.3; 127.2; 126.3; 126.1; 126.0; 56.0; 55.2; 54.9; 50.9; 46.8; 45.2; 44.9; 42.2; 41.7; 30.6; 28.4.

### Example 55

### 2-(4-Fluorophenyl)-N-(4-trifluoromethylbenzyl)-N-(1-methylpiperidin-4-yl)acetamide (50ELH14c)

Procedure as 50ELH14B.

Reaction-Step 2: 2-(4-Fluorophenyl)-N-(4-trifluoromethylbenzyl)-N-(1-methylpiperidin-4-yl)a cetamide (50ELH14c).

Starting materials: 50ELH2 (0.12 g, 0.44 mmol, 1.0 eq.), 4-fluorophenylacetylchloride (0.076 g, 1.0 eq.).

Product: Yield 69.7 mg (36%), UV/MS 100/98 (M⁺ 409), rₜ (A, MS) 3.839, R^{f} 0.3 (5% MeOH/CH₂Cl₂). ¹H-NMR (400 MHz, DMSO, rotamers 65/35) δ 10.80 and 10.60 (2s, 1H), 7.71 and 7.62 (2d, 2H), 7.47 and 7.38 (2d, 2H), 7.00-7.36 (t and m, 4H), 4.70 and 4.50 (2s, 2H), 4.30 (m, 1H), 3.93 and 3.56 (2s, 2H), 3.34 (s, 2H), 3.00 (brq, 2H), 2.64 (s, 3H), 2.08 (m, 2H), 1.68 and 1.58 (2d, 2H). ¹³C-NMR δ 176.8; 176.4; 167.6; 165.3; 150.0; 149.0; 136.6; 132.5; 131.0; 130.5; 120.6; 120.5; 120.5; 120.4; 58.1; 58.0; 57.0; 54.5; 52.0; 49.3; 47.6; 45.0; 32.4; 31.4.

### Example 56

### 2-(4-Methoxyphenyl)-N-(4-trifluoromethylbenzyl)-N-(1-methylpiperidin-4-yl)acetamide (50ELH14d)

Procedure as 50ELH14B.

Reaction-Step 2: 2-(4-Methoxyphenyl)-N-(4-trifluoromethylbenzyl)-N-(1-methylpiperidin-4-yl)acetamide (50ELH14d).

Starting materials: 50ELH2 (0.15 g, 0.55 mmol, 1.0 eq.), 4-methoxyphenylacetylchloride (0.1 g, 1.0 eq.).

Product: Yield 57.5 mg (29%), UV/MS 99/100 (M⁺ 421), rₜ (B, MS) 6.30, R^{f} 0.25 (3% MeOH/CH₂Cl₂). ¹H-NMR (400 MHz, CDCl₃) δ 12.4 (brs, 1H), 7.55 (d, 2H), 7.28 (d, 2H), 6.96 (d, 2H), 4.84 (brt, 1H), 4.59 (s, 2H), 3.72 (s, 3H), 3.46 (s, 2H), 3.38 (d, 2H), 2.78 (q, 2H), 2.64 (s, 3H), 2.38 (q, 2H), 1.70 (d, 2H). ¹³C-NMR δ 173.0; 159.0; 142.3; 130.0; 129.8; 126.3; 126.2; 114.7; 114.5; 55.5; 54.4; 48.7; 46.5; 43.6; 40.6; 26.3.

### Example 57

### 2-(4-Trifluoromethylphenyl)-N-(4-trifluoromethylbenzyl)-N-(1-methylpiperidin-4-yl)acetamide (50ELH14a)

Procedure as 50ELH14B.

Reaction-Step 2: 2-(4-Trifluoromethylphenyl)-N-(4-trifluoromethylbenzyl)-N-(1-methylpiperidin-4-yl)acetamide (50ELH14a).

Starting materials: 50ELH2 (0.12 g, 0.44 mmol, 1.0 eq.), 4-trifluoromethylphenylacetylchloride (0.1 g, 1.0 eq.).

Product: Yield 92.6 mg (42%), UV/MS 89/93 (M⁺ 458), rₜ (A, MS) 4.211, R^{f} 0.3 (5% MeOH/CH₂Cl₂). ¹H-NMR (400 MHz, CDCl₃) δ 12.7 (brs, 1H), 7.56 (d, 2H), 7.48 (d, 2H), 7.17 (d, 2H), 4.86 (m, 1H), 4.63 (s, 2H), 3.58 (s, 3H), 3.40 (d, 2H), 2.75 (q, 2H), 2.65 (d, 3H), 2.46 (dq, 2H), 1.73 (brs, 2H). ¹³C-NMR δ 171.8; 141.9; 138.4; 129.4; 127.9; 126.3; 126.3; 126.2; 125.9; 125.8; 54.4; 48.8; 46.6; 43.6; 40.9; 26.2.

### Example 58

### 2-(4-Fluorophenyl)-N-(4-fluorobenzyl)-N-(1-methylpiperidin-4-yl)acetamide (50ELH6) Procedure as 50ELH14B

Reaction-Step 1: 4-(4-Fluorobenzylamino)-1-methylpiperidine (50ELH4).

Starting materials: 1-Methyl-4-piperidone (1.13 g, 10.0 mmol, 1.0 eq.), 4-fluorobenzylamine (1.25 g, 1.0 eq.).

Product: Yield 2.154 g (97%), UV/MS 79/89 (M⁺223).

Reaction-Step 2: 2-(4-Fluorophenyl)-N-(4-fluorobenzyl)-N-(1-methylpiperidin-4-yl)acetamide (50ELH14a).

Starting materials: 50ELH4 (0.12 g, 0.54 mmol, 1.0 eq.), 4-fluorophenylacetylchloride (0.096 g, 1.0 eq.).

Product: Yield 57 mg (29%), UV/MS 100/100 (M⁺ 359), rₜ (A, MS) 3.763, R^{f} 0.25 (3% MeOH/CH₂Cl₂). ¹H-NMR (400 MHz, CDCl₃) 67 12.6 (brs, 11H), 7.2 (dd, 2H), 7.06 (m, 4H), 6.98 (t, 2H), 4.88 (tt, 1H), 4.58 (s, 4H), 3.45 (d, 2H), 2.81 (q, 2H), 2.72 (d, 3H), 2.48 (brq, 2H), 1.78 (brs, 2H). ¹³C-NMR δ 172.5; 163.4; 160.8; 133.4; 130.6; 130.2; 127.5; 127.4; 116.3; 116.1; 115.9; 115.7; 54.5; 48.8; 46.2; 43.6; 40.3; 26.3.

### Example 59

### 2-(4-Methoxyphenyl)-N-(4-fluorobenzyl)-N-(1-methylpiperidin-4-yl)acetamide (50ELH8)

Procedure as 50ELH14B

Reaction-Step 2:

Starting materials: 50ELH4 (0.12 g, 0.54 mmol, 1.0 eq.), 4-methoxyphenylacetylchloride (0.1 g, 1.0 eq.).

Product: Yield 54 g (26%), UV/MS 100/100 (M⁺ 371), rₜ (A, MS) 3.257, R^{f} 0.25 (3% MeOH/CH₂Cl₂). ¹H-NMR (400 MHz, CDCl₃) 67 12.2 (brs, 1H), 7.12 (m, 2H), 6.97 (m, 4H), 6.75 (d, 2H), 4.80 (brt, 1H), 4.49 (s, 2H), 3.71 (s, 3H), 3.47 (s, 2H), 3.37 (d, 2H), 2.8 (q, 2H), 2.64 (s, 3H), 2.35 (q, 2H), 1.69 (d, 2H). ¹³C-NMR δ 173.0; 163.5; 161.1; 158.9; 133.7; 133.6; 129.8; 127.6; 127.5; 126.5; 116.2; 116.0; 114.6; 114.5; 55.5; 54.4; 48.8; 46.2; 43.6; 40.5; 26.4.

### Example 60

### 2-(Phenyl)-N-(4-fluorobenzyl)-N-(1-methylpiperidin-4-yl)acetamide (50ELH10)

Procedure as 50ELH14B.

Reaction-Step 2: 2-(Phenyl)-N-(4-fluorobenzyl)-N-(1-methylpiperidin-4-yl)acetamide (50ELH10).

Starting materials: 50ELH4 (0.13 g, 0.59 mmol, 1.0 eq.), phenylacetylchloride (0.091 g, 1.0 eq.).

Product: UV/MS 100/94 (M⁺ 341), rₜ (A, MS) 3.127, R^{f} 0.25 (3% MeOH/CH₂Cl₂). ¹H-NMR (400 MHz, DMSO, rotamers 54/56) δ 12.38 (brs, 1H), 7.35-7.00 (m, 9H), 4.55 and 4.40 (2s, 2H), 4.50 and 4.25 (brt, 1H), 3.91 and 3.56 (2s, 2H), 3.30 (Hidden under water signal) (2H), 2.98 (d, 2H), 2.64 (s, 3H), 2.09 (brt, 2H), 1.66 and 1.45 (2brd, 2H). ¹³C-NMR δ 171.9; 171.6; 162.8; 160.4; 136.5; 136.2; 135.4; 129.9; 129.7; 129.5; 129.2; 129.0; 128.9; 128.7; 127.2; 127.1; 116.2; 116.0; 115.6; 53.2; 52.5; 49.8; 46.9; 44.0; 42.8; 40.9; 40.6; 40.4; 40.2; 40.0; 39.8; 39.6; 27.7; 26.6.

### Example 61

### 2-(4-Trifluoromethylphenyl)-N-(4-fluorobenzyl)-N-(1-methylpiperidin-4-yl)acetamide (50ELH12.sup.2)

Procedure as 50ELH14B.

Reaction Step 0: 4-Trifluoromethyphenylacetyl chloride (50ELH12.sup.1)

4-Trifluorophenylacetic acid (1.0 g) and thionyl chloride (15 ml) were refluxed for 1 h. The excess thionyl chloride was evaporated off. NMR showed complete conversion.

Reaction-Step 2: 2-(4-Trifluoromethlphenyl)-N-(4-fluorobenzyl)-N-(1-methylpiperidin-4-yl)acetamide (50ELH122).

Starting materials: 50ELH4 (0.12 g, 0.55 mmol, 1.0 eq.), 4-trifluoromethylphenylacetylchloride (50ELH12.sup. 1) (0.11 g, 0.5 mmol, 1.0 eq.).

Product: Yield 47.1 mg (24%), UV/MS 96/96 (M⁺ 409), rₜ (A, MS) 4.566, R^{f} 0.25 (3% MeOH/CH₂Cl₂). ¹H-NMR (400 MHz, CDCl₃) δ 7.52 (d, 2H), 7.22 (d, 2H), 7.17 (dd, 2H), 7.04 (t, 2H), 4.86 (brt, 1H), 4.58 (s, 2H), 3.64 (s, 2H), 3.45 (brd, 2H), 2.84 (brq, 2H), 2.71 (d, 3H), 2.45 (brq, 2H), 1.77 (brd, 2H). ¹³C-NMR δ 171.8; 163.6; 161.2; 138.7; 133.3; 129.8; 129.5; 127.5; 127.4; 125.8; 125.7; 116.4; 116.2; 54.4; 48.9; 46.3; 43.6; 40.8; 26.3.

### Example 62

### 4-(4-Methoxybenzylamino)-1-methylpiperidine (50ELH18)

Procedure as 50ELH27.

Starting materials: 1-Methyl-4-piperidone (1.13 g, 10.0 mmol, 1.0 eq.), 4-methoxybenzylamine (1.37 g, 1.0 eq.).

Product: UV/MS 95/95 (M⁺ 235), rₜ (A, MS) 3.509. ¹H-NMR (400 MHz, CDCl₃) δ 7.3-6.8 (m, 4H), 3.77 (s, 3H), 3.73 (s, 2H), 2.86 (m, 2H), 2.55 (m, 1H), 2.30 (s, 3H), 2.1 (t, 2H), 1.96 (dd, 2H), 1.50 (m, 2H).

### Example 63

### 2-(4-Trifluoromethylphenyl)-N-[4-(methoxycarbonyl)benzyl]-N-(1-methylpiperidin-4-yl)acetamide (50ELH20A)

Procedure as 50ELH14B.

Reaction-Step 1: Methyl 4-(N-[1-methylpiperidine-4-yl]aminomethyl)benzoate (50ELH19).

Starting materials: 1-Methyl-4-piperidone (1.13 g, 10.0 mmol, 1.0 eq.), methyl 4-(aminomethyl)benzoate hydrochloride (2.0 g, 1.0 eq.).

Product: UV/MS 81/88 (M⁺ 263), rₜ (A, MS) 3.060. ¹H-NMR (400 MHz, CDCl₃) δ 8.00 (d, 2H), 7.20 (d, 2H), 3.90 (s, 3H), 3.85 (s, 2H), 2.96 (dt, 2H), 2.7 (brs, 1H), 2.62 (m, 1H), 2.40 (s, 3H), 2.28 (t, 2H), 1.96 (m, 2H), 1.56 (m, 2H).

Reaction-Step 2: 2-(4-Trifluoromethylphenyl)-N-[4-(methoxycarbonyl)benzyl]-N-(1-methylpiperidin-4-yl)acetamide (50ELH20A).

Starting materials: 50ELH19 (0.20 g, 0.76 mmol, 1.0 eq.), 50ELH12¹ (0.169 g, 1.0 eq.).

Product: Yield 108.9 mg (32%), UV/MS 100/100 (M⁺ 448), rₜ (A, MS) 3.327, R^{f} 0.3 (5% MeOH/CH₂Cl₂). ¹H-NMR (400 MHz, DMSO, rotamers 56/44) δ 10.7 and 10.4 (2brs, 1H), 7.96-7.28 (m, 8H), 4.70 and 4.51 (2s, 2H), 4.30 (brt, 1H), 4.06 and 3.69 (2s, 2H), 3.83 and 3.81 (2s, 3H), 3.00 (m, 2H), 2.63 (m, 3H), 2.05 (brt, J=12 Hz, 2H), 1.69 (brt, J=12 Hz, 2H). ¹³C-NMR (CDCl₃) δ 171.9; 166.7; 142.9; 138.5; 130.7; 130.1; 129.7; 126.2; 125.9; 55.2; 52.5; 49.2; 47.4; 41.2; 32.1; 26.6; 22.9; 14.3.

### Example 64

### 2-Phenyl-N-[4-(methoxycarbonyl)benzyl]-N-(1-methylpiperidin-4-yl)acetamide (50ELH20B)

Procedure as 50ELH14B

Reaction-Step 2: 2-Phenyl-N-[4-(methoxycarbonyl)benzyl]-N-(1-methylpiperidin-4-yl)acetamide (50ELH20B)

Starting materials: 50ELH19 (0.2 g, 0.76 mmol, 1.0 eq.), phenylacetylchloride (0.117 g, 1.0 eq.).

Product: Yield 82.5 g (29%), UV/MS 100/100 (M⁺ 381), rₜ (A, MS) 2.652, R^{f} 0.25 (30% MeOH/CH₂Cl₂). ¹H-NMR (400 MHz, CDCl₃) δ 12.2 (brs, 1H), 8.00 (d, J=7.4, 2H), 7.4-7.2 (m, 4H), 7.08 (d, J=7.4, 2H), 4.89 (brt, 1H), 4.62 (s, 2H), 3.90 (s, 3H), 3.56 (s, 2H), 3.42 (d, J=11.0, 2H), 2.84 (q, J=11.0, 2H), 2.68 (d, J=3.6, 3H), 240 (q, J=11.0, 2H), 1.77 (brd, J=1.0, 2H). ¹³C-NMR δ 173.0; 168.0; 143.3; 136.7; 130.6; 129.0; 127.4; 125.9; 54.5; 52.4; 48.8; 43.6; 41.4; 26.3.

### Example 65

### 2-(4-Chlorophenyl)-N-[4-(methoxycarbonyl)benzyl]-N-(1-methylpiperidin-4-yl)acetamide (50ELH20C)

Procedure as 50ELH14B.

Reaction-Step 2: 2-(4-Chlorophenyl)-N-[4-(methoxycarbonal)benzyl]-N-(1-methylpiperidin-4-yl)acetamide (50ELH20C).

Starting materials: 50ELH19 (0.2 g, 0.76 mmol, 1.0 eq.), 4-chlorophenylacetylchloride (0.131 g, 1.0 eq.).

Product: Yield 79.2 g (26%), UV/MS 100/96 (M⁺ 399), rₜ (A, MS) 2.333. ¹H-NMR (400 MHz, DMSO, rotamers 62/38) δ 10.8 and 10.60 (2brs, 1H), 7.95 and 7.85 (2d, J=8.6, 2H), 7.4 and 7.28 (2d, 2H), 7.35 and 7.14 (2m, 4H), 4.67 and 4.50 (2s, 2H), 4.29 (m, 1H), 3.93 and 3.84 (2s, 2H), 3.81 (s, 3H), 3.21 (d, J=11.9, 2H), 3.00 (d, J=11.9, 2H), 2.63 (s, 3H), 2.06 (m, 2H), 1.68 and 1.56 (d, J=11.9, 2H). ¹³C-NMR (CDCl₃) δ 172.6; 166.7; 163.4; 161.0; 143.0; 130.7; 130.6; 130.5; 126.0; 115.9; 115.7; 54.7; 52.4; 48.9; 46.9; 44.0; 40.4; 26.4.

### Example 66

### 2-(4-Methoxyphenyl)-N-[4-(methoxycarbonyl)benzyl]-N-(1-methylpiperidin-4-yl)acetamide (50ELH20D)

Procedure as 50ELH14B.

Reaction-Step 2: 2-(4-Methoxyphenyl)-N-[4-(methoxycarbonyl)benzyl]-N-(1-methylpiperidin-4-yl) acetamide (50ELH20D).

Starting materials: 50ELH19 (0.2 g, 0.76 mmol, 1.0 eq.), 4-methoxyphenylacetylchloride (0.140 g, 1.0 eq.).

Product: Yield 108.6 g (26%), UV/MS 100/99 (M⁺ 410), rₜ (A, MS) 2.280. ¹H-NMR (400 MHz, CDCl₃) δ 12.38 (brs, 1H), 8.00 (d, J=7.2, 2H), 7.28 (d, J=7.2, 2H), 7.00 (d, J=7.2, 2H), 6.79 (d, J=7.2, 2H), 4.88 (brt, 1H), 4.61 (s, 2H), 3.90 (s, 3H), 3.75 (s, 3H), 3.42 (brd, J=10.7, 2H), 2.84 (q, J=10.7, 2H), 2.68 (d, J=3.6, 3H), 2.40 (brq, J=10.7, 2H1), 1.75 (d, J=10.7, 2H). ¹³C-NMR δ 173.0; 166.8; 159.0; 143.3; 130.5; 129.9; 129.8; 126.3; 125.9; 114.5; 55.5; 54.7; 52.4; 48.7; 46.7; 43.6; 40.6; 32.1; 26.3; 22.9; 14.3.

### Example 67

### 2-(4-TMethylphenyl)-N-[4-(methoxycarbonyl)benzyl]-N-(1-methylpiperidin-4-yl)acetamide (50ELH23)

Procedure as 50ELH14B.

Reaction-Step 2: 1-Phenyl-N-[2-(4-methylphenyl)ethyl]-N-(1-methylpiperidin-4-yl)amide (50ELH23).

Starting materials: 4-(2-Phenylethyl)amino-1-methylpiperidine (0.20 g, 0.86 mmol, 1.0 eq.), benzoylchloride (0.158 g, 1.0 eq.).

Product: Yield 159 mg (50%), UV/MS 100/100 (M⁺ 337), rₜ (A, MS) 3.289, R^{f} 0.55 (10% MeOH/CH₂Cl₂). ¹H-NMR (400 MHz, DMSO (80° C.)) δ 10.9 (brs, 1H), 7.44 (s, 2H), 7.34 (d, J=3.0 Hz, 2H), 7.04 (d, J=7.0 Hz, 2H), 6.95 (brs, 2H), 4.00 (brs, 1H), 3.40 (d, J=4.2 Hz, 2H), 3.35 (d, J=4.2 Hz, 2H), 2.95 (brs, 2H), 2.77 (t, J=3.2 Hz, 2H), 2.40 (q, J=6.4 Hz, 2H), 2.24 (s, 3H) 1.83 (d, J=6.4 Hz, 2H). ¹³C-NMR (CDCl₃) δ 171.6; 138.1; 136.3; 136.0; 129.8; 129.6; 129.1; 129.1; 126.7; 53.6; 52.4; 46.1; 42.9; 35.9; 27.3; 21.1.

### Example 68

### 2-(4-Methoxyphenyl)-N-(3-phenyl-1-propyl)-N-(1-methylpiperidin-4-yl)acetamide (50ELH65)

Procedure as 50ELH14B.

Reaction-Step 1: 4-(3-Phenylaminopropyl)piperidine (50ELH59)

Starting materials: 1-Methyl-4-piperidone (1.1 ml, 7.4 mmol, 1.0 eq.), 3-phenylpropylamine (1.35 g, 1.0 eq.).

Product. UV/MS 100/94 (M⁺ 233), rₜ (A, MS) 3.534). ¹H-NMR (400 MHz, CDCl₃) δ 7.28-7.12 (m, 5H), 3.40 (brs, 1H), 2.84 (dt, J=12.3 and 3.5 Hz, 2H), 2.94 (g, J=7.0 Hz, 4H), 2.51 (m, 1H), 2.27 (s, 3H), 2.05 (brt, J=12.3 Hz, 2H), 1.82 (m, 2H), 1.44 (m, 2H).

Reaction-Step 2: 2-(4-Methoxyphenyl)-N-(3-phenyl-1-propyl)-N-(1-methylpiperidin-4-yl)acetamide (50ELH65)

Starting materials: 50ELH59 (0.50 g, 2.2 mmol, 1.0 eq.), 4-methoxyphenylacetylchloride (0.398 g, 1.0 eq.).

Product: Yield 153 mg (43%), UV/MS 100/100 (M⁺ 381), rₜ (A, MS) 2.938. ¹H-NMR (400 MHz, DMSO, rotamers 55/45) δ 11.0 and 10.90 (2brs, 1H), 7.30-7.10 (m, J=7.9 Hz, 6H), 6.97 (d, J=7.9 Hz, 1H), 4.22 and 4.06 (2dt, dH), 3.70 (s, 3H), 3.35 (t, J=10.4 Hz, 2H), 3.15 (m, 2H), 3.00 (q, J=10.4 Hz, 2H), 2.66 (d, 3H), 2.52 (q, J=7.9 Hz, 2H), 2.17 (brq, J=12 Hz, 2H) 1.73 (m, 2H), 1.70 and 1.52 (2d, J=12 Hz, 2H). ¹³C-NMR (DMSO) δ 171.3; 171.0; 158.6; 142.2; 141.7; 130.0; 129.0; 128.0; 128.5; 128.2; 126.6; 114.5; 55.7; 55.7; 53.5; 53.3; 50.1; 44.5; 42.9; 41.9; 33.7; 33.1; 32.9; 31.4; 27.8; 26.8.

### Example 69

### 2-(4-Methoxyphenyl)-N-[2-(4-methylphenyl)ethyl)-N-(1-methylpiperidin-4-yl)acetamide (50ELH68)

Procedure as 50ELH14B

Reaction-Step 1: 4-[2-(4-Methylphenyl)ethylamino]-piperidin (50ELH58)

Starting materials: 1-Methyl-4-piperidone (1.1 ml, 7.4 mmol, 1.0 eq.), 2-(4-methylphenyl)ethylamine (1.0 g, 1.0 eq.).

Product: UV/MS 100/91 (M⁺ 233), rₜ (A, MS) 3.933). ¹H-NMR (400 MHz, CDCl₃) δ 7.4 (s, 5H), 3.27 (brs, 1H), 2.84 (d, J=7.0 Hz, 4H), 2.75 (m, 2H), 2.54 (m, 1H), 2.29 (2×s, 6H), 2.10 (brt, J=12.3 Hz, 2H), 1.86 (brd, 2H), 1.45 (m, 2H).

Reaction-Step 2: 2-(4-Methoxyphenyl)-N-[2-(4-methylphenyl)ethyl)-N-(1-methylpiperidin-4-yl)acetamide (50ELH68)

Starting materials: 50ELH58 (0.30 g, 1.3 mmol, 1.0 eq.), 4-methoxyphenylacetylchloride (0.238 g, 1.0 eq.).

Product: Yield 125 mg (26%), UV/MS 100/99 (M⁺ 381), rₜ (A, MS) 3.156. ¹H-NMR (400 MHz, DMSO, rotamers 50/50) δ 11.0 and 10.90 (2brs, 1H), 7.25-7.04 (m, J=8.7 Hz, 6H), 6.87 and 6.84 (2d, J=8.7 Hz, 2H), 4.30 and 4.09 (2dt, J=11.5 Hz, dH), 3.73 and 3.58 (2s, 2H), 3.71 and 3.70 (2s, 3H), 3.35 (m, (Underneath waterpeak) 3H), 3.24 (m, 1H), 3.02 (m, J=11.5 Hz, 2H), 2.80-2.62 (m, 5H), 2.32 and 2.20 (2q, J=11.5 Hz, 2H), 2.26 and 2.24 (2s, 3H) 1.78 and 1.49 (2d, J=11.5 Hz, 2H). ¹³C-NMR (DMSO) δ 171.5; 171.2; 158.6; 136.8; 136.2; 136.0; 135.8; 130.7; 130.5; 129.7; 129.6; 129.4; 129.2; 128.4; 128.3; 114.5; 55.8; 55.7; 53.3; 53.3; 52.2; 50.2; 46.8; 43.9; 42.9; 36.8; 35.2; 27.6; 26.8; 21.3.

### Example 70

### 2-(4-Methoxyphenyl)-N-[2-(2-thienyl)ethyl]-N-(1-methylpiperidin-4-yl)acetamide (50ELH71A)

Procedure as 50ELH14B

Reaction-Step 1: 4-[2-(2-Thienyl)ethylamino]piperidin (50ELH67A)

Starting materials: 1-Methyl-4-piperidone (0.5 g, 4.4 mmol, 1.0 eq.), thiophene-2-ethylamine (0.563 g, 1.0 eq.).

Product: UV/MS 94/93 (M⁺ 225).

Reaction-Step 2: 2-(4-Methoxyphenyl)-N-[2-(2-thienylethyl]-N-(1-methylpipieridin-4-yl)acetamide (50ELH71A)

Starting materials: 50ELH67A (0.243 g, 1.08 mmol, 1.0 eq.) 4-methoxyphenylacetylchloride (0.2 g, 1.0 eq.).

Product: Yield 80.7 mg (33%), UV/MS 100/100 (M⁺ 373), rₜ (A, MS) 2.613. ¹H-NMR (400 MHz, DMSO, rotamers 50/50) δ 10.8 and 10.6 (2brs, 1H), 7.36 and 7.31 (2d, J=4.7 Hz, 1H), 7.20 and 7.06 (2d, J=8.3 Hz, 2H), 7.00-6.92 (m, J=4.7 and 2.8 Hz, 2H), 6.87 and 6.40 (2d, J=8.3 Hz, 2H), 4.22 and 4.08 (2dt, J=12.2 Hz, 1H), 3.71 (s, 3H), 3.70 (s, 2H), 3.46-3.30 (m, 4H), 3.10-2.90 (m, 4H), 2.67 (m, 2H), 2.28 and 2.12 (2q, J=12 Hz, 2H), 1.80 and 1.50 (2d, J=12 Hz, 2H). ¹³C-NMR (DMSO) δ 172.5; 158.9; 139.6; 130.0; 129.6; 126.8; 124.5; 114.5; 55.5; 54.7; 49.3; 45.8; 43.8; 41.3; 31.9; 29.9

### Example 71

### 2-(4-Methoxyphenyl)-N-[2-(4-nitrophenyl)ethyl]-N-(1-methylpiperidin-4-yl)acetamide (50ELH71C)

Procedure as 50ELH14B

Reaction-Step 1: 4-[2-(4-nitrophenyl)ethylamino]-piperidin (50ELH67C)

Starting materials: 1-Methyl-4-piperidone (0.5 g, 4.4 mmol, 1.0 eq.), 4-nitrophenyl-2-ethylamine (0.897 g, 1.0 eq.).

Product: UV/MS 96/89 (M⁺ 264), rₜ (A, MS) 3.264.

Reaction-Step 2: 2-(4-Methoxyphenyl)-N-[2-(4-nitrophenyl)ethyl]-N-(1-methylpiperidin-4-yl)acetamide (50ELH71A)

Starting materials: 50ELH67C (0.285 g, 1.08 mmol, 1.0 eq.), 4-methoxyphenylacetylchloride (0.2 g, 1.0 eq.).

Product: Yield 130.9 mg (30%), UV/MS 100/100 (M⁺ 412), rₜ (A, MS) 2.219. ¹H-NMR (400 MHz, DMSO, rotamers 50/50) δ 10.8 and 10.6 (2brs, 1H), 8.17 and 8.12 (2d, J=8.6 Hz, 2H), 7.58 and 7.48 (2d, J=8.6 Hz, 2H), 7.2 and 7.1 (2d, J=8.6 Hz, 2H), 6.87 and 6.40 (2d, J=8.6 Hz, 2H), 4.25 and 4.10 (2dt, J=12 Hz, 1H), 3.72 (s, 3H), 3.70 (s, 2H), 3.48-3.30 (m, 4H), 3.10-2.84 (m, 4H), 2.69 and 2.67 (2d, J=4.7 Hz, 3H), 2.34 and 2.15 (2q, J=13.2 Hz, 2H), 1.79 and 1.47 (2d, J=13.2 Hz, 2H).

### Example 72

### 2-(4-Methoxyphenyl)-N-(2-thienylmethyl)-N-(1-methylpiperidin-4-yl)acetamide (50ELH73A)

Procedure as 50ELH14B.

Reaction-Step 1: 4-[(2-Thienylmethyl)amino]-1-methylpiperidine (50ELH66A)

Starting materials: 1-Methyl-4-piperidone (0.5 g, 4.4 mmol, 1.0 eq.), 2-thienylmethylamine (0.52 g, 1.0 eq.).

Product: UV/MS 77/86 (M⁺ 211), rₜ (A, MS) 2.739.

Reaction-Step 2: 2-(4-Methoxyphenyl)-N-(2-thienylmethyl)-N-(1-methylpiperidin-4-yl)acetamide (50ELH73A)

Starting materials: 50ELH66A (0.228 g, 1.08 mmol, 1.0 eq.), 4-methoxyphenylacetylchloride (0.2 g, 1.0 eq.).

Product: Yield 178.4 mg (50%), UV/MS 100/98 (M⁺ 359), rₜ (A, MS) 3.117. ¹H-NMR (400 MHz, DMSO) δ 10.9 and 10.6 (2brs, 1H), 7.47 and 7.32 (2d, J=4.5 Hz, 1H), 7.20 and 7.03 (2d, J=8.4 Hz, 2H), 7.03 and 6.98 (2m, 1H), 6.87 (m, 3H), 4.70 and 4.57 (2s, 2H), 4.42 and 4.16 (2t, J=11.9 Hz, 1H), 3.77 and 3.60 (2s, 2H), 3.51 (s, 3H), 3.15 (m, 2H), 2.98 (m, J=11.9 Hz, 2H), 2.65 (2d, J=4.5 Hz, 3H), 2.25 and 2.17 (2q, J=11.9 Hz, 2H), 1.69 and 1.44 (2d, J=111.9 Hz, 2H). ¹³C-NMR (DMSO) δ 171.4; 158.6; 143.2; 130.7; 128.1; 126.6; 126.3; 125.9; 114.5; 55.7; 53.3; 52.6; 50.0; 42.8; 27.7; 26.8.

### Example 73

### 2-(4-Methoxyphenyl)-N-(furfuryl)-N-(1-methylpiperidin-4-yl)acetamide (50ELH73B)

Procedure as 50ELH14B.

Reaction-Step 1: 4-(Furfurylamino)-1-methylpiperidin (50ELH66B)

Starting materials: 1-Methyl-4-piperidone (0.5 g, 4.4 mmol, 1.0 eq.), Furfurylamine (0.43 g, 1.0 eq.).

Product: UV/MS 77/92 (M⁺ 195), rₜ (A, MS) 2.812).

Reaction-Step 2: 2-(4-Methoxyphenyl)-N-(furfuryl)-N-(1-methylpiperidin-4-yl)acetamide (50ELH73B).

Starting materials: 50ELH66B (0.21 g, 1.08 mmol, 1.0 eq.), 4-methoxyphenylacetylchloride (0.2 g, 1.0 eq.).

Product: Yield 134 mg (36%), UV/MS 100/99 (M⁺ 343), rₜ (A, MS) 2.401. ¹H-NMR (400 MHz, DMSO, rotamers 57/43) δ 10.95 and 10.75 (2brs, 1H), 7.63 and 7.48 (s, 1H), 7.18 and 7.06 (2d, J=7.7 Hz, 2H), 6.85 (t, J=7.7 Hz, 2H), 6.44 and 6.33 (2d, J=7.7 Hz, 1H), 6.37 and 6.11 (2s, 1H) 4.5 and 4.34 (2s, 2H), 4.42 and 4.18 (2dt, J=11 and 2 Hz, 1H), 3.75 and 3.65 (2s, 2H) 3.70 (s, 3H), 3.33 (hidden, 2H), 3.0 (q, 2H), 2.64 (d, J=4.7 Hz, 3H), 2.15 (dq, J=11 and 2 Hz, 2H), 1.65 and 1.50 (2d, J=11 Hz, 2H).

### Example 74

### 2-(2-thienyl)-N-(4-methylphenylmethyl)-N-(1-methylpiperidin-4-yl)acetamide (50ELH82)

Procedure as 50ELH14B

Reaction-Step 2: 2-(2-thienyl)-N-(4-methylphenylmethyl)-N-(1-methylpiperidin-4-yl)acetamide (50ELH82)

Starting materials: 50ELH25 (0.30 g, 1.38 mmol, 1.0 eq.), thiophene-2-acetylchlorid (0.22 g, 1.0 eq.).

Product: Yield 235 mg (62%), UV/MS 97/93 (M⁺ 343), rₜ (A, MS) 2.795. ¹H-NMR (400 MHz, DMSO, rotamers 54/46) δ 10.8 and 10.60 (2brs, 1H), 7.4 and 7.35 (2d, 1H), 7.2-6.76 (m, 6H), 4.55 and 4.4 (2s, 2H), 4.49 and 4.26 (2dt, J=1 and 2 Hz, 2H), 4.15 and 3.79 (2s, 2H), 3.32 (d, J=1 Hz, 2H), 2.99 (q, 2H), 2.63 (s, 3H), 2.27 and 2.23 (2s, 3H), 2.09 (q, J=11 Hz, 2H), 1.66 and 1.55 (2d, J=11 Hz, 2H).

### Example 75

### 2-(4-Methoxyphenyl)-N-(4-methylbenzyl)-N-(1-cyclopentylpiperidin-4-yl)acetamide (42ELH75)

Procedure as for 42ELH80, except that the reaction was run at 60° C. for 3 days.

Starting materials: 50ELH87 (0.25 g, 0.71 mmol, 1.0 eq.), Cyclopentylbromide (0.288 g, 3.0 eq.).

Product: Yield 91.2 mg (34%), UV/MS 88/93 (M⁺ 421), rₜ (A, MS) 4.450.

### Example 76

### 2-(4-Methoxyphenyl)-N-(4-methylbenzyl)-N-(1-(3-(1,3-dihydro-2H-benzimidazol-2-one-1-yl)propyl)piperidine-4-yl)acetamide (50ELH89)

50ELH87 (0.05 g, 0.14 mmol, 1 eq.) was transferred to a 4 ml vial and dissolved in 1 ml of acetonitrile. Then, 1-(3-chloropropyl)-1,3-dihydro-2H-benzimidazol-2-one (0.032 g, 1.1 eq.), sodium carbonate (0.022 g, 1.1 eq.) and KI (one crystal) were added and the vial was sealed and shaken for 20 h at 82° C. The mixture was extracted with distilled water (pH˜10, sodium carbonate) and dichloromethane (3 times) the organic layers were dried with sodium sulfate and concentrated. The title compound was purified by HPLC and evaporated to dryness, forming a trifluoroacetic acid salt. Yield 8.8 mg (12%). UV/MS 100/100 (M⁺ 527), rₜ (A, MS) 2.851.

### Example 77

### 2-(4-Methoxyphenyl)-N-(4-methylbenzyl)-N-[1-(2-methylthiazol-4-ylmethyl)piperidin-4-yl]acetamide (63ELH1A)

50ELH87 (0.3 g, 0.852 mmol, 1.0 eq) and 4-(chloromethyl)-2-methylthiazole hydrochloride (0.235 g, 1.5 eq) were added to a 7 ml vial and dissolved in acetonitrile (3 ml). Potassium carbonate (141.3 g, 1.2 eq) and a crystal of potassium iodide were added and the vial was sealed and shaken for 20 h at 82° C. The reaction mixture was extracted with distilled water (made basic by potassium carbonate, pH 10) and dichloromethane. The crude product was dried with sodium sulfate and concentrated. After purification by HPLC the product was converted into the hydrochloride salt by dissolving the free base in 1 ml dichloromethane and adding 1 eq. HCl in ether (2M). This mixture was added drop-wise to an excess of heptane where the product precipitated. The solvent was removed by evaporation leaving a white powder as the product. yield 83.8 mg (21%), UV/MS 100/90 (M⁺ 463), rₜ (B, MS) 11.82.

### Example 78

### 2-(4-Methoxyphenyl)-N-(2-4-(fluorophenyl)ethyl)-N-(1-methylpiperidin-4-yl)acetamide (50ELH93A)

Procedure as 50ELH14B.

Reaction-Step 1: 4-[2-4-(Fluorophenyl)ethylamino]-1-methylpiperidine (50ELH92A)

Starting materials: 1-Methyl-4-piperidone (0.3 g, 2.65 mmol, 1.0 eq.), 4-(fluorophenyl)ethylamine (0.369 g, 1.0 eq.).

Product: UV/MS 60/92 (M⁺ 237), rₜ (A, MS) 3.422.

Reaction-Step 2: 2-(4-Methoxyphenyl)-N-(2-4-(fluorophenyl)ethyl)-N-(1-methylpiperidin-4-yl)acetamide (50ELH93A)

Starting materials: 50ELH92A (0.625 g, 2.65 mmol, 1.0 eq.), 4-methoxyphenylacetylchloride (0.488 g, app. 1.0 eq.).

Product: Yield 181 mg (18%), UV/MS 87/97 (M⁺ 385), rₜ (A, MS) 2.783. R^{f} 0.8 (10% MeOH/CH₂Cl₂). ¹H-NMR (400 MHz, DMSO, rotamers 50/50) δ 10.9 (brs, 1H), 7.56-6.8 (m, 8H), 4.26 and 4.02 (2brt, 2H), 3.70 and 3.95 (2s, 3H), 3.59 and 3.57 (2s, 2H), 3.4-3.15 (m, 5H), 2.96-2.66 (m, 5H), 2.62 and 2.56 (2s, 3H), 2.29 and 2.10 (2q, 2H), 1.73 and 1.41 (2d, 2H). ¹³C-NMR (DMSO) δ 172.5; 171.4; 171.3; 162.9; 162.7; 160.5; 160.3; 158.9; 158.6; 136.1; 136.1; 135.3; 131.4; 131.3; 131.1; 131.0; 131.0; 130.6; 130.5; 128.4; 128.4; 126.9; 115.9; 115.8; 115.7; 115.6; 114.5; 55.7; 53.7; 53.5; 52.7; 52.3; 50.7; 46.7; 43.8; 43.2; 43.0; 36.3; 34.7; 27.9; 26.9.

### Example 79

### 2-(4-Methoxyphenyl)-N-[2-(2,5-dimethoxyphenyl)ethyl]-N-(1-methylpiperidin-4-yl)acetamide (50ELH93C)

Procedure as 50ELH14B. A small amount was purified by HPLC and evaporated to dryness, forming the trifluoroacetic acid salt.

Reaction-Step 1: 4-[2-(2,5-dimethoxyphenyl)ethylamino]-1-methylpiperidine (50ELH92A)

Starting materials: Methyl-4-piperidone (0.3 g, 2.65 mmol, 1.0 eq.), 2,5-(dimethoxyphenyl)ethylamine (0.481 g, 1.0 eq.).

Product: UV/MS 81/90 (M⁺ 279), rₜ (A, MS) 2.868.

Reaction-Step 2: 2-(4-Methoxyphenyl)-N-[2-(2.5-dimethoxyphenyl)ethyl]-N-(1-methylpiperidin-4-yl)acetamide (50ELH93C)

Starting materials: 50ELH93C (0.737 g, 2.65 mmol, 1.0 eq.), 4-methoxyphenylacetylchloride (0.488 g, app. 1.0 eq.).

Product: UV/MS 82/100 (M⁺ 427), rₜ (B, MS) 8.44. R^{f} 0.8 (10% MeOH/CH₂Cl₂).

### Example 80

### 2-(4-Methoxyphenyl)-N-[2-(2,4-dichlorophenyl)ethyl]-N-(1-methylpiperidin-4-yl)acetamide (50ELH93D)

Procedure as 50ELH14B, but purified by HPLC and evaporated to dryness forming the trifluoroacetic acid salt.

Reaction-Step 1: 4-[2-(2,4-Dichlorophenyl)ethylamino]-1-methylpiperidine (50ELH92D)

Starting materials: 1-Methyl-4-piperidone (0.3 g, 2.65 mmol, 1.0 eq.), 2,5-(dichlorophenyl)ethylamine (0.50 g, 1.0 eq.).

Product: UV/MS 82/92 (M⁺ 287), rₜ (A, MS) 4.875.

Reaction-Step 2: 2-(4-Methoxyphenyl)-N-[2-(2,4-dichlorophenyl)ethyl]-N-(1-methylpiperidin-4-yl)acetamide (50ELH93D)

Starting materials: 50ELH93D (0.76 g, 2.65 mmol, 1.0 eq.), 4-methoxyphenylacetylchloride (0.488 g, app. 1.0 eq.).

Product: UV/MS 100/96 (M⁺ 435), rₜ (A, MS) 4.415. R^{f} 0.8 (10% MeOH/CH₂Cl₂).

### Example 81

### 2-(4-Methoxyphenyl)-N-[2-(3-chlorophenyl)ethyl]-N-(1-methylpiperidin-4-yl)acetamide (50ELH93E)

Procedure as 50ELH14B, but purified on HPLC and evaporated to dryness forming the trifluoroacetic acid salt.

Reaction-Step 1: 4-[(3-Chlorophenyl)ethyl)amino]-1-methylpiperidine (50ELH92E)

Starting materials: 1-Methyl-4-piperidone (0.3 g, 2.65 mmol, 1.0 eq.), 3-(chlorophenyl)ethylamine (0.413 g, 1.0 eq.).

Product: UV/MS 86/88 (M⁺ 253), rₜ (A, MS) 3.175.

Reaction-Step 2: 2-(4-Methoxyphenyl)-N-[2-(3-chlorophenyl)ethyl]-N-(1-methylpiperidin-4-yl)acetamide (50ELH93E)

Starting materials: 50ELH93E (0.67 g, 2.65 mmol, 1.0 eq.), 4-methoxyphenylacetylchloride (0.488 g, app. 1.0 eq.).

Product: UV/MS 100/100 (M⁺ 401), rₜ (A, MS) 3.464. R^{f} 0.8 (10% MeOH/CH₂Cl₂).

### Example 82

### 2-(4-Methoxyphenyl)-N-[2-(4-methoxyphenyl)ethyl]-N-(1-methylpiperidin-4-yl)acetamide (50ELH95B)

Procedure as 50ELH14B. Purified by HPLC and evaporated to dryness forming the trifluoroacetic acid salt.

Reaction-Step 1: 4-[(4-Methoxyphenyl)ethyl)amino]-1-methylpiperidine (50ELH94B)

Starting materials: 1-Methyl-4-piperidone (0.3 g, 2.65 mmol, 1.0 eq.), 4-methoxyphenylethylamine (0.40 g, 1.0 eq.).

Product: UV/MS 74/87 (M⁺ 249), rₜ (A, MS) 2.935.

Reaction-Step 2: 2-(4-Methoxyphenyl)-N-[2-(4-methoxyphenyl)ethyl]-N-(1-methylpiperidin-4-yl)acetamide (50ELH95B)

Starting materials: 50ELH94B (0.657 g, 2.65 mmol, 1.0 eq.), 4-methoxyphenylacetylchloride (0.488 g, app. 1.0 eq.).

Product: UV/MS 100/100 (M⁺ 397), rₜ (A, MS) 2.389. R^{f} 0.8 (10% MeOH/CH₂Cl₂).

### Example 83

### 2-(4-Methoxyphenyl)-N-[2-(3-fluorophenyl)ethyl]-N-(1-methylpiperidin-4-yl)acetamide (50ELH95D)

Procedure as 50ELH14B. Purified on HPLC and evaporated to dryness, forming the trifluoroacetic acid salt.

Reaction-Step 1: 4-[2-((3-Fluorophenyl)ethyl)amino]-1-methylpiperidine (50ELH94D)

Starting materials: 1-Methyl-4-piperidone (0.3 g, 2.65 mmol, 1.0 eq.), 3-fluorophenylethylamine (0.369 g, 1.0 eq.).

Product: UV/MS 74/89 (M⁺ 237), rₜ (A, MS) 2.946.

Reaction-Step 2: 2-(4-methoxyphenyl)-N-[2-(3-fluorophenyl)ethyl]-N-(1-methylpiperidin-4-yl)acetamide (50ELH95D)

Starting materials: 50ELH94D (0.625 g, 2.65 mmol, 1.0 eq.), 4-methoxyphenylacetylchloride (0.488 g, app. 1.0 eq.).

Product: UV/MS 100/95 (M⁺ 385), rₜ (A, MS) 2.946. R^{f} 0.8 (10% MeOH/CH₂Cl₂).

### Example 84

### 2-(4-ethoxyphenyl)-N-[2-(4-fluorophenyl)ethyl]-N-(1-methylpiperidin-4-yl)acetamide (63ELH20)

Reaction Step, 1: 4-Ethoxyphenylacetic acid chloride(63ELH19)

4-Ethoxyphenylacetic acid (0.5 g, 2.8 mmol) was transferred to a 7 ml vial and dissolved in thionylchloride (3 ml). The reaction mixture was shaken at 70° C. for 2½ hours. Thionylchloride was evaporated off and the resulting product was used unpurified.

Reaction Step 2: 2-(4-Ethoxyphenyl)-N-[2-(4-fluorophenyl)ethyl]-N-(1-methylpiperidin-4-yl)acetamide (63ELH20)

63ELH17 (0.11 g, 0.47 mmol) was transferred to a 4 ml vial and dissolved in dichloromethane. 63ELH19 (0.084 mg, 1 eq.) was added and the vial was sealed and the reaction shaken for 20 h. The product was extracted in distilled water (made basic with potassium carbonate, pH 10) and dichloromethane. Dried with sodium sulfate and concentrated. Purified by HPLC. The extraction, drying and concentration was repeated and the product re-dissolved in dichloromethane (1 ml) and HCl (1 eq., 2 M in ether) was added. The mixture was added drop-wise to an excess of heptane whereupon the salt precipitated. Yield 33.4 mg (18%), UV/MS: 92/100 (M⁺ 399), tᵣ (B, MS) 10.38.

### Example 85

### 2-(4-Ethoxyphenyl)-N-(4-fluorobenzyl)-N-(1-methylpiperidin-4-yl)acetamide (63ELH21)

50ELH4 (0.11 g, 0.49 mmol, 1.0 eq.) was transferred to a 4 ml vial and dissolved in dichloromethane. 63ELH19 (0.089 mg, 1.0 eq.) was added and the vial was sealed and the reaction shaken for 20 h. The product was extracted in distilled water (made basic with potassium carbonate, pH 10) and dichloromethane. Dried with sodium sulfate and concentrated. Purified by HPLC. The extraction, drying and concentration was repeated and the product dissolved in dichloromethane (1 ml) and HCl (1 eq., 2 M in ether)is added. This mixture was added drop-wise to an excess of heptane whereupon the salt precipitated. Yield 31.1 mg (16%), UV/MS: 94/100 (M⁺ 385), tᵣ (A, MS) 2.573.

### Example 86

### N-((4-methylphenyl)methyl)-N-(1-methylpiperidin-4-yl)-2-(3-hydroxy-4-methoxyphenyl)acetamide (57MBT12B)

N-((4-methylphenyl)methyl)-4-amino-1-methylpiperidine (50ELH25) (105 mg, 0.48 mmol) and 3-hydroxy-4-methoxyphenylacetic acid (88 mg, 0.48 mmol) were dissolved in DMF (10 ml). Diisopropylethylamine (DIEA, 250 μL, 1.44 mmol) was added followed by bromo-tris-pyrrolidino-phosphonium hexafluorophosphate (PyBrOP, 336 mg, 0.72 mmol), and the mixture was stirred at r.t for 1 h. Water (50 mL) was added, and the reaction mixture was extracted with EtOAc (2×50 mL). Drying by Na₂SO₄ and concentration yielded 514 mg crude material, which was purified by flash chromatography (0-30% MeOH in CH₂Cl₂). This gave 105 mg (57%)of the title compound as a white solid. R^{f}=0.20 (10% MeOH in CH₂Cl₂). HPLC-MS (method A) showed MH⁺=383. UV/MS (%)=100/92. ¹H-NMR (400 MHz, CD₃OD, Rotamers 52:48): δ 7.18-6.58 (m, 7H), 4.53 (s, 2H), 4.31 and 3.97 (2m, 1H), 3.82 and 3.81 (2s, 3H), 3.80 and 3.55 (2s, 2H), 3.04 and 2.85 (2m, 2H), 2.41 and 2.32 (2s, 3H), 2.35 and 2.12 (2m, 2H), 2.29 and 2.27 (2s, 3H), 1.83 and 1.74 (2m, 2H), 1.72 and 1.33 (2m, 2H)

### Example 87

### N-((4-methylphenyl)methyl)-N-(1-methylpiperidin-4-yl)-2-(3,4-dihydroxyphenyl)acetamide (57MBT24B)

N-((4-methylphenyl)methyl)-N-(1-methylpiperidine-4-yl)-2-(3-hydroxy-4-methoxyphenyl)acetamide (57MBT12B) (52 mg, 0.136 mmol) was dissolved in CH₂Cl₂ (1 mL) and cooled to −78° C. Boron tribromide (1M in CH₂Cl₂, 204 μl, 0.204 mmol) was added dropwise and the cooling bath was removed. After stirring for 2 h, methanol (2 mL) was added and the mixture was evaporated. The resulting oil was purified by preparative HPLC to give 24 mg (48%) of the title compound as a white solid. HPLC-MS (method A) showed MH⁺=369. UV/MS (%)=100/97. ¹H-NMR (400 MHz, CD₃OD, Rotamers 33:67): δ 7.19-6.47 (m, 7H), 4.54 and 4.53 (2s, 2H), 4.23 (m, 1H), 3.83 and 3.58 (2s, 2H), 3.46 and 3.40 (2br d, J=12 Hz, 2H), 3.02 and 2.95 (2br t, J=12 Hz, 2H), 2.79 (s, 3H), 2.33 and 2.28 (2s, 3H), 2.17 and 1.84 (2dq, J=4, 12 Hz, 2H), 1.87 and 1.48 (2br d, J=12 Hz, 2H)

### Example 88

### N-((3-hydroxy-4-methylphenyl)methyl)-N-(1-methylpiperidin-4-yl)-2-(4-methoxyphenyl)acetamide (57MBT54B)

N-((4-methoxyphenyl)methyl)-4-amino-1-methylpiperidine (1 g, 4.27 mmol) was dissolved in 4% formic acid in methanol (60 mL). 10% Pd/C (1 g) was added under argon and the reaction mixture was heated to reflux for 24 h. The mixture was filtered through celite and the filtrate was acidified with conc. HCl to pH˜1. Concentration yielded a yellow oil which was purified by flash chromatography (MeOH/CH₂Cl₂ 3:7+3.5% NH₄ OH)to give 249 mg (51%) of 4-amino-1-methylpiperidine (57-MBT36B) as a white solid. R_{f} 0.13 (10% MeOH in CH₂Cl₂+3.5% NH₄OH). HPLC-MS (method B) showed MH⁺=115. UV/MS (%)=−/100.

4-Amino-1-methylpiperidine (57MBT36B) (26 mg, 0.231 mmol) was dissolved in methanol (1 mL) and 3-hydroxy-4-methylbenzaldehyde (32 mg, 0.231 mmol) and acetic acid (33 μL) were added. The mixture was cooled to 0° C. NaBH₃ CN (29 mg, 0.462 mmol) was added and the cooling bath was removed. After 3 h the reaction mixture was evaporated and flash chromatography (0-30% MeOH in CH₂Cl₂) gave 27 mg (50%) of N-((3-hydroxy-4-methylphenyl)methyl)-4-amino-1-methylpiperidine (57MBT44C) as a white solid. R^{f}=0.27 (10% MeOH in CH₂Cl₂+3.5% NH₄₀H). HPLC-MS (method A) showed MH⁺=235. UV/MS (%)=99/99.

N-((3-hydroxy-4-methylphenyl)methyl)-4-amino-1-methylpiperidine (57MBT44C) (27 mg, 0.115 mmol) was dissolved in CH₂Cl₂ (2 mL). 4-Methoxyphenylacetyl chloride (17 μL, 0.115 mmol) was added dropwise under argon. After 3 h, n-heptane (3 mL) was added and the mixture was evaporated. Flash chromatography (0-20% MeOH in CH₂Cl₂) gave 14 mg (32%) of the title compound as a white solid. R^{f}=0.32 (10% MeOH in CH₂Cl₂+3.5% NH₄₀H). HPLC-MS (method A) showed MH⁺=383. UV/MS (%)=99/96. ¹H-NMR (400 MHz, CD₃OD, Rotamers 63:37): δ 7.28-6.55 (m, 7H), 4.48 (s, 2H), 4.37 and 3.95 (2m, 1H), 3.78 and 3.77 (2s, 3H), 3.06 and 2.89 (2br d, J=12 Hz, 2H), 2.42 and 2.32 (2s, 3H), 2.40 and 2.12 (2m, 2H), 2.18 and 2.12 (2s, 3H), 1.86 and 1.83 (2m, 2H), 1.75 and 1.35 (2br d, J=12 Hz, 2H)

### Example 89

### N-((4-methylphenyl)methyl)-N-(1-methylpiperidin-4-yl)-2-(4-bromophenyl)acetamide hydrochloride(57MBT70-1D)

4-Bromophenylacetic acid (54 mg, 0.252 mmol) was dissolved in CH₂Cl₂ (2 mL), and N-((4-methylphenyl)methyl)-4-amino-1-methylpiperidine (292 mg/mL stock solution in CH₂Cl₂, 171 μL, 0.229 mmol) and polystyrene supported diisopropylethylamine (PS-DIEA with a loading of 3.57 mmol/g, 192 mg, 0.687 mmol) was added followed by bromo-tris-pyrrolidino-phosphonium hexafluorophosphate (PyBrOP, 160 mg/mL stock solution, 1 mL, 0.334 mmol). The reaction mixture was shaken for 1 h at r.t. and filtered onto a prewashed (methanol) ion exchange column (0.88 mmol/g, 1 g). The column was washed with methanol (8×4 mL) and the remaining product was eluted off the column with 10% NH₄ OH in methanol (2×4 mL) and evaporated. The resulting oil was filtered through silica (H=4 cm, D=1 cm) with methanol/CH₂Cl₂ 1:9 (20 mL), evaporated and subjected to a second ion exchange column (0.88 mmol/g, 1 g). The column was washed with methanol (8*4 mL) and the remaining product was eluted off the column with 10% NH₄ OH in methanol (2*4 mL) and evaporated on rotavap and oil pump. The product was dissolved in CH₂Cl₂ (0.5 mL) and HCl in diethylether (1.0 M, 0.1 mL, 0.1 mmol) was added. The solution was added to n-heptane (3 mL) and evaporation afforded 29 mg (25%) of the title compound as a white solid. R^{f}=0.31 (10% MeOH in CH₂Cl₂). HPLC-MS (method B) showed MH⁺=416. UV/MS (%)=100/99.

### Example 90

### N-((4-methylphenyl)methyl)-N-(1-methylpiperidin-4-yl)-2-(4-iodophenyl)acetamide hydrochloride(57MBT70-2D)

The title compound was prepared according to example MBT04. Yield: 33 mg (26%). R^{f}=0.31 (10% MeOH in CH₂Cl₂). HPLC-MS (method B)showed MH⁺=463. UV/MS (%)=100/98.

### Example 91

### N-((4-methylphenyl)methyl)-N-(1-methylpiperidin-4-yl)-2-(4-(2-propyl)phenyl acetamide hydrochloride (57MBT70-3D)

The title compound was prepared according to example MBT04. Yield: 36 mg (34%). R^{f}=0.31 (10% MeOH in CH₂Cl₂). HPLC-MS (method B) showed MH⁺=379. UV/MS (%)=100/97.

### Example 92

### N-((4-methylphenyl)methyl)-N-(1-methylpiperidin-4-yl)-2-(4-trifluoromethoxy phenyl)acetamide hydrochloride(57MBT70-4D)

The title compound was prepared according to example MBT04. Yield: 35 mg (30%). R^{f}=0.27 (10% MeOH in CH₂Cl₂). HPLC-MS (method B) showed MH⁺=421. UV/MS (%)=100/99.

### Example 93

### N-((4-methylphenyl)methyl)-N-(1-methylpiperidin-4-yl)-2-(4-methylthiophenyl)acetamide hydrochloride(57MBT70-5D)

The title compound was prepared according to example MBT04. Yield: 35 mg (33%). R^{f}=0.30 (10% MeOH in CH₂Cl₂). HPLC-MS (method B) showed MH⁺=383. UV/MS (%)=100/99.

### Example 94

### N-((4-methylphenyl)methyl)-N-(1-methylpiperidin-4-yl)-2-(4-(N,N-dimethylamino)phenyl)acetamide hydrochloride(57MBT70-6D)

The title compound was prepared according to example MBT04.

Yield: 16 mg (15%). R^{f}=0.25 (10% MeOH in CH₂Cl₂). HPLC-MS (method A) showed MH⁺=380. UV/MS (%)=100/100.

### Example 95

### N-((4-methylphenyl)methyl)-N-(1-methylpiperidin-4-yl)-2-(4-nitrophenyl)acetamide hydrochloride(57MBT70-7D)

The title compound was prepared according to example MBT04. Yield: 28 mg (27%). R^{f}=0.27 (10% MeOH in CH₂Cl₂). HPLC-MS (method B) showed MH⁺=382. UV/MS (%)=100/100.

### Example 96

### N-((4-methylphenyl)methyl)-N-(1-methylpiperidin-4-yl)-2-(4-methoxy-3-methyl phenyl)acetamide hydrochloride(57MBT70-8D)

The title compound was prepared according to example MBT04. Yield: 34 mg (32%). R^{f}=0.30 (10% MeOH in CH₂Cl₂). HPLC-MS (method B) showed MH⁺=381. UV/MS (%)=100/99.

### Example 97

### N-((4-methylphenyl)methyl)-N-(1-methylpiperidin-4-yl)-2-(4-pyridyl)acetamide hydrochloride(57MBT70-9F)

The title compound was prepared according to example MBT04. Yield: 18 mg (17%). R^{f}=0.09 (10% MeOH in CH₂Cl₂). HPLC-MS (method A) showed MH⁺=338. UV/MS (%)=100/100.

### Example 98

### N-((4-methylphenyl)methyl)-N-(1-methylpiperidin-4-yl)-2-(4-methylphenyl)ace tamide hydrochloride(57MBT62B)

The title compound was prepared according to example MBT04. Yield: 10 mg (35%). R^{f}=(10% MeOH in CH₂Cl₂). HPLC-MS (method A) showed MH⁺=351. UV/MS (%)=100/100.

### Example 99

### N-((4-(hydroxymethyl)phenyl)methyl)-N-(1-methylpiperidin-4-yl)-2-(4-methoxy phenyl)acetamide hydrochloride(57MBT72D)

To a stirred suspension of LiAlH₄ (285 mg, 7.52 mmol) in diethylether (10 mL)at 0° C. was added a solution of 4-cyanobenzyl alcohol (0.5 g, 3.76 mmol) in diethylether (5 mL) over 15 min. The grey reaction mixture was heated to reflux for 3 h. After cooling to r.t., the mixture was treated successively with water (1 mL), 2M NaOH (2 mL) and water (2 mL) under vigorous stirring. The resulting white slurry was filtered and washed with CH₂Cl₂ (20 mL). Extraction with additional CH₂Cl₂ (20 mL) and n-butanol (20 mL) and evaporation yielded an oil, which upon flash chromatography (0-15% MeOH in CH₂Cl₂) gave 152 mg (29%)of 4-(aminomethyl)benzylalcohol (57MBT52B) as a white solid. R^{f}=0.51 (30% MeOH in CH₂Cl₂+3.5% NH₄₀H).

1-Methyl-4-piperidone (84 μL, 0.73 mmol) was dissolved in methanol (5 mL) and 4-(aminomethyl)benzylalcohol (57MBT52B) (100 mg, 0.73 mmol) was added followed by acetic acid (125 μL). NaBH₃ CN (92 mg, 1.46 mmol) was added and the mixture was stirred for 3 h. The reaction mixture was evaporated and 2M NaOH (5 mL) was added. Extraction with CH₂Cl₂ (4×5 mL), drying with Na₂SO₄ and evaporation gave 152 mg (87%) of N-((4-(hydroxymethyl)phenyl)methyl)-4-amino-1-methylpiperidine (57MBT56D) as a white solid. HPLC-MS (method B) showed MH⁺=235. UV/MS (%)=100/100.

N-((4-(Hydroxymethyl)phenyl)methyl)-4-amino-1-methylpiperidine (57MBT56D) (20 mg, 0.0853 mmol) was dissolved in CH₂Cl₂ (2 mL) and 4-methoxyphenylacetyl chloride (26 μL, 0.171 mmol) was added dropwise. The reaction mixture was stirred for 1 h and water (500 μL) was added followed by evaporation. A solution of sodium (5 mg, 0.179 mmol) in methanol (2 mL) was added. After stirring for 4 h, the solution was transferred to a prewashed (methanol) ion exchange column (0.88 mmol/g, 1 g) and washed with methanol (4×4 mL). The remaining product was eluted off the column with 10% NH₄ OH in methanol (2×4 mL) and evaporated. The resulting oil was filtered through silica (H=4 cm, D=1 cm) with methanol/CH₂Cl₂ 2:8 (20 mL), evaporated and subjected to a second ion exchange column (0.88 mmol/g, 1 g). The column was washed with methanol (8×4 mL) and the remaining product was eluted off the column with 10% NH₄ OH in methanol (2×4 mL) and evaporated on rotavap and oil pump. The product was dissolved in CH₂Cl₂ (0.5 mL) and HCl in diethylether (1.0 M, 0.1 mL, 0.1 mmol) was added. The solution was added to n-heptane (3 mL) and evaporation afforded 14 mg (39%) of the title compound as a white solid. R^{f}=0.16 (10% MeOH in CH₂Cl₂). HPLC-MS (method B) showed MH⁺=383. UV/MS (%)=100/96.

### Example 100

### 2-(4-Chlorophenyl)-N-(4-methylbenzyl)-N-(1-isopropylpiperidin-4-yl)acetamide (47AKU-7) 1-Trifluoroacetyl-4-piperidone (47AKU-2)

4-Piperidone hydrochloride monohydrate (3.85 g, 25 mmol) and Triethylamine (10.5 ml, 75 mmol) were partly dissolved in 100 ml of dichloromethane and stirred for 10 min. Reaction mixture was then cooled on ice-bath and trifluoroacetic anhydride (7.2 ml, 50 mmol) was slowly added over 10 min. Ice-bath was removed and mixture was stirred overnight. Additional trifluoroacetic anhydride (2 ml) was added and the mixture was stirred for 1 hr. Water (200 ml) was added. Phases were separated and aq. phase was re-extracted with dichloromethane. Combined organic phases were washed with brine, dried over MgSO₄ and concentrated (40° C.) giving 4.97 g (100%) 47AKU-2 as yellow crystals. TLC (5% methanol in dichloromethane): R^{f}=0.8. ¹H-NMR (400 MHz, CDCl₃): δ 3.87-3.99 (4H, m); 2.54-2.61 (4H, m). ¹³C-NMR (CDCl₃): δ 204.7, 118.0, 115.1, 44.2, 42.8, 41.2, 40.5.

### 4-(4-Methylbenzylamino)-1-trifluoroacetyl-piperidine (47AKU-3)

47AKU-2 (4.97 g, 25 mmol) was dissolved in 100 ml methanol and 4-methylbenzyl-amine (3.2 ml, 25 mmol) was added. Mixture was stirred and acetic acid (˜2 ml) was added until pH˜5. NaCNBH₃ (3.15 g, 50 mmol) was slowly added. After magnetic stirring for 20 hrs the methanol was partly removed on the rotary evaporator (40° C.). Dichloromethane, 2M NaOH and water were added until pH˜10. Phases were separated and aq. phase was then re-extracted twice with dichloromethane. Combined organic phases were washed with brine and dried over MgSO₄. Concentration(40° C.) yielded 6.94 g (92%) 47AKU-3. TLC (10% methanol in dichloromethane): R^{f}=0.6. HPLC-MS (Method A): M⁺=301.0 (UV/MS (%)=94/100).

### 2-(4-Chlorophenyl)-N-(4-methylbenzyl)-N-(1-trifluoroacetylpiperidin-4-yl)acetamide (47AKU-4)

47AKU-3 (3.01 g, 10 mmol) in 25 ml of dichloromethane was placed in a 100 ml flask. Triethylamine (1.4 ml, 10 mmol) was added and the mixture was cooled on an ice-bath and stirred for 10 min. 4-Chlorophenylacetyl chloride (1.90 g, 10 mmol) was dissolved in 10 ml dichloromethane and added slowly to the ice-cold mixture. After 15 min. the ice-bath was removed and the mixture was left for 1 hr. Precipitation was observed. The reaction mixture was then concentrated at aspirator pressure(40° C.). The crude product was purified by flash chromatography (0-50% ethylacetate in heptane) yielding 2.38 g (53%/) 47AKU-4. TLC (100% dichloromethane): R^{f}=0.6. HPLC-MS (Method A): M⁺=453.0 (UV/MS (%)=89/84).

### 2-(4-Chlorophenyl)-N-(4-methylbenzyl)-N-(piperidin-4-yl)acetamide (47AKU-6)

47AKU-4 (2.38 g; ˜5 mmol) was dissolved in 50 ml of methanol. K₂CO₃ (3.5 g; 25 mmol) was added in one portion. After magnetic stirring for 20 hrs, additional K₂CO₃ (1 g) was added. After 4 hrs magnetic stirring methanol was partly removed by evaporation(40° C.). Ethyl acetate (100 ml) and water (100 ml) were added. The phases were separated and the aq. phase was then re-extracted with ethylacetate. The combined organic phases were dried over MgSO₄ and concentrated (40° C.) giving 1.95 g (100%) 47AKU-6. TLC (20% methanol in dichloromethane): R^{f}=0.3. HPLC-MS (Method A): M⁺=357.1 (UV/MS (%)=84/95).

### 2-(4-Chlorophenyl)-N-(4-methylbenzyl)-N-(1-isopropylpiperidin-4-yl)-acetamide (47AKU-7)

47AKU-6 (358 mg, 1.0 mmol) was dissolved in 20 ml of acetonitrile. Triethylamine (1.4 ml, 10 mmol) was added and mixture was stirred for 10 min. Isopropyl bromide (370 mg, 3.0 mmol) was dissolved in 5 ml of acetonitrile and added to the reaction mixture which was stirred at room temp. for 20 hrs and then heated to 60° C. for 4 hrs. After cooling, ethylacetate (25 ml) and water (25 ml) were added. The phases were separated and the aq. phase was then re-extracted with ethylacetate. The combined organic phases were washed with brine, dried over MgSO₄ and concentrated (40° C.) giving 362 mg of crude product. Purification by flash chromatography (0-10% methanol in dichloromethane) and HCl-precipitation from 2M HCl/diethyl ether in dichloromethane/heptane gave 76 mg (18%) 47AKU-7. TLC (10% methanol in dichloromethane): R^{f}=0.4. Mp=223-224° C. HPLC-MS (Method A): M⁺=399.1 (UV/MS (%)=100/99). ¹H-NMR (400 MHz, CDCl₃): δ 7.03-7.29 (8H, m); 4.86 (1H, m); 4.61 (2H, m); 3.58 (2H, m); 3.37 (3H, m); 2.82 (2H, m); 2.64 (2H, m); 2.34 (3H, s); 1.80 (2H, m); 1.39 (6H, d). ¹³C-NMR (CDCl₃): δ 172.4 137.4, 134.8, 133.3, 133.1, 130.4, 129.9, 129.0, 125.8, 58.0, 49.5, 48.2, 46.6, 40.4, 26.0, 21.2, 17.0.

### Example 101

### 2-(4-Chlorophenyl)-N-(4-methylbenzyl)-N-(1-ethylpiperidin-4-yl)acetamide (47AKU-12)

47AKU-6 (358 mg, 1.0 mmol) was dissolved in 20 ml of acetonitrile. Triethylamine (1.4 ml, 10 mmol) was added and the mixture was stirred for 10 min. Ethyl bromide (370 μl, 5.0 mmol) was added. The mixture was then heated to 50° C. and stirred overnight. After cooling, water (25 ml) and ethylacetate (25 ml) were added. The phases were separated and the aq. phase was re-extracted with ethylacetate. The combined organic phases were washed with brine and dried over MgSO₄. Evaporation(40° C.) yielded 406 mg of crude product. Purification by ion exchange chromatography (washout with 10% aq. NH₄OH (25%) in methanol) gave 166 mg (43%) 47AKU-12. The HCl-salt was prepared from 2M HCl/diethylether in dichloromethane/heptane. TLC (10% methanol in dichloromethane): R^{f}=0.5. HPLC-MS (Method A): M⁺=385.1 (UV/MS (%)=100/99). ¹H-NMR (400 MHz, CDCl₃, rotamers): δ 7.02-7.34 (8H, m); 4.62 (1H, m); 4.46 and 4.53 (2H, 2s); 3.81 (1H, s); 3.55 (2H, s); 2.92 (2H, m); 2.34 (3H, s); 2.29 (1H, s); 1.98 (2H, m); 1.52-1.84 (4H, m); 1.03 (3H, t). ¹³C-NMR (CDCl₃): δ 171.7, 137.2, 135.4, 133.9, 132.8, 130.4, 129.7, 128.9, 125.8, 52.8, 52.4, 46.5, 40.8, 31.2, 29.8, 21.2, 12.4.

### Example 102

### 2-Phenyl-N-(4-methylbenzyl)-N-(1-methylpiperidin-4-yl)-acetamide (47AKU-13)

47AKU-5 (218 mg, 1.0 mmol) was dissolved in 2 ml of dichloromethane in a 50 ml flask. Phenylacetyl chloride (134 μl, 1.0 mmol) was added. After 3 hrs stirring at room temp. mixture was concentrated on Rotavapor (40° C.). Crude product was purified by ion exchange chromatography (washout with 10% aq. NH₄OH (25%) in methanol) and flash chromatography (0-10% methanol in dichloromethane) giving 48 mg (14%) 47AKU-13. HCl-salt was prepared from 2M HCl/diethylether in dichloromethane/heptane. TLC (10% methanol in dichloromethane): Rf=0.4. HPLC-MS (Method A): M+=337.1 (UV/MS (%)=98/98). ¹H-NMR (400 MHz, CDCl₃, rotamers): δ 7.01-7.40 (9H, m); 4.63 (1H, m); 4.53 and 4.45 (2H, 2s); 3.85 and 3.61 (2H, 2s); 2.86 and 2.77 (2H, 2m); 2.35 and 2.29 (3H, 2s); 2.25 and 2.20 (3H, 2s); 209 (2H, m); 1.61-1.86 (4H, m). ¹³C-NMR (CDCl₃): δ 72.2, 137.1, 135.5, 129.7, 128.9, 128.8, 127.2, 126.9, 125.8, 55.3, 51.6, 46.6, 46.1, 41.6, 29.5, 21.2.

### Example 103

### 2-(4-Chlorophenyl)-N-(4-methylbenzyl)-N-(1-methylpiperidin-4-yl)-acetamide (47AKU-8)

### 4-(4-Methylbenzylamino)-1-methyl-piperidine (47AKU-5)

1-Methyl-4-piperidone (1.13 g, 10 mmol) was dissolved in 20 ml of methanol and added to a 100 ml flask. 4-Methylbenzylamine (1.21 g, 10 mmol) in 10 ml of methanol was added. Acetic acid (˜1.5 ml) was added until pH˜5. NaCNBH₃ (1.26 g, 20 mmol) was slowly added. After 20 hrs magnetic stirring methanol was partly removed on Rotavapor (40° C.). Dichloromethane, water and 2M NaOH were added until pH˜10. The phases were separated and aq. phase was extracted twice with dichloromethane. The combined organic phases were washed with brine and dried over MgSO₄. Concentration on Rotavapor (40° C.) yielded 2.06 g crude (93%) 47AKU-5. TLC (20% methanol in dichloromethane): R^{f}=0.3. HPLC-MS (Method A): M⁺=219.1 (UV/MS (%)=89/98).

### 2-(4-Chlorophenyl)-N-(4-methylbenzyl)-N-(1-methylpiperidin-4-yl)-acetamide (47AKU-8)

47AKU-5 (437 mg, 2.0 mmol) was dissolved in 10 ml of dichloromethane in a 50 ml flask. Triethylamine (280 μl, 2.0 mmol) was added and the mixture was cooled to 0° C. on an ice bath and stirred for 10 min. 4-Chlorophenylacetyl chloride (380 mg, 2.0 mmol) was dissolved in 10 ml of dichloromethane and added to the cooled mixture. After 2 hrs stirring at room temp. additional dichloromethane (10 ml) and water (20 ml) were added. The phases were separated and the aq. phase was re-extracted with dichloromethane. The combined organic phases were dried over MgSO₄ and concentrated on the Rotavapor (40° C.) giving 755 mg of crude product. Purification by flash chromatography (0-10% methanol in dichloromethane)gave 485 mg (65%) product. Further purification by ion exchange chromatography (washout with 10% aq. NH₄OH (25%) in methanol) gave 239 mg (32%) 47AKU-8. The HCl-salt was prepared from 2M HCl/diethylether in dichloromethane/heptane. TLC (10% methanol in dichloromethane): R^{f}=0.4. Mp=217-219° C. HPLC-MS (Method A): M⁺=371.1 (UV/MS (%)=99/99). ¹H-NMR (400 MHz, CD₃OD): δ 7.05-7.39 (8H, m); 4.80 (3H, s); 4.62+4.56 (2H, 2s); 4.35 (1H, m); 4.00 (1H, s); 3.71 (1H, s); 3.46 (2H, m); 3.06 (2H, m); 2.80 (3H, s); 2.32+2.27 (3H, 2s); 2.19 (1H, m). ¹³C-NMR (CD₃OD): δ 173.0, 137.5, 134.5, 133.9, 132.6, 130.6, 129.5, 128.5, 126.2, 54.0, 51.4, 42.6, 40.2, 31.8, 26.6, 19.9.

### Example 104

### 2-(4-Chlorophenyl)-N-(4-methylbenzyl)-N-(1-cyclopentylpiperidin-4-yl)-acetamide (47AKU-11)

47AKU-6 (358 mg, 1.0 mmol) was dissolved in 20 ml of acetonitrile. Triethylamine (1.4 ml, 10 mmol) was added and mixture was stirred for 10 min. Cyclopentylbromide (540 μl, 5.0 mmol) was added and the mixture was stirred at room temp. After 20 hrs the mixture was heated to 50° C. for an additional 24 hrs. The reaction mixture was then cooled and water (25 ml) and ethylacetate (25 ml) were added. The phases were separated and the aq. phase was re-extracted with ethylacetate. The combined organic phases were washed with brine and dried over MgSO₄. Concentration on Rotavapor (45° C.) yielded 426 mg of crude product. Purification by ion exchange chromatography (washout with 10% aq. NH₄ OH (25%) in methanol) and flash chromatography (0-10% methanol in dichloromethane)gave 76 mg (18%) 47AKU-11. The HCl-salt was prepared from 2M HCl/diethylether in dichloromethane/heptane. TLC (10% methanol in dichloromethane): R^{f}=0.5. HPLC-MS (Method A): M⁺=425.1 (UV/MS (%)=100/97). ¹H-NMR (400 MHz, CDCl₃, rotamers): δ 7.01-7.34 (8H, m); 4.67 (1H, m); 4.49 and 4.52 (2H, 2s); 3.54 (2H, s); 3.15 and 3.02 (2H, 2m); 2.64 (1H, m); 2.27 and 2.34 (3H, 2s); 2.20 (1H, m); 1.85 (4H, m); 1.69 (4H, m); 1.53 (4H, m); 1.37 (1H, m). ¹³C-NMR (CDCl₃): δ 171.9, 137.2, 135.2, 133.8, 132.9, 130.4, 129.7, 128.9, 125.8, 67.7, 52.4, 52.1, 46.5, 40.7, 30.2, 28.8, 24.3, 21.2.

### Example 105

### 2-(4-Fluorophenyl)-N-(4-methylbenzyl)-N-(1-methylpiperidin-4-yl)-acetamide (47AKU-14)

47AKU-5 (218 mg, 1.0 mmol) was dissolved in 3 ml of dichloromethane in a 50 ml flask. 4-Fluorophenylacetyl chloride (150 μl, 1.1 mmol) was added. After 4 hrs stirring at room temp. the mixture was concentrated on Rotavapor (40° C.). The crude product was purified by flash chromatography (0-10% methanol in dichloromethane) giving 243 mg (68%) 47AKU-14. The HCl-salt was prepared from 2M HCl/diethylether in dichloromethane/heptane. TLC (10% methanol in dichloromethane): R^{f}=0.5. HPLC-MS (Method A): M⁺=355.1 (UV/MS (%)=100/100). ¹H-NMR (400 MHz, CDCl₃): δ 6.92-7.33 (8H, m); 4.73 (1H, m); 4.52 (2H, s); 3.56 (2H, 2s); 3.44 (5H, m); 3.25 (2H, m); 2.52-2.67 (4H, m); 2.33 (3H, s). ¹³C-NMR (CDCl₃): δ 172.5, 163.3, 160.9, 139.5, 134.8, 130.6, 129.8, 125.8, 115.8, 54.6, 50.8, 49.9, 46.7, 40.4, 27.2, 21.2.

### Example 106

### 2-(4-Chlorophenyl)-N-(4-methylbenzyl)-N-(1-2-hydroxyethyl)-piperidin-4-yl)-acetamide (47AKU-18)

47AKU-6-2 (358 mg, 1.0 mmol) was dissolved in 10 ml of acetonitrile in 50 ml flask. Triethylamine (1.4 ml, 10 mmol) was added and mixture was stirred for 10 min.

2-Bromoethanol (215 μl, 3.0 mmol) was added. Reaction mixture was then heated to 60° C. and stirred overnight. After cooling ethylacetate (25 ml) and water (25 ml) were added. Phases were separated and aq. phase was re-extracted with ethylacetate. Combined organic phases were washed with brine, dried over MgSO₄ and concentrated on Rotavapor (40° C.) giving 406 mg crude product. Purification by flash chromatography (0-10% methanol in dichloromethane) afforded 253 mg (63%) 47AKU-18. HCl-salt was prepared from 2M HCl/diethylether in dichloromethane/heptane. TLC (10% methanol in dichloromethane): R^{f}=0.4. HPLC-MS (Method A): M⁺=401.1 (UV/MS (%)=100/100). ¹H-NMR (400 MHz, CDCl₃, rotamers): δ 7.04-7.34 (8H, m); 4.60 (1H, m); 4.52 and 4.45 (2H, 2s); 3.55 (4H, m); 3.03 (1H, bs); 2.92 (2H, m); 2.52 (2H, m); 2.36 and 2.31 (3H, 2s); 2.19 (2H, m); 1.66 (4H, m). ¹³C-NMR (CDCl₃): δ 171.7, 137.3, 135.2, 133.8, 132.9, 130.4, 129.8, 128.9, 125.8, 59.4, 58.1, 53.1, 52.3, 46.8, 40.8, 29.7, 21.2.

### Example 107

### 2-(4-Chlorophenyl)-N-(4-methylbenzyl)-N-(1-cyclobutylpiperidin-4-yl)-acetamide (47AKU-19)

### 1-Cyclobutyl-4-piperidone (47AKU-15)

Partly dissolved quartenary salt (1.23 g, 3.7 mmol) (prepared according to the procedure outlined in the synthesis of 47AKU-47) was slowly added to a refluxing solution of Cyclobutylamine (178 mg, 2.5 mmol) and Potassium carbonate (48 mg, 0.34 mmol) in ethanol. The mixture was refluxed for 1.5 hrs. After cooling to room temp. water (10 ml) and dichloromethane (25 ml) were added. Phases were separated and aq. phase was re-extracted with dichloromethane. Combined organic phases were dried over MgSO₄ and concentrated on Rotavapor (40° C.) giving 419 mg crude 47AKU-15. TLC (10% methanol in dichloromethane): R^{f}=0.4. HPLC-MS (Method A): M⁺=154.1 (MS (%)=75).

### 4-(4-Methylbenzylamino)-1-cyclobutyl-piperidine (47AKU-16)

4-Methylbenzylamine (215 mg, 1.8 mmol) was dissolved in 5 ml methanol and placed in 50 ml flask. 47AKU-15 (270 mg, 1.8 mmol) in 5 ml methanol was added. Acetic acid (0.3 ml) was added until pH˜5. NaCNBH₃ (226 mg, 3.6 mmol) was slowly added. Gas evolution observed. After 24 hrs magnetic stirring dichloromethane, 2M NaOH and water were added until pH˜10. Phases were separated and aq. phase was then re-extracted with dichloromethane. Combined organic phases were dried over MgSO₄ and concentrated on Rotavapor (40° C.) yielding 419 mg crude 47AKU-16. TLC (10% methanol in dichloromethane): R^{f}=0.3. HPLC-MS (Method A): M⁺=259.1 (UV/MS (%)=44/87).

### 2-(4-Chlorophenyl)-N-(4-methylbenzyl)-N-(1-cyclobutylpiperidin-4-yl)-acetamide (47AKU-19)

47AKU-16 (209 mg, 0.8 mmol) was placed in 50 ml flask and 5 ml dichloromethane was added. 4-Chlorophenylacetyl chloride (171 mg, 0.9 mmol) in 5 ml dichloromethane was added. After 5 hrs magnetic stirring the reaction mixture was concentrated on Rotavapor (40° C.). Crude product was purified by flash chromatography (0-10% methanol in dichloromethane) giving 101 mg (31%) product. Further purification by ion exchange chromatography (washout with 10% aq. NH₄ OH (25%) in methanol) gave 55 mg (17%) 47AKU-19. Oxalate-salt was prepared from Oxalic acid (1.1 eq) in dichloromethane/heptane. TLC (10% methanol in dichloromethane): R^{f}=0.6. HPLC-MS (Method B): M⁺=411.2 (UV/MS (%)=91/86). ¹H-NMR (400 MHz, CDCl₃, rotamers): δ 7.33-7.01 (8H, m); 4.62 (1H, m); 4.52 and 4.46 (2H, 2s); 3.80 (1H, s); 3.45 and 3.54 (2H, 2s); 2.86 (2H, m); 2.66 (2H, m); 2.28 and 2.34 (3H, 2s); 1.98 (2H, m); 1.80 (2H, m); 1.70-1.52 (6H, m). ¹³C-NMR (CDCl₃): δ 171.7, 137.2, 135.4, 133.9, 132.9, 130.4, 129.7, 128.9, 125.7, 60.4, 52.3, 49.4, 46.5, 40.7, 29.4, 27.6, 21.2, 14.2.

### Example 108

### 2-(4-Methoxyphenyl)-N-(4-methylbenzyl)-N-(1-cyclobutylpiperidin-4-yl)acetamide (47AKU-20)

47AKU-16 (209 mg, 0.8 mmol) was placed in 50 ml flask and 5 ml dichloromethane was added. 4-Methoxyphenylacetyl chloride (167 mg, 0.9 mmol) in 5 ml dichloromethane was added. After 5 hrs magnetic stirring the reaction mixture was concentrated on Rotavapor (40° C.). Crude product was purified by flash chromatography (0-10% methanol in dichloromethane) giving 72 mg (22%) product. Further purification by ion exchange chromatography (washout with 10% aq. NH₄ OH (25%) in methanol) gave 67 mg (20%) 47AKU-20. Oxalate-salt was prepared from Oxalic acid (1.1 eq) in dichloromethane/heptane. TLC (10% methanol in dichloromethane): R^{f}=0.6. HPLC-MS (Method B): M⁺=407.3 (UV/MS (%)=93/77). ¹H-NMR (400 MHz, CDCl₃, rotamers): δ 7.26-6.79 (8H, m); 4.62 (1H, m); 4.52 and 4.45 (2H, 2s); 3.79 (1H, m); 3.77 (3H, s); 3.52 and 3.45 (2H, 2s); 2.84 (2H, m); 2.66 (2H, m); 2.34 and 2.28 (3H, 2s); 1.98 (2H, m); 1.81 (2H, m); 1.72-1.51 (6H, m). ¹³C-NMR (CDCl₃): δ 172.5, 158.7, 137.0, 135.7, 130.4, 129.8, 127.4, 125.8, 114.3, 60.4, 55.5, 52.1, 49.4, 46.4, 40.6, 29.4, 27.6, 21.2, 14.2.

### Example 109

### (47AKU-21) 2-(4-Methoxyphenyl)-N-(4-methylbenzyl)-N-(8-methyl-8-aza-bicyclo[3.2.1]oct-3-yl)-acetamide (47AKU-21)

### 4-(4-Methylbenzylamino)-tropane (47AKU-17)

4-Methylbenzylamine (607 mg, 5.0 mmol) was dissolved in 10 ml methanol and placed in 100 ml flask. Tropinone (697 mg, 5.0 mmol) in 10 ml methanol was added. Acetic acid (0.75 ml) was added until pH˜5. NaCNBH₃ (628 mg, 10 mmol) was slowly added. Gas evolution observed. After 20 hrs magnetic stirring dichloromethane, 2M NaOH and water were added until pH˜10. Phases were separated and aq. phase was then re-extracted with dichloromethane. Combined organic phases were dried over MgSO₄. Concentration on Rotavapor (40° C.) yielded 1.14 g crude 47AKU-17. TLC (10% methanol in dichloromethane): R^{f}=0.4. HPLC-MS (Method A): M⁺=245.2 (UV/MS (%)=65/96).

### 2-(4-Methoxyphenyl)-N-(4-methylbenzyl)-N-(8-methyl-8-aza-bicyclo[3.2.1]oct-3-yl)-acetamide (47AKU-21)

47AKU-17 (244 mg, 1.0 mmol) was placed in 50 ml flask and 5 ml dichloromethane was added. 4-Methoxyphenylacetyl chloride (203 mg, 1.1 mmol) in 10 ml dichloromethane was added. After 3 hrs magnetic stirring the reaction mixture was concentrated on Rotavapor (40° C.). Crude product was purified by ion exchange chromatography (washout with 10% aq. NH₄ OH (25%) in methanol) and flash chromatography (0-10% methanol in dichloromethane) giving 202 mg (51%) 47AKU-21. Oxalate-salt was prepared from Oxalic acid (1.1 eq) in dichloromethane/heptane. TLC (10% methanol in dichloromethane): R^{f} 0.4. HPLC-MS (Method B): M⁺=393.3 (UV/MS (%)=94/92). ¹H-NMR (400 MHz, CDCl₃, isomers): δ 7.02-7.17 (6H, m); 6.78-6.87 (2H, m); 4.74 (1H, s); 4.44 (1H, s); 3.78 and 3.77 (3H, 2s); 3.68 (1H, m); 3.66 and 3.55 (3H, 2s); 2.65 (2H, m); 2.56 (2H, m); 2.32 (3H, s); 2.12-2.26 (6H, m); 2.05 (2H, m). ¹³C-NMR (CDCl₃): δ 173.2, 171.4, 158.8, 137.1, 129.7, 127.6, 126.9, 126.0, 114.4, 63.4, 60.9, 55.5, 54.6, 47.5, 41.5, 40.4, 32.8, 31.1, 27.5, 24.9, 21.2.

### Example 110

### N-(4-Methylbenzyl)-N-(1-methylpiperidin-4-yl)-N′-benzyl-carbamide (47AKU-22)

47AKU-5 (219 mg, 1.0 mmol) was dissolved in 5 ml dichloromethane and placed in 50 ml flask. Benzylisocyanate (160 mg, 1.2 mmol) in 5 ml dichloromethane was added. After 16 hrs magnetic stirring the reaction mixture was concentrated on Rotavapor (40° C.). Crude product was purified by flash chromatography (0-10% methanol in dichloromethane) giving 236 mg (67%) 47AKU-22. Oxalate-salt was prepared from Oxalic acid (1.1 eq) in dichloromethane/heptane. TLC (10% methanol in dichloromethane): R^{f}=0.5. HPLC-MS (Method B): M⁺=352.3 (UV/MS (%)=100/100). ¹H-NMR (400 MHz, CDCl₃): δ 7.26-7.02 (9H, m); 4.61 (1H, m); 4.41 (1H, m); 4.33 (4H, m); 2.87 (2H, m); 2.32 (3H, s); 2.25 (3H, s); 2.09 (2H, m); 1.79-1.62 (4H, m). ¹³C-NMR (CDCl₃): δ 158.6, 139.7, 137.3, 135.4, 129.8, 128.6, 127.4, 127.2, 126.2, 55.5, 52.2, 46.2, 45.8, 45.0, 30.2, 21.2.

### Example 111

### N-(4-Methylbenzyl)-N-(1-methylpiperidin-4-yl)-N′-phenyl carbamide (47AKU-24)

47AKU-5 (219 mg, 1.0 mmol) was dissolved in 5 ml dichloromethane and placed in 50 ml flask. Phenylisocyanate (143 mg, 1.2 mmol) in 5 ml dichloromethane was added. After 4 hrs magnetic stirring the reaction mixture was concentrated on Rotavapor (40° C.). Crude product was purified by flash chromatography (0-10% methanol in dichloromethane) giving 181 mg (54%) 47AKU-24. HCl-salt was prepared from 2M HCl/diethylether in dichloromethane/heptane. TLC (10% methanol in dichloromethane): R^{f}=0.4. HPLC-MS (Method A): M⁺=338.3 (UV/MS (%)=100/100). ¹H-NMR (400 MHz, CDCl₃): δ 7.12-7.24 (8H, m); 6.93-6.98 (1H, m); 6.26 (1H, s); 4.45 (3H, s); 2.90 (2H, d); 2.36 (3H, s); 2.28 (3H, s); 2.12 (2H, m); 1.69-1.85 (4H, m). ¹³C-NMR (CDCl₃): δ 156.1, 139.3, 137.8, 134.9, 130.1, 128.9, 126.3, 123.1, 119.9, 55.5, 52.3, 46.3, 46.2, 30.3, 21.3.

### Example 112

### N-Phenethyl-N-(1-methylpiperidin-4-yl)-N′-benzyl-carbamide (47AKU-25)

4-(2-Phenylethyl)amino-1-methylpiperidine (110 mg, 0.5 mmol) was dissolved in 5 ml dichloromethane and placed in 50 ml flask. Benzylisocyanate (80 mg, 0.6 mmol) in 5 ml dichloromethane was added. After 20 hrs magnetic stirring the reaction mixture was concentrated on Rotavapor (40° C.). Crude product was purified by flash chromatography (0-10% methanol in dichloromethane) giving 164 mg (84%) 47AKU-25. HCl-salt was prepared from 2M HCl/diethylether in dichloromethane/heptane. TLC (10% methanol in dichloromethane): R^{f}=0.4. HPLC-MS (Method A): M⁺=352.3 (UV/MS (%)=100/100). ¹H-NMR (400 MHz, CDCl₃): δ 7.34-7.09 (10H, m); 4.52 (1H, m); 4.35 (2H, d); 4.08 (1H, m); 3.33 (2H, t); 2.92 (2H, m); 2.82 (2H, t); 2.28 (3H, s); 2.07 (2H, m); 1.84-1.66 (4H, m). ¹³C-NMR (CDCl₃): δ 157.9, 139.8, 139.1, 129.0, 128.9, 128.8, 127.8, 127.4, 126.9, 55.7, 52.8, 46.2, 45.3, 44.8, 37.5, 30.6.

### Example 113

### 2-Phenyl-N-(4-methoxybenzyl)-N-(1-methylpiperidin-4-yl)-acetamide (47AKU-26a)

50ELH-18 (118 mg, 0.5 mmol) was dissolved in 5 ml dichloromethane in 50 ml flask.

4-Fluorophenylacetyl chloride (104 mg, 0.6 mmol) was added. After 20 hrs stirring at room temp. mixture was concentrated on Rotavapor (40° C.). Crude product was purified by flash chromatography (0-10% methanol in dichloromethane) giving 87 mg (49%) 47AKU-26a. HCl-salt was prepared from 2M HCl/diethylether in dichloromethane/heptane. HPLC-MS (Method A): M⁺=353.1 (UV/MS (%)=96/88).

### Example 114

### 2-(4-Trifluoromethylphenyl)-N-(4-methoxybenzyl)-N-(1-methylpiperidin-4-yl)-acetamide (47AKU-26b)

50ELH-18 (118 mg, 0.5 mmol) was dissolved in 5 ml dichloromethane in 50 ml flask.

4-Trifluoromethylphenylacetyl chloride (134 mg, 0.6 mmol) was added. After 20 hrs stirring at room temp. mixture was concentrated on Rotavapor (40° C.). Crude product was purified by flash chromatography (0-10% methanol in dichloromethane) giving 81 mg (39%) 47AKU-26b. HCl-salt was prepared from 2M HCl/diethylether in dichloromethane/heptane. HPLC-MS (Method A): M⁺=421.1 (UV/MS (%)=90/100).

### Example 115

### 2-(4-Fluorophenyl)-N-(4-methoxybenzyl)-N-(1-methylpiperidin-4-yl)-acetamide (47AKU-26c)

50ELH-18 (118 mg, 0.5 mmol) was dissolved in 5 ml dichloromethane in 50 ml flask.

4-Fluorophenylacetyl chloride (104 mg, 0.6 mmol) was added. After 20 hrs stirring at room temp. mixture was concentrated on Rotavapor (40° C.). Crude product was purified by flash chromatography (0-10% methanol in dichloromethane) giving 68 mg (37%) 47AKU-26c. HCl-salt was prepared from 2M HCl/diethylether in dichloromethane/heptane. HPLC-MS (Method A): M⁺=371.1 (UV/MS (%)=100/97).

### Example 116

### 2-(4-Methoxyphenyl)-N-(4-methoxybenzyl)-N-(1-methylpiperidin-4-yl)-acetamide (47AKU-26d)

50ELH-18 (118 mg, 0.5 mmol) was dissolved in 5 ml dichloromethane in 50 ml flask.

4-Methoxyphenylacetyl chloride (111 mg, 0.6 mmol) was added. After 20 hrs stirring at room temp. mixture was concentrated on Rotavapor (40° C.). Crude product was purified by flash chromatography (0-10% methanol in dichloromethane) giving 77 mg (40%) 47AKU-26d. HCl-salt was prepared from 2M HCl/diethylether in dichloromethane/heptane. HPLC-MS (Method A): M⁺=383.1 (UV/MS (%)=100/100).

### Example 117

### 2-(4-Methylphenyl)-N-(4-chlorobenzyl)-N-(1-methylpiperidin-4-yl)-acetamide (47AKU-28)

### 4-(4-Chlorobenzylamine)-1-methyl-piperidine (47AKU-27)

1-Methyl-4-piperidone (566 mg, 5.0 mmol) was dissolved in 10 ml methanol and placed in 100 ml flask. 4-Chlorobenzylamine (708 mg, 5.0 mmol) was added. Mixture was stirred and Acetic acid (˜0.75 ml) was added until pH˜5. NaCNBH₃ (628 mg, 10 mmol) was slowly added. Gas evolution observed. After magnetic stirring for 16 hrs methanol was partly removed on Rotavapor (40° C.). Dichloromethane, 2M NaOH and water were added until pH˜10. Phases were separated and aq. phase was then re-extracted with dichloromethane. Combined organic phases were dried over MgSO₄. Concentration on Rotavapor (40° C.) yielded 1.14 g crude 47AKU-27. TLC (10% methanol in dichloromethane): R^{f}=0.3. HPLC-MS (Method A): M⁺=239.1 (MS (%)=96).

### 2-(4-Methylphenyl)-N-(4-chlorobenzyl)-N-(1-methylpiperidin-4-yl)-acetamide (47AKU-28)

p-Tolylacetic acid (1.50 g) was dissolved in 10 ml thionylchloride and placed in 50 ml flask. Mixture was heated to reflux for 2 hrs and then concentrated on Rotavapor (40° C.).

p-Tolylacetic chloride (202 mg, 1.2 mmol) in 5 ml dichloromethane was added to 47AKU-27 (239 mg, 1.0 mmol) in 5 ml dichloromethane. After 4 hrs magnetic stirring the reaction mixture was concentrated on Rotavapor (40° C.). Crude product was purified by flash chromatography (0-10% methanol in dichloromethane) giving 104 mg (28%) 47AKU-28. HCl-salt was prepared from 2M HCl/diethylether in dichloromethane/heptane. TLC (10% methanol in dichloromethane): R^{f}=0.5. HPLC-MS (Method A): M⁺=371.1 (UV/MS (%)=100/90). ¹H-NMR (400 MHz, CDCl₃, rotamers): δ 7.34-6.99 (8H, m); 4.57 (1H, m); 4.50 and 4.44 (2H, 2s); 3.80 (1H, s); 3.55 (1H, s); 2.96 and 2.82 (2H, 2m); 2.34 (1H, m); 2.32 (3H, s); 2.24 and 2.15 (3H, 2s); 1.91 (1H, m); 1.81-1.59 (4H, m). ¹³C-NMR (CDCl₃): δ 172.5, 138.2, 136.8, 133.4, 131.8, 129.7, 129.2, 128.6, 127.4, 54.9, 51.3, 46.7, 41.3, 30.6, 28.6, 21.2.

### Example 118

### 2-(4-Hydroxyphenyl)-N-(4-methylbenzyl)-N-(1-methylpiperidin-4-yl)-acetamide (47AKU-29)

42ELH-77 (41 mg, 0.1 mmol) was dissolved in 1 ml dry dichloromethane and placed in oven-dried 10 ml flask. Mixture was cooled to −78° C. on a dry-ice/isopropanol bath. Borontribromide (1.0 M in dichloromethane, 150 μl, 0.15 mmol) was slowly added at −78° C. Ice-bath was removed and mixture was left at room temp. for 2 hrs. Water (3 ml) and saturated NaCl (aq.) were added and aq. phase was extracted with dichloromethane, ethylacetate and n-butanol. Combined organic phases were dried over MgSO₄ and concentrated on Rotavapor (40° C.). Crude product was purified by flash chromatography (0-20% methanol in dichloromethane) giving 22 mg (63%) 47AKU-29. HCl-salt was prepared from 2M HCl/diethylether in dichloromethane/heptane. TLC (10% methanol in dichloromethane): R^{f}=0.3. HPLC-MS (Method A): M⁺=353.2 (UV/MS (%)=100/100). ¹H-NMR (400 MHz, CDCl₃, rotamers): δ 7.07-6.60 (8H, m); 4.48 (1H, m); 4.39 (2H, s); 3.76 and 3.66 (4H, 2bs); 3.41 (2H, s); 3.08 (2H, m); 2.49 (1H, m); 2.42 (2H, bs); 2.22 and 2.16 (3H, 2s); 1.96-1.82 (2H, m); 1.66-1.56 (1H, m). ¹³C-NMR (CDCl₃): δ 173.7, 156.0, 137.3, 134.6, 129.7, 129.6, 125.7, 125.4, 115.7, 54.4, 50.4, 46.8, 44.0, 40.5, 27.3, 20.9.

### Example 119

### N-Phenethyl-N-(1-methylpiperidin-4-yl)-N′-phenyl-carbamide (47AKU-30)

4-(2-Phenylethyl)amino-1-methylpiperidine (110 mg, 0.5 mmol) was dissolved in 5 ml dichloromethane and placed in 50 ml flask. Phenylisocyanate (71 mg, 0.6 mmol) in 5 ml dichloromethane was added. After 16 hrs magnetic stirring the reaction mixture was concentrated on Rotavapor (40° C.). Crude product was purified twice by flash chromatography (0-10% methanol in dichloromethane) giving 131 mg (78%) 47AKU-30. HCl-salt was prepared from 2M HCl/diethylether in dichloromethane/heptane. TLC (10% methanol in dichloromethane): R^{f}=0.4. HPLC-MS (Method A): M⁺=338.1 (UV/MS (%)=99/100). ¹H-NMR (400 MHz, CDCl₃): δ 7.36-6.93 (10H, m); 6.24 (1H, s); 4.31 (1H, m); 3.50 (2H, t); 3.20 (2H, d); 2.89 (2H, t); 2.57 (2H, m); 2.50 (3H, s); 2.26 (2H, m); 1.79 (2H, m). ¹³C-NMR (CDCl₃) δ 155.8, 139.2, 139.0, 129.4, 129.3, 128.9, 127.3, 123.2, 120.4, 54.9, 51.3, 45.5, 44.3, 37.6, 28.3.

### Example 120

### N-(3-Phenylpropyl)-N-(1-methylpiperidin-4-yl)-N′-benzyl-carbamide (47AKU-31)

4-(3-Phenylpropyl)amino-1-methylpiperidine (160 mg, 0.7 mmol) was dissolved in 5 ml dichloromethane and placed in 50 ml flask. Benzylisocyanate (107 mg, 0.8 mmol) in 5 ml dichloromethane was added. After 2 hrs magnetic stirring the reaction mixture was concentrated on Rotavapor (40° C.). Crude product was purified twice by flash chromatography (0-10% methanol in dichloromethane) giving 156 mg (61%) 47AKU-31. HCl-salt was prepared from 2M HCl/diethylether in dichloromethane/heptane. TLC (10% methanol in dichloromethane): R^{f}=0.3. HPLC-MS (Method A): M⁺=366.1 (UV/MS (%)=100/100). ¹H-NMR (400 MHz, CDCl₃): δ 7.34-7.07 (10H, m); 4.33 (3H, m); 4.14 (1H, m); 3.04 (2H, m); 2.89 (2H, d); 2.57 (2H, t); 2.28 (3H, s); 2.06 (2H, m); 1.87 (2H, m); 1.75-1.62 (4H, m). ¹³C-NMR (CDCl₃): δ 157.5, 141.0, 140.0, 129.0, 128.6, 128.3, 128.0, 127.6, 126.6, 55.6, 52.1, 46.3, 45.1, 41.6, 33.4, 32.2, 30.6.

### Example 121

### N-(3-Phenylpropyl)-N-(1-methylpiperidin-4-yl)-N′-phenyl-carbamide (47AKU-32)

4-(3-Phenylpropyl)amino-1-methylpiperidine (160 mg, 0.7 mmol) was dissolved in 5 ml dichloromethane and placed in 50 ml flask. Phenylisocyanate (95 mg, 0.8 mmol) in 5 ml dichloromethane was added. After 20 hrs magnetic stirring the reaction mixture was concentrated on Rotavapor (40° C.). Crude product was purified by flash chromatography (0-10% methanol in dichloromethane) giving 106 mg (43%) 47AKU-32. HCl-salt was prepared from 2M HCl/diethylether in dichloromethane/heptane. TLC (10% methanol in dichloromethane): R^{f}=0.3. HPLC-MS (Method A): M⁺=352.1 (UV/MS (%)=100/100). ¹H-NMR (400 MHz, CDCl₃): δ 7.35-6.95 (10H, m); 5.99 (1H, s); 4.18 (1H, m); 3.17 (2H, t); 2.91 (2H, d); 2.65 (2H, t); 2.28 (3H, s); 2.07 (2H, m); 1.97 (2H, m); 1.81-1.66 (4H, m). ¹³C-NMR (CDCl₃): δ 154.9, 141.0, 139.3, 129.2, 129.0, 129.0, 126.8, 123.1, 120.0, 55.6, 52.2, 46.2, 41.8, 33.4, 32.3, 30.6.

### Example 122

### 2-(4-Methoxyphenyl)-2,2-ethylene-N-(4-methylbenzyl)-N-(1-methylpiperidin-4-yl)acetamide (47AKU-33)

1-(4-Methoxyphenyl)-1-cyclopropane carboxylic acid (230 mg, 1.2 mmol) was dissolved in 2 ml thionylchloride and placed in 50 ml flask. Mixture was heated to reflux for 2 hrs and then concentrated on Rotavapor (40° C.). The acid chloride (250 mg, 1.2 mmol) in 5 ml dichloromethane was added to 47AKU-5 (220 mg, 1.0 mmol) in 5 ml dichloromethane. After 2 hrs magnetic stirring the reaction mixture was concentrated on Rotavapor (40° C.). Crude product was purified twice by flash chromatography (0-10% methanol in dichloromethane) giving 201 mg (51%) 47AKU-33. HCl-salt was prepared from 2M HCl/diethylether in dichloromethane/heptane. TLC (10% methanol in dichloromethane): R^{f}=0.6. HPLC-MS (Method A): M⁺=393.2 (UV/MS (%)=95/88).

¹H-NMR (400 MHz, CDCl₃, rotamers): δ 7.22-6.70 (8H, m); 4.44 (2H, s); 4.26 (1H, m); 3.74 (3H, s); 3.12 and 2.89 (2H, 2m); 2.51 (1H, m); 2.32 (3H, m); 2.26 (3H, s); 2.08-1.52 (4H, m); 1.36 (2H, bs); 1.15-0.95 (3H, m). ¹³C-NMR (CDCl₃) δ 172.9, 158.6, 136.6, 132.7, 129.2, 128.6, 127.9, 127.4, 114.4, 55.5, 55.1, 54.4, 45.2, 45.0, 29.8, 29.2, 21.2, 13.8.

### Example 123

### 2-(4-Methoxyphenyl)-N-(1-phenylethyl)-N-(1-methylpiperidin-4-yl)acetamide (47AKU-37)

### 4-Alpha-methylbenzylamino-1-methyl-piperidine (47AKU-36)

DL-Phenylethylamine (606 mg, 5.0 mmol) was dissolved in 10 ml methanol and 1-Methyl-4-piperidone (566 mg, 5.0 mmol) in 10 ml methanol was added. Mixture was stirred and Acetic acid (˜0.75 ml) was added until pH˜5. NaCNBH₃ (628 g, 10 mmol) was slowly added. Gas evolution observed. After magnetic stirring for 20 hrs methanol was partly removed on Rotavapor (40° C.). Ethylacetate, 2M NaOH and water were added until pH˜10. Phases were separated and aq. phase was then re-extracted with ethylacetate and dichloromethane. Combined organic phases were dried over MgSO₄. Concentration on Rotavapor (40° C.) yielded 838 mg crude 47AKU-36. TLC (10% methanol in dichloromethane): R^{f}=0.3. HPLC-MS (Method A): M⁺=219.1 (UV/MS (%)=100/94).

### 2-(4-Methoxyphenyl)-N-alpha-methylbenzyl-N-(1-methylpiperidin-4-yl)acetamide (47AKU-37)

47AKU-36 (218 mg, 1.0 mmol) was dissolved in 10 ml dichloromethane and placed in 50 ml flask. 4-Methoxyphenylacetyl chloride (185 mg, 1.2 mmol) in 10 ml dichloromethane was added. After 16 hrs magnetic stirring the reaction mixture was concentrated on Rotavapor (40° C.). Crude product was purified by flash chromatography (0-10% methanol in dichloromethane) giving 256 mg (70%) 47AKU-37. HCl-salt was prepared from 2M HCl/diethylether in dichloromethane/heptane. TLC (10% methanol in dichloromethane): R^{f}=0.5. HPLC-MS (Method A): M⁺=367.3 (UV/MS (%)=100/99).

¹H-NMR (400 MHz, CDCl₃, rotamers): δ 7.34-7.06 (7H, m); 6.84 (2H, d); 5.10 (1H, m); 3.77 (3H, s); 3.67 (2H, m); 3.17 (1H, m); 3.03-2.75 (3H, m); 2.64 (3H, s); 2.38 (2H, m); 1.77-1.05 (6H, m). ¹³C-NMR (CDCl₃): δ 172.0, 158.9, 139.9, 130.0, 129.0, 128.2, 127.1, 114.5, 55.5, 53.1, 51.4, 42.4, 41.3, 31.1, 29.5, 24.9, 18.1.

### Example 124

### 2-(4-Methoxyphenyl)-N-(4-methylbenzyl)-N-(8-methyl-8-aza-bicyclo[3.2.1]octen-3-yl)-acetamide (47AKU-39)

### 4-Methylbenzylimino-tropane (47AKU-38)

4-Methylbenzylamine (1.21 g, 10 mmol) and Tropinone (1.39 g, 10 mmol) were placed in 100 ml flask and dissolved in 50 ml toluene. Mixture was heated to reflux for 3 hrs and water was removed using a Dean/Stark water-separator. Crude product was concentrated on Rotavapor (40° C.) giving 47AKU-38. TLC (10% methanol in dichloromethane): R^{f}=0.3. ¹H-NMR (400 MHz, CDCl₃, isomers): 7.20-7.09 (4H, m); 4.47 (1H, m); 3.81 (1H, s); 3.42 (1H, m); 3.31 (1H, m); 2.77-2.56 (2H, m); 2.47 and 2.41 (3H, 2s); 2.33 and 2.31 (3H, 2s); 2.27-1.97 (4H, m); 1.69-1.54 (2H, m).

### 2-(4-Methoxyphenyl)-N-(4-methylbenzyl)-N-(8-methyl-8-aza-bicyclo[3.2.1]octen-3-yl)-acetamide (47AKU-39)

47AKU-38 (242 mg, 1.0 mmol) was dissolved in 5 ml dichloromethane and placed in 50 ml flask. 4-Methoxyphenylacetyl chloride (185 mg, 1.2 mmol) in 10 ml dichloromethane was added. After 16 hrs magnetic stirring the reaction mixture was concentrated on Rotavapor (40° C.). Crude product was purified by flash chromatography (0-10% methanol in dichloromethane) giving 69 mg (18%) 47AKU-39. HCl-salt was prepared from 2M HCl/diethylether in dichloromethane/heptane. TLC (10% methanol in dichloromethane): R^{f}=0.4. HPLC-MS (Method A): M⁺=391.2 (UV/MS (%)=91/86).

¹H-NMR (400 MHz, CDCl₃, rotamers): δ 7.22-6.82 (8H, m); 5.41 (1H, bs); 4.71-4.52 (2H, m); 3.78 (3H, s); 3.68 (2H, m); 3.44-3.24 (2H, m); 2.72-2.36 (5H, m); 2.32 (3H, s); 2.25-2.00 (2H, m); 1.80-1.54 (2H, m). ¹³C-NMR (CDCl₃): δ 170.8, 158.7, 137.4, 134.9, 130.1, 129.3, 128.9, 126.9, 114.2, 59.0, 58.0, 55.5, 49.5, 46.3, 39.7, 35.9, 33.8, 29.7, 21.3.

### Example 125

### 2-Phenyl-2-ethyl-N-(4-methylbenzyl)-N-(1-methylpiperidin-4-yl)acetamide (47AKU-40)

2-Phenylbutyric acid (197 mg, 1.2 mmol) was dissolved in 2 ml thionylchloride and placed in 50 ml flask. Mixture was heated to reflux for 2 hrs and then concentrated on Rotavapor (50° C.). The acid chloride (1.2 mmol) in 5 ml dichloromethane was added to 47AKU-5 (158 mg, 0.72 mmol) in 5 ml dichloromethane. After 20 hrs magnetic stirring the reaction mixture was concentrated on Rotavapor (40° C.). Crude product was purified by flash chromatography (0-10% methanol in dichloromethane) giving 196 mg (74%) 47AKU-40. HCl-salt was prepared from 2M HCl/diethylether in dichloromethane/heptane. TLC (10% methanol in dichloromethane): R^{f}=0.5. HPLC-MS (Method A): M⁺=365.4 (UV/MS (%)=99/100). ¹H-NMR (400 MHz, CDCl₃, rotamers): δ 7.32-6.98 (8H, m); 4.77 (1H, bs); 4.50 (1H, d); 4.29 (1H, d); 3.43 and 3.21 (3H, 2m); 2.72 (2H, m); 2.62 (3H, s); 2.43 (1H, m); 2.32 (3H, s); 2.2 (3H, m); 2.04 (2H, m); 1.67 (3H, m); 0.92-0.72 (3H, m). ¹³C-NMR (CDCl₃): δ 174.7, 139.9, 137.3, 135.2, 129.7, 129.0, 127.8, 127.3, 125.8, 54.5, 51.6, 49.4, 46.0, 43.8, 28.9, 26.7, 26.3, 21.2, 12.7.

### Example 126

### (47AKU-44)N-(4-Methylbenzyl)-N-(1-methylpiperidin-4-yl)-N′-(4-methoxybenzyl)-carbamide (47AKU-44)

47AKU-5 (219 mg, 1.0 mmol) was dissolved in 5 ml dichloromethane and placed in 50 ml flask. 4-Methoxybenzylisocyanate (196 mg, 1.2 mmol) in 10 ml dichloromethane was added. After 16 hrs magnetic stirring the reaction mixture was concentrated on Rotavapor (40° C.). Crude product was purified by flash chromatography (0-10% methanol in dichloromethane) giving 192 mg (50%) 47AKU-44. HCl-salt was prepared from 2M HCl/diethylether in dichloromethane/heptane. TLC (10% methanol in dichloromethane): R^{f}=0.3. HPLC-MS (Method A): M⁺=382.3 (UV/MS (%)=100/94). ¹H-NMR (400 MHz, CDCl₃): δ 7.10 (4H, m); 6.98 (2H, m); 6.76 (2H, m); 4.58 (1H, t); 4.45 (1H, m); 4.33 (2H, s); 4.25 (2H, d); 3.76 (3H, s); 2.97 (2H, m); 2.34 (3H, s); 2.32 (3H, s); 2.24 (2H, m); 1.78 (4H, m). ¹³C-NMR (CDCl₃): δ 158.9, 158.5, 137.3, 135.2, 131.8, 129.8, 128.8, 126.2, 114.1, 55.5, 55.4, 51.7, 45.8, 45.7, 44.5, 29.7, 21.2.

### Example 127

### 2-(3,4-dimethoxyphenyl)-N-(4-methylbenzyl)-N-(1-methylpiperidin-4-yl)acetamide (47AKU-45)

3,4-Dimethoxyphenylbutyric acid (235 mg, 1.2 mmol) was dissolved in 2 ml thionylchloride and placed in 50 ml flask. Mixture was heated to reflux for 2 hrs and then concentrated on Rotavapor (50° C.). The acid chloride (1.2 mmol) in 5 ml dichloromethane was added to 47AKU-5 (219 mg, 1.0 mmol) in 10 ml dichloromethane. After 16 hrs magnetic stirring the reaction mixture was concentrated on Rotavapor (40° C.). Crude product was purified by flash chromatography (0-10% methanol in dichloromethane) giving 129 mg (33%) 47AKU-45. HCl-salt was prepared from 2M HCl/diethylether in dichloromethane/heptane. TLC (10% methanol in dichloromethane): R^{f}=0.4. HPLC-MS (Method A): M⁺=397.4 (UV/MS (%)=98/89). ¹H-NMR (400 MHz, CDCl₃, rotamers): δ 7.17-6.60 (7H, m); 4.75 (1H, m); 4.51 (2H, s); 3.83 (3H, s); 3.79 (3H, s); 3.53 (2H, s); 3.27 (2H, d); 2.65 (2H, t); 2.58 (3H, s); 2.32 (3H, s); 2.24 (2H, m); 1.72 (2H, d). ¹³C-NMR (CDCl₃): δ 172.8, 149.3, 148.3, 137.4, 135.0, 129.8, 127.4, 125.8, 121.0, 112.2, 111.6, 56.2, 56.1, 54.6, 49.6, 46.7, 44.0, 40.9, 27.0, 21.2.

### Example 128

### 2-(3,4-Methylenedioxyphenyl)-N-(4-methylbenzyl)-N-(1-methylpiperidin-4-yl)a cetamide (47AKU-46)

3,4-Methylenedioxyphenylacetic acid (216 mg, 1.2 mmol) was dissolved in 2 ml thionylchloride and placed in 50 ml flask. Mixture was heated to reflux for 2 hrs and then concentrated on Rotavapor (50° C.). The acid chloride (1.2 mmol) in 5 ml dichloromethane was added to 47AKU-5 (219 mg, 1.0 mmol) in 10 ml dichloromethane. After 2 hrs magnetic stirring the reaction mixture was concentrated on Rotavapor (40° C.). Crude product was purified by flash chromatography (0-10% methanol in dichloromethane) giving 188 mg (49%) product. Further purification by ion exchange chromatography (washout with 10% aq. NH₄ OH (25%) in methanol) yielded 149 mg (39%) 47AKU-46. HCl-salt was prepared from 2M HCl/diethylether in dichloromethane/heptane. TLC (10% methanol in dichloromethane): R^{f}=0.4. HPLC-MS (Method A): M⁺=381.2 (UV/MS (%)=96/95). ¹H-NMR (400 MHz, CDCl₃, rotamers): δ 7.17-7.02 (4H, m); 6.77-6.51 (3H, m); 5.91 and 5.93 (2H, 2s); 4.70 (1H, m); 4.52 and 4.49 (2H, 2s); 3.51 (2H, s); 3.26 (2H, d); 2.49 (3H, s); 2.33 (3H, s); 2.14-1.66 (6H, m) ¹³C-NMR (CDCl₃): δ 172.5, 148.1, 146.8, 137.3, 135.1, 129.8, 128.6, 125.8, 121.9, 109.4, 108.5, 101.2, 54.8, 50.2, 46.7, 44.6, 41.1, 27.7, 21.2.

### Example 129

### 2-(4-Methoxyphenyl)-N-(4-methylbenzyl)-N-(1-t-butylpiperidin-4-yl)-acetamide (47AKU-49)

### 1-t-Butyl-4-piperidone (47AKU-47)

1-Benzyl-4-piperidone (1.89 g, 10 mmol) was dissolved in 15 ml acetone. Methyliodide (0.90 ml, 15 mmol) was slowly added over 5 min. After 2 hrs magnetic stirring additional Methyliodide (1.8 ml, 30 mmol) was added. After 1 hr magnetic stirring 20 ml diethyl-ether was added. Crude product was collected by filtration and washed with acetone/diethylether. White crystals were dried under vacuum giving 806 mg quartenary salt. TLC (10% methanol in dichloromethane): R^{f}=0.7. Partly dissolved salt in 5 ml water was added to 50° C. hot mixture of t-Butylamine (120 mg, 1.6 mmol) and Potassiumcarbonate (32 mg, 0.22 mmol) in 3 ml ethanol. The resulting mixture was stirred and heated to reflux (˜80° C.) for 1 hr. After cooling water (20 ml) and dichloromethane (20 ml) were added. Phases were separated and aq. phase was re-extracted with dichloromethane and ethylacetate. Combined organic phases were dried over MgSO₄ and concentrated on Rotavapor (40° C.) giving 496 mg 47AKU-47. TLC (10% methanol in dichloromethane): R^{f}=0.3. ¹H-NMR (400 MHz, CDCl₃): δ 2.82 (4H, t); 2.41 (4H, t); 1.12 (9H, s). ¹³C-NMR (CDCl₃): δ 210.2, 54.3, 46.4, 42.4, 26.6. Crude product contained ˜25% (¹H-NMR) starting material (1-Benzyl-4-piperidone).

### 4-(4-Methylbenzylamino)-1-t-butyl-piperidine (47AKU-48)

4-Methylbenzylamine (268 mg, 2.2 mmol) was dissolved in 5 ml methanol and placed in 50 ml flask. 47AKU-47 (305 mg, 2.0 mmol) in 5 ml methanol was added. Acetic acid (0.3 ml) was added until pH˜5. NaCNBH₃ (250 mg, 4.0 mmol) was slowly added. Gas evolution observed. After 4 hrs magnetic stirring dichloromethane, 2M NaOH and water were added until pH˜10. Phases were separated and aq. phase was then re-extracted with dichloromethane and ethylacetate. Combined organic phases were dried over MgSO₄. Concentration on Rotavapor (40° C.) yielded 556 mg crude 47AKU-48. TLC (20% methanol in dichloromethane): R^{f}=0.4. HPLC-MS (Method A): M⁺=261.2 (MS (%)=57).

### 2-(4-Methoxyphenyl)-N-(4-methylbenzyl)-N-(1-t-butylpiperidin-4-yl)-acetamide (47AKU-49)

47AKU-48 (556 mg, 2.1 mmol) was placed in 50 ml flask and 5 ml dichloromethane was added. 4-Methoxyphenylacetyl chloride (739 mg, 4.0 mmol) in 10 ml dichloromethane was added. After 4 hrs magnetic stirring the reaction mixture was concentrated on Rotavapor (40° C.). Crude product was purified by flash chromatography (0-10% methanol in dichloromethane) giving 124 mg (15%) product. Further purification by ion exchange chromatography (washout with 10% aq. NH₄ OH (25%) in methanol) gave 91 mg (11%) 47AKU-49. HCl-salt was prepared from 2M HCl/diethylether in dichloromethane/heptane. TLC (10% methanol in dichloromethane): R^{f}=0.5. HPLC-MS (Method A): M⁺=409.4 (UV/MS (%)=100/90). ¹H-NMR (400 MHz, CDCl₃): δ 7.11 (4H, m); 7.03 (2H, d); 6.79 (2H, d); 4.78 (1H, m); 4.56 (2H, s); 3.76 (3H, s); 3.53 (2H, s); 3.43 (2H, m); 2.63 (2H, m); 2.47 (2H, m); 2.31 (3H, s); 1.74 (2H, d); 1.36 (9H, s). ¹³C-NMR (CDCl₃): δ 173.0, 158.8, 137.1, 135.3, 129.8, 129.7, 127.0, 125.8, 114.3, 55.6, 55.5, 49.8, 46.5, 46.4, 40.5, 26.7, 25.1, 21.2.

### Example 130

### N-(4-Methylbenzyl)-N-(1-methylpiperidin-4-yl)-N′-phenylethyl-carbamide (58AKU-1)

47AKU-5-2 (219 mg, 1.0 mmol) was dissolved in 5 ml dichloromethane and placed in 50 ml flask. Phenethylisocyanate (177 mg, 1.2 mmol) in 5 ml dichloromethane was added. After 6 hrs magnetic stirring the reaction mixture was concentrated on Rotavapor (40° C.). Crude product was purified by flash chromatography (0-15% methanol in dichloromethane) giving 134 mg (37%) 58AKU-1. HCl-salt was prepared from 2M HCl/diethylether in dichloromethane/heptane. TLC (10% methanol in dichloromethane): R^{f}=0.5. HPLC-MS (Method A): M⁺=366.3 (UV/MS (%)=99/96). ¹H-NMR (400 MHz, CDCl₃): δ 7.21-6.97 (9H, m); 4.33 (1H, m); 4.26 (1H, m); 4.21 (2H, s); 3.39 (2H, q); 2.85 (2H, m); 2.67 (2H, t); 2.31 (3H, s); 2.24 (3H, s); 2.06 (2H, m); 1.73-1.57 (4H, m). ¹³C-NMR (CDCl₃): δ 158.7, 139.5, 137.0, 135.4, 129.7, 128.8, 128.6, 126.3, 126.1, 55.6, 52.2, 46.2, 45.8, 42.2, 36.4, 30.2, 21.2.

### Example 131

### N-Phenylethyl-N-(1-methylpiperidin-4-yl)-N′-phenethyl-carbamide (58AKU-2)

4-(2-Phenylethyl)amino-1-methylpiperidine (131 mg, 0.6 mmol) was dissolved in 5 ml dichloromethane and placed in 50 ml flask. Phenethylisocyanate (103 mg, 0.7 mmol) in 5 ml dichloromethane was added. After 4 hrs magnetic stirring the reaction mixture was concentrated on Rotavapor (45° C.). Crude product was purified by flash chromatography (0-10% methanol in dichloromethane) giving 198 mg (90%) 58AKU-1. HCl-salt was prepared from 2M HCl/diethylether in dichloromethane/heptane. TLC (10% methanol in dichloromethane): R^{f}=0.3. HPLC-MS (Method A): M⁺=366.3 (UV/MS (%)=100/100). ¹H-NMR (400 MHz, CDCl₃): δ 7.33-7.16 (8H, m); 7.01 (2H, m); 4.23 (1H, t); 4.04 (1H, m); 3.47 (2H, q); 3.17 (2H, t); 2.89 (2H, m); 2.78 (2H, t); 2.66 (2H, t); 2.28 (3H, s); 2.05 (2H, m); 1.79-1.59 (4H, m). ¹³C-NMR (CDCl₃): δ 157.8, 139.6, 139.0, 129.0, 128.9, 128.8, 126.8, 126.7, 55.7, 52.5, 46.2, 44.6, 42.0, 37.3, 36.4, 30.5.

### Example 132

### N-(4-Methylbenzyl)-N-(1-t-butylpiperidin-4-yl)-N′-(4-methoxybenzyl)carbamide (58AKU-3)

47AKU-5-2 (404 mg, 1.6 mmol) was dissolved in 5 ml dichloromethane and placed in 50 ml flask. 4-Methoxybenzylisocyanate (326 mg, 2.0 mmol) in 5 ml dichloromethane was added. After 20 hrs magnetic stirring the reaction mixture was concentrated on Rotavapor (45° C.). Crude product was purified three times by flash chromatography (0-20% methanol in dichloromethane and 0-30% methanol in ethylacetate) giving 155 mg (23%) 58AKU-3. HCl-salt was prepared from 2M HCl/diethylether in dichloromethane/heptane. TLC (10% methanol in dichloromethane): R^{f}=0.3. HPLC-MS (Method A): M⁺=424.2 (UV/MS (%)=92/83). ¹H-NMR (400 MHz, CDCl₃): δ 7.10 (4H, m); 6.99 (2H, m); 6.76 (2H, m); 4.53 (1H, m); 4.35 (3H, s); 4.26 (2H, d); 3.77 (3H, s); 3.09 (2H, m); 2.32 (3H, s); 2.22 (2H, m); 1.81-1.54 (4H, m); 1.06 (9H, s). ¹³C-NMR (CDCl₃): δ 158.9, 158.6, 137.1, 135.6 131.9, 129.7, 128.8, 126.2, 114.0, 62.6, 55.5, 53.0, 45.9, 45.7, 44.5, 31.0, 26.3, 21.2.

### Example 133

### 2-(4-Ethoxyphenyl)-N-(4-methylbenzyl)-N-(1-methylpiperidin-4-yl)acetamide (58AKU-4)

4-Ethoxyphenylacetic acid (270 mg, 1.5 mmol) was dissolved in 2 ml thionylchloride and placed in 50 ml flask. Mixture was heated to reflux for 2 hrs and then concentrated on Rotavapor (45° C.). The acid chloride (1.5 mmol) in 5 ml dichloromethane was added to 47AKU-5-2 (262 mg, 1.2 mmol) in 5 ml dichloromethane. After 20 hrs magnetic stirring the reaction mixture was concentrated on Rotavapor (40° C.). Crude product was purified by flash chromatography (0-10% methanol in dichloromethane) giving 272 mg (60%) 58AKU-4. HCl-salt was prepared from 2M HCl/diethylether in dichloromethane/heptane. TLC (10% methanol in dichloromethane): R^{f}=0.4. HPLC-MS (Method A): M⁺=381.2 (UV/MS (%)=98/91). ¹H-NMR (400 MHz, CDCl₃): δ 7.17-6.99 (6H, m); 6.82-6.76 (2H, m); 4.73 (1H, m); 4.48 (2H, s); 3.98 (2H, q); 3.52 (2H, s); 3.22 (2H, d); 2.61 (2H, t); 2.54 (3H, s); 2.32 (3H, s); 2.14 (2H, s); 1.71 (2H, d): 1.38 (3H, t). ¹³C-NMR (CDCl₃): δ 172.9, 158.2, 137.3, 135.0, 129.9, 129.8, 126.8, 125.8, 114.9, 63.7, 54.6, 49.8, 46.7, 44.1, 40.6, 27.2, 21.2, 15.0.

### Example 134

### 2-(4-Butoxyphenyl)-N-(4-methylbenzyl)-N-(1-methylpiperidin-4-yl)acetamide (58AKU-5)

4-Butoxyphenylacetic acid (317 mg, 1.5 mmol) was dissolved in 2 ml thionylchloride and placed in 50 ml flask. Mixture was heated to reflux for 2 hrs and then concentrated on Rotavapor (45° C.). The acid chloride (1.5 mmol) in 5 ml dichloromethane was added to 47AKU-5-2 (262 mg, 1.2 mmol) in 5 ml dichloromethane. After 20 hrs magnetic stirring the reaction mixture was concentrated on Rotavapor (40° C.). Crude product was purified by flash chromatography (0-10% methanol in dichloromethane) giving 230 mg (47%) 58AKU-5. HCl-salt was prepared from 2M HCl/diethylether in dichloromethane/heptane. TLC (10% methanol in dichloromethane): R^{f}=0.5. HPLC-MS (Method A): M⁺=409.2 (UV/MS (%)=98/93). ¹H-NMR (400 MHz, CDCl₃): δ 7.15-6.96 (6H, m); 6.78 (2H, m); 4.74 (1H, m); 4.48 (2H, s); 3.91 (2H, t); 3.52 (2H, s); 3.27 (2H, d); 2.72 (2H, t); 2.58 (3H, s); 2.32 (3H, s); 2.23 (2H, m); 1.72 (4H, d); 1.45 (2H, m); 0.95 (3H, t). ¹³C-NMR (CDCl₃): δ 173.0, 158.4, 137.3, 135.0, 129.8, 126.6, 125.8, 115.0, 67.9, 54.4, 49.5, 46.7, 43.8, 40.6, 31.5, 26.8, 21.2, 19.4, 14.0.

### Example 135

### 2-(4-i-Propoxyphenyl)-N-(4-methylbenzyl)-N-(1-methylpiperidin-4-yl)acetamide (58AKU-6)

47AKU-29-2 (245 mg, 0.7 mmol) was dissolved in 10 ml dimethylformamide and placed in 50 ml flask. KOH (196 mg, 3.5 mmol) and Isopropylbromide (200 μl, 2.1 mmol) were added. Mixture was heated to 50° C. and stirred for 24 hrs. After cooling water and ethylacetate were added. Phases were separated and aq. phase was then re-extracted with dichloromethane. Combined organic phases were washed with brine, dried over MgSO₄ and concentrated on Rotavapor (40° C.) giving 188 mg. Crude product was purified by flash chromatography (0-10% methanol in dichloromethane) yielding 136 mg (49%) 58AKU-6. HCl-salt was prepared from 2M HCl/diethylether in dichloromethane/heptane. TLC (10% methanol in dichloromethane): R^{f}=0.3. HPLC-MS (Method B): M⁺=395 (UV/MS (%)=95/91). ¹H-NMR (400 MHz, CDCl₃, rotamers): δ 7.23-7.01 (6H, m); 6.79 (2H, m); 4.60 (1H, m); 4.51 (1H, m); 4.44 (1H, s); 3.77 (1H, s); 3.52 (1H, s); 2.83 (2H, m); 2.76 (2H, m); 2.28 and 2.34 (3H, 2s); 2.19 and 2.22 (3H, 2s); 2.05 (1H, m); 1.86-1.55 (4H, m); 1.32 (6H, d). ¹³C-NMR (CDCl₃): δ 172.6, 157.0, 137.1, 135.6, 129.8, 129.7, 125.8, 116.2, 70.1, 55.3, 51.6, 46.6, 46.1, 40.8, 29.6, 22.3, 21.2.

Pharmalogical Data

### Example 136

### Receptor Selection and Amplification (R-SAT) Assays

The functional receptor assay, Receptor Selection and Amplification Technology (R-SAT), was used (with minor modifications from that previously described U.S. Pat. No. 5,707,798) to screen compounds for efficacy at the 5-HT2A receptor. The RSAT assay was conducted as described herein. The results obtained for several compounds of the invention are presented in Table 3, below.

TABLE 3
Efficiency And pIC50 Of Compounds At The 5-HT2A Receptor
Compared To Ritanserin.

| Compound | Percent Efficacy | pIC50 |
|---|---|---|
| 26HCH17 | 94 | 8.3 |
| 26HCH65 | 103 | 8.2 |
| 26HCH66-05 | 126 | 8.1 |
| 26HCH79-5 | 94 | 8.2 |
| 6HCH79-6 | 83 | 8.3 |
| 26HCH79-10 | 102 | 7.8 |
| 26HCH71B | 124 | 7.9 |
| 42ELH45 | 108 | 9.0 |
| 50ELH27 | 108 | 8.7 |
| 47AKU-7 | 120 | 8.1 |
| 42ELH80 | 122 | 8.5 |
| 42ELH79 | 110 | 8.5 |
| 42ELH91 | 108 | 8.0 |
| 42ELH85 | 118 | 7.8 |
| 42ELH75 | 109 | 8.3 |
| 47AKU-12 | 112 | 8.1 |
| 47AKU-8 | 113 | 8.1 |
| 47AKU-22 | 117 | 7.9 |
| 47AKU-21 | 117 | 7.9 |
| 47AKU-20 | 120 | 8.0 |
| 50ELH8 | 129 | 7.8 |
| 50ELH68 | 96 | 8.4 |
| 50ELH65 | 92 | 7.9 |
| 47AKU-44 | 112 | 8.5 |
| 57MBT12B | 75 | 7.7 |
| 58AKU-4 | 110 | 9.6 |
| 58AKU-3 | 111 | 8.1 |
| 58AKU-5 | 99 | 9.5 |
| 58AKU-6 | 101 | 9.8 |
| 57MBT54B | 95 | 7.9 |
| 50ELH95B | 119 | 8.0 |
| 50ELH93E | 72 | 8.1 |
| 50ELH93D | 58 | 7.8 |
| 50ELH93A | 106 | 8.7 |
| 63ELH1A | 104 | 8.3 |
| 50ELH89 | 111 | 9.7 |
| 63ELH20 | 95 | 9.0 |
| 57MBT70-8D | 119 | 7.7 |
| 57MBT70-5D | 105 | 8.4 |
| 57MBT70-4D | 98 | 8.5 |
| 57MBT70-3D | 87 | 8.9 |
| 57MBT70-2D | 105 | 8.2 |
| 57MBT70-1D | 120 | 7.9 |
| 63ELH21 | 100 | 8.5 |
| 57MBT62B | 119 | 7.9 |
| 57MBT70-6E | 115 | 8.0. |

### Example 137

### Selectivity Profile for 2-(4-Methoxyphenyl)-N-(4-methylbenzyl)-N-(1-methylpiperidin-4-yl)acetamide hydrochloride

The R-SAT assay (described above in example 136) was used to investigate the selectivity of 2-(4-Methoxyphenyl)-N-(4-methylbenzyl)-N-(1-methylpiperidin-4-yl)acetamide hydrochloride. The results from a broad profiling of this compound at a variety of receptors are reported in Table 4 below. NR means No Response, i.e. the compound investigated showed no effect at the receptor studied.

TABLE 4
Selectivity of 2-(4-methoxyphenyl)-n-(4-methylbenzyl)-n-
(1-methylpiperidin-4-yl) acetamide.

| RECEPTOR | ASSAY | pEC50/pIC50 |
|---|---|---|
| 5-HT_{1A} | agonist | NR |
| | Antagonist | NR |
| 5-HT_{1B} | agonist | NR |
| | Antagonist | NR |
| 5-HT_{1D} | agonist | NR |
| | Antagonist | NR |
| 5-HT_{1E} | agonist | NR |
| | Antagonist | NR |
| 5-HT_{1F} | agonist | NR |
| | Antagonist | NR |
| 5-HT₂A | agonist | NR |
| | inverse agonist | 8.8 |
| 5-HT₂B | agonist | NR |
| | inverse agonist | 6.9 |
| 5-HT₂C | agonist | NR |
| | inverse agonist | 7 |
| 5-HT₄ | agonist | NR |
| | inverse agonist | NR |
| 5-HT₆ | agonist | NR |
| | Inv. Agonist | 6.8 |
| 5-HT7 | agonist | NR |
| | inverse agonist | 6.9 |
| m1 | agonist | NR |
| | antagonist | NR |
| m2 | agonist | NR |
| | antagonist | NR |
| m3 | agonist | NR |
| | antagonist | NR |
| m4 | agonist | NR |
| | antagonist | NR |
| m5 | agonist | NR |
| | antagonist | NR |
| D1 | agonist | NR |
| | antagonist | NR |
| D2 | agonist | NR |
| | antagonist | NR |
| D3 | agonist | NR |
| | antagonist | NR |
| D5 | agonist | NR |
| | antagonist | NR |
| Histamine 1 | agonist | NR |
| | Inv. Agonist | NR |
| Histamine 2 | agonist | NR |
| | Antagonist | NR |
| Histamine 3 | agonist | NR |
| | Antagonist | NR |
| alpha-1A(a/c) | agonist | NR |
| | antagonist | NR |
| alpha-1B | agonist | NR |
| | In. Agonist | NR |
| alpha-2A | agonist | NR |
| | antagonist | NR |
| alpha-2B | agonist | NR |
| | antagonist | NR |
| alpha-2C | agonist | NR |
| | antagonist | NR |
| beta 1 | agonist | NR |
| | antagonist | NR |
| beta 2 | agonist | NR |
| | antagonist | NR |
| endothelinB | agonist | NR |
| CCK-A | agonist | NR |
| NK-1 | agonist | NR |
| Vasopressin1A | agonist | NR |
| K-opiod | agonist | NR. |

HPLC/LCMS Method for Examples 138 to 252

The analysis was performed on a combined prep/analytical Waters/Micromass system consisting of a ZMD single quadropole mass spectrometer equipped with electrospray ionization interface. The HPLC system consisted of a Waters 600 gradient pump with on-line degassing, a 2700 sample manager and a 996 PDA detector. Separation was performed on an X-Terra MS C18, 5 μm 4.6×50 mm column. Buffer A: 10 mM ammoniumacetate in water, buffer B: 10 mM ammoniuacetate in acetonitrile/water 95/5. A gradient was run from 30% B to 100% B in 7 min, hold at 100% B for 1 min and re-equilibrated for 5.5 min. The system was operated at 1 ml/min.

### Preparation of Hydrochloride Salts

Typically, the tertiary amines were dissolved in dichloromethane, treated with an excess of 1M HCl in diethylether and precipitated from n-heptane. The solvents were removed in vacuo and after drying, the hydrochloride salts were obtained as colorless solids.

### Preparation of Oxalate or Tartrate Salts

Typically, the tertiary amines were dissolved in methanol, treated with 1 eq. of the appropriate acid, the solvent removed and the salt redissolved in dichloromethane and precipitated from n-heptane. The solvents were removed in vacuo affording the salts as colorless solids.

### Preparation of Phenylacetyl Chloride Derivatives

The phenylacetic acid derivative (15 mmol) was dissolved in dichloromethane (100 mL), and oxalylchloride (45 mmol) was added slowly. The reaction mixture was stirred for 4 hours and then evaporated to dryness. The product was obtained as a colorless oil and used immediately after preparation in the acylation step.

### Example 138

### 4-Isobutoxyphenylacetic acid (128NLS28)

Methyl 4-hydroxyphenyl acetate (14.6 g, 0.0885 mol) was dissolved in DMF (200 mL), potassium carbonate (31.0 g, 0.224 mmol) added and the mixture was stirred for 1 h at rt. 1-Bromo-2-methylpropane (19.2 mL, 0.177 mol) was added and the mixture was heated at 80° C. for 3 days under vigorous stirring. The mixture was cooled to rt, filtered, the solvent removed and the residue partitioned between 1.5M NaOH and ethyl acetate. The organic layer was evaporated, the residue dissolved in methanol (100 mL) and water (100 mL), KOH (10 g, 0.178 mol) added and the mixture stirred overnight at rt. The methanol was removed by evaporation, the mixture extracted with dichloromethane. The organic layer was discarded, the aqueous layer acidified with 4M HCl to pH2-3 and extracted twice with dichloromethane. The combined organic layers were dried over Na₂SO₄, filtered and evaporated to give the title compound (16.9 g, 92%) as a colorless solid.

### Example 139

### 4-Propoxyphenylacetic acid (98AF77-66)

Prepared as described for 128NLS28 using propylbromide as the alkylating agent.

### Example 140

### 4-Isopropoxyphenylacetic acid (130AF24-163)

Prepared as described for 128NLS28 using isopropylbromide as the alkylating agent.

### Example 141

### N-{1-[2-(1,3-Dioxolan-2-yl)ethyl]piperidin-4-yl}-N-(4-fluorobenzyl)-N′-(4-isobutoxybenzyl)carbamide, hydrochloride (80MBT86-2C)

4-Piperidone hydrochloride monohydrate (4.0 g, 26.0 mmol) was dissolved in dichloromethane (130 mL). After addition of triethylamine (8.66 g, 85.8 mmol), the mixture was stirred for 10 min and then cooled to 0° C. Trifluoroacetic anhydride (12.0 g, 57.2 mmol) was added dropwise under stirring. After 2 h at room temperature, the reaction was stopped by addition of water (100 mL), and the aqueous phase was extracted with dichloromethane (2×100 mL). The combined organic phases were dried over Na₂SO₄, filtered and concentrated to give 1-(trifluoroacetyl)-4-piperidone (5.07 g, 100%).

4-Fluorobenzylamine (3.14 g, 25.9 mmol) was dissolved in methanol (150 mL). 1-(trifluoroacetyl)-4-piperidone (5.07 g, 25.9 mmol) was added, and the pH was adjusted to ˜5 with acetic acid. The reaction mixture was stirred for 5 min and NaBH₃CN (2.46 g, 38.9 mmol) was added slowly under stirring. After 20 h at room temperature the reaction was concentrated. 2 M NaOH (100 mL) was added and the aqueous phase was extracted with dichloromethane (2×100 mL). The combined organic phases were dried over Na₂SO₄, filtered and concentrated to give N-(4-fluorobenzyl)-1-(trifluoroacetyl)piperidin-4-amine (50ELH85, 2.91 g, 37%).

4-isobutoxyphenylacetic acid (7.6 g, 36.5 mmol) was dissolved in THF (50 mL). Proton Sponge™ (8.2 g, 38 mmol) was added, and the mixture was stirred for 15 min. Diphenylphosphoryl azide (10.6 g, 38 mmol) was added dropwise and the mixture was heated to reflux for 4 h. The mixture was cooled to room temperature and placed in the freezer at −18° C. for 20 h. The resulting white precipitate was vigorously stirred with diethyl ether (250 mL) for 15 min and filtered. The filtrate was evaporated to give crude 4-isobutoxybenzyl isocyanate (1.97 g, 9.6 mmol), which was dissolved in dichloromethane (50 mL) and added to a solution of 50ELH85 (2.91 g, 9.6 mmol) in dichloromethane (50 mL). The reaction mixture was stirred for 20 h and concentrated. The crude product was purified by flash chromatography (0-5% methanol in dichloromethane) to give N-(4-fluorobenzyl)-N-[1-(trifluoroacetyl)piperidin-4-yl]-N′-(4-isobutyloxybenzyl)carbamide (76ELH17, 3.90 g, 91%).

The compound 76ELH17 (3.90 g, 8.7 mmol) was dissolved in methanol (12 ml) and added to a 2 M solution of potassium carbonate in methanol (100 mL) under stirring. After 4 h the methanol was evaporated, and the aqueous phase was extracted with dichloromethane (2×100 mL). The combined organic phases were dried over Na₂SO₄, filtered and concentrated to give a semi-pure solid (2.95 g), which was purified by flash chromatography (10% methanol in dichloromethane with 1% triethylamine) to give N-(4-fluorobenzyl)-N-(piperidin-4-yl)-N′-(4-isobutyloxybenzyl)carbamide (76ELH18, 1.40 g, 39%) as a colorless solid. LCMS m/z 414 [M+H]⁺. ¹H-NMR (CDCl₃): δ 7.21-6.75 (m, 8H), 4.47-4.42 (m, 1H), 4.39 (t, J=5 Hz, 1H), 4.35 (s, 2H), 4.27 (d, J=5 Hz, 2H), 3.68 (d, J=6 Hz, 2H), 3.13-3.06 (m, 2H), 2.74-2.66 (m, 2H), 2.11-1.99 (m, 1H), 1.78-1.71 (m, 3H), 1.58-1.46 (m, 2H), 1.00 (d, J=6 Hz, 6H).

The compound 76ELH18 (200 mg, 0.484 mmol) was dissolved in acetonitrile (20 mL). Potassium carbonate (74 mg, 0.553 mmol) and sodium iodide (80 mg, 0.553 mmol) was added followed by 2-(2-bromoethyl)-1,3-dioxolane (100 mg, 0.553 mmol). The reaction mixture was heated to reflux for 20 h. The mixture was concentrated, water (50 mL) was added, and the aqueous phase was extracted with dichloromethane (2×50 mL). The combined organic phases were dried over Na₂SO₄, filtered and evaporated. The resulting oil was purified twice by flash chromatography (5% methanol in dichloromethane) to give a colorless oil (50 mg, 20%). R^{f}=0.70 (MeOH/CH₂Cl₂ 1:9). LCMS m/z 514 [M+H]⁺. 1H-NMR (CDCl₃): δ 7.21-6.75 (m, 8H), 4.94 (t, J=4.5 Hz, 1H), 4.73-4.62 (m, 1H), 4.58 (t, J=5.5 Hz, 1H), 4.41 (s, 2H), 4.26 (d, J=5.5 Hz, 2H), 4.00-3.80 (m, 4H), 3.68 (d, J=6.0 Hz, 2H), 3.43-3.35 (m, 2H), 2.94-2.87 (m, 2H), 2.68-2.57 (m, 2H), 2.45-2.32 (m, 2H), 2.20-2.13 (m, 2H), 2.10-2.00 (m, 1H), 1.88-1.81 (m, 2H), 1.00 (d, J=6.0 Hz, 6H). HPLC t_{R}=8.1 min.

The collected compound was converted into its hydrochloride salt, which was obtained as a colorless solid (80MBT86-2C).

### Example 142

### N-{1-[2-(1,3-Dioxan-2-yl)ethyl]piperidin-4-yl}-N-(4-fluorobenzyl)-2-[4-(2-hydroxy-2-methylpropoxy)phenyl]acetamide, tartrate (106MBT54-D)

Methyl (4-hydroxyphenyl)acetate (500 mg, 3.0 mmol) was dissolved in DMF (3 mL). K₂CO₃ (829 mg, 6.0 mmol) was added followed by isobutylene oxide (800 μL, 9.0 mmol). The mixture was heated to 150° C. by microwave irradiation for 30 min and concentrated. The residue was dissolved in a 1:1 mixture of methanol and water (20 mL). NaOH (1 g) was added and the mixture was stirred for 30 min. Methanol was removed by rotary evaporation. The aqueous phase was acidified by 4 M HCl and extracted with dichloromethane (2×50 mL). The combined organic phases were extracted with 2 M NaOH (2×50 mL). The combined aqueous phases were subsequently acidified by 4 M HCl and extracted with dichloromethane (2×50 mL). The combined organic phases were dried over Na₂SO₄, filtered and evaporated to afford [4-(2-hydroxy-2-methylpropoxy)phenyl]acetic acid (106MBT52-D, 470 mg, 70%) as a colorless solid. ¹H-NMR (CDCl₃): δ 7.19 (m, 2H), 6.88 (m, 2H), 3.78 (s, 2H), 3.57 (s, 2H), 1.34 (s. 6H).

The acid 106MBT52-D (150 mg, 0.67 mmol) was dissolved in dichloromethane (10 mL). N-{1-[2-(1,3-dioxan-2-yl)ethyl]piperidin-4-yl}-N-(4-fluorobenzyl)amine (118AF52-95, 180 mg, 0.56 mmol) was added followed by triethylamine (235 μL, 0.84 mmol). Bromo-tris-pyrrolidino-phosphonium hexafluorophosphate (PyBroP, 392 mg, 0.84 mmol) was added, and the mixture was stirred at room temperature for 2 h. The mixture was concentrated and passed onto a prewashed (methanol) ion exchange column (0.88 mmol/g, 1 g). The column was washed with methanol (8×4 mL) and the remaining product was eluted off the column with 10% NH₄OH in methanol (2×4 mL) and evaporated. The resulting oil was dissolved in dichloromethane (20 mL) and washed with saturated aqueous NaHCO₃ (5×20 mL). The organic phase was dried over Na₂SO₄, filtered and evaporated. The resulting oil was purified by flash chromatography (0-5% methanol in dichloromethane) to give a colorless oil (110 mg, 31%). R^{f}=0.64 (MeOH/CH₂Cl₂ 1:9). LCMS m/z 529 [M+H]⁺.¹H-NMR (CDCl₃, rotamers 0.4:0.6): δ 7.25-6.82 (m, 8H), 4.64-4.48 (m, 2.4H), 4.44 (s, 1.2H), 4.10-4.03 (m, 2H), 3.79-3.67 (m, 5.2H), 3.50 (s, 1.2H), 2.90-2.81 (m, 2H), 2.42-3.95 (m, 2H), 2.12-1.98 (m, 2.2H), 1.87-1.79 (m, 0.8H), 1.76-1.48 (m, 5.2H), 1.36-1.27 (m, 7.8H). HPLC t_{R}=6.1 min.

The collected compound was converted into its tartrate salt, which was obtained as a colorless solid (106MBT54-D).

### Example 143

### N-(4-Fluorobenyzl)-N-(piperidin-4-yl)-2-(4-isobutoxyphenyl)acetamide (103NLS56)

To a solution of the amine 118AF93-51 (10.37 g, 30.3 mmol) and triethylamine (9.36 mL, 60.6 mmol) in dichloromethane (200 mL) a solution of 4-isobutoxyphenylacetyl chloride 128NLS28 (8.93 g, 39.4 mmol) in dichloromethane (100 mL) is added dropwise at 0° C. The solution is stirred at rt for 3 h, then water is added and the mixture washed with sat. aq. NaHCO₃. The organic layer was washed with 5% HCl, water and brine, dried over sodium sulfate, filtered and evaporated in vacuo. The residue was purified by silica gel column chromatography eluting with a stepwise gradient of 0-50% ethyl acetate in n-heptane, affording N-(4-fluorobenyzl)-N-[1-(benzyloxycarbonyl)piperidin-4-yl]-2-(4-isobutoxyphenyl)acetamide as a colorless oil.

This compound was dissolved in abs. ethanol (200 mL) and hydrogenated overnight at rt using Pd/C (10%, 1 g) as a catalyst. The mixture was filtered over Celite, the solvent removed and the residue dried in vacuo to give a colorless oil (7.02 g, 58% over both steps). This compound was used without further purification. LCMS m/z 399 [M+H]⁺. HPLC t_{R}=8.8 min.

### Example 144

### N-{1-[3-(3,5-Dimethylpiperidin-1-yl)propyl]piperidin-4-yl}-N-(4-fluorobenzyl)-2-(4-isobutoxyphenyl)acetamide, dihydrochloride (103NLS45-B)

To 3,5-dimethylpiperidine (43 μL, 0.33 mmol) in DMF (1 mL) was added potassium carbonate (132 mg, 1.0 mmol), followed by 1-chloro-3-iodopropane (32 μmol, 0.30 mmol) and the mixture stirred at 50° C. for 2 h. After cooling to rt, a solution of 103NLS56 (100 mg, 0.25 mmol) in DMF (0.5 mL) was added, followed by sodium iodide (45 mg, 0.30 mmol). The mixture was shaken for 20 h at 60° C., filtered, evaporated to dryness and purified by silica gel column chromatography, eluting with a stepwise gradient of 0-10% methanol in dichloromethane. The residue was further purified by passage over a reversed phase C₁₈ SPE cartridge, giving the desired compound (35 mg, 25%), which was converted into its dihydrochloride salt.

R_{f}=0.61 (MeOH/CH₂Cl₂ 1:9). LCMS m/z 552 [M+H]⁺. HPLC t_{R}=8.7 min.

### Example 145

### 1-[3-(4-{(4-Fluorobenzyl)-[2-(4-isobutoxyphenyl)acetyl]amino}piperidin-1-yl)propyl]piperidine-4-carboxylic acid methyl ester, dihydrochloride (103NLS45-E)

Prepared following the same method as described for 103NLS45-B, using piperidine-4-carboxylic acid methyl ester (44 μL, 0.33 mmol). Yield: 7 mg, 5%.

LCMS m/z 582 [M+H]⁺. HPLC t_{R}=7.8 min.

### Example 146

### N-(4-Fluorobenzyl)-2-(4-isobutoxyphenyl)-N-{1-[2-(1-methylpyrrolidin-2-yl)ethyl]piperidin-4-yl}acetamide, dioxalate (103NLS63-G)

To a solution of the amine 103NLS56 (15 mg, 0.038 mmol) in DMF (0.3 mL) was added a solution of 2-(2-chloroethyl)-1-methylpyrrolidine hydrochloride (8.4 mg, 0.045 mmol) in DMF (0.1 mL), followed by caesium carbonate (50 mg, 0.15 mmol) and sodium iodide (6.8 mg, 0.045 mmol). The mixture was stirred overnight at 60° C., partitioned between dichloromethane and sat. aq. NaHCO₃ solution. The organic layer was dried over sodium sulfate, filtered and evaporated. The residue was purified by preparative reversed phase (C₁₈) HPLC and the obtained compound (10.5 mg, 54%) converted into its dioxalate salt.

LCMS m/z 510 [M+H]⁺. HPLC t_{R}=8.1 min.

### Example 147

### N-{1-[3-(2,6-Dimethylmorpholin-4-yl)propyl]piperidin-4-yl}-N-(4-fluorobenzyl)-2-(4-isobutoxyphenyl)acetamide, dioxalate (103NLS69-A)

To a solution of 2,6-dimethylmorpholine (6.1 μL, 49 μmol) in DMF (0.3 mL) 1-chloro-3-iodopropane (4.9 μL, 45 μmol) in DMF (0.05 mL) was added, followed by caesium carbonate (50 mg, 0.15 mmol). The mixture was shaken at 50° C. for 3 h. After cooling to rt, the piperidine derivative 103NLS56 (15 mg, 38 μmol) in DMF (0.1 mL) and sodium iodide (6.8 mg, 45 μmol) were added and stirring maintained overnight at 60° C. The mixture was partitioned between dichloromethane and sat. aq. NaHCO₃ solution. The organic layer was dried over sodium sulfate, filtered and evaporated. The residue was purified by preparative reversed phase (C₁₈) HPLC and the obtained compound (6.3 mg, 30%) converted into its dioxolate salt.

LCMS m/z 554 [M+H]⁺. HPLC t_{R}=8.7 min.

### Example 148

### N-(4-Fluorobenzyl)-N-{1-[3-(3-hydroxypiperidin-1-yl)propyl]piperidin-4-yl}-2-(4-isobutoxyphenyl)acetamide, dioxalate (103NLS69-B)

Prepared following the same method as described for 103NLS69-A, using 3-hydroxypiperidine hydrochloride (6.8 mg, 49 μmol). Yield: 7.9 mg, 30%. LCMS m/z 540 [M+H]⁺. HPLC t_{R}=8.1 min.

### Example 149

### N-(4-Fluorobenzyl)-2-(4-isobutoxyphenyl)-N-{1-[3-(2-methylpiperidin-1-yl)propyl]piperidin-4-yl}acetamide, dioxalate (103NLS69-C)

Prepared following the same method as described for 103NLS69-A, using 2-methylpiperidine (5.8 μL, 49 μmol). Yield: 5.2 mg, 26%. LCMS m/z 538 [M+H]⁺. HPLC t_{R}=8.7 min.

### Example 150

### N-(4-Fluorobenzyl)-2-(4-isobutoxyphenyl)-N-[1-(3-pyrrolidin-1-yl-propyl)piperidin-4-yl]acetamide, dioxalate (103NLS69-D

Prepared following the same method as described for 103NLS69-A, using pyrrolidine (5.0 μL, 49 μmol). Yield: 4.6 mg, 24%. LCMS m/z 510 [M+H]⁺. HPLC t_{R}=8.4 mm.

### Example 151

### N-{1-[3-(2,5-Dimethylppyrrolidin-1-yl)propyl]piperidin-4-yl}-N-(4-fluorobenzyl)-2-(4-isobutoxyphenyl)acetamide, dioxalate (103NLS69-E)

Prepared following the same method as described for 103NLS69-A, using 2,5-dimethylpyrrolidine (6.0 μL, 49 μmol). Yield: 3.4 mg, 17%. LCMS m/z 538 [M+H]⁺. HPLC t_{R}=8.7 min.

### Example 152

### N-(4-Fluorobenzyl)-N-{1-[3-(3-hydroxymethylpiperidin-1-yl)propyl]piperidin-4-yl}-2-(4-isobutoxyphenyl)acetamide, dioxalate (103NLS69-F)

Prepared following the same method as described for 103NLS69-A, using 3-hydroxymethylpiperidine (5.5 μL, 49 μmol). Yield: 5.5 mg, 26%. LCMS m/z 554 [M+H]⁺. HPLC t_{R}=8.0 min.

### Example 153

### (4S)-3-(3-chloropropyl)-4-isopropyloxazolidinon-2-one (103NLS94)

Sodium hydride (60% suspension in oil, 288 mg, 7.2 mmol) was added to a solution of (S)-4-isopropyl-2-oxazolidinone (775 mg, 6.0 mmol) in dry tetrahydrofuran (50 mL) under argon atmosphere. The suspension was stirred for 15 min at rt, then 1-bromo-3-chloropropane (1.18 mL, 12.0 mmol) was added dropwise over 30 min. The mixture was refluxed overnight, filtered and the filtrate evaporated in vacuo. Purification of the residue by silica gel column chromatography, eluting with a stepwise gradient of 0-4% methanol in dichloromethane afforded (4S)-3-(3-chloropropyl)-4-isopropyloxazolidinon-2-one (824 mg, 67%) as a colorless oil.

### Example 154

### N-(4-Fluorobenzyl)-2-(4-isobutoxyphenyl)-N-{1-[3-(4-(S)-isopropyl-2-oxo-oxazolidin-3-yl)propyl]piperidin-4-yl}acetamide, oxalate (117NLS01)

To a solution of 103NLS56 (207 mg, 0.52 mmol) potassium carbonate (215 mg, 1.56 mmol) was added, followed by the alkylating agent 103NLS94 (127 mg, 0.62 mmol) and sodium iodide (93 mg, 0.62 mmol). The mixture was stirred at 65° C. overnight, the solvent removed and the residue partitioned between ethyl acetate and water. The organic layer was dried over Na₂SO₄, filtered and evaporated. The residue was purified by silica gel column chromatography, eluting with a stepwise gradient of 0-4% methanol in dichloromethane. Further purification of the compound was performed by passage over an acidic ion exchange SPE cartridge, affording the desired compound (209 mg, 71%) as a colorless oil, which was converted into its oxalate salt.

R_{f}=0.35 (MeOH/CH₂Cl₂ 5:95). LCMS m/z 568 [M+H]⁺. ¹H-NMR (CDCl₃, rotamers 0.6:0.4) δ 7.21-6.80 (m, 8H, Ar—H), 4.60-4.53 (m, 0.6H, pip-H), 4.49 and 4.43 (2s, 2H, benzyl-H), 4.19-4.14 (m, 1H, oxa-CH₂), 4.06-4.01 (m, 1H, oxa-CH₂), 3.77-3.67 (m, 4.2H, pip-H, oxa-NCH, CH_{2OiBu}, benzyl-H), 3.53-3.46 (m, 2.2H, benzyl-H, OCONCH₂), 2.98-2.85 (m, 3H, pip-H, OCONCH₂), 2.39-2.25 (m, 2H, NCH₂), 2.10-2.00 (m, 3.2H, CH(CH₃)₂, pip-H, CH_{OiBu}), 1.85-1.50 (m, 6H, pip-H, NCH₂CH₂), 1.29 (m, 0.8H, pip-H), 1.01-0.99 (m, 6H, CH_{3OiBu}), 0.89-0.83 (m, 6H, CH(CH₃)₂). HPLC t_{R}=8.9 min.

### Example 155

### N-[2-(4-Fluorophenyl)ethyl]-2-(4-isobutoxyphenyl)-N-{1-[3-(4-(S)-isopropyl-2-oxo-oxazolidin-3-yl)propyl]piperidin-4-yl}acetamide, oxalate (117NLS03-A)

Prepared following the same method as described for 117NLS01 using N-[2-(4-fluorophenyl)ethyl]-2-(4-isobutoxyphenyl)-N-(piperidin-4-yl)acetamide (111 mg, 0.27 mmol, prepared by the procedure described for 103NLS56). Yield: 90 mg, 57%.

R_{f}=0.30 (MeOH/CH₂Cl₂ 5:95). LCMS m/z 582 [M+H]⁺. ¹H-NMR (CDCl₃, rotamers 0.6:0.4) δ 7.18-6.80 (m, 8H, Ar—H), 4.40-4.35 (m, 0.4H, pip-H), 4.20-4.15 (m, 1H, oxa-CH₂), 4.05-4.01 (m, 1H, oxa-CH₂), 3.75-3.46 (m, 6.6H, pip-H, oxa-NCH, CH_{2OiBu}, benzyl-H, OCONCH₂), 3.36 (m, 2H, ArCH₂CH₂N), 3.02-2.84 (m, 3H, pip-H, OCONCH₂), 2.81-2.75 (m, 2H, ArCH₂), 2.37-2.25 (m, 2H, NCH₂), 2.09-1.98 (m, 2.8H, CH(CH₃)₂, pip-H, CH_{OiBu}), 1.85-1.62 (m, 6H, pip-H, NCH₂CH₂), 1.31 (m, 1.2H, pip-H), 1.00-0.97 (m, 6H, CH_{3OiBu}), 0.89-0.84 (m, 6H, CH(CH₃)₂). HPLC t_{R}=9.1 min.

### Example 156

### N-[2-(4-Fluorophenyl)ethyl]-N-{1-[3-(4-(S)-isopropyl-2-oxo-oxazolidin-3-yl)propyl]piperidin-4-yl}-2-(4-propoxyphenyl)acetamide, oxalate (117NLS03-B)

Prepared following the same method as described for 117NLS01 using N-[2-(4-fluorophenyl)ethyl]-N-(piperidin-4-yl)-2-(4-propoxyphenyl)acetamide (108 mg, 0.27 mmol, prepared by the procedure described for 103NLS56). Yield: 76 mg, 50%.

R_{f}=0.33 (MeOH/CH₂Cl₂ 5:95). LCMS m/z 568 [M+H]⁺. ¹H-NMR (CDCl₃, rotamers 0.6:0.4) δ 7.17-6.81 (m, 8H, Ar—H), 4.40-4.35 (m, 0.4H, pip-H), 4.20-4.15 (m, 1H, oxa-CH₂), 4.05-4.01 (m, 1H, oxa-CH₂), 3.90-3.85 (m, 2H, OCH_{2OPr}), 3.72-3.48 (m, 4.6H, pip-H, oxa-NCH, benzyl-H, OCONCH₂), 3.36-3.30 (m, 2H, ArCH₂CH₂N), 2.99-2.86 (m, 3H, pip-H, OCONCH₂), 2.80-2.74 (m, 2H, ArCH₂), 2.38-2.26 (m, 2H, NCH₂), 2.11-2.03 (m, 1.8H, CH(CH₃)₂, pip-H), 1.87-1.64 (m, 8H, pip-H, CH_{2OPr}, NCH₂CH₂), 1.31 (m, 1.2H, pip-H), 1.03-0.98 (m, 3H, CH_{3OPr}), 0.88-0.83 (m, 6H, CH(CH₃)₂). HPLC t_{R}=8.5 min.

### Example 157

### N-(4-Fluorobenzyl)-N-{1-[3-(4-(S)-isopropyl-2-oxo-oxazolidin-3-yl)propyl]piperidin-4-yl}-2-(4-propoxyphenyl)acetamide, oxalate (117NLS03-C)

Prepared following the same method as described for 117NLS01 using N-(4-fluorobenzyl)-N-(piperidin-4-yl)-2-(4-propoxyphenyl)acetamide (104 mg, 0.27 mmol, prepared by the procedure described for 103NLS56). Yield: 120 mg, 80%.

R_{f}=0.36 (MeOH/CH₂Cl₂ 5:95). LCMS m/z 554 [M+H]⁺. ¹H-NMR (CDCl₃, rotamers 0.6:0.4) δ 7.19-6.78 (m, 8H, Ar—H), 4.57-4.48 (m, 0.6H, pip-H), 4.48 and 4.42 (2s, 2H, benzyl-H), 4.18-4.12 (m, 1H, oxa-CH₂), 4.04-4.00 (m, 1H, oxa-CH₂), 3.91-3.85 (m, 2H, OCH_{2OPr}), 3.75-3.66 (m, 2.2H, pip-H, oxa-NCH, benzyl-H), 3.49-3.43 (m, 2.2H, benzyl-H, OCONCH₂), 2.98-2.80 (m, 3H, pip-H, OCONCH₂), 2.33-2.25 (m, 2H, NCH₂), 2.05-1.50 (m, 10.2H, CH(CH₃)₂, NCH₂CH₂, pip-H, CH_{2OPr}), 1.27 (m, 0.8H, pip-H), 1.18-0.98 (m, 3H, CH_{3OPr}), 0.87-0.81 (m, 6H, CH(CH₃)₂). HPLC t_{R}=8.3 min.

### Example 158

### N-{1-[2-(1,3-Dioxan-2-yl)ethyl]piperidin-4-yl}-N-(4-fluorobenzyl)-2-(4-isobutoxyphenyl)acetamide, oxalate (103NLS63-F)

Prepared following the same method as described for 117NLS01 using and 103NLS56 (262 mg, 0.657 mmol) and 2-(2-bromoethyl)-1,3-dioxane as the alkylating agent. No sodium iodide was required. Yield: 152 mg, 45%.

R_{f}=0.35 (MeOH/CH₂Cl₂ 1:9). LCMS m/z 513 [M+H]⁺. ¹H-NMR (CDCl₃, rotamers 0.6:0.4) δ 7.26-6.80 (m, 8H, Ar—H), 4.63-4.39 (m, 3.6H, pip-H, dioxane-H, benzyl-H), 4.09-4.01 (m, 2H, dioxane-H), 3.78-3.64 (m, 5.2H, pip-H, dioxane-H, CH_{2OiBu}, benzyl-H), 3.50 (s, 1.2H, benzyl-H), 2.92-2.79 (m, 2H, pip-H), 2.43-2.34 (m, 2H, NCH₂), 2.10-1.96 (m, 3.2H, dioxane-H, pip-H, CH_{OiBu}), 1.88-1.48 (m, 6H, pip-H, NCH₂CH₂), 1.35-1.24 (m, 1.8H, dioxane-H, pip-H), 1.01 (m, 6H, CH_{3OiBu}). HPLC t_{R}=8.8 min.

### Example 159

### N-{1-[2-(1,3-Dioxan-2-yl)ethyl]piperidin-4-yl}-N-[2-(4-fluorophenyl)ethyl]-2-(4-isobutoxyphenyl)acetamide, oxalate (117NLS03-D)

Prepared following the same method as described for 117NLS03-A using 2-(2-bromoethyl)-1,3-dioxane as the alkylating agent. No sodium iodide was required. Yield: 99 mg, 70%.

R_{f}=0.35 (MeOH/CH₂Cl₂ 5:95). LCMS m/z 527 [M+H]⁺. ¹H-NMR (CDCl₃, rotamers 0.7:0.3) δ 7.18-6.80 (m, 8H, Ar—H), 4.58-4.54 (m, 1H, dioxane-H), 4.48-4.41 (m, 0.3H, pip-H), 4.10-4.06 (m, 2H, dioxane-H), 3.77-3.66 (m, 5.4H, dioxane-H, benzyl-H, CH_{2OiBu}), 3.64-3.52 (m, 1.3H, benzyl-H, pip-H), 3.37-3.32 (m, 2H, CH₂NCO), 2.99 and 2.89 (2m, 2H, pip-H), 2.82-2.76 (m, 2H, ArCH₂), 2.49-2.39 (m, 2H, NCH₂), 2.12-2.00 (m, 2.6H, dioxane-H, pip-H, CH_{OiBu}), 1.88-1.67 (m, 6H, pip-H, CH_{2OiBu}, NCH₂CH₂), 1.35-1.31 (m, 2.4H, dioxane-H, pip-H), 1.00 (t, 6H, J=6.6, CH_{3OiBu}). HPLC t_{R}=8.8 min.

### Example 160

### N-{1-[2-(1,3-Dioxan-2-yl)ethyl]piperidin-4-yl}-N-[2-(4-fluorophenyl)ethyl]-2-(4-propoxyphenyl)acetamide, oxalate (117NLS03-E)

Prepared following the same method as described for 117NLS03-B using 2-(2-bromoethyl)-1,3-dioxane as the alkylating agent. No sodium iodide was required. Yield: 90 mg, 65%.

R_{f}=0.23 (MeOH/CH₂Cl₂ 5:95). LCMS m/z 513 [M+H]⁺. ¹H-NMR (CDCl₃, rotamers 0.7:0.3) δ 7.21-6.81 (m, 8H, Ar—H), 4.58-4.54 (m, 1H, dioxane-H), 4.48-4.42 (m 0.3H, pip-H), 4.10-4.06 (m, 2H, dioxane-H), 3.91-3.86 (m, 2H, CH_{2OPr}), 3.77-3.69 (m, 3.4H, dioxane-H, benzyl-H), 3.63-3.56 (m, 1.3H, benzyl-H, pip-H), 3.38-3.31 (m, 2H, CH₂NCO), 2.99 and 2.89 (2m, 2H, pip-H), 2.82-2.76 (m, 2H, ArCH₂), 2.49-2.39 (m, 2H, NCH₂), 2.12-2.00 (m, 1.6H, dioxane-H, pip-H), 1.87-1.65 (m, 8H, pip-H, CH_{2OPr}, NCH₂CH₂), 1.35-1.31 (m, 2.4H, dioxane-H, pip-H), 1.05-1.00 (m, 3H, CH_{3OPr}). HPLC t_{R}=8.0 min.

### Example 161

### N-{1-[2-(1,3-Dioxan-2-yl)ethyl]piperidin-4-yl}-N-(4-fluorobenzyl)-2-(4-propoxyphenyl)acetamide, tartrate (117NLS03-F)

Prepared following the same method as described for 117NLS03-C using 2-(2-bromoethyl)-1,3-dioxane as the alkylating agent. No sodium iodide was required. Yield: 107 mg, 79%.

R_{f}=0.41 (MeOH/CH₂Cl₂ 5:95). LCMS m/z 499 [M+H]⁺. ¹H-NMR (CDCl₃, rotamers 0.6:0.4) δ 7.20-6.80 (m, 8H, Ar—H), 4.62-4.56 (m, 0.6H, pip-H), 4.54-4.51 (m, 1H, dioxane-H), 4.49 and 4.43 (2s, 2H, benzyl-H), 4.08-4.04 (m, 2H, dioxane-H), 3.92-3.87 (m, 2H, OCH_{2OPr}), 3.76-3.68 (m, 3.2H, pip-H, dioxane-H, benzyl-H), 3.50 (s, 1.2H, benzyl-H), 2.90-2.83 (m, 2H, pip-H), 2.43-2.36 (m, 2H, NCH₂), 2.10-1.98 (m, 2.2H, dioxane-H, pip-H), 1.86-1.51 (m, 8H, pip-H, CH_{2OPr}, NCH₂CH₂), 1.32-1.27 (m, 1.8H, dioxane-H, pip-H), 1.05-0.99 (m, 3H, CH₃). HPLC t_{R}=7.6 min.

### Example 162

### N-{1-[2-(1,3-Dioxan-2-yl)ethyl]piperidin-4-yl}-N-(4-fluorobenzyl)-N′-(4-isobutoxybenzyl)carbamide, tartrate (117NLS25)

Prepared following the same method as described for 117NLS01 using 2-(2-bromoethyl)-1,3-dioxane (24 μL, 0.18 mmol) as the alkylating agent and N-(4-fluorobenzyl)-N′-(4-isobutoxybenzyl)-N-(piperidin-4-yl)carbamide (76ELH18, 50 mg, 0.12 mmol). No sodium iodide was required. Yield: 38 mg, 60%.

R_{f}=0.32 (MeOH/CH₂Cl₂ 1:9). LCMS m/z 528 [M+H]⁺. ¹H-NMR (CDCl₃) δ 7.18-6.74 (m, 8H, Ar—H), 4.53 (t, 1H, J=5.1, dioxane-H), 4.46 (t, 1H, J=5.3, NH), 4.33-4.25 (m, 5H, pip-H, benzyl-H), 4.08-4.04 (m, 2H, dioxane-H), 3.75-3.68 (m, 2H, dioxane-H), 3.66 (d, 2H, J=6.6, CH_{2OiBu}), 2.93-2.88 (m, 2H, pip-H), 2.43-2.39 (m, 2H, NCH₂), 2.09-1.98 (m, 4H, CH_{OiBu}, dioxane-H, pip-H), 1.77-1.56 (m, 6H, pip-H, NCH₂CH₂), 1.32-1.28 (m, 1H, dioxane-H), 0.99 (d, 6H, J=6.6, CH_{3OiBu}). HPLC t_{R}=8.7 min.

### Example 163

### N-{1-[2-(1,3-Dioxan-2-yl)ethyl]piperidin-4-yl}-N-(4-fluorobenzyl)-2-(4-fluorophenyl)acetamide, tartrate (117NLS87-A)

To a solution of 118AF52-95 (300 mg, 0.93 mmol) and triethylamine (0.52 mL, 3.72 mmol) in dry THF (10 mL) at 0° C. a solution of 4-fluorophenylacetyl chloride (0.19 mL, 1.39 mmol) in THF (5 mL) was added dropwise and stirring was continued at rt for 3 h. The reaction mixture was filtered and the filtrate evaporated to dryness. The residue was partitioned between ethyl acetate and 1M NaOH, the organic layer washed with brine, dried over Na₂SO₄, filtered and evaporated. Purification by silica gel column chromatography, eluting with a stepwise gradient of 0-8% methanol in dichloromethane, followed by purification of the compound by passage over an acidic ion exchange SPE cartridge, afforded the desired compound (131 mg, 31%), which was converted to its tartrate form as described above.

R_{f}=0.39 (MeOH/CH₂Cl₂ 1:9). LCMS m/z 459 [M+H]⁺.¹H-NMR (CDCl₃, rotamers 0.6:0.4) δ 7.25-6.88 (m, 8H, Ar—H), 4.58-4.52 (m, 0.6H, pip-H), 4.50 (t, 1H, J=5.1, dioxane-H), 4.48 and 4.44 (2s, 2H, benzyl-H), 4.06-4.02 (m, 2H, dioxane-H), 3.78 and 3.50 (2s, 2H, benzyl-H), 3.72-3.64 (m, 2.4H, pip-H, dioxane-H), 2.84 (m, 2H, pip-H), 2.40-2.35 (m, 2H, NCH₂), 2.07-1.99 (m, 2.2H, dioxane-H, pip-H), 1.85-1.50 (m, 6H, pip-H, NCH₂CH₂), 1.30-1.25 (m, 1.8H, dioxane-H, pip-H). HPLC t_{R}=6.9 min.

### Example 164

### N-{1-[2-(1,3-Dioxan-2-yl)ethyl]piperidin-4-yl}-N-(4-fluorobenzyl)-2-p-tolylacetamide, tartrate (117NLS87-B)

Prepared following the same method as described for 117NLS87-A using 4-methylphenylacetyl chloride and 118AF52-95 (300 mg, 0.93 mmol). Yield: 119 mg, 28%.

R_{f}=0.43 (MeOH/CH₂Cl₂ 1:9). LCMS m/z 455 [M+H]⁺.¹H-NMR (CDCl₃, rotamers 0.5:0.5) δ 7.17-6.87 (m, 8H, Ar—H), 4.60-4.53 (m, 0.5H, pip-H), 4.50 (t, 1H, J=5.1, dioxane-H), 4.48 and 4.41 (2s, 2H, benzyl-H), 4.05-4.01 (m, 2H, dioxane-H), 3.77-3.66 (m, 3.5H, pip-H, benzyl-H, dioxane-H), 3.50 (s, 1H, benzyl-H), 2.87-2.80 (m, 2H, pip-H), 2.40-2.34 (m, 2H, NCH₂), 2.30 and 2.28 (2s, 3H, CH₃), 2.07-1.95 (m, 2H, dioxane-H, pip-H), 1.83-1.50 (m, 6H, pip-H, NCH₂CH₂), 1.29-1.25 (m, 2H, dioxane-H, pip-H). HPLC t_{R}=7.7 min.

### Example 165

### 2-Benzofuran-5-yl-N-{1-[2-(1,3-dioxan-2-yl)ethyl]piperidin-4-yl}-N-(4-fluorobenzyl)acetamide, tartrate (128NLS22-A)

Benzofuran-5-yl-acetic acid was prepared adapting a procedure by Dunn et al. (J. Med. Chem., 1986, 29, 2326) and converted into the corresponding acetyl chloride by treatment with oxalylchloride. The title compound was prepared from 118AF52-95 (58 mg, 0.18 mmol) following the same method as described for 117NLS87-A. Yield: 27 mg, 43%.

R_{f}=0.52 (MeOH/CH₂Cl₂ 1:9). LCMS m/z 481 [M+H]⁺. ¹H-NMR (CDCl₃, rotamers 0.6:0.4) δ 7.64-6.68 (m, 9H, Ar—H), 4.62-4.54 (m, 0.6H, pip-H) 4.53-4.44 (m, 3H, dioxane-H, benzyl-H), 4.07-4.03 (m, 2H, dioxane-H), 3.82-3.61 (m, 3.2H, pip-H, benzyl-H, dioxane-H), 3.45 (s, 1.2H, benzyl-H), 2.91-2.80 (m, 2H, pip-H), 2.44-2.35 (m, 2H, NCH₂), 2.08-1.98 (m, 2.2H, dioxane-H, pip-H), 1.85-1.56 (m, 6H, pip-H, NCH₂CH₂), 1.32-1.27 (m, 1.8H, dioxane-H, pip-H). HPLC t_{R}=6.6 min.

### Example 166

### 2-(2,3-Dihydrobenzofuran-5-yl)-N-{1-[2-(1,3-dioxan-2-yl)ethyl]piperidin-4-yl}-N-(4-fluorobenzyl)acetamide, tartrate (128NLS22-B)

The compound (2,3-Dihydrobenzofuran-5-yl)acetic acid was prepared adapting a procedure by Dunn et al. (J. Med. Chem., 1986, 29, 2326) and converted into the corresponding acetyl chloride by treatment with oxalylchloride. The title compound was prepared from 118AF52-95 (58 mg, 0.18 mmol) following the same method as described for 117NLS87-A. Yield: 27 mg, 31%.

R_{f}=0.50 (MeOH/CH₂Cl₂ 1:9). LCMS m/z 483 [M+H]⁺. ¹H-NMR (CDCl₃, rotamers 0.6:0.4) δ 7.10-6.60 (m, 7H, Ar—H), 4.55-4.40 (m, 5.6H, pip-H, dioxane-H. benzyl-H, ArOCH₂), 4.01-3.97 (m, 2H, dioxane-H), 3.72-3.62 (m, 3.2H, pip-H, benzyl-H, dioxane-H), 3.41 (s, 1.2H, benzyl-H), 3.14-3.06 (m, 2H, OCH₂CH₂), 2.80 (m, 2H, pip-H), 2.35-2.30 (m, 2H, NCH₂), 1.99-1.93 (m, 2.2H, dioxane-H, pip-H), 1.80-1.44 (m, 6H, pip-H, NCH₂CH₂), 1.27-1.22 (m, 1.8H, dioxane-H, pip-H). HPLC t_{R}=6.9 min.

### Example 167

### N-{1-[2-(2,2-Dimethyl-1,3-dioxolan-4-yl)ethyl]piperidin-4-yl}-N-(4-fluorobenzyl)-2-(4-isobutoxyphenyl)acetamide, tartrate (117NLS37)

1-(2′,2′-Dimethyl-1′,3′-dioxolan-4′-yl)ethanol was prepared according to literature procedures (Carman R. M et al., Aust. J. Chem., 1998, 51, 955) and oxidized to the aldehyde by treatment with pyridinium chlorochromate. The crude aldehyde (80 mg, 0.55 mmol) was added to a solution of 103NLS56 (184 mg, 0.46 mmol) in methanol (5 mL). Acetic acid (0.05 mL) was added, followed by sodium cyanoborohydride (58 mg, 0.92 mmol) and the mixture stirred overnight at rt. The solvent was removed and the residue partitioned between dichloromethane and 1M NaOH. The organic layer was washed with sat. NH₄Cl, dried over Na₂SO₄, filtered and evaporated. Purification by silica gel column chromatography eluting with 0-5% methanol in dichloromethane afforded the desired compound (50 mg, 21%), which was converted into its tartrate salt.

R_{f}=0.39 (MeOH/CH₂Cl₂ 1:9). LCMS m/z 527 [M+H]⁺.¹H-NMR (CDCl₃, rotamers 0.6:0.4) δ 7.22-6.79 (m, 8H, Ar—H), 4.62-4.54 (m, 0.6H, pip-H), 4.49 and 4.42 (2s, 2H, benzyl-H), 4.06-3.98 (m, 1H, dioxolane-H), 3.75-3.66 (m, 4.4H, pip-H, CH_{2OiBu}, benzyl-H), 3.48 (m, 2H, dioxolane-H), 2.89-2.83 (m, 2H, pip-H), 2.45-2.25 (m, 2H, NCH₂), 2.07-1.99 (m, 2.2H, pip-H, CH_{OiBu}), 1.85-1.51 (m, 6H, pip-H, NCH₂CH₂), 1.36-1.28 (m, 6.8H, C(CH₃)₂, pip-H), 1.02-0.99 (m, 6H, CH_{3OiBu}). HPLC t_{R}=9.3 min.

### Example 168

### 4-[2-(Tosyloxy)ethyl]-1,3-dioxane (128NLS46-B)

A suspension of 1,3,5-pentanetriol (1.01 g, 8.33 mmol), paraformaldehyde (0.46 g) and methanesulfonic acid (0.33 mL) in DMF (3 mL) is heated for 10 min at 130° C. under microwave irradiation. The mixture was partitioned between ethyl acetate and water, the organic layer dried over Na₂SO₄, filtered and evaporated. The residue was dissolved in methanol (3 mL), conc. HCl (0.09 mL) added, and the mixture heated at 80° C. for 10 min under microwave irradiation. Ethyl acetate and 2M NaOH were added, the aqueous layer extracted twice with ethyl acetate and the combined organic layers washed with brine, dried over Na₂SO₄, filtered and evaporated. The crude product was treated with p-tosylchloride and DMAP following literature procedures (Moune et al., J. Org. Chem., 1997, 62, 3332). The title compound (1.18 g, 49% overall crude yield) was obtained as a yellowish oil, which was used without purification.

### Example 169

### N-{1-[2-(1,3-Dioxan-4-yl)ethyl]piperidin-4-yl}-N-(4-fluorobenzyl)amine (128NLS52)

To a suspension of 4-piperidone monohydrate hydrochloride (1.26 g, 8.23 mmol) in acetonitrile (100 mL), potassium carbonate (3.4 g, 24.6 mmol) was added, followed by the tosylate 128NLS46-B (3.54 g, 12.36 mmol) and sodium iodide (1.85 g, 12.35 mmol) and stirring was continued overnight at 60° C. The mixture was filtered, the filtrate evaporated in vacuo and the residue partitioned between 1M NaOH and ethyl acetate. The organic layer was separated, the aqueous layer extracted twice with ethyl acetate and the combined organic layers dried over sodium sulphate, filtered and evaporated to dryness. Purification of the residue by silica gel column chromatography, eluting with a stepwise gradient of 0-4% methanol in dichloromethane, afforded 1-[2-(1,3-dioxan-4-yl)ethyl]piperidin-4-one (128NLS50, 1.73 g, 98%).

To a solution of 128NLS50 (1.73 g, 8.13 mmol) in methanol (100 mL) was added dropwise 4-fluorobenzylamine (0.93 mL, 8.13 mmol) and acetic acid. Sodium cyanoborohydride (2.15 g, 40 mmol) was added slowly to the mixture at 0° C. and stirring was continued at rt overnight. The reaction mixture was concentrated in vacuo and the residue partitioned between dichloromethane and 1M NaOH, the aqueous layer extracted twice with dichloromethane and the combined organic layers dried over Na₂SO₄, filtered and evaporated to dryness. Purification of the residue by a short silica gel column chromatography eluting with 0-30% methanol in dichloromethane gave the title compound (1.51 g, 58%) as a colorless solid.

### Example 170

### N-{1-[2-(1,3-Dioxan-4-yl)ethyl]piperidin-4-yl}-N-(4-fluorobenzyl)-2-(4-isobutoxyphenyl)acetamide, tartrate (128NLS62)

Prepared following the same method as described for 117NLS87-A using 4-isobutoxyphenylacetyl chloride and 128NLS52 (480 mg, 1.49 mmol). Yield: 458 mg, 60%.

R_{f}=0.36 (MeOH/CH₂Cl₂ 1:9). LCMS m/z 513 [M+H]⁺. ¹H-NMR (CDCl₃, rotamers 0.6:0.4) δ 7.21-6.80 (m, 8H, Ar—H), 5.01 (d, 1H, J=6.1, dioxane-H), 4.66-4.56 (m, 1.6H, pip-H, dioxane-H) 4.51 and 4.44 (2s, 2H, benzyl-H), 4.09-4.05 (m, 1H, dioxane-H), 3.77 and 3.51 (2s, 2H, benzyl-H), 3.70-3.57 (m, 4.4H, pip-H, dioxane-H, CH_{2OiBu}), 2.91-2.83 (m, 2H, pip-H), 2.45-2.34 (m, 2H, NCH₂), 2.10-2.00 (m, 2.2H, pip-H, CH_{OiBu}), 1.85-1.26 (m, 8.8H, pip-H, dioxane-H, NCH₂CH₂), 1.03-1.00 (m, 6H, CH_{3OiBu}). HPLC t_{R}=8.8 min.

### Example 171

### N-{1-[2-(1,3-Dioxan-4-yl)ethyl]piperidin-4-yl}-N-(4-fluorobenzyl)-2-(4-trifluoromethylphenyl)acetamide, tartrate (128NLS54-A)

Prepared following the same method as described for 117NLS87-A using 4-trifluorophenylacetyl chloride and 128NLS52 (116 mg, 0.32 mmol). Yield: 52 mg, 32%.

R_{f}=0.42 (MeOH/CH₂Cl₂ 1:9). LCMS m/z 509 [M+H]⁺. ¹H-NMR (CDCl₃, rotamers 0.6:0.4) δ 7.60-6.90 (m, 8H, Ar—H), 4.99 (d, 1H, J=6.1, dioxane-H), 4.65-4.54 (m, 1.6H, pip-H, dioxane-H), 4.52 and 4.47 (2s, 2H, benzyl-H), 4.07-4.04 (m, 1H, dioxane-H), 3.88 (s, 0.8H, benzyl-H), 3.69-3.56 (m, 3.6H, benzyl-H, pip-H, dioxane-H), 2.89 (m, 2H, pip-H), 2.49-2.31 (m, 2H, NCH₂), 2.07-1.99 (m, 1.2H, pip-H), 1.89-1.36 (m, 8.8H, pip-H, dioxane-H, NCH₂CH₂). HPLC t_{R}=7.3 min.

### Example 172

### 2-(4-Cyanophenyl)-N-{1-[2-(1,3-dioxan-4-yl)ethyl]piperidin-4-yl}-N-(4-fluorobenzyl)acetamide, tartrate (128NLS54-C)

4-Cyanophenylacetic acid was synthesized according a method by Jaeger et al. (J. Chem. Soc., 1941, 744-747) and converted to the corresponding acetyl chloride by treatment with oxalylchloride. The title compound was prepared following the same method as described for 117NLS87-A using 4-cyanophenylacetyl chloride and 128NLS52 (116 mg, 0.32 mmol). Yield: 60 mg, 40%.

R_{f}=0.40 (MeOH/CH₂Cl₂ 1:9). LCMS m/z 466 [M+H]⁺. ¹H-NMR (CDCl₃, rotamers 0.7:0.3) δ 7.62-6.89 (m, 8H, Ar—H), 4.97 (d, 1H, J=6.1, dioxane-H), 4.63 (m, 1H, dioxane-H), 4.59-4.47 (m, 2.7H, pip-H, benzyl-H), 4.06-4.02 (m, 1H, dioxane-H), 3.86 (s, 0.6H, benzyl-H), 3.69-3.55 (m, 3.7H, benzyl-H, pip-H, dioxane-H), 2.91-2.86 (m, 2H, pip-H), 2.47-2.30 (m, 2H, NCH₂), 2.05-1.39 (m, 10H, pip-H, dioxane-H, NCH₂CH₂). HPLC t_{R}=4.3 min.

### Example 173

### N-(4-Fluorobenzyl)-2-(4-isobutoxyphenyl)-N-{1-[2-(2-oxo-imidazolidin-1-yl)ethyl]piperidin-4-yl}acetamide, hydrochloride (69NLS97)

Prepared following the same method as described for 117NLS01 using 103NLS56 (240 mg, 0.60 mmol) and 1-(2-tosyloxyethyl)-2-imidazolidinone as the alkylating agent. Yield: 95 mg, 31%.

LCMS m/z 511 [M+H]⁺. ¹H-NMR (CD₃OD, rotamers 0.6:0.4) δ 7.24-6.81 (m, 8H, Ar—H), 4.56 and 4.52 (2s, 2H, benzyl-H), 4.41-4.37 and 3.93-3.88 (m, 1H, pip-H), 3.84 and 3.56 (2s, 2H, benzyl-H), 3.73-3.69 (m, 2H, CH_{2OiBu}), 3.46-3.20 (m, 6H, imid-CH₂, NCH₂CH₂), 2.99-2.85 (m, 2H, pip-H), 2.44 (m, 2H, NCH₂), 2.10-1.96 (m, 3.2H, pip-H, CH_{OiBu}), 1.67-1.62 (m, 3H, pip-H), 1.30 (m, 0.8H, pip-H), 1.03-0.99 (m, 6H, J=6.6, CH_{3OiBu}).HPLC t_{R}=9.5 min.

Choosing the appropriate secondary amines (prepared in analogy to the method described for 103NLS56), following compounds were prepared using a similar procedure:

### Example 174

### 2-(4-Methoxyphenyl)-N-(4-methylbenzyl)-N-{1-[2-(2-oxo-imidazolidin-1-yl)ethyl]piperidin-4-yl}acetamide, hydrochloride (63ELH39-B)

LCMS m/z 465 [M+H]⁺. ¹H-NMR (CDCl₃, rotamers 0.6:0.4) δ 7.30-6.80 (m, 8H), 4.60-4.53 (m, 0.6H), 4.50 and 4.43 (2s, 2H), 3.78 (m, 4.2H), 3.51 (s, 1.2H), 3.46-3.24 (m, 6H), 2.92-2.79 (m, 2H), 2.46-2.40 (m, 2H), 2.35 and 2.29 (2s, 3H), 2.11-2.05 (m, 1.2H), 1.92-1.86 (m, 0.8H), 1.65-1.50 (m, 3.2H, partly covered by HDO signal), 1.31 (m, 0.8H).

### Example 175

### N-(4-Fluorobenzyl)-2-(4-isopropoxyphenyl)-N-{1-[3-(3-methyl-2-oxo-2,3-dihydro-benzoimidazol-1-yl)propyl]piperidin-4-yl}acetamide; hydrochloride (103NLS39)

Prepared following the same method as described for 117NLS01 using N-(4-fluorobenzyl)-N-(piperidin-4-yl)-2-(4-isoproxyphenyl)acetamide (229 mg, 0.59 mmol) and 1-(3-chloropropyl)-3-methyl-1,3-dihydrobenzimidazol-2-one as the alkylating agent.

Yield: 205 mg, 61%. R_{f}=0.29 (MeOH/CH₂Cl₂ 5:95). LCMS m/z 573 [M+H]⁺. ¹H-NMR (CDCl₃, rotamers 0.5:0.5) δ 7.18-6.78 (m, 12H, Ar—H), 4.59-4.43 (m, 3.5H, pip-H, OCH, benzyl-H), 3.88 (t, 2H, J=6.8, NCONCH₂), 3.74 (m, 1.5H, pip-H, benzyl-H), 3.49 (s, 1H, benzyl-H), 3.38 and 3.37 (2s, 3H, NCH₃), 2.93-2.79 (m, 2H, pip-H), 2.36-2.29 (m, 2H, NCH₂), 2.02-1.95 (m, 1H, pip-H), 1.90-1.46 (m, 6H, pip-H, NCH₂CH₂), 1.31-1.25 (m, 7H, pip-H, CH(CH₃)₂). HPLC t_{R}=8.0 min.

Choosing the appropriate secondary amines (prepared in analogy to the method described for 103NLS56) and alkylating agents, following compounds were prepared using a similar procedure:
N-{1-[2-(2,4-Dioxo-1,4-dihydro-2H-quinazolin-3-yl)ethyl]piperidin-4-yl}-2-(4-methoxyphenyl)-N-(4-methylbenzyl)acetamide, hydrochloride (63ELH29A).
2-(4-Methoxyphenyl)-N-(4-methylbenzyl)-N-{1-[3-(2-oxo-2,3-dihydrobenzoimidazol-1-yl)propyl]piperidin-4-yl}-acetamide, hydrochloride (50ELH89).
N-(4-Fluorobenzyl)-2-(4-isopropoxyphenyl)-N-{1-[4-(2-oxo-2,3-dihydrobenzoimidazol-1-yl)butyl]piperidin-4-yl}acetamide, hydrochloride (63ELH91).
N-{1-[2-(2,4-Dioxo-1,4-dihydro-2H-quinazolin-3-yl)ethyl]piperidin-4-yl}-N-(4-fluorobenzyl)-2-(4-isopropoxyphenyl)acetamide, hydrochloride (63ELH89).

### Example 176

### 4-(4-Fluorobenzylamino)-piperidine-1-carboxylic acid benzyl ester (118AF93-51)

A solution of 4-fluorobenzylamine (5.48 g, 43.8 mmol) in a mixture of methanol and acetic acid (5:1, 60 mL) was added dropwise to a solution of benzyl 4-oxo-1-piperidine carboxylate (10.2 g, 43.8 mmol) in methanol (150 mL) at rt. To this mixture sodium cyanoborohydride (5.50 g, 87.5 mmol) was slowly added. After 20 hours stirring at rt the reaction mixture was neutralized and the solvent was removed by evaporation under reduced pressure. The residue was partitioned between dichloromethane and water. The organic layer was dried over sodium sulphate, filtered and evaporated to dryness. Purification of the residue by silica gel column chromatography, eluting with 7% methanol in dichloromethane, afforded the desired compound (9.0 g, 60%).

R_{f} 0.56 (MeOH/CH₂Cl₂ 5:95). LCMS m/z 343 [M+H]⁺. HPLC t_{R}=6.2 min.

### Example 177

### N-(1-Benzyloxycarbonylpiperidin-4-yl)-N-(4-fluorobenzyl)-N′-(4-isopropoxybenzyl)carbamide (118AF97-120)

1,8-Bis(dimethylamino)-naphtalene (3.19 g, 14.9 mmol) was added to a solution of 4-(isopropoxy)phenyl acetic acid (2.89 g, 14.9 mmol) in dry tetrahydrofuran (18 mL) at rt under argon atmosphere. After 25 minutes stirring at rt diphenylphosphoryl azide (4.10 g, 14.9 mmol) was added dropwise and the mixture refluxed for 6 hours. It was allowed to cool to rt and then stored at −20° C. overnight to precipitate out the ammonium phosphate salt. A mixture of diethyl ether and ethyl acetate (1:1 v/v, 25 mL) was added to the cold reaction mixture. The precipitate was filtered from the reaction mixture and washed with diethyl ether: ethyl acetate (1:1 v/v, 20 mL). The filtrate was evaporated to dryness giving 1-isocyanatomethyl-4-isopropoxybenzene as an oil (3.2 g), which was used in the next step without further purification.

Sodium carbonate (3.5 g, 25.3 mmol) was added to the solution of 4-(4-fluorobenzyl amino)-piperidine-1-carboxylic acid benzyl ester 118AF93-51 (5.7 g, 16.7 mmol) in dry tetrahydrofuran (20 mL). To this suspension a solution of 1-isocyanatomethyl-4-isopropoxybenzene (3.2 g, 16.7 mmol) in dry tetrahydrofuran (10 mL) was added under argon atmosphere. The reaction mixture was stirred overnight at rt. Afterwards the mixture was partitioned between dichloromethane and water. The organic layer was dried over sodium sulphate, filtered and evaporated to dryness. Purification of the residue by silica gel column chromatography, eluting with 8% methanol in dichloromethane afforded the desired compound (2.0 g, 22%).

R_{f}=0.36 (MeOH/CH₂Cl₂, 5:95). LCMS m/z 534 [M+H]⁺. HPLC t_{R}=10.2 min.

### Example 178

### N-(4-Fluorobenzyl)-N′-(4-isopropoxybenzyl)-N-piperidin-4-yl-carbamide, oxalate (118AF99-121)

The desired compound was obtained by hydrogenation of 118AF97-120 (2.0 g, 3.75 mmol) in absolute ethanol (100 mL) using palladium on carbon as a catalyst. The product was purified by column chromatography on silica gel eluting with stepwise gradient of 5-10% methanol in dichloromethane. Yield: 1.16 g, 77%.

R_{f}=0.10 (MeOH/CH₂Cl₂ 10:90). LCMS m/z 400 [M+H]⁺+H NMR (CDCl₃) δ 7.19 (m, 2H, Ar—H), 7.01-6.69 (m, 4H, Ar—H), 6.76 (m, 2H, Ar—H), 4.51-4.40 (m, 3H, pip-H, OCH(CH₃), NH), 4.35 (s, 2H, benzyl-H), 4.28 (s, 1H, benzyl-H), 4.27 (s, 1H, benzyl-H), 3.14-3.07 (m, 2H, pip-H), 2.74-2.68 (m, 2H, pip-H), 2.10 (broad s, 1H, NH), 1.78-1.70 (m, 2H, pip-H), 1.58-1.48 (m, 2H, pip-H), 1.31 (d, 6H, J=6.0, OCH(CH₃)). HPLC t_{R}=5.9 min.

### Example 179

### N-{1-[2-(1,3-Dioxolan-2-yl)ethyl]piperidin-4-yl}-N-(4-fluorobenzyl)-N′-(4-isopropoxy-benzyl)carbamide, oxalate (130AF10-147)

Potassium carbonate (0.21 g, 1.50 mmol) was added to a solution of 118AF99-121 (0.3 g, 0.75 mmol) in dry N,N-dimethylformamide (2 mL). The suspension was shaken for 30 minutes at 58° C. A solution of 2-(2-bromoethyl)-1,3-dioxolane (0.163 g, 0.90 mmol) in dry N,N-dimethylformamide (0.4 mL) was added dropwise to the warm suspension and the heating was continued overnight. The mixture was allowed to cool to rt, then filtered and partitioned between water and dichloromethane. The organic layer was washed with a aqueous solution of 4% magnesium sulphate and evaporated to dryness. Purification of the residue by silica gel column chromatography, eluting with 4% methanol in dichloromethane, afforded the desired compound (197 mg, 53%). The product was converted to its oxalate form as described above.

R_{f}=0.39 (MeOH/CH₂Cl₂ 4:94). LCMS m/z 500 [M+H]⁺. ¹H NMR (CDCl₃) δ 7.17 (m, 2H, Ar—H), 7.00-6.95 (m, 4H, Ar—H), 6.76 (m, 2H, Ar—H), 4.88 (t, 1H, J=4.8, dioxolane-H), 4.51-4.44 (m, 2H, NH, CH(CH₃)₂), 4.36-4.26 (m, 5H, benzyl-H, pip-H), 3.95-3.80 (m, 4H, dioxolane-H), 2.98-2.91 (m, 2H, pip-H), 2.48-2.43 (m, 2H, NCH₂), 2.10-2.01 (m, 2H, pip-H), 1.85-1.79 (m, 2H, NCH₂CH₂), 1.76-1.58 (m, 4H, pip-H), 1.30 (d, 6H, J=6.0, CH(CH₃)₂). HPLC t_{R}=6.9 min.

Choosing the appropriate secondary amines (prepared in analogy to the method described for 103NLS56), following compounds were prepared using the same procedure:
N-{1-[2-(1,3-Dioxolan-2-yl)ethyl]piperidin-4-yl}-2-(4-methoxyphenyl)-N-(4-methylbenzyl)acetamide, hydrochloride (63ELH29B).
N-{1-[2-(1,3-Dioxolan-2-yl)ethyl]piperidin-4-yl}-N-(4-fluorobenzyl)-2-(4-isobutoxyphenyl)acetamide, hydrochloride (74AKU06-2).
N-{1-[2-(1,3-Dioxolan-2-yl)ethyl]piperidin-4-yl}-2-(4-isopropoxyphenyl)-N-(4-methylbenzyl)acetamide, hydrochloride (76ELH07).
N-{1-[2-(1,3-Dioxolan-2-yl)ethyl]piperidin-4-yl}-N-(4-fluorobenzyl)-2-(4-propoxyphenyl)acetamide, tartrate (38PH50).

### Example 180

### N-(4-Fluorobenzyl)-N′-(4-isopropoxybenzyl)-N-{1-[2-((S)-4-methyl-1,3-dioxolane-2-yl)ethyl]piperidin-4-yl carbamide, oxalate (130AF12-148)

4 M HCl (0.5 mL) and water (0.5 mL) were added to a solution of 130AF10-147 (50 mg, 0.10 mmol) in 1.4-dioxane (1 mL). The mixture was stirred in a sealed flask for 10 minutes under microwave irradiation at 120° C. Afterwards the mixture was partitioned between dichloromethane and water. The organic layer was dried over sodium sulphate, filtered and evaporated to dryness. The residue was dissolved in 1.4-dioxane (1 mL) and a solution of (S)-(+)-propylene glycol (39 mg, 0.51 mmol) in 1.4-dioxane (0.5 mL) was added. After addition of HCl (4M in dioxane, 0.5 mL) the mixture was stirred in a sealed flask for 20 minutes under microwave irradiation at 120° C. The mixture was partitioned between saturated sodium bicarbonate solution and dichloromethane. The organic layer was evaporated to dryness. Purification of the residue by silica gel column chromatography, eluting with a stepwise gradient of 4-8% methanol in dichloromethane afforded the desired compound (2.1 mg, 4%). The product was converted to its oxalate form as described above.

R_{f}=0.36 (MeOH/CH₂Cl₂ 4:94). LCMS m/z 514 [M+H]⁺. HPLC t_{R}=7.2 min.

### Example 181

### N-(4-Fluorobenzyl)-N′-(4-isopropoxybenzyl)-N-[1-(3-morpholin-4-yl-propyl)piperidin-4-yl]carbamide, oxalate (130AF09-145)

A solution of 1-chloro-3-bromopropane in dry tetrahydrofuran (2 mL) was added to a cold suspension of morpholine (200 mg, 2.29 mmol) and sodium carbonate (0.63 g, 4.56 mmol) in dry tetrahydrofuran (8 mL) at 0° C. The mixture was stirred at 45° C. overnight. The mixture was allowed to cool to rt, filtered and evaporated to dryness. Purification of the residue by silica gel column chromatography, eluting with a mixture of ethyl acetate and n-heptane (70:30), afforded 3-chloro-1-morpholin-4-yl-propane (156 mg, 42

A solution of 3-chloro-1-morpholin-4-yl-propane (7.6 mg, 0.046 mmol) in dry N,N-dimethylformamide (0.10 mL) was added to a solution of 118AF99-121 (15 mg, 0.037 mmol) and caesium carbonate (40 mg, 0.123 mmol) in a mixture of dry N,N-dimethylformamide and acetonitrile (1:2, 0.30 mL). After addition of sodium iodide (7.0 mg, 0.047 mmol) the mixture was shaken overnight at 60° C. The mixture was allowed to cool to rt. Acetonitrile was removed by evaporation under reduced pressure and the residue was partitioned between dichloromethane (2 mL) and water (1 mL). The organic layer was evaporated to dryness. Purification of the residue by preparative reversed phase HPLC (C₁₈) afforded the desired compound (6.1 mg, 32%).

LCMS m/z 527 [M+H]⁺. HPLC t_{R}=6.2 min.

Choosing the appropriate secondary amines (prepared in analogy to the method described for 103NLS56), following compounds were prepared using a similar procedure:
2-(4-Methoxyphenyl)-N-(4-methylbenzyl)-N-[1-(2-morpholin-4-ylethyl)piperidin-4-yl]acetamide, dihydrochloride (63ELH40-2).
2-(4-Methoxyphenyl)-N-(4-methylbenzyl)-N-[1-(3-morpholin-4-ylpropyl)piperidin-4-yl]acetamide, dihydrochloride (63ELH41-2).
N-(4-Fluorobenzyl)-2-(4-isobutoxyphenyl)-N-[1-(3-morpholin-4-ylpropyl)piperidin-4-yl]acetamide, dihydrochloride (74AKU07-2).
N-(4-Fluorobenzyl)-2-(4-isopropoxyphenyl)-N-[1-(3-morpholin-4-yl-propyl)piperidin-4-yl]acetamide, dihydrochloride (76ELH14-A).

### Example 182

### N-(4-Fluorobenzyl)-N′-(4-isopropoxybenzyl)-N-[1-(3-piperidin-1-yl-propyl)piperidin-4-yl]carbamide, oxalate (130AF09-146)

The desired compound was synthesized from piperidine, 1-chloro-3-bromopropane and 118AF99-121 (15 mg, 0.037 mmol) using the same method as for preparation of 130AF09-145. Yield: 5.8 mg, 30%.

LCMS m/z 525 [M+H]⁺. HPLC t_{R}=6.8 min.

### Example 183

### N-(4-Fluorobenzyl)-N′-(4-isopropoxybenzyl)-N-[1-(3-((S)-4-isopropyl-2-oxazolidinon-1-yl-propyl)piperidin-4-yl]carbamide, tartrate (130AF14-152)

The desired compound was synthesized from (4S)-3-(3-chloropropyl)-4-isopropyloxazolidinon-2-one 103NLS94 (7.4 mg, 0.045 mmol) and 118AF99-121 (15 mg, 0.037 mmol) using the same method as for preparation of 130AF09-145. Yield: 3.3 mg, 16%.

LCMS m/z 569 [M+H]⁺. HPLC t_{R}=8.2 min.

### Example 184

### N-(4-Fluorobenzyl)-N′-(4-isopropoxybenzyl)-N-{1-[2-(2,5,5-trimethyl-1,3-dioxan-2-yl)ethyl}piperidin-4-yl]carbamide, oxalate (130AF07-143)

The desired compound was synthesized from 2-bromo-1-(2,5,5-trimethyl-1,3-dioxan-2-yl)-ethane (10.7 mg, 0.045 mmol) and 118AF99-121 (15 mg, 0.037 mmol) using the same method as for preparation of 130AF09-145. Yield: 8.3 mg, 15%.

LCMS m/z 556 [M+H]⁺. HPLC t_{R}=9.6 min.

### Example 185

N-{1-[3-(1,3-Dioxolan-2-yl)propyl]piperidin-4-yl}-N-(4-fluorobenzyl)-N′-(4-isopropoxybenzyl)carbamide, oxalate (130AF07-131).

The desired compound was synthesized from 3-chloro-1-(1,3-dioxolan-2-yl)-propane (6.79 mg, 0.045 mmol) and 118AF99-121 (15 mg, 0.037 mmol) using the same method as for preparation of 130AF09-145. Yield: 5.6 mg, 11%.

LCMS m/z 514 [M+H]⁺. HPLC t_{R}=8.3 min.

### Example 186

### N-[1-(2,2-Dimethyl-1,3-dioxan-5-yl)piperidin-4-yl]-N-(4-fluorobenzyl)-N′-(4-isopropoxybenzyl)carbamide, oxalate (130AF05-129)

A solution of 2,2-dimethyl-1,3-dioxan-5-one (9.75 mg, 0.075 mmol) in methanol (0.10 mL) was added to a solution of 118AF99-121 (15 mg, 0.037 mmol) in methanol (0.10 mL). The reaction mixture was stirred at rt after addition of acetic acid (60 μL of 1 M solution in methanol). After 2 h stirring a solution of sodium cyanoborohydride (5 mg, 0.079 mmol) in methanol (0.10 mL) was added and stirring was continued overnight at rt. The solvent was removed by evaporation under reduced pressure and the residue partitioned between 2 M aq. sodium hydroxide and dichloromethane. The layers were separated by filtration over PTFE filter. The organic layer was evaporated to dryness. Purification of the residue by preparative reversed phase HPLC (C₁₈) afforded the desired compound (2.3 mg, 12%).

LCMS m/z 514 [M+H]⁺. HPLC t_{R}=9.0 min.

### Example 187

### N-(4-Fluorobenzyl)-N′-(4-isopropoxybenzyl)-N-{[2-(1-methylpyrrolidin-2-yl)ethyl]-piperidin-4-yl carbamide, oxalate (130AF07-135)

The desired compound was synthesized from 2-(2-chloroethyl)-1-methylpyrrolidine hydrochloride (7.7 mg, 0.041 mmol) and 118AF99-121 (15 mg, 0.037 mmol) using the same method as for the preparation of 130AF09-145. Yield: 4.4 mg, 23%.

LCMS m/z 511 [M+H]⁺. HPLC t_{R}=7.0 min.

### Example 188

### N-[1-(2,2-Dimethyl-1,3-dioxan-5-yl)piperidin-4-yl]-N-(4-fluorobenzyl)-2-(4-isobutoxyphenyl)acetamide, oxalate (130AF22-105)

A solution of 2,2-dimethyl-1,3-dioxan-5-one (81 mg, 0.62 mmol) in methanol (10 mL) was added dropwise to a solution of 103NLS56 (179 mg, 0.45 mmol) in methanol (10 mL). The reaction mixture was stirred at rt after addition of acetic acid (200 μL). After 2 hours sodium cyanoborohydride (56 mg, 0.90 mmol) was slowly added and stirring was continued overnight at rt. The mixture was neutralized with few drops of 2 M aq sodium hydroxide. The solvent was removed by evaporation under reduced pressure and the residue partitioned between water and dichloromethane. The organic layer was dried over sodium sulphate, filtered and evaporated to dryness. Purification of the residue by silica gel column chromatography, eluting with 6% methanol in dichloromethane, afforded the desired compound (98 mg, 43%).

R_{f}=0.32 (MeOH/CH₂Cl₂, 6:94). LCMS m/z 513 [M+H]⁺. ¹H NMR (CDCl₃, rotamers 0.4:0.6) δ 7.26-6.79 (m, 8H, Ar—H), 4.63-4.54 (m, 0.6H, pip-H), 4.50 & 4.43 (2s, 2H, benzyl-H), 3.91 & 3.88 (2d, 1H, J=5.6, dioxane-H), 3.79-3.67 (m, 6.2H, dioxane-H, benzyl-H, pip-H, CH_{2OiBu}), 3.51 (s, 1.2H, benzyl-H), 2.98-2.88 (m, 2H, pip-H), 2.64-2.52 (m, 1H, dioxane-H), 2.38-2.28 (m, 1.2H, pip-H), 2.17-2.00 (m, 1.8H, CH(CH₃)₂, pip-H), 1.72-1.47 (m, 3.2H, pip-H), 1.43 (m, 0.8H, pip-H), 1.38-1.22 (m, 6H, dioxane-CH₃), 1.01 (m, 6H, CH(CH₃)₂). HPLC t_{R}=10.0 min.

### Example 189

### N-[1-(1,3-Dioxan-5-yl)-piperidin-4-yl)-N-(4-fluorobenzyl)-2-(4-isobutoxyphenyl)acetamide, tartrate (130AF26-164)

3 M aq HCl (1 mL) and water (1 mL) were added to a solution of 130AF22-105 (98.2 mg, 0.19 mmol) in 1.4-dioxane (2 mL) and the mixture stirred in a sealed flask under microwave irradiation for 10 minutes at 120° C. The mixture was partitioned between water and dichloromethane and the organic layer dried over sodium sulphate, filtered and evaporated to dryness. The residue was dissolved in 1.4-dioxane (2 mL). To this solution a solution of formaldehyde (37% water solution, 101 mg, 1.16 mmol) in 1.4-dioxane (0.5 mL) was added. The reaction mixture was stirred in a sealed flask for 30 minutes under microwave irradiation at 120° C. Molecular sieves (4A) were added to the reaction mixture at rt and removed after 24 hours. The mixture was heated for an additional 20 minutes at 120° C. under microwave irradiation and partitioned between dichloromethane and sodium bicarbonate. The organic layer was dried over sodium sulphate, filtered and evaporated to dryness. Purification of the residue by silica gel column chromatography, eluting with 6% methanol in dichloromethane, afforded the desired compound (17 mg, 18%). The product was converted to its tartrate form as described above.

R_{f}=0.30 (MeOH/CH₂Cl₂, 6:94). LCMS m/z 485 [M+H]⁺. ¹H NMR (CDCl₃, rotamers 0.4:0.6) δ 7.21-6.80 (m, 8H, Ar—H), 4.88 (m, 1H, dioxane-H), 4.61-4.56 (m, 1.6H, dioxane-H, pip-H), 4.50 & 4.43 (2s, 2H, benzyl-H), 4.12-4.06 (m, 2H, dioxane-H) 3.85-3.60 (m, 5.2H, dioxane-H, benzyl-H, pip-H, CH_{2OiBu}), 3.51 (s, 1.2H, benzyl-H), 2.94-2.86 (m, 2H, pip-H), 2.59-2.48 (m, 1H, dioxane-H), 2.37-2.28 (m, 1.2H, pip-H), 2.17-2.01 (m, 1.8H, CH(CH₃)₂, pip-H), 1.68-1.46 (m, 3.2H, pip-H), 1.46-1.30 (m, 0.8H, pip-H), 1.02 (m, 6H, CH(CH₃)₂). HPLC t_{R}=9.5 min.

### Example 190

### N-[1-(2,2-Dimethyl-1,3-dioxan-5-yl)piperidin-4-yl]-N-(4-fluorobenzyl)-2-(4-fluorophenyl)acetamide, tartrate (130AF35-168)

The desired compound was synthesized from 2,2-dimethyl-1,3-dioxan-5-one (59 mg, 0.45 mmol) and N-(4-fluorobenzyl)-2-(4-fluorophenyl)-N-piperidin-4-yl-acetamide (83 mg, 0.24 mmol) using the same method as for preparation of 130AF22-105. The starting material N-(4-fluorobenzyl)-2-(4-fluorophenyl)-N-piperidin-4-yl-acetamide was prepared in the same way as 103NLS56.

R_{f}=0.26 (MeOH/CH₂Cl₂, 5:95). LCMS m/z 459 [M+H]⁺. ¹H NMR (CDCl₃, rotamers 0.4:0.6) δ 7.28-6.91 (m, 8H, Ar—H), 4.63-4.52 (m, 0.6H, pip-H), 4.51 & 4.46 (2s, 2H, benzyl-H), 3.92-3.88 (m, 2H, dioxane-H), 3.82-3.69 (m, 3.2H, dioxane-H, benzyl-H, pip-H), 3.54 (s, 1.2H, benzyl-H), 2.99-2.90 (m, 2H, pip-H), 2.62-2.51 (m, 1H, dioxane-H), 2.39-2.28 (m, 1.2H, pip-H), 2.18-2.10 (m, 0.8H, pip-H), 1.72-1.50 (m, 3.2H, pip-H), 1.42-1.31 (m, 6.8H, pip-H, dioxane-CH₃). HPLC t_{R}=7.9 min.

### Example 191

### N-{1-[2-(1,3-Dioxan-4-yl)ethyl]piperidin-4-yl}-N-(4-fluorobenzyl)-2-(4-fluorophenyl)acetamide, tartrate (130AF41-171)

The starting material N-(4-fluorobenzyl)-2-(4-fluorophenyl)-N-piperidin-4-yl-acetamide was prepared in the same way as 103NLS56.

Potassium carbonate (64 mg, 0.46 mmol) was added to a solution of N-(4-fluorobenzyl)-2-(4-fluorophenyl)-N-piperidin-4-yl-acetamide (79.4 mg, 0.23 mmol) in dry N,N-dimethylformamide (3 mL). To this suspension a solution of 4-[2-(tosyloxy)ethyl]-1,3-dioxane 128NLS46B (99 mg, 0.35 mmol) in dry N,N-dimethylformamide (1 mL) was added dropwise at rt. The reaction mixture was stirred overnight at 60° C. and it was partitioned between dichloromethane and water. The organic layer was dried over sodium sulphate, filtered and evaporated to dryness. Purification of the residue by silica gel column chromatography, eluting with stepwise gradient of 2-5% methanol in dichloromethane, afforded the desired product (71 mg, 67%). The product was converted to its tartrate form as described above.

R_{f}=0.41 (MeOH/CH₂Cl₂, 6:96). LCMS m/z 459 [M+H]⁺. ¹H NMR (CDCl₃, rotamers 0.4:0.6) δ 7.28-6.90 (m, 8H, Ar—H), 4.99 (m, 1H, dioxane-H), 4.77-4.64 (m, 1.6H, pip-H, dioxane-H), 4.52 (s, 2H, benzyl-H), 3.80-3.56 (m, 4.4H, dioxane-H, benzyl-H, pip-H), 3.21-3.08 (m, 1.2H, pip-H), 2.96-2.88 (m, 0.8H, pip-H), 2.75-2.56 (m, 1.2H, NCH₂), 2.52-2.24 (m, 2H, pip-H, NCH₂), 2.04-1.30 (m, 9.8H, pip-H, NCH₂CH₂, dioxane-H). HPLC t_{R}=6.4 min.

### Example 192

### N-{1-[2-(1,3-Dioxan-4-yl)ethyl]piperidin-4-yl}-N-(4-fluorobenzyl)-2-(4-trifluoromethoxyphenyl)acetamide, tartrate (130AF80-186)

Triethylamine (125 μL, 0.89 mmol) was added to a solution of N-{1-[2-(1,3-dioxan-4-yl)ethyl]piperidin-4-yl}-N-(4-fluorobenzyl)amine 128NLS52 (96 mg, 0.30 mmol) in dry dichloromethane (5 mL) at rt. The solution was cooled to −10° C. and a solution of (4-trifluoromethoxyphenyl)acetyl chloride (71 mg, 0.30 mmol) in dry dichloromethane (1 mL) was added dropwise. The reaction mixture was stirred overnight at rt. The solvent was removed by evaporation under reduced pressure. The residue was suspended in tetrahydrofuran and filtered. The filtrate was evaporated to dryness and the residue was purified by silica gel column chromatography, eluting with 4% methanol in dichloromethane, to give the desired compound (46 mg, 30%). The compound was converted to its tartrate form as described above.

R_{f}=0.33 (MeOH/CH₂Cl₂, 6:94). LCMS m/z 459 [M+H]⁺. ¹H NMR (CDCl₃, rotamers 0.4:0.6) δ 7.34-6.91 (m, 8H, Ar—H), 5.01 (d, 1H, J=6.0, dioxane-H), 4.66-4.54 (m, 1.6H, pip-H, dioxane-H), 4.52 & 4.49 (2s, 2H, benzyl-H), 4.09-4.05 (m, 1H, dioxane-H), 3.83 (s, 0.8H, benzyl-H), 3.72-3.56 (m, 3.6H, dioxane-H, benzyl-H, pip-H), 2.94-2.86 (m, 2H, pip-H), 2.50-2.32 (m, 2H, NCH₂), 2.11-2.00 (m, 1.2H, pip-H), 1.90-1.52 (m, 8.8H, pip-H, NCH₂CH₂, dioxane-H). HPLC t_{R}=7.6 min.

### Example 193

### N-{1-[2-(1,3-Dioxan-4-yl)ethyl]piperidin-4-yl}-N-(4-fluorobenzyl)-2-(4-propoxyphenyl)acetamide, tartrate (130AF71-184)

Triethylamine (163 μL, 1.17 mmol) was added to a solution of N-{1-[2-(1,3-dioxan-4-yl)ethyl]piperidin-4-yl}-N-(4-fluorobenzyl)amine 128NLS52 (126 mg, 0.39 mmol) in dry dichloromethane (5 mL) at rt. The solution was cooled to −15° C. and a solution of (4-propoxyphenyl)acetyl chloride (92 mg, 0.43 mmol) in dry dichloromethane (2 mL) was added dropwise. The reaction mixture was stirred for 2 hours at rt. The solvent was removed by evaporation under reduced pressure. The residue was suspended in tetrahydrofuran and filtered. The filtrate was evaporated to dryness and the residue was purified by silica gel column chromatography, eluting with a stepwise gradient of 0-4% methanol in dichloromethane, to give the desired compound (66 mg, 34%). The product was converted to its tartrate form as described above.

R_{f}=0.16 (MeOH/CH₂Cl₂, 4:96). LCMS m/z 499 [M+H]⁺. ¹H NMR (CDCl₃, rotamers 0.4:0.6) δ 7.21-6.78 (m, 8H, Ar—H), 5.00 (m, 1H, dioxane-H), 4.66-4.54 (m, 1.6H, pip-H, dioxane-H), 4.50 & 4.44 (2s, 2H, benzyl-H), 4.10-4.03 (m, 1H, dioxane-H), 3.92-3.87 (m, 2H, OCH_{2OPr}), 3.78-3.50 (m, 4.4H, dioxane-H, benzyl-H, pip-H), 2.92-2.82 (m, 2H, pip-H), 2.50-2.29 (m, 2H, NCH₂), 2.09-1.98 (m, 1.2H, pip-H), 1.88-1.27 (m, 10.8H, pip-H, NCH₂CH₂, dioxane-H, CH_{2OPr}), 1.05-099 (m, 3H, CH_{3OPr}). HPLC t_{R}=7.6 min.

### Example 194

### N-(4-Fluorobenzyl)-2-(4-isobutoxyphenyl)-N-[1-(tetrahydropyran-4-yl)piperidin-4-yl]acetamide, tartrate (130AF33-166)

A solution of tetrahydro-4H-pyran-4-one (43 mg, 0.42 mmol) in methanol (1 mL) was added to a solution of 103NLS56 (57 mg, 0.14 mmol) in methanol (2 mL). After addition of acetic acid (100 μL) the reaction mixture was stirred for 15 minutes in a sealed flask under microwave irradiation at 100° C. Afterwards sodium cyanoborohydride (26 mg, 0.42 mmol) was added to the mixture and stirring was continued for additional 60 min under microwave irradiation at 80° C. The mixture was passed over an acidic ion-exchange SPE cartridge. Further purification of the product by silica gel column chromatography, eluting with a stepwise gradient of 2-5% methanol in dichloromethane, afforded the desired compound (19.2 mg, 28%). The compound was converted to its tartrate form as described above.

R_{f}=0.18 (MeOH/CH₂Cl₂, 5:95). LCMS m/z 483 [M+H]⁺. ¹H NMR (CDCl₃, rotamers 0.4:0.6) δ 7.21-6.80 (m, 8H, Ar—H), 4.64-4.56 (m, 0.6H, pip-H), 4.51& 4.45 (2s, 2H, benzyl-H), 4.02-3.96 (m, 2H, THP—H), 3.77-3.68 (m, 3.2H, benzyl-H, CH_{2OiBu}, Pip-H), 3.51 (s, 1.2H, benzyl-H), 3.30 (t, 2H, J=12.0, THP—H), 2.98-2.88 (m, 2H, pip-H), 2.46-2.34 (m, 1H, THP—H), 2.28-2.19 (m, 1.2H, pip-H), 2.10-1.99 (m, 1.8H, CH_{OiBu}, pip-H), 1.73-1.47 (m, 7.2H, pip-H, THP—H), 1.39-1.33 (m, 0.8H, pip-H), 1.01 (m, 6H, CH_{3OiBu}). HPLC t_{R}=8.0 min.

### Example 195

### N-(4-Fluorobenzyl)-2-(4-isobutoxyphenyl)-N-[1-(tetrahydropyran-4-ylmethyl)piperidin-4-yl]acetamide, tartrate (130AF82-187)

The title compound was synthesized from N-(4-fluorobenzyl)-2-(4-isobutoxyphenyl)-N-piperidin-4-yl-acetamide103NLS56 (110 mg, 0.27 mmol) and tetrahydro-2H-pyran-4-yl carbaldehyde (63 mg, 0.55 mmol) using the same method as for preparation of 130AF33-166. Yield: 18 mg, 13%.

R_{f}=0.30 (MeOH/CH₂Cl₂, 5:95). LCMS m/z 497 [M+H]⁺. HPLC t_{R}=8.4 min.

### Example 196

### N-(4-Fluorobenzyl)-2-(4-isobutoxyphenyl)-N-{1-[2-(tetrahydropyran-4-yl)ethyl]piperidin-4-yl]acetamide, tartrate (130AF83-188)

The title compound was synthesized from N-(4-fluorobenzyl)-2-(4-isobutoxyphenyl)-N-piperidin-4-yl-acetamide103NLS56 (110 mg, 0.27 mmol) and tetrahydro-2H-pyran-4-yl acetaldehyde (70.5 mg, 0.55 mmol) using the same method as for preparation of 130AF33-166. Yield: 40 mg, 29%.

R_{f}=0.30 (MeOH/CH₂Cl₂, 5:95). LCMS m/z 511 [M+H]⁺. ¹H NMR (CDCl₃, rotamers 0.4:0.6) δ 7.21-6.80 (m, 8H, Ar—H), 4.65-4.55 (m, 0.6H, pip-H), 4.51& 4.44 (2s, 2H, benzyl-H), 3.95-3.89 (m, 2H, THP—H), 3.78-3.66 (m, 3.2H, benzyl-H, CH_{2OiBu}, Pip-H), 3.51 (s, 1.2H, benzyl-H), 3.34 (t, 2H, J=12.0, THP—H), 2.92-2.82 (m, 2H, pip-H), 2.34-2.26 (m, 2H, NCH₂CH₂), 2.11-1.96 (m, 2.2H, pip-H, CH_{OiBu}), 1.84-1.20 (m, 11.8H, pip-H, THP—H, CH₂CH₂N), 1.02 (m, 6H, CH_{3OiBu}). HPLC t_{R}=8.2 min.

### Example 197

### N-(4-Fluorobenzyl)-2-(4-fluorophenyl)-N-[1-(tetrahydropyran-4-yl)piperidin-4-yl]acetamide, tartrate (130AF37-169)

The desired compound was synthesized from tetrahydro-4H-pyran-4-one and N-(4-fluorobenzyl)-2-(4-fluorophenyl)-N-piperidin-4-yl-acetamide using the same method as for preparation of 130AF33-166. The starting material N-(4-fluorobenzyl)-2-(4-fluorophenyl)-N-piperidin-4-yl-acetamide was prepared in the same way as 103NLS56.

R_{f}=0.29 (MeOH7CH₂Cl₂, 5:95). LCMS m/z 429 [M+H]⁺. ¹H NMR (CDCl₃, rotamers 0.4:0.6) δ 7.29-6.91 (m, 8H, Ar—H), 4.64-4.55 (m, 0.6H, pip-H), 4.52 & 4.48 (2s, 2H, benzyl-H), 4.02-3.95 (m, 2H, THP—H), 3.80 (s, 0.8H, benzyl-H), 3.75-3.64 (m, 0.4H, pip-H), 3.54 (s, 1.2H, benzyl-H), 3.34 (t, 2H, J=12.0, THP—H), 2.99-2.90 (m, 2H, pip-H), 2.48-2.36 (m, 1H, THP—H), 2.26-2.20 (m, 1.2H, pip-H), 2.08-2.00 (m, 0.8H, pip-H), 1.76-1.47 (m, 7.2H, pip-H, THP—H), 1.41-1.34 (m, 0.8H, pip-H). HPLC t_{R}=5.6 min.

### Example 198

### N-[1-((S)-3,5-Dihydroxypentyl)piperidine-4-yl]-N-(4-fluorobenzyl)-2-(4-isobutoxyphenyl)acetamide, tartrate (130AF65-182)

The compound (R)-5-[(4-methylbenzenesulfonyl)oxy]pentane-1,3-diol was synthesized according to Moune et al (J. Org. Chem., 1997, 62, 3332-3339). Potassium carbonate (83 mg, 0.60 mmol) was added to a solution of 103NLS56 (94 mg, 0.24 mmol) in dry N,N-dimethylformamide (3 mL). To this suspension a solution of (R)-5-[(4-methyl-benzenesulfonyl)oxy]pentane-1,3-diol (82 mg, 0.28 mmol) in dry N,N-dimethylformamide (1 mL) was added, followed by addition of sodium iodide (43 mg, 0.29 mmol). The reaction mixture was stirred overnight at 60° C. It was allowed to cool to rt, filtered and evaporated to dryness. The residue was partitioned between dichloromethane and 2M aq sodium hydroxide. The organic layer was dried over sodium sulphate, filtered and evaporated to dryness. Purification of the residue by silica gel column chromatography, eluting with a stepwise gradient of 6-10% methanol in dichloromethane, afforded the desired compound (35 mg, 29%), which was converted to its tartrate form as described above.

R_{f}=0.48 (MeOH/CH₂Cl₂, 10:90). LCMS m/z 501 [M+H]⁺. HPLC t_{R}=7.4 min.

### Example 199

### N-{1-[2-((4S)-1,3-Dioxane-4-yl)ethyl]piperidine-4-yl}-N-(4-fluorobenzyl)-2-(4-isobutoxyphenyl)acetamide, tartrate (130AF67-183)

Paraformaldehyde (9 mg, 0.28 mmol) and hydrochloric acid (4M in 1.4-dioxane, 0.5 mL) were added to a solution of tartaric acid salt of 130AF65-182 (37 mg, 0.056 mmol) in 1.4-dioxane. The reaction mixture was stirred for 2 hours in a sealed flask under microwave irradiation at 120° C. and partitioned between dichloromethane and sodium bicarbonate. The organic layer was washed with brine, dried over sodium sulphate, filtered and evaporated to dryness. Purification of the residue by acidic ion-exchange SPE cartridge afforded the desired compound (9.0 mg, 31%), which was converted to its tartrate form as described above. The enantiomeric excess (ee) was determined to be 94% using chiral HPLC analysis (Chiralpak AD column, 4.6×250 mm; heptane/I—PrOH 50:50, 0.3% DEA; 0.5 mL/min; t_{R} 20.5 min).

R_{f}=0.41 (MeOH/CH₂Cl₂, 8:92). LCMS m/z 513 [M+H]⁺. ¹H NMR (CDCl₃, rotamers 0.4:0.6) δ 7.21-6.80 (m, 8H, Ar—H), 5.00 (m, 1H, dioxane-H), 4.68-4.54 (m, 1.6H, pip-H, dioxane-H), 4.51 & 4.45 (2s, 2H, benzyl-H), 4.06 (m, 1H, dioxane-H), 3.77-3.48 (m, 6.4H, dioxane-H, benzyl-H, CH_{2OiBu}, pip-H), 2.98-2.79 (m, 2H, pip-H), 2.50-2.58 (m, 2H, NCH₂), 2.14-1.99 (m, 2.2H, CH_{OiBu}, pip-H), 1.90-1.25 (m, 8.8H, pip-H, NCH₂CH₂, dioxane-H), 1.02 (m, 6H, CH_{3OiBu}). HPLC t_{R}=8.7 min.

### Example 200

### N-{1-[2-(1,3-Dioxan-2-yl)ethyl]piperidin-4-yl}-N-(4-fluorobenzyl)amine (118AF52-95)

Sodium carbonate (17.4 g, 125.9 mmol) was added to a solution of 4-piperidone monohydrate hydrochloride (6.45 g, 42.0 mmol) in acetonitrile (200 mL). After 30 minutes stirring at rt a solution of 2-(2-bromoethyl)-1,3-dioxane (8.45 g, 43.3 mmol) in acetonitrile (50 mL) was added dropwise to the reaction mixture and stirring was continued overnight at rt and at reflux for an additional 2 hours. The solvent was removed by evaporation under reduced pressure and the residue was partitioned between water and dichloromethane. The organic layer was dried over sodium sulphate, filtered and evaporated to dryness. Purification of the residue by silica gel column chromatography, eluting with 7% methanol in dichloromethane, afforded 1-[2-(1,3-dioxan-2-yl)ethyl]piperidin-4-one (6.19 g, 69%).

A solution of 1-[2-(1,3-dioxan-2-yl)ethyl]piperidin-4-one (6.19 g, 29 mmol) in methanol (80 mL) was added dropwise to a solution of 4-fluorobenzylamine (3.9 mL, 34 mmol) in methanol (100 mL) under argon atmosphere at rt. After 30 minutes stirring at rt the reaction mixture was acidified (pH=5) with acetic acid and cooled to 0° C. Sodium cyanoborohydride (2.15 g, 40 mmol) was added slowly to the cold mixture and stirring was continued at rt overnight. The reaction mixture was basified with 2M NaOH and concentrated in vacuo. The residue was partitioned between ethyl acetate and water. The organic layer was dried over sodium sulphate, filtered and evaporated to dryness. The residue was dissolved in abs. ethanol (57 mL). A solution of maleic acid (3.31 g, 28.5 mmol) in abs. ethanol (60 mL) was added to this solution resulting in precipitate formation. The precipitate was collected by filtration and converted to the free base by a basic extraction. Yield: 8.5 g, 91%.

R_{f}=0.29 (MeOH/CH₂Cl₂, 7:93). LCMS m/z 323 [M+H]⁺.¹H NMR (CDCl₃) δ 7.25 (m, 2H, Ar—H), 6.95 (m, 2H, Ar—H), 4.54 (t, 1H, J=5.6, dioxane-H), 4.07-4.02 (m, 2H, dioxane-H), 3.73-3.67 (m, 4H, dioxane-H, benzyl-H), 2.85-2.79 (m, 2H, pip-H), 2.49-2.37 (m, 3H, NCH₂, pip-H), 2.05-1.72 (m, 7H, pip-H, NCH₂CH₂, dioxane-H), 1.44-1.25 (m, 4H, dioxane-H, pip-H, NH). HPLC t_{R}=1.4 min.

### Example 201

### 2-(4-Benzyloxyphenyl)-N-{1-[2-(1,3-dioxan-2-yl)ethyl]piperidin-4-yl}-N-(4-fluorobenzyl)acetamide, tartrate (118AF66-102)

A solution of triethylamine (0.89 mL, 6.38 mmol) and 118AF52-95 (0.80 g, 2.48 mmol) in dry THF (10 mL) was cooled to 0° C. A solution of 4-benzyloxyphenylacetyl chloride (0.72 g, 2.76 mmol) was added dropwise to the cold reaction mixture and stirring was continued at rt for 2 h. The reaction mixture was filtered and the filtrate evaporated to dryness. Purification of the residue by silica gel column chromatography, eluting with a stepwise gradient of 0-6% methanol in dichloromethane afforded the desired compound (0.53 g, 39%), which was converted to its tartrate form as described above.

R_{f}=0.27 (MeOH/CH₂Cl₂, 7:93). LCMS m/z 547 [M+H]⁺. ¹H NMR (CDCl₃, rotamers 0.4:0.6) δ 7.46-6.86 (m, 13H, Ar—H), 5.08-5-02 (m, 2H, PhCH₂O), 4.64-4.42 (m, 3.6H, pip-H, benzyl-H, dioxane-H), 4.11-4.02 (m, 2H, dioxane-H), 3.79-3.67 (m, 3.2H, dioxane-H, benzyl-H, pip-H), 3.50 (s, 1.2H, benzyl-H), 2.94-2.80 (m, 2H, pip-H), 2.46-2.34 (m, 2H, NCH₂), 2.12-1.98 (m, 2.2H, dioxane-H, pip-H), 1.87-1.50 (m, 6H, pip-H, NCH₂CH₂), 1.36-1.24 (m, 1.8H, pip-H, dioxane-H). HPLC t_{R}=8.9 min.

### Example 202

### N-{1-[2-(1,3-Dioxan-2-yl)ethyl]piperidin-4-yl}-N-(4-fluorobenzyl)-2-(4-hydroxyphenyl)-acetamide, tartrate (118AF67-103)

The desired compound was afforded by hydrogenation of 118AF66-102 (0.50 g, 0.92 mmol) in absolute ethanol (200 mL) using palladium on carbon as a catalyst. The product was purified by column chromatography on silica gel eluting with a stepwise gradient of 3-6% methanol in dichloromethane. The desired compound (0.22 g, 53%) was converted to its tartrate form as described above.

R_{f}=0.30 (MeOH/CH₂Cl₂, 6:94). LCMS m/z 457 [M+H]⁺. ¹H NMR (CDCl₃,rotamers 0.4:0.6) δ 7.13-6.86 (m, 6H, Ar—H), 6.72-6.64 (m, 2H, Ar—H), 4.66-4.57 (m, 0.6H, pip-H), 4.54 (m, 1H, dioxane-H), 4.48 & 4.37 (2s, 2H, benzyl-H), 4.08-4.01 (m, 2H, dioxane-H), 3.80-3.66 (m, 3.2H, dioxane-H, benzyl-H, pip-H), 3.47 (m, 1.2H, benzyl-H), 2.94-2.82 (m, 2H, pip-H), 2.47-2.39 (m, 2H, NCH₂), 2.10-1.97 (m, 2.2H, dioxane-H, pip-H), 1.88-1.53 (m, 6H, pip-H, NCH₂CH₂), 1.34-1.25 (m, 1.8H, pip-H, dioxane-H). HPLC t_{R}=3.0 min.

### Example 203

### N-{1-[2-(1,3-Dioxan-2-yl)ethyl]piperidin-4-yl}-N-(4-fluorobenzyl)-2-(4-methoxyphenyl)-acetamide, tartrate (118AF60-96)

A solution of triethylamine (0.57 mL, 4.09 mmol) and 118AF52-95 (328 mg, 1.02 mmol) in dry THF (5 mL) was cooled to 0° C. A solution of 4-methoxyphenylacetyl chloride (376 mg, 2.04 mmol) was added dropwise to the cold reaction mixture and stirring was continued for 20 h at rt. The reaction mixture was partitioned between 2M NaOH and water. The organic layer was dried over sodium sulphate, filtered and evaporated to dryness. The residue was purified by silica gel column chromatography, eluting with a stepwise gradient of 0-6% methanol in dichloromethane. Final purification of the product by acidic ion-exchange SPE cartridge afforded the desired compound (153 mg, 33%), which was converted to its tartrate form as described above.

R_{f}=0.40 (MeOH/CH₂Cl₂, 4:96). LCMS m/z 471 [M+H]⁺. ¹H NMR (CDCl₃, rotamers 0.4:0.6) δ 7.24-6.79 (m, 8H, Ar—H), 4.63-4.54 (m, 0.6H, pip-H), 4.52 (t, 1H, J=5.2, dioxane-H), 4.49 & 4.44 (2s, 2H, benzyl-H), 4.09-4.01 (m, 2H, dioxane-H), 3.79-3.68 (m, 6.2H, dioxane-H, benzyl-H, pip-H, OCH₃), 3.50 (m, 1.2H, benzyl-H), 2.91-2.80 (m, 2H, pip-H), 2.43-2.36 (m, 2H, NCH₂), 2.10-1.98 (m, 2.2H, dioxane-H, pip-H), 1.86-1.51 (m, 6H, pip-H, NCH₂CH₂), 1.34-1.26 (m, 1.8H, pip-H, dioxane-H). HPLC t_{R}=7.0 min.

### Example 204

### N-{1-[2-(1,3-Dioxan-2-yl)ethyl]piperidin-4-yl}-N-(4-fluorobenzyl)-2-(4-isopropylphenyl)-acetamide, tartrate (118AF63-100)

The desired compound was synthesized from 118AF52-95 (400 mg, 1.24 mmol) and 4-isopropylphenylacetyl chloride (340 mg, 1.73 mmol) using the same method as for preparation of 118AF66-102. Further purification by acidic ion-exchange SPE cartridge was performed. Yield: 273 mg, 46%.

R_{f}=0.34 (MeOH/CH₂Cl₂, 7:93). LCMS m/z 483 [M+H]⁺. ¹H NMR (CDCl₃, rotamers 0.4:0.6) δ 7.22-6.89 (m, 8H, Ar—H), 4.64-4.43 (m, 3.6H, pip-H, dioxane-H, benzyl-H), 4.09-4.02 (m, 2H, dioxane-H), 3.79 (s, 0.8H, benzyl-H), 3.76-3.66 (m, 2.4H, dioxane-H, pip-H), 3.54 (m, 1.2H, benzyl-H), 2.92-2.79 (m, 3H, pip-H, CH(CH₃)₂), 2.41-2.35 (m, 2H, NCH₂), 2.12-1.98 (m, 2.2H, dioxane-H, pip-H), 1.85-1.49 (m, 6H, pip-H, NCH₂CH₂), 1.34-1.19 (m, 7.8H, pip-H, dioxane-H, CH(CH₃)₂). HPLC t_{R}=8.6 min.

### Example 205

### N-{1-[2-(1,3-Dioxan-2-yl)ethyl]piperidin-4-yl}-N-(4-fluorobenzyl)-2-(4-trifluoromethoxy-phenyl)acetamide, tartrate (118AF58-98)

The desired compound was synthesized from 118AF52-95 (328 mg, 1.02 mmol) and 4-trifluoromethoxyphenylacetyl chloride (345 mg, 1.44 mmol) using the same method as for preparation of 118AF66-102. Yield: 267 mg, 49%.

R_{f}=0.31 (MeOH/CH₂Cl₂, 4:96). LCMS m/z 525 [M+H]⁺. ¹H NMR (CDCl₃, rotamers 0.4:0.6) δ 7.30-6.90 (m, 8H, Ar—H), 4.63-4.48 (m, 3.6H, pip-H, dioxane-H, benzyl-H), 4.05 (m, 2H, dioxane-H), 3.82 (s, 0.8H, benzyl-H), 3.76-3.62 (m, 2.4H, dioxane-H, pip-H), 3.55 (m, 1.2H, benzyl-H), 2.92-2.84 (m, 2H, pip-H), 2.43-2.36 (m, 2H, NCH₂), 2.10-1.96 (m, 2.2H, dioxane-H, pip-H), 1.88-1.79 (m, 0.8H, pip-H), 1.76-1.52 (m, 5.2H, pip-H, NCH₂CH₂), 1.38-1.26 (m, 1.8H, pip-H, dioxane-H). HPLC t_{R}=8.4 min.

### Example 206

### N-{1-[2-(1,3-Dioxan-2-yl)ethyl]piperidin-4-yl}-N-(4-fluorobenzyl)-2-(4-ethoxyphenyl)-acetamide, oxalate (118AF68-104)

The desired compound was synthesized from 118AF52-95 (400 mg, 1.24 mmol) and 4-ethoxyphenylacetyl chloride (300 mg, 1.51 mmol) using the same method as for preparation of 118AF66-102. Further purification by acidic ion-exchange SPE cartridge was performed. Yield: 0.15 g, 25%.

R_{f}=0.26 (MeOH/CH₂Cl₂, 6:94). LCMS m/z 485 [M+H]⁺. ¹H NMR (CDCl₃, rotamers 0.4:0.6) δ 7.20-6.79 (m, 8H, Ar—H), 4.64-4.54 (m, 0.6H, pip-H), 4.52 (t, 1H, J=5.2, dioxane-H), 4.49 & 4.43 (2s, 2H, benzyl-H), 4.07-3.97 (m, 4H, dioxane-H, OCH₂), 3.76-3.66 (m, 3.2H, dioxane-H, pip-H, benzyl-H), 3.49 (s, 1.2H, benzyl-H), 2.91-2.80 (m, 2H, pip-H), 2.42-2.32 (m, 2H, NCH₂), 2.10-1.97 (m, 2.2H, dioxane-H, pip-H), 1.86-1.48 (m, 6H, pip-H, NCH₂CH₂), 1.42-1.36 (m, 3H, CH₃), 1.34-1.24 (m, 1.8H, pip-H, dioxane-H). HPLC t_{R}=7.6 min.

### Example 207

### N-{1-[2-(1,3-Dioxan-2-yl)ethyl]piperidin-4-yl}-N-(4-fluorobenzyl)-2-(4-isopropoxyphenyl)-acetamide, oxalate (118AF73-107)

The desired compound was synthesized from 118AF52-95 (400 mg, 1.24 mmol) and 4-isopropoxyphenylacetyl chloride (340 mg, 1.60 mmol) using the same method as for preparation of 118AF66-102. Further purification by acidic ion-exchange SPE cartridge was performed. Yield: 91 mg, 15%.

R_{f}=0.58 (MeOH/CH₂Cl₂, 8:92). LCMS m/z 499 [M+H]⁺. ¹H NMR (CDCl₃, rotamers 0.4:0.6) δ 7.19-6.78 (m, 8H, Ar—H), 4.64-4.42 (m, 4.6H, pip-H, dioxane-H, benzyl-H, CH_{OiPr}), 4.07 (m, 2H, dioxane-H), 3.76-3.68 (m, 3.2H, dioxane-H, pip-H, benzyl-H), 3.49 (s, 1.2H, benzyl-H), 2.91-2.80 (m, 2H, pip-H), 2.42-2.35 (m, 2H, NCH₂), 2.10-1.99 (m, 2.2H, dioxane-H, pip-H), 1.85-1.51 (m, 6H, pip-H, NCH₂CH₂), 1.31 (m, 7.8H, OCH(CH₃)₂, pip-H, dioxane-H). HPLC t_{R}=8.1 min.

### Example 208

### N-{1-[2-(1,3-Dioxan-2-yl)ethyl]piperidin-4-yl}-N-(4-fluorobenzyl)-2-phenylacetamide, oxalate (118AF77-109)

The desired compound was synthesized from 118AF52-95 (300 mg, 0.93 mmol) and phenylacetyl chloride (197 mg, 1.27 mmol) using the same method as for preparation of 118AF66-102. Further purification by acidic ion-exchange SPE cartridge was performed. Yield: 68 mg, 17%.

R_{f}=0.28 (MeOH/CH₂Cl₂, 5:95). LCMS m/z 441 [M+H]⁺. ¹H NMR (CDCl₃, rotamers 0.4:0.6) δ 7.33-6.89 (m, 9H, Ar—H), 4.65-4.44 (m, 3.6H, pip-H, dioxane-H, benzyl-H), 4.09-4.03 (m, 2H, dioxane-H), 3.84 (s, 0.8H, benzyl-H), 3.76-3.67 (m, 2.4H, dioxane-H, pip-H), 3.57 (s, 1.2H, benzyl-H), 2.92-2.79 (m, 2H, pip-H), 2.44-2.34 (m, 2H, NCH₂), 2.10-1.98 (m, 2.2H, dioxane-H, pip-H), 1.86-1.51 (m, 6H, pip-H, NCH₂CH₂), 1.34-1.23 (m, 1.8H, pip-H, dioxane-H). HPLC t_{R}=6.1 min.

### Example 209

### N-{1-[2-(1,3-Dioxan-2-yl)ethyl]piperidin-4-yl}-N-(4-fluorobenzyl)-2-[4-(2-fluoroethoxy)-phenyl]acetamide, oxalate (118AF85-113)

The desired compound was synthesized from 118AF52-95 (360 mg, 1.11 mmol) and 4-(2-fluoroethoxy)phenylacetyl chloride (282 mg, 1.30 mmol) using the same method as for preparation of 118AF66-102. Further purification by acidic ion-exchange SPE cartridge was performed. Yield: 84 mg, 15%.

R_{f}=0.36 (MeOH/CH₂Cl₂, 5:95). LCMS m/z 503 [M+H]⁺. ¹H NMR (CDCl₃, rotamers 0.4:0.6) δ 7.27-6.84 (m, 8H, Ar—H), 4.80 (m, 1H, OCH₂CH₂F), 4.68 (m, 1H, OCH₂CH₂F), 4.65-4.45 (m, 3.6H, pip-H, dioxane-H, benzyl-H), 4.22 (m, 1H, OCH₂CH₂F), 4.16 (m, 1H, OCH₂CH₂F), 4.10-4.03 (m, 2H, dioxane-H), 3.79-3.68 (m, 3.2H, dioxane-H, pip-H, benzyl-H), 3.51 (s, 1.2H, benzyl-H), 2.92-2.82 (m, 2H, pip-H), 2.44-2.36 (m, 2H, NCH₂), 2.12-1.99 (m, 2.2H, dioxane-H, pip-H), 1.88-1.51 (m, 6H, pip-H, NCH₂CH₂), 1.35-1.26 (m, 1.8H, pip-H, dioxane-H). HPLC t_{R}=7.0 min.

### Example 210

### N-{1-[2-(5,5-Dimethyl-1,3dioxan-2-yl)ethyl]piperidin-4-yl}-N-(4-fluorobenzyl)-2-(4-isobutoxyphenyl)acetamide, oxalate (118AF27-83)

The desired compound was synthesized from N-{1-[2-(1,3-dioxan-2-yl)ethyl]piperidin-4-yl}-N-(4-fluorobenzyl)-2-(4-isobutoxyphenyl)acetamide 103NLS63F (22 mg, 0.042 mmol) and 2,2-dimethyl-1,3-propandiol (33 mg, 0.38 mmol) using the same method as for preparation of 130AF12-148. Purification of the product by reversed phase HPLC (C₁₈) afforded the title compound (2.8 mg, 12%). LCMS m/z 541 [M+H]⁺. HPLC t_{R}=9.9 min.

### Example 211

### N-(4-Fluorobenzyl)-2-(4-isobutoxyphenyl)-N-{1-[2-((R)-4-methyl-1,3-dioxan-2-yl)ethyl]-piperidin-4-yl}acetamide, oxalate (118AF29-84)

The desired compound was synthesized from N-{1-[2-(1,3-dioxan-2-yl)ethyl]piperidin-4-yl}-N-(4-fluorobenzyl)-2-(4-isobutoxyphenyl)acetamide 103NLS63F (38 mg, 0.074 mmol) and (R)-(−)-1,3-butandiol (33 mg, 0.38 mmol) using the same method as for preparation of 130AF12-148. Purification of the product by reversed phase HPLC (C₁₈) afforded the title compound (11.6 mg, 28%). LCMS m/z 527 [M+H]⁺. HPLC t_{R}=8.7 min.

### Example 212

### N-(4-Fluorobenzyl)-2-(4-isobutoxyphenyl)-N-{1-[2-((S)-4-methyl-1,3-dioxolan-2-yl)ethyl]-piperidin-4-yl}acetamide, oxalate (118AF31-85)

The desired compound was synthesized from N-{1-[2-(1,3-dioxan-2-yl)ethyl]piperidin-4-yl}-N-(4-fluorobenzyl)-2-(4-isobutoxyphenyl)acetamide 103NLS63F (40 mg, 0.078 mmol) and (S)-(+)-propylene glycol (30 mg, 0.39 mmol) using the same method as for preparation of 130AF12-148. Purification of the product by reversed phase HPLC (C₁₈) afforded the title compound (21 mg, 53%). LCMS m/z 513 [M+H]⁺. HPLC t_{R}=9.9 min.

### Example 213

### N-{1-[2-(4,6-Dimethyl-1,3-dioxan-2-yl)ethyl]piperidin-4-yl}-N-(4-fluorobenzyl)-2-(4-isobutoxyphenyl)acetamide, oxalate (118AF37-88)

The desired compound was synthesized from N-{1-[2-(1,3-dioxan-2-yl)ethyl]piperidin-4-yl}-N-(4-fluorobenzyl)-2-(4-isobutoxyphenyl)acetamide 103NLS63F (40 mg, 0.078 mmol) and 2,4-pentandiol (41 mg, 0.39 mmol) using the same method as for preparation of 130AF12-148. Purification of the product by reversed phase HPLC (C₁₈) afforded the title compound (9 mg, 21%). LCMS m/z 541 [M+H]⁺. HPLC t_{R}=10.5 min.

### Example 214

### N-(4-Fluorobenzyl)-N-{1-[2-((S)-4-methyl-1,3-dioxolan-2-yl)ethyl]piperidin-4-yl}-2-(4-trifluoromethoxyphenyl)acetamide, oxalate (118AF87-114)

The desired compound was synthesized from N-{1-[2-(1,3-dioxan-2-yl)ethyl]piperidin-4-yl}-N-(4-fluorobenzyl)-2-(4-trifluoromethoxyphenyl)acetamide 118AF58-98 (70 mg, 0.13 mmol) and (S)-(+)-propylene glycol (53 mg, 0.69 mmol) using the same method as for preparation of 130AF12-148. Purification of the product by silica gel column chromatography, eluting with a stepwise gradient of 0-4% methanol in dichloromethane, afforded the title compound (31 mg, 46%). R_{f}=0.17 (MeOH/CH₂Cl₂ 4:96). LCMS m/z 525 [M+H]⁺. ¹H NMR (CDCl₃, rotamers 0.4:0.6) δ 7.34-6.91 (m, 8H, Ar—H), 5.03 & 4.92 (2t, 1H, J=4.8, dioxolane-H), 4.66-4.56 (m, 0.6H, pip-H), 4.52 & 4.49 (2s, 2H, benzyl-H), 4.22-4.07 (m, 1.4H, dioxolane-H), 3.95-3.89 (m, 0.6H, dioxolane-H), 3.84 (s, 0.8H, benzyl-H), 3.74-3.64 (m, 0.4H, pip-H), 3.57 (s, 1.2H, benzyl-H), 3.40-3.33 (m, 1H, dioxolane-H), 2.78-2.86 (m, 2H, pip-H), 2.49-2.38 (m, 2H, NCH₂), 2.10-2.01 (m, 1.2H, pip-H), 1.70-1.53 (m, 6H, pip-H, NCH₂CH₂), 1.40-1.22 (m, 3.8H, pip-H, CH₃). HPLC t_{R}=8.7 mm.

### Example 215

### N-(4-Fluorobenzyl)-2-(4-isopropylphenyl)-N-{1-[2-((S)-4-methyl-1,3-dioxolan-2-yl)ethyl]-piperidin-4-yl}acetamide, oxalate (118AF91-117)

The desired compound was synthesized from N-{1-[2-(1,3-dioxan-2-yl)ethyl]piperidin-4-yl}-N-(4-fluorobenzyl)-2-(4-isopropylphenyl)acetamide 118AF63-100 (150 mg, 0.31 mmol) and (S)-(+)-propylene glycol (95 mg, 1.24 mmol) using the same method as for preparation of 130AF12-148. Purification by silica gel column chromatography, eluting with a stepwise gradient of 0-4% methanol in dichloromethane, afforded the title compound (51.2 mg, 34%).

R_{f}=0.19 (MeOH/CH₂Cl₂, 4:96). LCMS m/z 483 [M+H]⁺. ¹H NMR (CDCl₃, rotamers 0.4:0.6) δ 7.24-6.90 (m, 8H, Ar—H), 5.03 & 4.92 (2t, 1H, J=4.8, dioxolane-H), 4.67-4.55 (m, 0.6H, pip-H), 4.51 & 4.47 (2s, 2H, benzyl-H), 4.21-4.07 (m, 1.4H, dioxolane-H), 3.94-3.89 (m, 0.6H, dioxolane-H), 4.81-3.50 (m, 1.2H, benzyl-H, pip-H), 3.55 (s, 1.2H, benzyl-H), 3.40-3.33 (m, 1H, dioxolane-H), 2.94-2.83 (m, 3H, pip-H, CH(CH₃)₂), 2.47-2.38 (m, 2H, NCH₂), 2.09-2.01 (m, 1.2, pip-H), 1.86-1.52 (m, 6H, pip-H, NCH₂CH₂), 1.31-1.19 (m, 9.8H, pip-H, CH₃, CH(CH₃)₂). HPLC t_{R}=8.6 min.

### Example 216

### N-(4-Fluorobenzyl)-N-{1-[2-((R)-4-methyl-1,3-dioxan-2-yl)ethyl]piperidin-4-yl}-2-(4-trifluoromethoxyphenyl)acetamide, oxalate (118AF75-108)

The desired compound was synthesized from N-{1-[2-(1,3-dioxan-2-yl)ethyl]piperidin-4-yl}-N-(4-fluorobenzyl)-2-(4-trifluoromethoxyphenyl)acetamide 118AF58-98 (70 mg, 0.13 mmol) and (R)-(−)-1,3-butandiol (60 mg, 0.66 mmol) using the same method as for preparation of 130AF12-148. Purification of the product by silica gel column chromatography, eluting with a stepwise gradient of 0-4% methanol in dichloromethane, afforded the title compound (28 mg, 40%).

R_{f}=0.24 (MeOH/CH₂Cl₂, 5:95). LCMS m/z 539 [M+H]⁺. ¹H NMR (CDCl₃, rotamers 0.4:0.6) δ 7.33-6.91 (m, 8H, Ar—H), 4.64-4.48 (m, 3.6H, benzyl-H, dioxane-H, pip-H), 4.04 (m, 1H, dioxane-H), 3.83 (s, 0.8H, benzyl-H), 3.75-3.63 (m, 2.4H, dioxane-H, pip-H), 3.56 (s, 1.2H, benzyl-H), 2.92-2.83 (m, 2H, pip-H), 2.44-2.38 (m, 2H, NCH₂), 2.09-2.01 (m, 1.2 pip-H), 1.89-1.53 (m, 7H, dioxane-H, pip-H, NCH₂CH₂), 1.44-1.31 (m, 1.8H, dioxane-H, pip-H), 1.19 (m, 3H, CH₃). HPLC t_{R}=9.0 min.

### Example 217

### N-(4-Fluorobenzyl)-2-(4-isobutoxyphenyl)-N-{1-[2-(2,5,5-trimethyl-1,3-dioxan-2-yl)ethyl]piperidin-4-yl}acetamide, oxalate (118AF33-86)

The desired compound was synthesized from N-(4-fluorobenzyl)-2-(4-isobutoxyphenyl)-N-piperidin-4-yl-acetamide103NLS56 (145 mg, 0.36 mmol) and 2-(2-bromoethyl)-2,5,5-trimethyl-1,3-dioxane (104.5 mg, 0.44 mmol) using the same method as for synthesis of 130AF65-182. Purification of the product by silica gel column chromatography, eluting with 5% methanol in dichloromethane, afforded the title compound (119 mg, 58%).

R_{f}=0.15 (MeOH/CH₂Cl₂, 5:95). LCMS m/z 555 [M+H]⁺. ¹H NMR (CDCl₃, rotamers 0.4:0.6) δ 7.20-6.79 (m, 8H, Ar—H), 4.66-4.56 (m, 0.6H, pip-H), 4.49 & 4.43 (2s, 2H, benzyl-H), 3.76-3.68 (m, 3.2H, pip-H, benzyl-H, CH_{2OiBu}), 3.52-3.47 (m, 4H, dioxane-H), 3.41 (m, 1.2H, benzyl-H), 2.93-2.84 (m, 2H, pip-H), 2.48-2.40 (m, 2H, NCH₂), 2.11-2.00 (m, 2.2H, CH_{OiBu}, pip-H), 1.87-1.80 (m, 2.8H, pip-H, NCH₂CH₂), 1.72-1.50 (m, 3.2H, pip-H), 1.33 (s, 3.8H, CH₃, pip-H), 1.02-0.87 (m, 12H, CH₃). HPLC t_{R}=9.8 min.

### Example 218

### N-(4-Fluorobenzyl)-2-(4-isobutoxyphenyl)-N-{1-[2-(2-methyl-1,3-dioxolan-2-yl)ethyl]-piperidin-4-yl}acetamide, oxalate (118AF35-87)

The desired compound was synthesized from N-(4-fluorobenzyl)-2-(4-isobutoxyphenyl)-N-piperidin-4-yl-acetamide 103NLS56 (311 mg, 0.78 mmol) and 2-(2-bromoethyl)-2-methyl-1,3-dioxolane (188 mg, 0.96 mmol) using the same method as for synthesis of 130AF65-182. Purification by silica gel column chromatography, eluting with 5% methanol in dichloromethane, afforded the title compound (61 mg, 15%).

R_{f}=0.20 (MeOH/CH₂Cl₂, 5:95). LCMS m/z 513 [M+H]⁺. ¹H NMR (CDCl₃, rotamers 0.4:0.6) δ 7.17-6.76 (m, 8H, Ar—H), 4.64-4.52 (m, 0.6H, pip-H), 4.47 & 4.41 (2s, 2H, benzyl-H), 3.93-3.82 (m, 4H, dioxolane-H), 3.76-3.63 (m, 3.2H, benzyl-H, CH_{2OiBu}, pip-H), 3.47 (s, 1.2H, benzyl-H), 2.94-2.83 (m, 2H, pip-H), 2.43-2.32 (m, 2H, NCH₂), 2.12-1.97 (m, 2.2H, CH_{OiBu}, pip-H), 1.84-1.72 (m, 2.8H, pip-H, NCH₂CH₂), 1.70-1.50 (m, 3.2H, pip-H), 1.27 (s, 3.8H, CH₃, pip-H), 0.98 (m, 6H, CH_{3OiBu}). HPLC t_{R}=8.8 min.

### Example 219

### N-(4-Fluorobenzyl)-2-(4-isobutoxyphenyl)-N-{1-[3-(1,3-dioxolan-2-yl)propyl]piperidin-4-yl}acetamide, tartrate (118AF79-39)

The desired compound was synthesized from N-(4-fluorobenzyl)-2-(4-isobutoxyphenyl)-N-piperidin-4-yl-acetamide 103NLS56 (156 mg, 0.39 mmol) and 2-(3-chloropropyl)-1,3-dioxolane (62 μL, 0.47 mmol) using the same method as for synthesis of 130AF65-182. Purification by silica gel column chromatography, eluting with a stepwise gradient of 0-4% methanol in dichloromethane, afforded the title compound (49 mg, 25%).

R_{f}=0.45 (MeOH/CH₂Cl₂, 7:93). LCMS m/z 513 [M+H]⁺. ¹H NMR (CDCl₃, rotamers 0.4:0.6) δ 7.21-6.79 (m, 8H, Ar—H), 4.84 (t, 1H, J=4.4, dioxolane-H), 4.66-4.56 (m, 0.6H, pip-H), 4.50 & 4.44 (2s, 2H, benzyl-H), 3.95-3.90 (m, 2H, dioxolane-H), 3.84-3.67 (m, 5.2H, benzyl-H, CH_{2OiBu}, pip-H, dioxolane-H), 3.50 (s, 1.2H, benzyl-H), 2.94-2.84 (m, 2H, pip-H), 2.34-2.27 (m, 2H, NCH₂), 2.10-1.98 (m, 2.2H, CH_{OiBu}, pip-H), 1.84-1.78 (m, 0.8H, pip-H), 1.71-1.50 (m, 7.2H, pip-H, NCH₂CH₂), 1.34-1.25 (m, 0.8H, pip-H), 1.01 (m, 6H, CH_{3OiBu}). HPLC t_{R}=8.0 min.

### Example 220

### N-(4-Fluorobenzyl)-2-(4-isobutoxyphenyl)-N-{1-(3-piperidin-1-yl-propyl)piperidin-4-yl}-acetamide, dihydrochloride (98AF36-43)

The desired compound was synthesized from N-(4-fluorobenzyl)-2-(4-isobutoxyphenyl)-N-piperidin-4-yl-acetamide103NLS56 (189 mg, 0.47 mmol), 1-piperidine (61 μL, 0.61 mmol) and 1-chloro-3-iodopropane (61 μL, 0.57 mmol) using the same method as for synthesis of 130AF09-145. Purification of the product by silica gel column chromatography, eluting with 10% methanol in dichloromethane, afforded the title compound (75.6 mg, 31%).

R_{f}=0.13 (MeOH/CH₂Cl₂, 1:4). LCMS m/z 524 [M+H]⁺. ¹H NMR (CDCl₃, rotamers 0.4:0.6) δ 7.21-6.81 (m, 8H, Ar—H), 4.66-4.54 (m, 0.6H, pip-H), 4.51 & 4.45 (2s, 2H, benzyl-H), 3.78-3.68 (m, 3.2H, benzyl-H, CH_{2OiBu}, pip-H), 3.52 (s, 1.2H, benzyl-H), 2.93-2.83 (m, 2H, pip-H), 2.40-2.23 (m, 8H, NCH₂), 2.15-1.26 (m, 15H, pip-H, CH(CH₃)₂, CH₂), 1.02 (m, 6H, CH(CH₃)₂). HPLC t_{R}=8.0 min.

### Example 221

### N-(4-Fluorobenzyl)-2-(4-isobutoxyphenyl)-N-{1-[2-(tetrahydropyran-2-yloxy)ethyl]-piperidin-4-yl}acetamide, oxalate (98AF41-44)

The desired compound was synthesized from N-(4-fluorobenzyl)-2-(4-isobutoxyphenyl)-N-piperidin-4-yl-acetamide 103NLS56 (185 mg, 0.46 mmol) and 2-(2-chloroethoxy)-tetrahydro-2H-pyran (75 μL, 0.51 mmol) using the same method as for synthesis of 130AF09-145. Purification of the product by silica gel column chromatography, eluting with 4.5% methanol in dichloromethane, afforded the title compound (96 mg, 40%).

R_{f}=0.18 (MeOH/CH₂Cl₂, 4:96). LCMS m/z 527 [M+H]⁺. ¹H NMR (CDCl₃, rotamers 0.4:0.6) δ 7.21-6.78 (m, 8H, Ar—H), 4.67-4.56 (m, 0.6H, pip-H), 4.54 (m, 1H, THP), 4.49 & 4.44 (2s, 2H, benzyl-H), 3.86-3.66 (m, 5.2H, benzyl-H, CH_{2OiBu}, pip-H, CHO), 3.58-3.43 (m, 3.2H, benzyl-H, CHO), 3.01-2.89 (m, 2H, pip-H), 2.62 & 2.55 (2t, 2H, J=6.0, NCH₂CH₂O), 2.26-2.17 (m, 1.2H, pip-H), 2.12-1.96 (m, 1.8H, CH_{OiBu}, pip-H), 1.82-1.44 (m, 9.2H, pip-H, THP), 1.33-1.26 (m, 0.8H, pip-H), 1.01 (m, 6H, CH(CH₃)₂). HPLC t_{R}=7.2 min.

### Example 222

### N-(4-Fluorobenzyl)-2-(4-isobutoxyphenyl)-N-{1-[3-(2-oxo-piperidin-1-yl)propyl]piperidin-4-yl}acetamide (98AF73-64)

Sodium hydride (60% suspension in oil, 26 mg, 0.65 mmol) was added to a solution of 2-piperidone (54 mg, 0.54 mmol) in dry THF (2 mL) under argon atmosphere. After 15 minutes stirring at rt the reaction mixture was cooled to 0° C. over 15 minutes. A solution of 1-bromo-3-chloropropane (160 μL, 1.62 mmol) was added dropwise to the cold mixture and stirring was continued overnight at rt. The mixture was partitioned between water and ethyl acetate, the organic layer dried over sodium sulphate, filtered and evaporated to dryness. Purification of the residue on silica gel column chromatography, eluting with a stepwise gradient of 60-80% ethyl acetate in n-heptane, afforded 1-(3-chloropropyl)-piperidin-2-one (33 mg, 35%).

R^{f}=0.22 (ethyl acetate/n-heptane 8:2). LCMS m/z 176 [M+H]⁺. HPLC t_{R}=1.8 min.

A solution of 1-(3-chloropropyl)-piperidin-2-one (32 mg, 0.18 mmol) in dry DMF (2 mL) was added to a suspension of potassium carbonate (52 mg, 0.38 mmol) and N-(4-fluorobenzyl)-2-(4-isobutoxyphenyl)-N-piperidin-4-yl-acetamide 103NLS56 (62 mg, 0.15 mmol) in dry DMF (2 mL). After addition of sodium iodide (25 mg, 0.17 mmol) the mixture was stirred overnight at 48° C. Afterwards it was partitioned between water and dichloromethane. The organic layer were dried over sodium sulphate, filtered and evaporated to dryness. Purification of the residue by reversed phase HPLC (C₁₈) afforded the desired compound (2.6 mg, 3%).

R_{f}=0.11 (MeOH/CH₂Cl₂ 5:95). LCMS m/z 538 [M+H]⁺. HPLC t_{R}=8.2 min.

### Example 223

### N-(4-Fluorobenzyl)-2-(4-isobutoxyphenyl)-N-{1-[3-(2-oxo-pyrrolidin-1-yl)propyl]piperidin-4-yl}acetamide, hydrochloride (98AF76-65)

The desired compound was synthesized from N-(4-fluorobenzyl)-2-(4-isobutoxyphenyl)-N-piperidin-4-yl-acetamide 103NLS56 (107 mg, 0.27 mmol), 2-pyrrolidone and 1-bromo-3-chloropropane using the same method as for synthesis of 98AF73-64. Purification of the product by silica gel column chromatography, eluting with a stepwise gradient of 4-8% methanol in dichloromethane, afforded the title compound (15 mg, 11%).

R_{f}=0.39 (MeOH/CH₂Cl₂ 1:9). LCMS m/z 524 [M+H]⁺.¹H NMR (CDCl₃, rotamers 0.4:0.6) δ 7.20-6.80 (m, 8H), 4.65-4.53 (m, 0.6H), 4.50 & 4.44 (2s, 2H), 3.76-3.67 (m, 3.2H), 3.51 (m, 1.2H), 3.34 (t, 2H, J=7.2), 3.26 (t, 2H, J=7.2), 2.95-2.82 (m, 2H), 2.38-2.25 (m, 4H), 2.12-1.96 (m, 4.2H), 1.86-1.56 (m, 6H), 1.29 (m, 0.8H), 1.01 (m, 6H). HPLC t_{R}=7.6 min.

### Example 224

### N-(4-Fluorobenzyl)-2-(4-isobutoxyphenyl)-N-{1-[3-((R)-4-isopropyl-2-oxo-oxazolidin-3-yl)propyl]piperidin-4-yl}acetamide, oxalate (98AF100-73)

Sodium hydride (55% suspension in oil, 144 mg, 3.31 mmol) was added to a solution of (R)-4-isopropyl-2-oxazolidinone (356 mg, 2.75 mmol) in dry tetrahydrofuran (17 mL) under argon atmosphere. The suspension was stirred for 1 hour at rt, then cooled to 0° C. and a solution of 1-bromo-3-chloropropane in dry tetrahydrofuran (3 mL) was added dropwise. After 48 h stirring at 58° C. the mixture was quenched with water. The solvent was removed by evaporation under reduced pressure and the residue partitioned between water and dichloromethane. The organic layer was evaporated to dryness. Purification of the residue by silica gel column chromatography, eluting with a mixture of ethyl acetate and n-Heptane (70:30), afforded (4R)-3-(3-chloropropyl)-4-isopropyloxazolidinon-2-one (401 mg, 71%).

A solution of (4R)-3-(3-chloropropyl)-4-isopropyloxazolidinon-2-one (160 mg, 0.78 mmol) in dry DMF (2 mL) was added to a suspension of potassium carbonate (217 mg, 1.57 mmol) and N-(4-fluorobenzyl)-2-(4-isobutoxyphenyl)-N-piperidin-4-yl-acetamide 103NLS56 (250 mg, 0.63 mmol) in dry DMF (6 mL). After addition of sodium iodide (113 mg, 0.75 mmol) the mixture was stirred overnight at 62° C. and partitioned between water and dichloromethane. The organic layer were dried over sodium sulphate, filtered and evaporated to dryness. Purification of the residue by silica gel column chromatography, eluting with 5% methanol in dichloromethane, afforded the desired compound (143 mg, 40%).

R_{f}=0.28 (MeOH/CH₂Cl₂ 6:96). LCMS m/z 568 [M+H]⁺. ¹H NMR (CDCl₃, rotamers 0.4:0.6) δ 7.20-6.78 (m, 8H, Ar—H), 4.61-4.51 (m, 0.6H, pip-H), 4.48 & 4.42 (2s, 2H, benzyl-H), 4.15 (t, 1H, J=8.8, oxa-H), 4.01 (m, 1H, oxa-H), 3.78-3.64 (m, 4.2H, pip-H, benzyl-H, oxa-H, CH_{2OiBu}), 3.48 (m, 2.2H, benzyl-H, CONCHCH₂), 2.92-2.79 (m, 3H, pip-H, CONCHCH₂), 2.34-2.22 (m, 2H, NCH₂CH₂CH₂), 2.10-1.96 (m, 3.2H, pip-H, CH_{iPr}, CH_{OiBu}), 1.76-1.50 (m, 6H, pip-H, NCH₂CH₂), 1.32-1.26 (m, 0.8H, pip-H), 0.99 (m, 6H, CH_{3OiBu}), 0.81-0.87 (m, 6H, CH_{3iPr}). HPLC t_{R}=9.1 min.

### Example 225

### N-(4-Fluorobenzyl)-2-(4-isobutoxyphenyl)-N-{1-[3-(2-oxo-oxazolidin-3-yl)propyl]piperidin-4-yl}acetamide, oxalate (98AF94-71)

The desired compound was synthesized from N-(4-fluorobenzyl)-2-(4-isobutoxyphenyl)-N-piperidin-4-yl-acetamide 103NLS56 (298 mg, 0.75 mmol), 2-oxazolidone and 1-bromo-3-chloropropane using the same method as for synthesis of 98AF100-73. Purification of the product by silica gel column chromatography, eluting with 5% methanol in dichloromethane, afforded the title compound (157 mg, 40%).

R_{f}=0.23 (MeOH/CH₂Cl₂ 5:95). LCMS m/z 526 [M+H]⁺. ¹H NMR (CDCl₃, rotamers 0.4:0.6) δ 7.20-6.78 (m, 8H, Ar—H), 4.61-4.50 (m, 0.6H, pip-H), 4.48 & 4.42 (2s, 2H, benzyl-H), 4.29-4-24 (m, 2H, oxa-OCH₂), 3.78-3.65 (m, 3.2H, pip-H, benzyl-H, CH_{2OiBu}), 3.52-3.48 (m, 3.2H, benzyl-H, oxa-NCH₂), 3.25 (t, 2H, J=7.2, CONCH₂CH₂CH₂N), 2.89-2.80 (m, 2H, pip-H), 2.33-2.26 (m, 2H, NCH₂CH₂CH₂NCO), 2.09-1.76 (m, 3H, pip-H, CH_{OiBu}), 1.71-1.49 (m, 5.2H, pip-H, NCH₂CH₂CH₂), 1.33-1.27 (m, 0.8H, pip-H), 1.00 (m, 6H, CH_{3OiBu}). HPLC t_{R}=7.8 min.

### Example 226

### (S)-4-Methyl-oxazolidin-2-one (118AF10-77)

Triethylamine (0.94 mL, 6.65 mmol) was added dropwise to a solution of L-alaminol (500 mg, 6.65 mmol) and 1,1-carbonyldiimidazole (1.29 g, 7.98 mmol) in dry THF (10 mL) at rt, under argon atmosphere. The reaction mixture was stirred overnight at 60° C. The solvent was removed by evaporation under reduced pressure. Purification of the residue by silica gel column chromatography, eluting with 6% methanol in dichloromethane, afforded the desired compound (450 mg, 67%).

R_{f}=0.39 (MeOH/CH₂Cl₂ 6:94). ¹H NMR (CDCl₃) δ 6.74 (m, 1H), 4.45-4.34 (m, 1H), 4.98-4.77 (m, 2H), 1.17 (m, 3H).

### Example 227

### N-(4-Fluorobenzyl)-2-(4-isobutoxyphenyl)-N-{1-[3-((S)-4-methyl-2-oxo-oxazolidin-3-yl)propyl]piperidin-4-yl}acetamide, tartrate (118AF18-81)

The desired compound was synthesized from N-(4-fluorobenzyl)-2-(4-isobutoxyphenyl)-N-piperidin-4-yl-acetamide 103NLS56 (205 mg, 0.52 mmol), (S)-4-methyl-oxazolidin-2-one (118AF10-77) and 1-bromo-3-chloropropane using the same method as for synthesis of 98AF100-73. Further purification by acidic ion-exchange SPE cartridge was performed. Yield: 106 mg, 38%.

R_{f}=0.22 (MeOH/CH₂Cl₂ 6:94). LCMS m/z 540 [M+H]⁺. ¹H NMR (CDCl₃, rotamers 0.4:0.6) δ 7.20-6.78 (m, 8H, Ar—H), 4.61-4.50 (m, 0.6H, pip-H), 4.48 & 4.42 (2s, 2H, benzyl-H), 4.34 (m, 1H, oxa-H), 3.84-3.66 (m, 5.2H, pip-H, benzyl-H, oxa-H, CH_{2OiBu}), 3.49 (s, 1.2H, benzyl-H), 3.42-3.34 (m, 1H, CONCH₂), 3.09-3.00 (m, 1H, CONCH₂), 2.92-2.79 (m, 2H, pip-H), 2.33-2.26 (m, 2H, NCH₂), 2.10-1.98 (m, 2.2H, pip-H, CH_{OiBu}), 1.86-1.76 (m, 0.8H, pip-H), 1.72-1.48 (m, 5.2H, pip-H, NCH₂CH₂), 1.29 (m, 0.8H, pip-H), 1.22 (m, 3H, oxa-CH₃), 0.99 (m, 6H, CH_{3OiBu}). HPLC t_{R}=8.4 min.

### Example 228

### (S)-4-Ethyl-oxazolidin-2-one (118AF08-76)

Triethylamine (0.80 mL, 5.74 mmol) was added dropwise to a solution of (S)-(+)-2-amino-1-butanol (515 mg, 5.77 mmol) and 1,1-carbonyldiimidazole (1.10 g, 6.78 mmol) in dry THF (10 mL) at rt under argon atmosphere. The reaction mixture was stirred overnight at rt. The solvent was removed by evaporation under reduced pressure. Purification of the residue by silica gel column chromatography, eluting with 6% methanol in dichloromethane, afforded the desired compound (485 mg, 73%). R_{f}=0.42 (MeOH/CH₂Cl₂ 6:94).

### Example 229

### N-(4-Fluorobenzyl)-2-(4-isobutoxyphenyl)-N-{1-[3-((S)-4-ethyl-2-oxo-oxazolidin-3-yl)-propyl]piperidin-4-yl}acetamide, oxalate (118AF16-80)

The desired compound was synthesized from N-(4-fluorobenzyl)-2-(4-isobutoxyphenyl)-N-piperidin-4-yl-acetamide 103NLS56 (202 mg, 0.51 mmol), (S)-4-ethyl-oxazolidin-2-one (118AF08-76) and 1-bromo-3-chloropropane using the same method as for synthesis of 98AF100-73. Purification of the product by acidic ion-exchange SPE cartridge afforded the title compound (126 mg, 44%).

R_{f}=0.28 (MeOH/CH₂Cl₂ 6:94). LCMS m/z 554 [M+H]⁺. ¹H NMR (CDCl₃, rotamers 0.4:0.6) δ 7.20-6.78 (m, 8H, Ar—H), 4.61-4.52 (m, 0.6H, pip-H), 4.48 & 4.42 (2s, 2H, benzyl-H), 4.32-4.26 (m, 1H, oxa-H), 3.94-3.88 (m, 1H, oxa-H), 3.76-3.66 (m, 4.2H, pip-H, benzyl-H, oxa-H, CH_{2OiBu}), 3.49 (s, 1.2H, benzyl-H), 3.46-3.37 (m, 1H, CONCH₂), 3.04-2.96 (m, 1H, CONCH₂), 2.90-2.78 (m, 2H, pip-H), 2.33-2.24 (m, 2H, NCH₂), 2.11-1.96 (m, 2.2H, pip-H, CH_{OiBu}), 1.82-1.75 (m, 0.8H, pip-H), 1.74-1.42 (m, 7.2H, pip-H, NCH₂CH₂, CH₂CH₃), 1.29 (m, 0.8H, pip-H), 1.00 (m, 6H, CH_{3OiBu}), 0.85 (m, 3H, CH₂CH₃). HPLC t_{R}=8.7 min.

### Example 230

### 2-(2-Bromoethyl)-1,3-oxothiolane (121JP11)

Adapting a procedure by Yamada et al (Tetrahedron Lett., 1998, 39, 7709-7712), boron trifluoride ether complex (5 mL, 40 mmol) was added dropwise to a mixture of 2-(2-bromoethyl)-1,3-dioxolane (1.45 g, 8.0 mmol) and 2-mercaptoethanol (2.81 mL, 40 mmol) in dichloromethane (15 mL) at rt under Ar atmosphere and stirred at rt overnight. Sat. aq. NaHCO₃ (100 mL) was added to the crude mixture, followed by extraction using Et₂O (3×100 mL), drying (Na₂SO₄) and evaporation in vacuo. Purification by Kugelrohr distillation (90° C., 1.0 mmHg) afforded 1.08 g of the title compound as a yellow oil. The purity of this material after distillation was 71% (determined by GC analysis) and it was used as such in the alkylation step (121JP12).

### Example 231

### N-(4-Fluorobenzyl)-2-(4-isobutoxyphenyl)-N-{1-[2-(1,3-oxothiolan-2-yl)ethyl]piperidin-4-yl}acetamide, L-tartrate (121JP12)

The title compound was prepared by the general procedure described above for 103NLS63-F using 103NLS56 (130 mg, 0.33 mmol) and 121JP11 (85 mg, 0.43 mmol) as the alkylating agent. Workup as in 121JP11 followed by vacuum filtration chromatography over silica gel (VFC, ethyl acetate/n-heptane 0:1 ethyl acetate/n-heptane 1:0→ethyl acetate/MeOH 4:1) gave 85 mg (51%) of 121JP12 as colorless thick oil. The L-tartrate salt was prepared as described above.

R_{f}=0.57 (MeOH/CH₂Cl₂ 1:10). LCMS m/z 515 [M+H]⁺. ¹H-NMR (CDCl₃, rotamers 0.5:0.5) δ 7.20-6.76 (m, 8H), 5.10-5.00 (m, 1H, oxothiolane-H), 4.66-4.54 (m, 0.5H, pip-H), 4.48 and 4.42 (2s, 2H, benzyl-H), 4.30-4.22 (m, 1H, oxothiolane-H), 3.78-3.64 (m, 4.5H, pip-H, benzyl-H, oxothiolane-H, OCH_{2OiBu}), 3.48 (s, 1H, benzyl-H), 3.01-2.82 (m, 4H, pip-H, oxothiolane-H), 2.60-2.34 (m, 2H, NCH₂), 2.21-1.56 (m, 8H, pip-H, NCH₂CH₂, CH_{OiBu}), 1.32-1.22 (m, 1H, pip-H), 1.04-0.96 (m, 6H, CH_{3OiBu}. HPLC t_{R}=10.1 min.

### Example 232

### 2-(4-Bromophenyl)-N-{1-[2-(1,3-dioxan-2-yl)ethyl)piperidin-4-yl}-N-(4-fluorobenzyl)-acetamide, L-tartrate (121JP13)

The title compound was prepared by the procedure described above for 117NLS87-A using 118AF52-95 (200 mg, 0.62 mmol) and 4-bromophenylacetic acid (500 mg, 2.32 mmol). Sat. aq. NaHCO₃ (100 mL) was added to the crude mixture, followed by extraction using CH₂Cl₂ (3×100 mL), drying (Na₂SO₄) and evaporation in vacuo. VFC over silica gel (ethyl acetate/n-heptane 1:1→ethyl acetate/n-heptane 1:0→ethyl acetate/MeOH 2:1) gave 250 mg (78%) of 121JP12 as a thick oil. The L-tartrate salt was prepared as described above.

R_{f}=0.49 (MeOH/CH₂Cl₂ 1:10). LCMS m/z 521 [M+H]⁺. ¹H-NMR (CDCl₃, rotamers 0.6:0.4) δ 7.50-6.88 (m, 8H), 4.62-4.57 (m, 0.4H, pip-H), 4.50 (t, 1H, J=4.9, dioxane-H) 4.48 and 4.42 (2s, 2H, benzyl-H), 4.06-4.00 (m, 2H, dioxane-H), 3.76 and 3.50 (2s, 2H, benzyl-H), 3.75-3.60 (m, 2.6H, pip-H, dioxane-H), 3.01 and 2.90 (2d, 2H, J=10.5, pip-H), 2.52 and 2.41 (2t, 2H, J=8.0, NCH₂), 2.10-1.98 (m, 2.2H, dioxane-H, pip-H), 1.97-1.58 (m, 6H, pip-H, NCH₂CH₂), 1.38-1.20 (m, 1.8H, dioxane-H, pip-H). HPLC t_{R}=8.3 min.

### Example 233

### N-{1-[2-(1,3-Dioxan-2-yl)ethyl)piperidin-4-yl}-N-(4-fluorobenzyl)-2-(4-isobutylamino-phenyl)acetamide, L-tartrate (121JP27)

Adapting a protocol by Buchwald et al (J. Am. Chem. Soc., 1996, 118, 7215-7216), 121JP13 (100 mg, 192 μmol), isobutylamine (17 mg, 230 μmol), Pd₂ dba₃ (11.6 mg, 19.2 μmol), BINAP (12.0 mg, 38.4 μmol) and NaOtBu (25.8 mg, 269 μmol) were weighed into a flask, toluene (2 mL) was added and the resulting mixture was stirred at 80° C. for 18 h. Workup as in 121JP13 followed by preparative reversed-phase (C₁₈) HPLC afforded 25.7 mg (27.0%) of 121JP27 as a thick colorless oil. The L-tartrate salt was prepared as described above.

R_{f}=0.30 (MeOH/CH₂Cl₂ 1:10). LCMS m/z 512 [M+H]⁺. ¹H-NMR (CDCl₃, rotamers 0.5:0.5) δ 7.10-6.81 (m, 6H), 6.59-6.49 (m, 2H), 4.65-4.55 (m, 0.5H, pip-H), 4.55-4.50 (m, 1H, dioxane-H), 4.50 and 4.43 (2s, 2H, benzyl-H), 4.10-4.02 (m, 2H, dioxane-H), 3.80-3.67 (m, 3.5H, pip-H, benzyl-H, dioxane-H) 3.45 (s, 1H, benzyl-H), 2.95-2.85 (m, 4H, pip-H, NHCH₂CH(CH₃)₂)), 2.45-2.35 (m, 2H, NCH₂), 2.09-1.99 (m, 2H, dioxane-H, pip-H), 1.91-1.50 (m, 7H, NCH₂CH₂, pip-H, NHCH₂CH(CH₃)₂), 1.38-1.25 (m, 2H, dioxane-H, pip-H), 0.98 (m, 6H, NHCH₂CH(CH₃)₂). HPLC t_{R}=8.2 min.

### Example 234

### N-{1-[2-(1,3-Dioxan-2-yl)ethyl)piperidin-4-yl}-N-(4-fluorobenzyl)-2-(4-propylamino-phenyl)acetamide, L-tartrate (121JP28)

Prepared identically as described in the protocol for the synthesis of 121JP27, using propylamine (16 mg, 230 μmol) instead of isobutylamine to afford 24 mg (25%) of 121JP28 as a thick oil. The L-tartrate salt was prepared as described above.

R_{f}=0.33 (MeOH/CH₂Cl₂ 1:10). LCMS m/z 498 [M+H]⁺. ¹H-NMR (CDCl₃, rotamers 0.5:0.5) δ 7.11-6.82 (m, 6H), 6.53-6.43 (m, 2H), 4.58-4.49 (m, 0.5H, pip-H), 4.48-4.45 (m, 1H, dioxane-H), 4.42 and 4.35 (2s, 2H, benzyl-H), 4.05-3.95 (m, 2H, dioxane-H), 3.70-3.60 (m, 3.5H, pip-H, benzyl-H, dioxane-H), 3.40 (s, 1H, benzyl-H), 3.05-2.95 (m, 2H, pip-H), 2.85-2.70 (m, 2H, NHCH₂CH₂CH₃), 2.48-2.38 (m, 2H, NCH₂), 2.05-1.90 (m, 2H, dioxane-H, pip-H), 1.92-1.40 (m, 8H, NCH₂CH₂, pip-H, NHCH₂CH₂CH₃), 1.40-1.28 (m, 2H, dioxane-H, pip-H), 0.98 (m, 3H, NHCH₂CH₂CH₃). HPLC t_{R}=7.3 min.

### Example 235

### N-{1-[2-(1,3-Dioxan-2-yl)ethyl)piperidin-4-yl}-N-(4-fluorobenzyl)-2-(4-(1-nitropropyl)-phenyl)acetamide, L-tartrate (121JP34)

Adapting a protocol by Vogl & Buchwald (J. Org. Chem., 2002, 67, 106-111), 121JP13 (135 mg, 0.26 mmol), 1-nitropropane (47 mg, 0.52 mmol), Cs₂CO₃ (95 mg, 0.29 mmol), 2-di-tert-butylphosphinobiphenyl (15.5 mg, 52 μmol) and Pd₂ dba₃ (11.9 mg, 13 μmol) were weighed into a flask, DME (2 mL) was added and the reaction was stirred at 60° C. for 20 h. Workup as in 121JP13 followed by preparative TLC (CH₂Cl₂/MeOH, 15:1, 10× eluted) afforded 22 mg (16%) of 121JP34 as a thick colorless oil. The L-tartrate salt of the title compound was prepared as described above.

R_{f}=0.58 (MeOH/CH₂Cl₂ 1:10). LCMS m/z 528 [M+H]⁺. HPLC t_{R}=8.1 min.

### Example 236

### N-{1-[2-(1,3-Dioxan-2-yl)ethyl)piperidin-4-yl}-N-(4-fluorobenzyl)-2-[4-(2-oxoppyrrolidin-1-yl)phenyl)acetamide, L-tartrate (121JP31)

Adapting a protocol by Yin & Buchwald (J. Am. Chem. Soc., 2002, 124, 6043-6048), 121JP13 (124 mg, 0.24 mmol), pyrrolidone (24.7 mg, 0.29 mmol), Cs₂CO₃ (111 mg, 0.34 mmol), Xantphos (20.8 mg, 0.036 mmol) and Pd₂ dba₃ (11.0 mg, 0.012 mmol) were weighed into a flask, dioxane (2 mL) was added and the reaction was stirred at 90° C. for 70 h. Workup as in 121JP13 and purification as in 121JP27 afforded 8 mg (7%) of 121JP31 as a thick colorless oil. The L-tartrate salt was prepared as described above.

R_{f}=0.31 (MeOH/CH₂Cl₂ 1:10). LCMS m/z 524 [M+H]⁺. ¹H-NMR (CDCl₃, rotamers 0.6:0.4) δ 7.60-6.80 (m, 8H), 4.60-4.50 (m, 0.4H, pip-H), 4.47 (t, 1H, J=5.1, dioxane-H) 4.42 and 4.38 (2s, 2H, benzyl-H), 4.04-3.97 (m, 2H, dioxane-H), 3.82-3.60 (m, 5.4H, pip-H, dioxane-H, benzyl-H, pyrrol-H), 3.25 (s, 1.2H, benzyl-H), 2.90-2.72 (m, 2H, pip-H), 2.60-2.50 (m, 2H, pyrrol-H), 2.39-2.32 (m, 2H, NCH₂) 2.18-1.90 (m, 4.2H, dioxane-H, pip-H, pyrrol-H), 1.81-1.40 (m, 6H, pip-H, NCH₂CH₂), 1.32-1.18 (m, 1.8H, dioxane-H, pip-H). HPLC t_{R}=4.9 min.

### Example 237

### N-{1-[2-(1,3-Dioxan-2-yl)ethyl)piperidin-4-yl}-N-(4-fluorobenzyl)-2-(4-isobutylsulfanyl-phenyl)acetamide, L-tartrate (121JP33)

Adapting a protocol by Li (J. Org. Chem., 2002, 67, 3643-3650), 121JP13 (120 mg, 0.231 mmol), 2-methyl-1-propanethiol (25 mg, 0.28 mmol), [(t-Bu)₂P(OH)]₂PdCl₂ (11.6 mg, 0.0231 mmol) and NaOtBu (44 mg, 0.46 mmol) were weighed into a flask, toluene (2 mL) was added and the reaction was stirred at 110° C. for 16 h. Workup as in 121JP13 and purification as in 121JP27 afforded 1.7 mg (1.4%) of 121JP33 as a thick colorless oil. The L-tartrate salt of the title compound was prepared as described above.

R_{f}=0.46 (MeOH/CH₂Cl₂ 1:10). LCMS m/z 529 [M+H]⁺. ¹H-NMR (CDCl₃, rotamers 0.6:0.4) δ 7.24-6.82 (m, 8H), 4.57-4.48 (m, 0.4H, pip-H), 4.47 (t, 1H, J=5.1, dioxane-H), 4.45 and 4.38 (2s, 2H, benzyl-H), 4.05-3.95 (m, 2H, dioxane-H), 3.72 (s, 0.8H, benzyl-H), 3.70-3.60 (m, 2.6H, pip-H, dioxane-H) 3.44 (s, 1.2H, benzyl-H), 2.87-2.75 (m, 2H, pip-H), 2.72 (t, 2H, J=6.5, SCH₂CH(CH₃)₂)), 2.38-2.28 (m, 2H, NCH₂), 2.05-1.88 (m, 2.2H, dioxane-H, pip-H), 1.81-1.48 (m, 7H, NCH₂CH₂, pip-H, SCH₂CH(CH₃)₂), 1.30-1.20 (m, 1.8H, dioxane-H, pip-H), 0.98 (t, 6H, J=6.7, SCH₂CH(CH₃)₂). HPLC t_{R}=8.8 min.

### Example 238

### N-{1-[2-(1,3-Dioxan-2-yl)ethyl)piperidin-4-yl}-N-(4-fluorobenzyl)-2-(4-iodophenyl)-acetamide, L-tartrate (121JP40)

The title compound was prepared by the procedure described above 117NLS87-A using 118AF52-95 (400 mg, 1.24 mmol) and 4-iodophenylacetic acid (1.22 g, 4.64 mmol). Workup as in 121JP13 and purification as in 121JP34 gave 320 mg (46%) of 121JP40 as a colorless thick oil. The L-tartrate salt was prepared as described above.

R_{f}=0.52 (MeOH/CH₂Cl₂ 1:10). LCMS m/z 567 [M+H]⁺. ¹H-NMR (CDCl₃, rotamers 0.6:0.4) δ 7.65-7.55 (m, 2H), 7.16-6.85 (m, 6H), 4.59-4.50 (m, 0.6H, pip-H), 4.51 (t, 1H, J=5.0, dioxane-H), 4.50 and 4.42 (2s, 2H, benzyl-H), 4.09-4.00 (m, 2H, dioxane-H), 3.75 and 3.49 (2s, 2H, benzyl-H), 3.74-3.54 (m, 2.4H, pip-H, dioxane-H), 2.85 (d, 2H, J=10.6, pip-H), 2.41-2.35 (m, 2H, NCH₂), 2.08-1.95 (m, 2.2H, dioxane-H, pip-H), 1.88-1.50 (m, 6H, pip-H, NCH₂CH₂), 1.39-1.27 (m, 1.8H, dioxane-H, pip-H). HPLC t_{R}=8.6 mm.

### Example 239

### 2-(4-Acetophenyl)-N-{1-[2-(1,3-dioxan-2-yl)ethyl)piperidin-4-yl}-N-(4-fluorobenzyl)-acetamide, L-tartrate (121JP44)

Adapting a protocol by Cacchi et al (Org. Lett, 2003, 5, 289-293), 121JP40 (68 mg, 0.12 mmol), acetic anhydride (61 mg, 0.6 mmol), Pd₂ dba₃ (1.4 mg, 1.5 μmol), lithium chloride (26 mg, 0.6 mmol) and EtNiPr₂ (31 mg, 0.24 mmol) were weighed into a flask, DMF (0.9 mL) was added and the resulting mixture was stirred at 100° C. for 18 h. Workup as in 121JP13 and purification as in 121JP34 afforded 19 mg (33%) of 121JP44 as a thick colorless oil. The L-tartrate salt was prepared as described above.

R_{f}=0.50 (MeOH/CH₂Cl₂ 1:10). LCMS m/z 483 [M+H]⁺. ¹H-NMR (CDCl₃, rotamers 0.6:0.4) 7.88-7.78 (m, 2H), 7.36-6.84 (m, 6H), 4.58-4.49 (m, 0.4H, pip-H), 4.48-4.46 (m, 1H, dioxane-H), 4.45 and 4.38 (2s, 2H, benzyl-H), 4.05-3.95 (m, 2H, dioxane-H), 3.81 and 3.55 (2s, 2H, benzyl-H), 3.70-3.60 (m, 2.6H, pip-H, dioxane-H) 2.85-2.75 (m, 2H, pip-H), 2.54 and 2.52 (2s, 3H, CH₃), 2.38-2.27 (m, 2H, NCH₂), 2.05-1.92 (m, 2.2H, dioxane-H, pip-H), 1.81-1.45 (m, 6H, NCH₂CH₂, pip-H), 1.32-1.22 (m, 1.8H, dioxane-H, pip-H). HPLC t_{R}=5.5 min.

### Example 240

### 2-[4-(1-Hydroxyiminoethyl)phenyl]-N-{1-[2-(1,3-dioxan-2-yl)ethyl)piperidin-4-yl}-N-(4-fluorobenzyl)acetamide, L-tartrate (121JP48)

121JP44 (14 mg, 29 μmol), pyridine (4.6 mg, 58 μmol) and ethanol (5 mL) were placed in a flask, to which hydroxylamine hydrochloride (4.1 mg, 58 μmol) was added and the resulting mixture was stirred at rt for 5 h. Workup as in 121JP13 and purification as in 121JP34 afforded 7 mg (49%) of 121JP48 as a thick colorless oil. The L-tartrate salt was prepared as described above.

R_{f}=0.40 (MeOH/CH₂Cl₂ 1:10). LCMS m/z 498 [M+H]⁺. ¹H-NMR (CDCl₃, rotamers 0.6:0.4) 7.63-7.51 (m, 2H), 7.33-6.88 (m, 6H), 4.66-4.58 (m, 0.4H, pip-H), 4.56-4.53 (m, 1H, dioxane-H), 4.51 and 4.40 (2s, 2H, benzyl-H), 4.10-4.04 (m, 2H, dioxane-H), 3.85 and 3.58 (2s, 2H, benzyl-H), 3.78-3.67 (m, 2.6H, pip-H, dioxane-H) 2.97-2.83 (m, 2H, pip-H), 2.47-2.37 (m, 2H, NCH₂), 2.26 and 2.24 (2s, 3H, CH₃), 2.12-1.98 (m, 2.2H, dioxane-H, pip-H), 1.88-1.58 (m, 6H, NCH₂CH₂, pip-H), 1.37-1.29 (m, 1.8H, dioxane-H, pip-H). HPLC t_{R}=4.0 min.

### Example 241

### N-{1-[2-(1,3-Dioxan-2-yl)ethyl)piperidin-4-yl}-N-(4-fluorobenzyl)-2-(4-morpholin-4-yl-phenyl)acetamide, L-tartrate (121JP49)

Adapting a protocol by Buchwald et al (Org. Lett., 2002, 4, 581-584), 121JP40 (50 mg, 88 μmol), morpholine (9.2 mg, 106 μmol), CuI (1.7 mg, 8.8 μmol) and K₃PO₄ (37.6 mg, 177 μmol) were weighed into a flask in air atmosphere, ethylene glycol (2 mL) was added and the resulting mixture was stirred at 80° C. for 16 h under air atmosphere. Workup as in 121JP13 and purification as in 121JP34 afforded 4.7 mg (10%) of 121JP49 as a thick colorless oil. The L-tartrate salt was prepared as described above.

R_{f}=0.33 (MeOH/CH₂Cl₂ 1:10). LCMS m/z 526 [M+H]⁺.¹H-NMR (CDCl₃, rotamers 0.6:0.4) δ 7.18-6.72 (m, 8H), 4.62 and 4.37 (2s, 2H, benzyl-H), 4.57-4.50 (m, 0.4H, pip-H), 4.50-4.42 (m, 1H, dioxane-H), 4.05-3.95 (m, 2H, dioxane-H), 3.82-3.75 (m, 4H, morph-H), 3.69 and 3.43 (2s, 2H, benzyl-H), 3.68-3.61 (m, 2.6H, pip-H, dioxane-H), 3.12-3.03 (m, 4H, morph-H), 2.85-2.75 (m, 2H, pip-H), 2.38-2.27 (m, 2H, NCH₂), 2.07-1.90 (m, 2.2H, dioxane-H, pip-H), 1.82-1.45 (m, 6H, NCH₂CH₂, pip-H), 1.30-1.22 (m, 1.8H, dioxane-H, pip-H). HPLC t_{R}=6.2 min.

### Example 242

### N-{1-[2-(1,3-Dioxan-2-yl)ethyl)piperidin-4-yl}-N-(4-fluorobenzyl)-2-(4-pyrazol-1-yl-phenyl)acetamide, L-tartrate (121JP56)

Adapting a protocol by Buchwald et al (J. Am. Chem. Soc., 2001, 123, 7727-7729), 121JP40 (48 mg, 85 μmol), pyrazole (7 mg, 102 μmol), CuI (0.4 mg, 1.7 μmol), racemic trans-1,2-cyclohexanediamine (1.0 mg, 8.5 μmol), and K₂CO₃ (25 mg, 181 μmol) were weighed into a flask, dioxane (1.5 mL) was added and the resulting mixture was stirred at 110° C. for 60 h. Workup as in 121JP13 and purification as in 121JP34 afforded 3.9 mg (9%) of 121JP56 as a thick colorless oil. The L-tartrate salt was prepared as described above.

R_{f}=0.27 (MeOH/CH₂Cl₂ 1:10). LCMS m/z 507 [M+H]⁺. ¹H-NMR (CDCl₃, rotamers 0.6:0.4) δ 7.86 and 7.82 (2d, 1H, J=2.2, pyraz-H), 7.64 (d, 1H, J=4.4, pyraz-H), 7.62-6.83 (m, 8H), 6.42-6.36 (m, 1H, pyraz-H), 4.60-4.49 (m, 0.6H, pip-H), 4.48 (t, 1H, J=5.1, dioxane-H), 4.45 and 4.38 (2s, 2H, benzyl-H), 4.05-3.95 (m, 2H, dioxane-H), 3.80 and 3.54 (2s, 2H, benzyl-H), 3.70-3.61 (m, 2.4H, pip-H, dioxane-H) 2.85-2.75 (m, 2H, pip-H), 2.38-2.28 (m, 2H, NCH₂), 2.07-1.90 (m, 2.2H, dioxane-H, pip-H), 1.82-1.45 (m, 6H, NCH₂CH₂, pip-H), 1.35-1.22 (m, 1.8H, dioxane-H, pip-H). HPLC t_{R}=6.4 min.

### Example 243

### 2-(2-Bromopropyl)-1,3-dioxane (121JP80)

Adapting a procedure of Büchi and Wüest (J. Org. Chem., 1969, 34, 1122-1123), crotonaldehyde (3 g, 43 mmol) was added dropwise to conc. aq. HBr (5.2 g, 64 mmol) over 5 min at 5° C. under air atmosphere. After 15 min of stirring at 5° C., during which time the mixture changed from colorless to brownish, 1,3-propanediol (8.1 g, 107 mmol) was added and the reaction was stirred at 5° C. for further 0.5 h before allowing it warm to rt, and finally stirring it at rt for 2 h. The crude reaction mixture was then n-heptane extracted (2×200 mL), the combined n-heptane extracts were Na₂SO₄ dried, evaporated in vacuo and 121JP80 was isolated by Kugelrohr distillation (75° C., 0.18 mmHg) to obtain 124 mg (1.4%) of the title compound as a colorless liquid.

Characterization Data: ¹H-NMR (CDCl₃) δ 4.73 (dd, 1H, J=7.2, 3.4), 4.26-4.18 (m, 1H), 4.15-4.06 (m, 2H), 3.83-3.75 (m, 2H), 2.15-1.97 (m, 3H), 1.71 (d, 3H, J=6.6), 1.38-1.32 (m, 1H).

### Example 244

### N-{1-[2-(1,3-Dioxan-2-yl)-1-methylethyl]piperidin-4-yl}-N-(4-fluorobenzyl)-2-(4-iso-butoxyphenyl)-acetamide, L-tartrate (121JP84)

The title compound was prepared by the procedure described above 103NLS63-F using 103NLS56 (202 mg, 0.51 mmol) and 121JP80 (124 mg, 0.59 mmol) as the alkylating agent. Workup as in 121JP13 and purification as in 121JP34 gave 1.9 mg (0.7%) of 121JP84 as a thick oil. The L-tartrate salt was prepared as described above.

R_{f}=0.43 (MeOH/CH₂Cl₂ 1:10). LCMS m/z 527 [M+H]⁺. ¹H-NMR (CDCl₃, rotamers 0.5:0.5) δ 7.19-6.80 (m, 8H), 4.50-4.33 (m, 3.5H, dioxane-H, benzyl-H, pip-H), 4.04-3.93 (m, 2H, dioxane-H), 3.72-3.55 (m, 5.5H, dioxane-H, benzyl-H, pip-H, OCH_{2OiBu}), 3.42 (s, 1H, benzyl-H), 2.78-2.59 (m, 2H, pip-H), 2.34-2.16 (m, 1H, NCH), 2.08-1.89 (m, 2H, pip-H, CH_{OiBu}), 1.79-0.77 (m, 17H, CH_{3OiBu}, NCHCH₃, NCHCH₂, pip-H, dioxane-H). HPLC t_{R}=8.5 min.

### Example 245

### 4-Iodophenylacetic acid ethyl ester (121JP58)

4-Iodophenylacetic acid (3 g), ethanol (20 mL) and conc. H₂SO₄ (5 mL) were refluxed overnight. Ca. 15 mL ethanol was then evaporated, the residue was extracted with dichloromethane (3×100 mL), the combined organic extracts were washed with sat. aq. NaHCO₃, dried over Na₂SO₄ and evaporated in vacuo to afford 2.97 g (90%) of 121JP58 as a yellow oil.

Characterization Data: ¹H-NMR (CDCl₃) δ 7.62 (d, 2H, J=8.4), 7.02 (d, 2H, J=8.4), 4.07 (q, 2H, J=7.0), 3.59 (s, 2H), 1.12 (t, 3H, J=7.0).

### Example 246

### Pyrazol-1-ylphenylacetic acid ethyl ester (121JP64)

121JP58 (290 mg, 1.0 mmol) was treated identically as 121JP40 for the synthesis of 121JP56. After heating the reaction to 110° C. for 72 h and workup as in 121JP13, the crude mixture was purified by VFC(CH₂Cl₂/MeOH 1:0→20:1) to furnish 180 mg (78%) of 121JP64 as a yellow oil.

Characterization Data: ¹H-NMR (CDCl₃) δ 7.92 (dd, 1H, J=2.3, 1.0), 7.72 (d, 1H, J=1.3), 7.62 (d, 2H, J=8.7), 7.39 (d, 2H, J=8.7), 6.42 (dd, 1H, J=2.5, 1.9), 4.18 (q, 2H, J=7.0), 3.61 (s, 2H), 1.22 (t, 3H, J=7.1).

### Example 247

### 4-Pyrazol-1-ylphenylacetic acid (121JP68, 87)

121JP64 (180 mg, 0.78 mmol), lithium hydroxide monohydrate (164 mg, 3.9 mmol), H₂O (10 mL) and THF (10 mL) were stirred overnight at rt. The crude mixture was then extracted with dichloromethane (3×150 mL), the pH of the aqueous phase was adjusted to ca. pH 3 using 4M HCl and extracted with dichloromethane (3×150 mL). The combined organic layers were dried over Na₂SO₄, filtered and evaporated in vacuo to provide 128 mg (81%) of 121JP68 as a yellow solid.

Characterization Data: ¹H-NMR (CDCl₃) δ 7.90 (m, 1H), 7.75 (m, 1H), 7.63 (d, 2H, J=8.6), 7.38 (d, 2H, J=8.6), 6.45 (m, 1H), 3.68 (s, 2H).

### Example 248

### N-{1-[2-(1,3-Dioxan-4-yl)ethyl)piperidin-4-yl}-N-(4-fluorobenzyl)-2-(4-pyrazol-1-yl-phenyl)acetamide, L-tartrate (121JP9 1)

The title compound was prepared by the general procedure described above 117NLS87-A using 128NLS52 (87 mg, 0.27 mmol) and 121JP87 (60 mg, 0.27 mmol). Workup as in 121JP13 and purification as in 121JP34 gave 25 mg (18%) of 121JP91 as a colorless oil. The L-tartrate salt was prepared as described above.

R_{f}=0.34 (MeOH/CH₂Cl₂ 1:10). LCMS m/z 507 [M+H]⁺. ¹H-NMR (CDCl₃, rotamers 0.5:0.5) δ 7.92 and 7.88 (2d, 1H, J=2.2, pyraz-H), 7.71 (d, 1H, J=4.7, pyraz-H), 7.69-6.90 (m, 8H), 6.48-6.42 (m, 1H, pyraz-H), 5.00 (d, 1H, J=6.3, dioxane-H), 4.65 (d, 1H, J=6.4, dioxane-H), 4.63-4.55 (m, 0.5H, pip-H), 4.52 and 4.46 (2s, 2H, benzyl-H), 4.10-4.02 (m, 1H, dioxane-H), 3.86 and 3.57 (2s, 2H, benzyl-H), 3.78-3.55 (m, 2.5H, pip-H, dioxane-H) 2.93-2.82 (m, 2H, pip-H), 2.49-2.30 (m, 2H, NCH₂), 2.10-1.98 (m, 1H, pip-H), 1.90-1.33 (m, 9H, NCH₂CH₂, pip-H, dioxane-H). HPLC t_{R}=5.2 min.

### Example 249

### N-[1-((R)-3,5-Dihydroxypentyl)piperidine-4-yl]-N-(4-fluorobenzyl)-2-(4-isobutoxyphenyl)acetamide, tartrate (130AF93-189)

The desired compound was synthesized from (S)-5-[(4-methylbenzenesulfonyl)oxy]pentane-1,3-diol (Moune et al, J. Org. Chem., 1997, 62, 3332-3339) and 103NLS56 using the same method as described for the preparation of 130AF65-182.

R_{f}=0.48 (MeOH/CH₂Cl₂, 10:90). LCMS m/z 501 [M+H]⁺. HPLC t_{R}=7.4 min.

### Example 250

### N-{1-[2-((4R)-1,3-Dioxane-4-yl)ethyl]piperidine-4-yl}-N-(4-fluorobenzyl)-2-(4-isobutoxyphenyl)acetamide, tartrate (130AF95-190)

The desired compound (7.9 mg, 55%) was synthesized from 130AF93-189 (18.6 mg, 0.028 mmol) using the same method as for the synthesis of 130AF67-183. The enantiomeric excess was determined to be 99% using chiral HPLC analysis (Chiralpak AD column, 4.6×250 mm; heptane/I—PrOH 50:50, 0.3% DEA; 0.5 mL/min; t_{R} 22.7 min). The ¹H NMR and LCMS data were identical with 130AF67-183.

### Example 251

### 4-(1,2,4-Triazol-4-yl)phenylacetic acid (141JP01)

Adapting a protocol by Catarzi et al (J. Med. Chem., 2001, 44, 3157-3165), diformylhydrazine (352 mg, 4.0 mmol) and then dropwise trimethylsilyl chloride (2.53 mL, 20 mmol) and Et₃N (1.30 mL, 9.3 mmol) were added to a suspension of 4-aminophenylacetic acid (201 mg, 1.33 mmol) in anhydrous pyridine. The mixture was heated at 100° C. overnight, volatiles were removed at reduced pressure and the resulting solid was treated with water (6 mL), collected, washed with H₂O and dried in vacuo to provide 251 mg (93%) of 141JP01 as a light brown solid. LCMS m/z 204 [M+H]⁺. ¹H-NMR (DMSO-d₆) δ 9.05 (s, 2H), 7.62 (d, 1H, J=8.6), 7.40 (d, 1H, J=8.2), 3.61 (s, 2H).

### Example 252

### N-{1-[2-(1,3-Dioxan-2-yl)ethyl]piperidin-4-yl}-N-(4-fluorobenzyl)-2-[4(1,2,4-triazol-4-yl)phenyl]acetamide, L-tartrate (141JP13)

The acid 141JP01 (35 mg, 0.17 mmol), N-{1-[2-(1,3-dioxan-2-yl)ethyl]piperidin-4-yl}-N-(4-fluorobenzyl)amine (118AF52-95, 55 mg, 0.17 mmol) and diisopropylethylamine (52 mg, 0.51 mmol) were dissolved in DMF (5 mL). Bromo-tris-pyrrolidino-phosphonium hexafluorophosphate (PyBroP, 119 mg, 0.25 mmol) was added, and the mixture was stirred at rt for 2 h. The mixture was concentrated and passed onto an acidic ion exchange SPE cartridge. The cartridge was washed with methanol (8×4 mL) and the remaining product was eluted off the column with 10% NH₄OH in methanol (2×4 mL) and evaporated. The resulting oil was purified as in 121JP34 to give 47 mg (54%) of 141JP13 as a colorless oil. The L-tartrate salt was prepared as described above.

R_{f}=0.26 (MeOH/CH₂Cl₂ 1:10). LCMS m/z 508 [M+H]⁺. ¹H-NMR (CDCl₃, rotamers 0.5:0.5) δ 8.47 and 8.41 (2s, 1H, —H), 7.48-6.89 (m, 8H, Ar—H), 4.62-4.56 (m, 0.6H, pip-H), 4.56-4.49 (m, 3H, dioxane-H, benzyl-H), 4.10-4.01 (m, 2H, dioxane-H), 3.79 and 3.61 (2s, 2H, benzyl-H), 3.77-3.67 (m, 2.4H, pip-H, dioxane-H), 2.94-2.84 (m, 2H, pip-H), 2.45-2.35 (m, 2H, NCH₂), 2.10-1.43 (m, 9H, dioxane-H, NCH₂CH₂, pip-H), 1.37-1.27 (m, 1H, dioxane-H).

Pharmalogical Data

### Example 253

### Receptor Selection and Amplification (R-SAT) Assays

The functional receptor assay, Receptor Selection and Amplification Technology (R-SAT™), was used (with minor modifications from the procedure described previously (Brann, M. R. U.S. Pat. No. 5,707,798, 1998; Chem. Abstr. 1998, 128, 111548) to screen compounds for efficacy at the 5-HT_{2A} receptor. The RSAT assay was conducted as described herein. The results are summarized below in Table 5.

TABLE 5
Efficiency and pIC50 of Compounds at the 5-HT2A Receptor Compared to Ritanserin.

| | | | | MED in vivo po |
|---|---|---|---|---|
| | | 5HT2A % INH | 5HT2A pIC50 | (mg/kg) |
| 103NLS45-B | 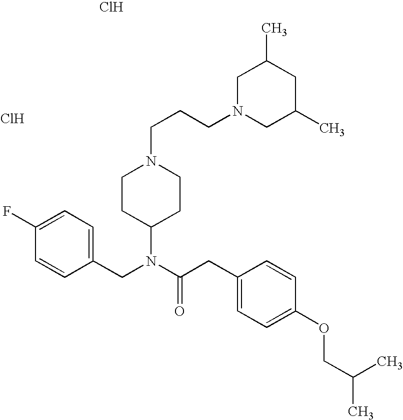 | 84 | 7.6 | |
| 117NLS01 | 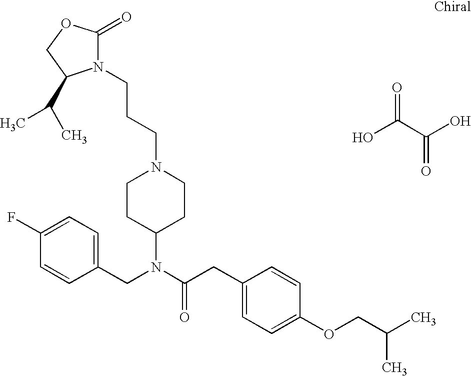 | 104 | 9.7 | 1 |
| 103NLS63-F | 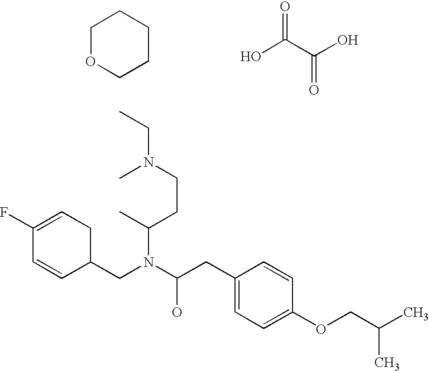 | 101 | 9.5 | |
| 103NLS69-A | 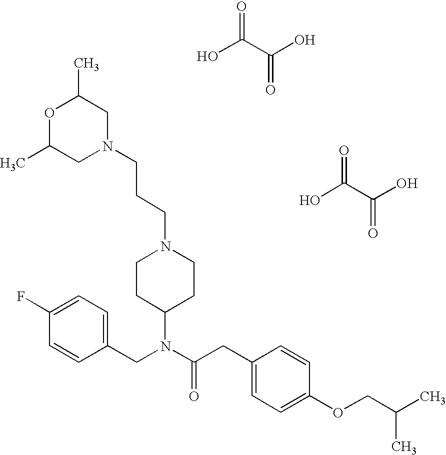 | 96 | 8.7 | |
| 117NLS03-B | 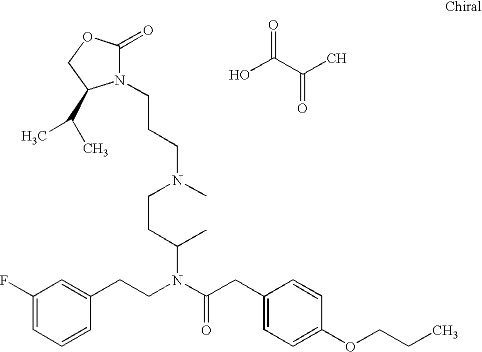 | 85 | 8.4 | |
| 117NLS25 | 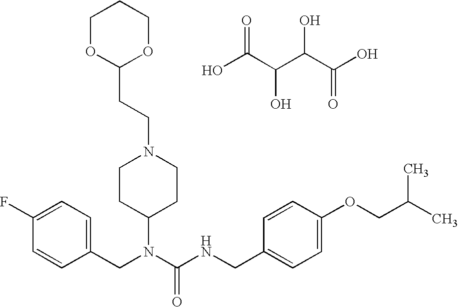 | 94 | 8.9 | |
| 128NLS22-A | 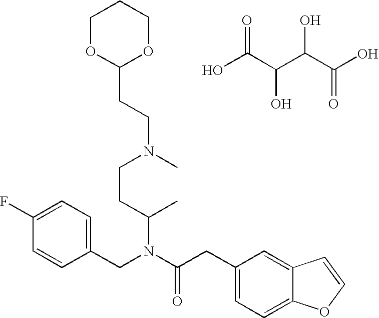 | 98 | 8.5 | |
| 118AF37-88 | 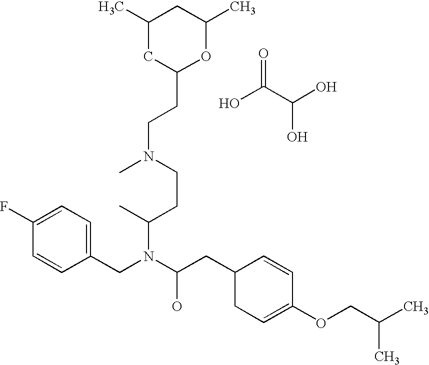 | 105 | 9.2 | |
| 098AF76-65 | 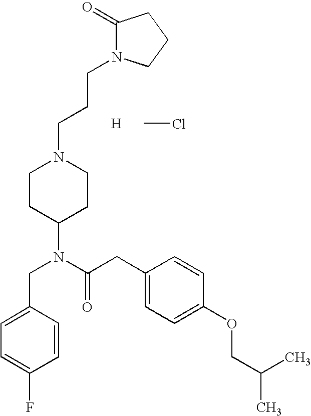 | 100 | 9.1 | |
| 118AF16-80 | 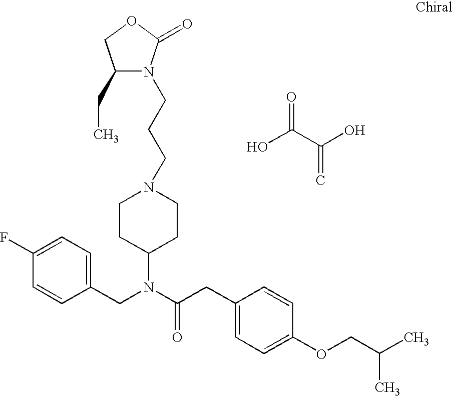 | 103 | 9.1 | |

### Example 254

### In Vivo Determination of Behavioral Effects Animals and Apparatus

Male NSA mice (Harlan; San Deigo, Calif.) were used as subjects. Mice weighed 20-30 g. Animals were housed 8/cage in the One Cage system (One Cage; Lab Products, Inc., Seaford, Del.) with bedding (⅛ inch Bed “O” Cob; Harlan Teklad, Madison, Wis.) in a room with controlled temperature 22±3° C. and a 12 hour light:dark cycle (lights on 6 am). Water and standard rodent chow (Harlan Teklad) were continuously available in the home cage. For testing, plastic locomotor activity cages (20×20×30 cm; AccuScan Instruments, Columbus, Ohio) were equipped with photocell beams for monitoring horizontal activity. Data were collected using Versamax computer software (AccuScan Instruments).

Procedure.

For determination of spontaneous activity, test compounds were administered alone (s.c. 10 min or p.o. 30 min before the session). For hyperactivity experiments, mice were injected with 0.3 mg/kg MK-8011.p. 15 min precession (the peak dose for producing hyperactivity in an inverted-U dose-effect curve as determined in pilot experiments) in combination with vehicle or test compound. Motor activity data were collected during a 15 min session in a lit room. Mice had no prior exposure to the motor cages. Each dose or dose combination was tested in a separate group of mice (n=8).

Data Analysis.

Distance traveled (cm) was calculated and averaged across animals in a group. An analysis of variance (ANOVA) and post-hoc Dunnett's t-test comparisons to vehicle control were conducted for each dose-response function. The lowest dose found to be significantly different from vehicle control was defined as the minimum effective dose (MED).

General LC-MS Procedure for Example 255 to 287.

HPLC/MS analyses were performed using either of two general methods (Method A or Method B).

Method A: Agilent HP1100 HPLC/MSD.

G1312A binary pump, G1313A autosampler, G1316A column compartment, G1315A diode array detector (190-450 nm), 1946A MSD, electrospray ionization.

Chromatography: mobile phase: 8 mM ammoniumacetate in water/acetonitrile. Gradient start at 70% org. up to 100% org. over 12 min, down to 70% org. over 0.5 min, held for 3.5 min. Total runtime 16 min. Flowrate 1 mL/min

Column: Phenomenex Luna C₁₈(2) 3 μm, 75×4.6 mm.

MS parameters: Drying gas, 10 L/min. Nebulizer pressure, 40 psig. Gas temp, 350° C. VCap, 4000.

Method B: Waters/Micromass HPLC/MS.

600 LC-pump, 2700 sample manager, 996 diode array detector (190-450 nm), Micromass ZMD-mass-spectrometer, electrospray ionization.

Chromatography: mobile phase: 10 mM ammoniumacetate in water/acetonitrile. Gradient start at 30% org. for 0.5 min, up to 100% org. over 9.5 min, held for 2 min, down to 30% org. over 0.5 min, held for 5.5 min. Total run time 18 min. Flowrate, 1 mL/min.

Column: Phenomenex Luna C₁₈(2) 3 μm, 75×4.6 mm.

MS parameters: Desolvation gas, 404 L/h. Capillary, 5.3 kV. Cone, 36 V. Extractor, 3 V. Source block temp, 130° C. Desolvation temp, 250° C.

### Example 255

### 4-(4-Fluorobenzyl)-3-(4-isobutoxybenzyl)-8-methyl-1-oxa-3,8-diazaspiro[4.5]decan-2-one, hydrochloride (69NLS75)

### Preparation of 4-[1-Carboxy-2-(4-fluorophenyl)-ethyl]-4-hydroxy-1-methyl-piperidine-(69NLS42)

n-Butyllithium (1.6 M in hexane, 18.8 mL, 30 mmol) was added dropwise under stirring over 5 min to a cooled solution of N,N-diisopropylamine (4.2 mL, 30 mmol) in THF (25 mL) at −40° C. The solution was warmed to 0° C. and a solution of 3-(4-fluoro-phenyl)propionic acid (2.53 g, 15 mmol) in THF (20 mL) was added. The mixture was stirred for 30 min at rt, cooled to −78° C. and N-methylpiperidone (2.2 mL, 18 mmol) added dropwise over 15 min. The solution was allowed to warm to rt and poured into a stirred mixture of diethylether (100 mL) and water (60 mL). The organic layer was discarded, the aqueous layer extracted with another portion of diethylether. The aqueous layer was acidified with 4 M aq. HCl and extracted once with dichloromethane and with n-butanol (3×100 mL). The combined n-butanol-extracts were concentrated in vacuo, giving 69NLS42 as a light-yellow oil, which is used without further purification.

R_{f}=0.13 (MeOH/CH₂Cl₂ 1:9).

### Preparation of 4-(4-Fluorobenzyl)-8-methyl-1-oxa-3,8-diaza-spiro[4.5]decan-2-one (69NLS44)

The crude piperidine derivative 69NLS42 (ca. 15 mmol) was dissolved in toluene (200 mL), triethylamine (4.18 mL, 30 mmol) and diphenylphosphoryl azide (3.88 mL, 18 mmol) were added, and the mixture refluxed overnight. The solvent was removed in vacuo, the residue dissolved in dichloromethane (200 mL) and extracted with 1M HCl (3×100 mL). The combined aqueous extracts were basified with 20% aq. KOH solution and extracted with dichloromethane (3×150 mL). The combined organic layers were dried over Na₂SO₄, filtered and evaporated to give 69NLS44 (2.06 g, 49% over two steps) as a yellow solid, which was used without further purification.

R_{f}=0.25 (MeOH/CH₂Cl₂ 1:9). LCMS m/z 278 [M+H]⁺. HPLC t_{R}=1.9 min (method B).

### Preparation of 4-isobutoxy benzyl bromide (69NLS69)

The alkylating agent was obtained according to literature procedures from p-cresol by Williamson ether synthesis, followed by a Wohl-Ziegler bromination.

4-(4-Fluorobenzyl)-3-(4-isobutoxybenzyl)-8-methyl-1-oxa-3,8-diaza-spiro[4.5]decan-2-one, hydrochloride (69NLS75)

The oxazolidinone 69NLS44 (crude, ca. 7 mmol) was dissolved in DMF/THF (1:9, 50 mL), sodium hydride (50% in oil, 0.67 g, 14 mmol) added and the suspension stirred at rt for 30 min, before the dropwise addition of the bromide 69NLS69 (1.5 g, 7 mmol). The mixture was stirred at rt for 4 h and then partitioned between ethyl acetate and water. The organic layer was separated and the aqueous layer extracted twice with ethyl acetate. The combined organic extracts were dried over Na₂SO₄, filtered and concentrated in vacuo. The residue was purified by column chromatography on silica gel, eluting with a stepwise gradient of 0-6% methanol in dichloromethane. Repurification of the pooled fractions on an acidic ion-exchange SPE cartridge afforded the free amine of the title compound 69NLS75 (1.10 g, 34%) as a colorless oil. The compound was dissolved in dichloromethane, treated with an excess of 2 M HCl in diethylether, precipitated from n-heptane, to afford the hydrochloride salt as a colorless solid in quantitative yield.

R_{f}=0.39 (MeOH/CH₂Cl₂ 1:9). LCMS m/z 441 [M+H]⁺. ¹H NMR (CDCl₃, free amine) δ 0.96 (d, 6H, J=6.8, CH₃), 1.24-1.32 (m, 1H, pip-H), 1.50 (m, 1H, pip-H), 1.62-1.70 (m, 1H, pip-H), 1.83 (m, 1H, pip-H), 1.96-2.06 (m, 1H, CH(CH₃)₂), 2.17 (s, 3H, NCH₃), 2.26-2.34 (m, 2H, pip-H), 2.46 (m, 1H, pip-H), 2.60 (m, 1H, pip-H), 2.70 (dd, 1H, J=7.3, 14.2, CH₂Ar_{F}), 2.85 (dd, 1H, J=6.6, 14.2, CH₂Ar_{F}), 3.34 (t, 1H, J=7.0, H-4), 3.57 (d, 1H, J=15.2, CH₂Ar_{OiBu}), 3.63 (d, 2H, J=6.6, OCH₂CH), 4.69 (d, 1H, J=15.2, CH₂Ar_{OiBu}), 6.73-6.97 (m, 8H, Ar—H). ¹³C NMR (CDCl₃) δ 19.5, 28.5, 31.3, 34.2, 36.8, 46.1, 46.3, 51.2, 51.3, 63.5, 74.7, 79.6, 114.9, 116.0 (d, J_{C-F}=21.0), 127.7, 129.6, 130.6 (d, J_{C-F}=7.7), 132.9 (d, J_{C-F}=3.4), 157.3, 159.2, 162.0 (d, J_{C-F}=244.0). HPLC t_{R}=10.4 min (method B).

### Example 256

### 3-(4-Cyclopropylmethoxybenzyl)-4-(4-fluorobenzyl)-8-methyl-1-oxa-3,8-diaza-spiro[4.5]decan-2-one, hydrochloride (69NLS52)

The title compound was obtained by a procedure similar to the one described for 69NLS75, from 69NLS44 and the appropriate benzylbromide derivative.

Alternatively, 69NLS44 can be first alkylated with 4-acetoxy benzylbromide, followed by basic treatment and alkylation of the resulting free hydroxyl function with cyclopropylmethyl bromide.

R_{f}=0.45 (MeOH/CH₂Cl₂ 1:9). LCMS m/z 439 [M+H]⁺. ¹H NMR (CD₃OD, free amine) δ 0.33 and 0.60 (2m, 4H, CH(CH₂)₂), 1.20-1.26 (m, 1H, CH(CH₂)₂), 1.37-1.45 (m, 1H, pip-H), 1.58 (m, 1H, pip-H), 1.79-1.86 (m, 1H, pip-H), 1.98 (m, 1H, pip-H), 2.26 (s, 3H, NCH₃), 2.34-2.42 (m, 2H, pip-H), 2.63 (m, 1H, pip-H), 2.77 (m, 1H, pip-H), 2.87 (dd, 1H, J=7.0, 14.2, CH₂Ar_{F}), 2.98 (dd, 1H, J=7.0, 14.2, CH₂Ar_{F}), 3.58 (t, 1H, J=7.0, H-4), 3.72 (d, 1H, J=15.2, CH₂Ar_{OMecPr}), 3.80 (d, 2H, J=6.8, OCH₂), 4.61 (d, 1H, J=15.2, CH₂Ar_{OMecPr}), 6.84-7.21 (m, 8H, Ar—H). HPLC t_{R}=6.5 min (method A).

### Example 257

### 4-(4-Fluorobenzyl)-3-(4-isobutoxybenzyl)-2-oxo-1-oxa-3,8-diazaspiro[4.5]decane-8-carboxylic acid tert-butyl ester (69NLS77)

### Preparation of 4-[1-Carboxy-2-(4-fluoro-phenyl)-ethyl]-4-hydroxy-piperidine-1-carboxylic acid tert-butyl ester (69NLS56)

n-Butyllithium (1.6 M in hexane, 8.8 mL, 14.0 mmol) was added dropwise under stirring over 5 min to a cooled solution of N,N-diisopropylamine (2.0 mL, 14.0 mmol) in THF (10 mL) at −40° C. The solution was warmed to 0° C. and a solution of 3-(4-fluoro-phenyl)propionic acid (1.18 g, 7.0 mmol) in THF (8 mL) was added. The mixture was stirred for 30 min at rt, cooled to −78° C. and N—BOC-4-piperidone (1.68 g, 8.4 mmol) in THF (7 mL) added dropwise over 15 min. The solution was allowed to warm to rt and poured into a stirred mixture of diethylether (100 mL) and water (50 mL). The organic layer was discarded, the aqueous layer extracted with another portion of diethylether. The aqueous layer was acidified with 2 M aq. HCl to pH 3.5 and extracted with dichloromethane (3×100 mL). The combined extracts were dried over Na₂SO₄, filtered and concentrated in vacuo, giving 69NLS56 as a yellow solid, which is used without further purification.

R_{f}=0.42 (MeOH/CH₂Cl₂ 1:19). LCMS m/z 268 [M-BOC+2H]⁺. HPLC t_{R}=3.7 min (method B).

### Preparation of 4-(4-fluorobenzyl)-2-oxo-1-oxa-3,8-diaza-spiro[4.5]decane-8-carboxylic acid tert-butyl ester (69NLS58)

The crude piperidine derivative 69NLS56 (ca. 7 mmol) was dissolved in toluene (100 mL), triethylamine (2.0 mL, 14.0 mmol) and diphenylphosphoryl azide (1.8 mL, 14.0 mmol) were added, and the mixture refluxed overnight. The solvent was removed in vacuo, the residue dissolved in ethyl acetate (200 mL) and washed with water and brine. The organic layer was dried over Na₂SO₄, filtered and evaporated to give 69NLS58. Purification of the residue on silica gel column chromatography, eluting with a stepwise gradient of 0-2% methanol in dichloromethane, afforded 69NLS58 as a yellow solid (1.47 g, 58% overall yield).

R_{f}=0.63 (MeOH/CH₂Cl₂ 1:19). R_{f}=0.33 (ethyl acetate/n-heptane 1:1). LCMS m/z 265 [M-BOC+2H]⁺. HPLC t_{R}=10.3 min (method B).

### Preparation of 4-(4-Fluorobenzyl)-3-(4-isobutoxybenzyl)-2-oxo-1-oxa-3,8-diaza-spiro[4.5]decane-8-carboxylic acid tert-butyl ester (69NLS77)

The title compound was obtained by alkylation of 4-(4-fluorobenzyl)-2-oxo-1-oxa-3,8-diaza-spiro[4.5]decane-8-carboxylic acid tert-butyl ester 69NLS58(7.40 g, 20.3 mmol) with 4-isobutoxybenzylbromide (4.88 g, 20.3 mmol) following the same procedure as described for 69NLS75. After purification of the crude product on silica gel column chromatography, using a stepwise gradient of 0-60% ethyl acetate in n-heptane, the collected compound was dissolved in a minimum amount of ethyl acetate and precipitated from n-heptane, giving after filtration 69NLS77 (5.86 g, 55%) as a colorless solid.

R_{f}=0.64 (ethyl acetate/n-heptane 1:1). LCMS m/z 544 [M+NH₄]⁺. ¹H NMR (CDCl₃) δ 1.02 (d, 6H, J=6.6, CH₃), 1.36-1.60 (m, 12H, C(CH₃)₃, pip-H), 1.88 (m, 1H, pip-H), 2.05-2.11 (m, 1H, CH(CH₃)₂), 2.74 (dd, 1H, J=7.6, 14.2, CH₂Ar_{F}), 2.95 (dd, 1H, J=6.6, 14.2, CH₂Ar_{F}), 3.08 (m, 2H, pip-H), 3.39 (t, 1H, J=7.2, H-4), 3.66 (d, 1H, J=15.0, CH₂Ar_{OiBu}), 3.71 (d, 2H, J=6.6, OCH₂CH), 3.82 (m, 1H, pip-H), 3.92 (m, 1H, pip-H), 4.77 (d, 1H, J=15.0, CH₂Ar_{OiBu}), 6.82-7.03 (m, 8H, Ar—H). HPLC t_{R}=14.8 min (method B).

### Example 258

### 8-(2-[1,3]Dioxolan-2-yl-ethyl)-4-(4-fluorobenzyl)-3-(4-isobutoxybenzyl)-1-oxa-3,8-diaza-spiro[4.5]decan-2-one, hydrochloride (69NLS79-II)

69NLS77 (300 mg, 0.57 mmol) was N-—BOC deprotected by treatment with a solution of TFA (2 mL) in dichlormethane (2 mL) at rt for 1.5 h. The solvent was removed in vacuo, the residue coevaporated twice with acetonitrile and redissolved in the same solvent (10 mL). Potassium carbonate (110 mg, 0.80 mmol) and 2-(2-bromoethyl)-1,3-dioxolane (80 μL, 0.68 mmol) were added, followed by sodium iodide (102 mg, 0.68 mmol) and the mixture stirred for 3 days at 50° C. Partitioning of the mixture between water and dichloromethane, extraction of the aqueous layer twice with dichloromethane, drying of the combined organic layers over Na₂SO₄, filtering and evaporation of the solvent gave crude 69NLS79-II. The residue was purified by silica gel column chromatography, eluting with a stepwise gradient of 0-4% methanol in dichloromethane, affording 69NLS79-II (127 mg, 0.24 mmol) as a colorless oil. The compound was converted to its HCl form by treatment with 2M HCl in diethylether as described above for 69NLS75, giving the salt as a colorless powder.

R_{f}=0.61 (MeOH/CH₂Cl₂ 1:9). LCMS m/z 527 [M+H]⁺. ¹H NMR (CD₃OD, free amine) δ 1.00 (d, 6H, J=6.6, CH₃), 1.34-1.42 (m, 1H, pip-H), 1.56 (m, 1H, pip-H), 1.72-1.81 (m, 3H, pip-H, O₂CHCH₂), 1.92 (m, 1H, pip-H), 1.97-2.06 (m, 1H, CH(CH₃)₂), 2.27-2.35 (m, 1H, pip-H), 2.44 (m, 2H, NCH₂), 2.63 (m, 1H, pip-H), 2.76 (m, 1H, pip-H), 2.85 (dd, 1H, J=7.0, 14.2, CH₂Ar_{F}), 2.95 (dd, 1H, J=7.0, 14.2, CH₂Ar_{F}), 3.60 (m, 1H, H-4), 3.69 (d, 2H, J=6.5, OCH₂CH), 3.70 (d, 1H, J=15.2, CH₂Ar_{OiBu}), 3.76-3.82 and 3.85-3.91 (2m, 4H, OCH₂), 4.60 (d, 1H, J=15.2, CH₂Ar_{OiBu}), 4.82 (t, 1H, J=4.7, O₂CH), 6.83-7.19 (m, 8H, Ar—H). HPLC t_{R}=11.3 min (method B).

### Example 259

### 4-(4-Fluorobenzyl)-3-(4-isobutoxybenzyl)-8-(3-morpholin-4-yl-propyl)-1-oxa-3,8-diaza-spiro[4.5]decan-2-one, dihydrochloride (69NLS83)

69NLS77 (300 mg, 0.57 mmol) was N-—BOC deprotected as described in the preparation of 69NLS79-II and dissolved in acetonitrile (3 mL) and DMF (1 mL). To a solution of morpholine (65 μL, 0.74 mmol) in acetonitrile (3 mL) and DMF (1 mL) was added dropwise 1-chloro-3-iodopropane (73 μL, 0.68 mmol) and potassium carbonate (300 mg, 2.17 mmol). The mixture was heated at 50° C. for 3 h before the addition of the solution containing the deprotected spiropiperidine followed by sodium iodide (102 mg, 0.68 mmol). The mixture was stirred overnight at 50° C. and worked up as described for 69NLS79-II. Purification of the residue by silica gel column chromatography, eluting with a stepwise gradient of 0-6% methanol in dichloromethane, afforded the desired compound (127 mg, 40%) as a colorless oil. Treatment of the product in dichloromethane with 2 M HCl in diethylether as described for 69NLS75 gave the corresponding dihydrochloride salt as a colorless solid.

R_{f}=0.48 (MeOH/CH₂Cl₂ 1:9). LCMS m/z 554 [M+H]⁺. ¹H NMR (CD₃OD, free amine) δ 1.01 (d, 6H, J=6.9, CH₃), 1.36-1.43 (m, 1H, pip-H), 1.56-1.83 (m, 4H, pip-H, NCH₂CH₂), 1.94-2.06 (m, 2H, OCH₂CH, pip-H), 2.30-2.49 (m, 10H, pip-H, NCH₂CH₂O, NCH₂CH₂CH₂N), 2.68 (m, 1H, pip-H), 2.79-2.88 (m, 2H, pip-H, CH₂Ar_{F}), 2.97 (dd, 1H, J=6.8, 14.3, CH₂Ar_{F}), 3.56-3.74 (m, 8H, H-4, OCH₂CH, CH₂Ar_{OiBu}, NCH₂CH₂O), 4.60 (d, 1H, J=15.0, CH₂Ar_{OiBu}), 6.84-7.20 (m, 8H, Ar—H). HPLC t_{R}=10.2 min (method B).

### Example 260

### 4-(4-Fluorobenzyl)-3-(4-isobutoxybenzyl)-8-isopropyl-1-oxa-3,8-diaza-spiro[4.5]decan-2-one, hydrochloride (69NLS85)

69NLS77 (320 mg, 0.60 mmol) was N-—BOC deprotected as described in the preparation of 69NLS79-II and dissolved in DMF (3 mL). Potassium carbonate (250 mg, 1.80 mmol) was added, followed by isopropylbromide (68 μL, 0.73 mmol)) and sodium iodide (110 mg, 0.73 mmol) and the mixture stirred overnight at 50° C. Workup was carried out as for 69NLS79-II. The residue was purified by silica gel column chromatography, eluting with a stepwise gradient of 0-6% methanol in dichloromethane, followed by repurification of the compound by acidic ion-exchange column, affording 69NLS85 (150 mg, 53%) as a colorless oil. The compound was converted to its hydrochloride form by treatment with 2M HCl in diethylether as described above, giving the salt as a colorless powder.

R_{f}=0.75 (MeOH/CH₂Cl₂ 1:9). LCMS m/z 469 [M+H]⁺. ¹H NMR (CD₃OD, free amine) δ 1.00 (d, 6H, J=6.7, CH₃), 1.01 (d, 6H, J=6.4, CH₃), 1.35-1.42 (m, 1H, pip-H), 1.56-1.60 (m, 1H, pip-H), 1.72-1.79 (m, 1H, pip-H), 1.93-2.06 (m, 2H, OCH₂CH, pip-H), 2.44-2.51 (m, 2H, pip-H), 2.59-2.75 (m, 3H, pip-H, NCH(CH₃)₂), 2.82-2.97 (m, 2H, CH₂Ar_{F}), 3.56 (t, 1H, J=6.8, H-4), 3.69-3.73 (m, 3H, OCH₂CH, CH₂Ar_{OiBu}), 4.61 (d, 1H, J=15.2, CH₂Ar_{OiBu}), 6.83-7.19 (m, 8H, Ar—H). HPLC t_{R}=11.0 min (method B).

### Example 261

### 4-(4-Fluorobenzyl)-3-(4-isobutoxybenzyl)-8-[2-(2-oxo-imidazolidin-1-yl)-ethyl]-1-oxa-3,8-diaza-spiro[4.5]decan-2-one, hydrochloride (38PH17-HCl)

The title compound was obtained as a colorless solid in 29% yield from 69NLS77 (180 mg, 0.34 mmol) following the same procedure as described for 69NLS85. Toluene-4-sulfonic acid 2-(2-oxo-imidazolidin-1-yl)-ethyl ester was used as the alkylating agent.

R_{f}=0.63 (MeOH/CH₂Cl₂ 1:9). LCMS m/z 539 [M+H]⁺. ¹H NMR (CD₃OD, free amine) δ 1.01 (d, 6H, J=6.8, CH₃), 1.34-1.42 (m, 1H, pip-H), 1.56 (m, 1H, pip-H), 1.77-1.85 (m, 1H, pip-H), 1.94 (m, 1H, pip-H), 1.99-2.07 (m, 1H, CH(CH₃)₂), 2.33-2.42 (m, 2H, pip-H), 2.55 (t, 2H, J=6.6, NCH₂CH₂), 2.71 (m, 1H, pip-H), 2.83-2.89 (m, 2H, pip-H, CH₂Ar_{F}), 2.97 (dd, 1H, J=7.0, 14.2, CH₂Ar_{F}), 3.25 (t, 2H, J=6.6, NCH₂CH₂), 2.89-3.36, 3.42-3.46 (2m, 4H, CONCH₂), 3.56 (t, 1H, J=7.0, H-4), 3.71 (d, 2H, J=6.4, OCH₂CH), 3.70 (d, 1H, J=15.0, CH₂Ar_{OiBu}), 4.60 (d, 1H, J=15.0, CH₂Ar_{OiBu}), 6.84-7.20 (m, 8H, Ar—H). HPLC t_{R}=10.0 min (method B).

### Example 262

### 4-(4-Fluorobenzyl)-3-(4-isobutoxybenzyl)-1-oxa-3,8-diaza-spiro[4.5]decan-2-one, hydrochloride (69NLS81)

69NLS77 (300 mg, 0.57 mmol) was N-—BOC deprotected as described in the preparation of 69NLS79-II. The residue was purified by silica gel column chromatography, eluting with a stepwise gradient of 0-6% methanol in dichloromethane, and purification on an acidic ion-exchange SPE cartridge, afforded 69NLS81 (127 mg, 52%) as a colorless oil. Formation of the hydrochloride salt was carried out as before for 69NLS75 giving the title compound as a colorless solid.

R_{f}=0.29 (MeOH/CH₂Cl₂ 1:9). LCMS m/z 427 [M+H]⁺. ¹H NMR (CD₃OD, free amine) δ 1.00 (d, 6H, J=6.8, CH₃), 1.25-1.33 (m, 1H, pip-H), 1.52 (m, 1H, pip-H), 1.65-1.73 (m, 1H, pip-H), 1.91 (m, 1H, pip-H), 1.98-2.06 (m, 1H, CH(CH₃)₂), 2.73-2.97 (m, 6H, pip-H, CH₂Ar_{F}), 3.53 (t, 1H, J=7.0, H-4), 3.68-3.71 (m, 3H, OCH₂CH, CH₂Ar_{OiBu}), 4.59 (d, 1H, J=15.2, CH₂Ar_{OiBu}), 6.83-7.19 (m, 8H, Ar—H). HPLC t_{R}=9.7 min (method B).

### Example 263

### 8-Cyclopropylmethyl-4-(4-fluorobenzyl)-3-(4-isobutoxybenzyl)-1-oxa-3,8-diaza-spiro[4.5]decan-2-one, hydrochloride (38PH16-HCl)

The title compound was obtained as a colorless solid in 10% yield from 69NLS77 (180 mg, 0.34 mmol) following the same procedure as described for 69NLS85.

R_{f}=0.64 (MeOH/CH₂Cl₂ 1:9). LCMS m/z 481 [M+H]⁺. ¹H NMR (CD₃OD, free amine) δ 0.25, 0.62 (2m, 4H, CH(CH₂)₂), 0.92-0.99 (m, 1H, CH(CH₂)₂), 1.03 (d, 6H, J=6.6, CH₃), 1.52-1.59 (m, 1H, pip-H), 1.71 (m, 1H, pip-H), 1.89-2.15 (m, 3H, pip-H, CH(CH₃)₂), 2.60 (d, 2H, J=6.8, NCH₂), 2.73-2.80 (m, 2H, pip-H), 2.90 (dd, 1H, J=7.2, 14.4, CH₂Ar_{F}), 3.03 (dd, 1H, J=7.0, 14.4, CH₂Ar_{F}), 3.25-3.13 (m, 2H, pip-H), 3.66 (t, 1H, J=7.2, NCH), 3.73 (d, 1H, J=6.4, OCH₂CH), 3.76 (d, 1H, J=15.0, CH₂Ar_{OiBu}), 4.63 (d, 1H, J=15.0, CH₂Ar_{OiBu}), 6.85-7.23 (m, 8H, Ar—H). HPLC t_{R}=11.4 min (method B).

### Example 264

### 4-(4-Fluorobenzyl)-3-(4-isobutoxybenzyl)-8-ethyl-1-oxa-3,8-diaza-spiro[4.5]decan-2-one, hydrochloride (38-PH20)

69NLS77 (190 mg, 0.45 mmol) was N-—BOC deprotected as described in the preparation of 69NLS79-II and dissolved in DMF (3 mL). Potassium carbonate (250 mg, 1.80 mmol) was added, followed by ethylbromide (50 μL, 0.45 mmol)) and the mixture stirred overnight at rt. Workup was carried out as for 69NLS79-II. The residue was purified by silica gel column chromatography, eluting with a stepwise gradient of 0-6% methanol in dichloromethane, followed by repurification of the compound by acidic ion-exchange SPE cartridge, to give 38-PH20(77 mg, 38%) as a colorless oil. The compound was converted to its hydrochloride form by treatment with 2M HCl in diethylether as described above, giving the salt as a colorless powder.

R_{f}=0.52 (MeOH/CH₂Cl₂ 1:9). LCMS m/z 455 [M+H]⁺. ¹H NMR (CDCl₃, salt) δ 0.96 (d, 6H, J=6.6, CH₃), 1.39-1.55 (m, 6H, NCH₂CH₃, pip-H), 1.98-2.04 (m, 2H, pip-H, CH(CH₃)₂), 2.75-2.86 (m, 2H, CH₂Ar_{F}), 2.98 (m, 4H, NCH₂CH₃, pip-H), 3.19 (d, 1H, J=14.6, CH₂Ar_{OiBu}), 3.25-3.50 (m, 3H, pip-H, H-4), 3.62 (d, 2H, J=6.3, OCH₂CH), 4.60 (d, 1H, J=14.6, CH₂Ar_{OiBu}), 6.70-7.19 (m, 8H, Ar—H). HPLC t_{R}=4.7 min (method A).

### Example 265

### 3-(4-Difluoromethoxybenzyl)-4-(4-fluorobenzyl)-8-methyl-1-oxa-3,8-diaza-spiro[4.5]decan-2-one, hydrochloride (84AF8-30)

The title compound was obtained as a colorless solid in 25% yield from 69NLS44 (85 mg, 0.30 mmol) following the same procedure as described for 69NLS75.

R_{f}=0.44 (MeOH/CH₂Cl₂ 6:94). LCMS m/z 434 [M+H]⁺. ¹H NMR (CDCl₃, free amine) δ 1.42-1.50 (m, 1H, pip-H), 1.58-1.63 (m, 1H, pip-H), 1.76-1.83 (m, 1H, pip-H), 1.90-1.95 (m, 1H, pip-H), 2.28 (m, 3H, NCH₃), 2.37-2.45 (m, 2H, pip-H), 2.60 (m, 1H, pip-H), 2.72 (m, 1H, pip-H), 2.80 (dd, 1H, J=6.8, 14.4, CH₂Ar_{F}), 2.87 (dd, 1H, J=7.6, 14.4, CH₂Ar_{F}), 3.45 (t, 1H, J=6.8, NCH), 3.73 (d, 1H, J=15.2, CH₂Ar_{OiBu}), 4.71 (d, 1H, J=15.2, CH₂Ar_{OiBu}), 6.50 (t, 1H, J_{C-F}=73.6, CF₂H), 6.96-7.06 (m, 8H, Ar—H). HPLC t_{R}=2.7 min (method A).

### 8-Methyl-4-(4-methylbenzyl)-3-(4-trifluoromethoxybenzyl)-1-oxa-3,8-diaza-spiro[4.5]decan-2-one, hydrochloride (69NLS21)

### Example 266

### Preparation of 4-[1-Carboxy-2-(4-methylphenyl)-ethyl]-4-hydroxy-1-methyl-piperidine (69NLS13)

The title compound was obtained in analogy with the procedure described for 69NLS42 starting with 3-(4-methylphenyl)propionic acid (0.70 g, 4.26 mmol).

R_{f}=0.14 (MeOH/CH₂Cl₂ 1:9).

### Example 267

### Preparation of 4-(4-Methylbenzyl)-8-methyl-1-oxa-3,8-diaza-spiro[4.5]decan-2-one (69NLS15)

The title compound was obtained from 69NLS13 in analogy with the procedure described for 69NLS44.

R_{f}=0.5 (MeOH/CH₂Cl₂ 1:9). LCMS m/z 275 [M+H]⁺. HPLC t_{R}=3.8 min (method B).

### Example 268

### Preparation of 8-Methyl-4-(4-methylbenzyl)-3-(4-trifluoromethoxybenzyl)-1-oxa-3,8-diaza-spiro[4.5]decan-2-one, hydrochloride (69NLS21)

The title compound was obtained in 24% overall yield by alkylation of 69NLS15 with p-trifluoromethoxy benzylbromide, as described in the procedure for 69NLS75. Conversion of the free amine into the hydrochloride salt was performed as above, giving the title compound as a colorless solid.

R_{f}=0.26 (MeOH/CH₂Cl₂ 1:9). LCMS m/z 449 [M+H]⁺. ¹H NMR (CD₃OD, free amine) δ 1.46-1.53 (m, 1H, pip-H), 1.64 (m, 1H, pip-H), 1.81-1.89 (m, 1H, pip-H), 1.97 (m, 1H, pip-H), 2.24, 2.30 (2s, 6H, Ar—CH₃, NCH₃), 2.30-2.38 (m, 2H, pip-H), 2.63 (m, 1H, pip-H), 2.74 (m, 1H, pip-H), 2.85 (dd, 1H, J=6.7, 14.2, CH₂Ar_{Me}), 2.92 (dd, 1H, J=7.4, 14.2, CH₂Ar_{Me}), 3.67 (t, 1H, J=7.0, H-4), 3.90 (d, 1H, J=15.2, CH₂Ar_{OCF3}), 4.59 (d, 1H, J=15.2, CH₂Ar_{OCF3}), 7.03-7.21 (m, 8H, Ar—H). HPLC t_{R}=10.9 min (method B).

### Example 269

### 3-(4-Methoxybenzyl)-8-methyl-4-(4-methylbenzyl)-1-oxa-3,8-diaza-spiro[4.5]decan-2-one, hydrochloride (69NLS35)

The title compound was obtained in 20% yield in analogy with the procedure described for 69NLS75 by alkylation of 69NLS15 with p-methoxy benzylchloride. In this case however, the reaction was performed at reflux for 3 h. Conversion of the free amine into the hydrochloride salt was performed as above, gave the title compound as a colorless solid.

R_{f}=0.45 (MeOH/CH₂Cl₂ 1:9). LCMS m/z 395 [M+H]⁺. ¹H NMR (CD₃OD, free amine) δ 1.33-1.41 (m, 1H, pip-H), 1.51-1.56 (m, 1H, pip-H), 1.77-1.85 (m, 1H, pip-H), 1.92-1.97 (m, 1H, pip-H), 2.22 and 2.30 (2s, 6H, Ar—CH₃, NCH₃), 2.27-2.36 (m, 2H, pip-H), 2.56 (m, 1H, pip-H), 2.70 (m, 1H, pip-H), 2.81 (dd, 1H, J=7.2, 14.2, CH₂Ar_{Me}), 2.94 (dd, 1H, J=7.0, 14.2, CH₂Ar_{Me}), 3.56 (t, 1H, J=7.0, H-4), 3.72 (d, 1H, J=15.2, CH₂Ar_{OMe}), 3.76 (s, 3H, OCH₃), 4.59 (d, 1H, J=15.2, CH₂Ar_{OMe}), 6.84-7.13 (m, 8H, Ar—H). HPLC t_{R}=6.2 min (method A).

### Example 270

### (4R)- and (4S)-4-(4-fluorobenzyl)-3-(4-isobutoxybenzyl)-8-methyl-1-oxa-3,8-diaza-spiro[4.5]decan-2-one, hydrochloride (78NLS59 and 78NLS62)

The resolution of the racemate 69NLS44 was achieved by transient introduction of a camphanoyl chiral auxiliary. The resulting two diastereomers were separated by fractional crystallization, the camphanic acid substituent removed and the resulting spiropiperidines alkylated with the appropriate benzylbromide derivative.

### Example 271

### Preparation of 3-[(−)-Camphanoyl)]-4-(4-fluorobenzyl)-8-methyl-1-oxa-3,8-diaza-spiro[4.5]decan-2-one and Separation of the Two Diastereomers (78NLS52-crys and 78NLS52-fil)

n-Butyllithium (2.7 M in heptane, 0.53 mL, 1.42 mmol) was added to a cooled solution of 69NLS44 (360 mg, 1.29 mmol) in THF (10 mL) at −78° C., and the mixture stirred for 30 min. A solution of (−)-camphanic acid chloride (307 mg, 1.42 mmol) in THF (2 mL) was added dropwise at −78° C., the solution stirred for 15 min at −78° C. and then 3 h at rt. Saturated ammonium chloride solution (5 mL) was added and the mixture extracted three times with ethyl acetate. The combined organic extracts were washed with sat. NaHCO₃ and brine, dried over Na₂SO₄, filtered and evaporated. The residue was crystallized from ethyl acetate/n-heptane at rt. The light-yellow crystals 78NLS52-crys (155 mg, 26%, >98% de, determined by ¹H NMR) were filtered off, the mother liquor concentrated and purified by silica gel column chromatography, eluting with a stepwise gradient of 0-2% methanol in dichloromethane. The second diastereomer is accumulated in the head fractions, which were evaporated and the residue recrystallized as before. Evaporation of the mother liquor afforded 78NLS52-fil (28 mg, 5%, 98% de determined by ¹H NMR) as a colorless oil.

78NLS52-crys (>98% de): R_{f}=0.40 (MeOH/CH₂Cl₂ 1:9). LCMS m/z 459 [M+H]⁺.¹H NMR (CDCl₃) δ 0.87, 1.09 and 1.15 (3s, 3×3H, CH₃), 1.45-1.53 (m, 1H, pip-H), 1.70-1.80 (m, 3H, pip-H, camph-H), 1.83-1.92 (m, 1H, camph-H), 1.97-2.02 (m, 1H, pip-H), 2.13-2.20 (m, 1H, camph-H), 2.24 (s, 3H, NCH₃), 2.29-2.38 (m, 2H, pip-H), 2.55-2.61 (m, 2H, pip-H, camph-H), 2.66 (m, 1H, pip-H), 2.80 (dd, 1H, J=9.0, 14.2, CH₂Ar), 3.06 (dd, 1H, J=4.9, 14.2, CH₂Ar), 4.59 (dd, 1H, J=4.9, 9.0, H-4), 6.98-7.25 (m, 4H, Ar—H). HPLC t_{R}=8.1 min (method B).

78NLS52-fil (98% de): R_{f}=0.40 (MeOH/CH₂Cl₂ 1:9). LCMS m/z 459 [M+H]⁺.¹H NMR (CDCl₃) δ 0.98, 1.09 and 1.13 (3s, 3×3H, CH₃), 1.59-1.70 (m, 2H, pip-H), 1.73-1.80 (m, 1H, camph-H), 1.84-1.91 (m, 1H, camph-H), 1.97 (m, 2H, pip-H), 2.08-2.15 (m, 1H, camph-H), 2.25 (s, 3H, NCH₃), 2.24-2.37 (m, 2H, pip-H), 2.50-2.60 (m, 2H, pip-H), 2.94-3.01 (m, 2H, CH₂Ar, pip-H), 3.12 (dd, 1H, J=4.1, 14.2, CH₂Ar), 4.32 (dd, 1H, J=4.1, 9.0, H-4), 6.95-7.24 (m, 4H, Ar—H). HPLC t_{R}=8.7 min (method B).

### Example 272

### Preparation of (4R)- and (4S)-4-(4-fluorobenzyl)-8-methyl-1-oxa-3,8-diaza-spiro[4.5]decan-2-one (78NLS57 and 78NLS61).

Lithium hydroxide monohydrate (20 mg, 0.47 mmol) was added to a solution of 78NLS52-crys (100 mg, 0.22 mmol) in THF/water (4 mL, 3:1) at 0° C. After stirring for 1 h at 0° C., sat. aq. NaHCO₃ was added (3 mL), the solution extracted three times with diethylether and the combined organic extracts dried over Na₂SO₄, filtered and evaporated. The crude compound 78NLS57 (60 mg) was used without further purification. In the same way, treatment of 78NLS52-fil (28 mg, 61 μmol) with lithiumhydroxide afforded 78NLS61.

### Example 273

### Preparation of (4R)- and (4S)-4-(4-fluorobenzyl)-3-(4-isobutoxybenzyl)-8-methyl-1-oxa-3,8-diaza-spiro[4.5]decan-2-one, hydrochloride (78NLS59 and 78NLS62)

Alkylation of 78NLS57 (ca. 0.22 mmol) following the method described for 69NLS75 gave the enantiomer 78NLS59 (45 mg, 48%, over two steps). In the same way, alkylation of the second enantiomer 78NLS61 (ca. 61 μmol) gave 78NLS62 (7 mg, 25%, over two steps). Spectrochemical data for both compounds were identical with those determined for 69NLS75. The enantiomeric excess (ee) was determined to be 98% for 78NLS59 and 93% for 78NLS62 using chiral HPLC analysis (Chiracel OD-H column, 4.6×250 mm; hexane/I—PrOH/DEA 95:5:0.2; 0.5 mL/min; t_{R} 20.8 and 23.6 min for 78NLS59 and 78NLS62 respectively).

Subsequent X-ray diffraction analysis of the intermediate 78NLS52-crys allowed the assignment of the (S)-configuration to the early eluting enantiomer (78NLS59, Formula II). Biological evaluation in vitro demonstrated that the (S)-enantiomer is the eutomer at the 5-HT2A receptor (See Table 6).

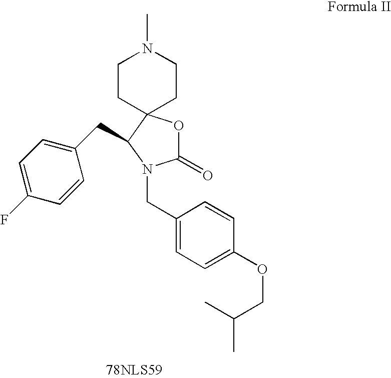

### Example 274

### [2-(4-Fluorophenyl)ethyl]triphenylphosphonium bromide (78NLS66)

4-Fluorophenylethyl bromide (700 mg, 3.44 mmol) was dissolved in toluene (4 mL), triphenylphosphine (904 mg, 3.44 mmol) added and the solution heated for 10 min in a sealed flask at 200° C. under microwave heating. After cooling to rt, the solvent was decanted and the title compound was obtained quantitatively as the remaining glassy solid, which was used without further purification.

R_{f}=0.70 (MeOH/CH₂Cl₂ 1:9). ¹H NMR (CDCl₃) δ 2.99-3.06 (m, 2H, PCH₂CH₂), 4.16-4.23 (m, 2H, PCH₂), 6.86-7.35 (m, 4H, Ar_{F}-H), 7.67-7.90 (m, 15H, PPh₃).

### Example 275

### 4-[2-(4-Fluorophenyl)ethylidenel-piperidine-1-carboxylic acid benzyl ester (103NLS05)

To a suspension of phosphonium bromide 78NLS66 (4.69 g, 10.1 mmol) in THF (100 mL) n-butyllithium (1.6 M in hexane, 6.3 mL, 10.1 mmol) was added at 0° C., giving a deep red solution. After stirring for 1 h at rt, a solution of benzyl 4-oxo-1-piperidinecarboxylate (2.24 g, 9.6 mmol) in THF (10 mL) was added dropwise over 30 min. The mixture was stirred at rt for 20 h, then water was added and the mixture extracted three times with diethylether. The combined organic layers were dried over Na₂SO₄, filtered and concentrated in vacuo. Purification of the residue by silica gel column chromatography, eluting with a stepwise gradient of 0-10% ethyl acetate in n-heptane, afforded the title compound (0.56 g, 17%) as a colorless oil.

R_{f}=0.63 (ethyl acetate/n-heptane 2:3). LCMS m/z 362 [M+Na]⁺, 340 [M+H]⁺. ¹H NMR (CDCl₃) δ 2.24 (m, 2H, pip-H), 2.35 (m, 2H, pip-H), 3.37 (d, 2H, J=7.2, CH₂Ar_{F}), 3.55 (m, 4H, pip-H), 5.16 (s, 2H, OCH₂), 5.43 (t, 1H, J=7.2, CH═), 6.96-7.40 (m, 9H, Ar—H). HPLC t_{R}=10.7 min (method B).

### Example 276

### 2-(4-Fluorobenzyl)-1-oxa-6-aza-spiro[2.5]octane-6-carboxylic acid benzyl ester (103NLS09)

To a solution of the olefin 103NLS05 (79 mg, 0.23 mmol) in dichloromethane (3 mL) at 0° C. was added dropwise a solution of m-chloroperbenzoic acid (70% in H₂O, 69 mg, 0.28 mmol). The mixture was stirred at rt for 24 h, diluted with dichloromethane and washed with 10% aq. NaHCO₃ solution. The organic layer was dried over Na₂SO₄, filtered and evaporated to dryness. The title compound was obtained quantitatively and was used without further purification.

R_{f}=0.53 (ethyl acetate/n-heptane 2:3). LCMS m/z 356 [M+H]⁺. ¹H NMR (CDCl₃) δ 1.39 (m, 1H, pip-H), 1.66 (m, 1H, pip-H), 1.83-1.90 (m, 2H, pip-H), 2.79-2.92 (m, 2H, CH₂Ar_{F}), 3.03 (m, 1H, OCH), 3.39-3.45 (m, 2H, pip-H), 3.83-3.89 (m, 2H, pip-H), 5.16 (s, 2H, OCH₂), 6.98-7.36 (m, 9H, Ar—H). HPLC t_{R}=9.3 min (method B).

### Example 277

### 4-[1-Amino-2-(4-fluorophenyl)-ethyl]-4-hydroxypiperidine-1-carboxylic acid benzyl ester (103NLS28)

To a solution of ammonia in methanol (7 N, 5 mL) the crude epoxide 103NLS09 (520 mg, 1.40 mmol) was added and the solution heated for 20 h in a sealed flask at 100° C. After cooling to rt, the solvent was removed and the residue purified by C₁₈ reversed phase solid-phase extraction, eluting with a stepwise gradient of 0-80% methanol in dichloromethane, giving the aminoalcohol 103NLS28 (459 mg, 88%).

R_{f}=0.62 (MeOH/CH₂Cl₂ 1:9). LCMS m/z 373 [M+H]⁺.¹H NMR (CDCl₃) δ 1.52-1.67 (m, 4H, pip-H), 2.37 (dd, 1H, J=11.3, 13.8, CH₂Ar_{F}), 2.70 (m, 3H, NH₂, OH), 2.81 (dd, 1H, J=2.9, 11.3, CH₂Ar_{F}), 2.97 (dd, 1H, J=2.9, 13.8, CHNH₂), 3.20-3.26 (m, 2H, pip-H), 4.03-4.08 (m, 2H, pip-H), 5.15 (s, 2H, OCH₂), 6.98-7.38 (m, 9H, Ar—H). HPLC t_{R}=5.5 min (method B).

### Example 278

### 5-(4-Fluorobenzyl)-3-oxo-1-oxa-4,9-diaza-spiro[5.5]undecane-9-carboxylic acid benzyl ester (103NLS30C)

The title compound was prepared from aminoalcohol 103NLS28 (303 mg, 0.81 mmol) according to literature procedures (Clark et al., J. Med. Chem. 26:855-861 (1983)), by acylation of the amino function with chloroacetylchloride, followed by halogen exchange with NaI and ring closure of the iodide derivative in presence of tert-butoxide. Purification of the crude product by silica gel column chromatography, eluting with a stepwise gradient of 0-100% ethyl acetate in n-heptane, afforded the title compound (64 mg, 19% overall yield) as a colorless solid.

R_{f}=0.19 (ethyl acetate/n-heptane 3:2). LCMS m/z 458 [M+H]⁺. ¹H NMR (CDCl₃) δ 1.52-1.74 (m, 2H, pip-H), 1.87-1.98 (m, 2H, pip-H), 2.47 (dd, 1H, J=11.7, 13.3, CH₂Ar_{F}), 2.81 (dd, 1H, J=2.7, 13.3, CH₂Ar_{F}), 3.04-3.22 (m, 2H, pip-H), 3.40-3.45 (m, 1H, H-5), 4.05-4.20 (m, 4H, H-2, pip-H), 5.15 (s, 2H, OCH₂Ph), 5.55 (s, 1H, NH), 6.99-7.37 (m, 9H, Ar—H). HPLC t_{R}=12.4 min (method B).

### Example 279

### 5-(4-Fluorobenzyl)-4-(4-isobutoxybenzyl)-3-oxo-1-oxa-4,9-diaza-spiro[5.5]undecane-9-carboxylic acid benzyl ester (103NLS33)

Alkylation of the spirocycle 103NLS30C (61 mg, 0.148 mmol) was performed as described in the preparation of 69NLS75 using 4-isobutoxybenzyl bromide 69NLS69 as the alkylating agent. After purification of the residue by silica gel column chromatography eluting with a stepwise gradient of 0-70% ethyl acetate in n-heptane, the title compound 103NLS33 (62 mg, 73%) was obtained as a colorless oil.

R_{f}=0.37 (ethyl acetate/n-heptane 1:1). LCMS m/z 575 [M+H]⁺. ¹H NMR (CDCl₃) δ 0.79-0.88 (m, 1H, pip-H), 1.02 (d, 6H, J=6.4, CH₃), 1.32-1.37 (m, 1H, pip-H), 1.69-1.81 (m, 2H, pip-H), 2.03-2.09 (m, 1H, CH(CH₃)₂), 2.72 (d, 1H, J=14.3, CH₂Ar_{OiBu}), 2.86-2.95 and 3.07-3.15 (2m, 5H, H-5,CH₂Ar_{F}, pip-H), 3.70 (d, 2H, J=6.4, OCH₂CH), 3.80-3.85 (m, 2H, pip-H), 4.16 (AB, 2H, J=17.6, H-2), 5.06 (s, 2H, OCH₂Ph), 5.35 (d, 1H, J=14.3, CH₂Ar_{OiBu}), 6.78-7.36 (m, 13H, Ar—H). HPLC t_{R}=10.9 min (method B).

### Example 280

### 5-(4-Fluorobenzyl)-4-(4-isobutoxybenzyl)-9-methyl-1-oxa-4,9-diaza-spiro[5.5]undecan-3-one (103NLS35)

The spirocycle 103NLS33 (62 mg, 0.107 mmol) in ethanol (5 mL) was N-CBz-deprotected by hydrogenation in presence of Pd/C (10%, 40 mg) under H₂-balloon pressure. The catalyst was filtered off and the filtrate evaporated under reduced pressure to give crude 5-(4-fluorobenzyl)-4-(4-isobutoxybenzyl)-1-oxa-4,9-diaza-spiro[5.5]undecan-3-one. The residue was dissolved in methanol (3 mL), formaldehyde (37% in water, 0.017 mL) added, followed by the addition of acetic acid (0.03 mL) and sodium cyanoborohydride (60 mg, 0.95 mmol). The solution was stirred at rt for 5 h, then water was added and the mixture basified with 2 N aq. NaOH solution. The mixture was extracted three times with dichloromethane, the combined organic layers washed with sat. ammonium chloride solution and brine, dried over Na₂SO₄, filtered and evaporated to dryness. The residue was purified by column chromatography on silica gel, eluting with a stepwise gradient of 0-7% methanol in dichloromethane, giving 103NLS35B (43 mg, 88% over both steps) as a colorless oil. The compound was converted to its HCl form by treatment with 2 M HCl in diethylether as described above for 69NLS75, affording the salt as a colorless powder.

R_{f}=0.50 (MeOH/CH₂Cl₂ 1:9). LCMS m/z 455 [M+H]⁺. ¹H NMR (CDCl₃) δ 0.94-1.06 (m, 7H, pip-H, CH₃), 1.50-1.57 (m, 1H, pip-H), 1.69-1.76 (m, 2H, pip-H), 2.00-2.09 (m, 2H, pip-H, CH(CH₃)₂), 2.22-2.29 (m, 4H, NCH₃, pip-H), 2.37-2.40 (m, 1H, pip-H), 2.55 (m, 1H, pip-H), 2.62 (d, 1H, J=14.3, CH₂Ar_{OiBu}), 2.87 (dd, 1H, J=7.8, 13.5, CH₂Ar_{F}), 3.06-3.14 (m, 2H, H-5,CH₂Ar_{F}, pip-H), 3.66 (d, 2H, J=6.4, OCH₂CH), 4.12 (AB, 2H, J=17.6, H-2), 5.29 (d, 1H, J=14.3, CH₂Ar_{OiBu}), 6.75-7.18 (m, 8H, Ar—H). HPLC t_{R}=7.5 min (method B).

### Example 281

### 3-Oxo-1,2,8-triaza-spiro[4.5]decane-8-carboxylic acid tert-butyl ester (84AF15)

A solution of hydrazine monohydrate (5.28 mL, 108.8 mmol) in n-butanol (20 mL) was added dropwise to a solution of N-—BOC-4-methoxycarbonyl-methylenepiperidine (1.39 g, 5.44 mmol) in n-butanol (120 mL) at rt. The mixture was stirred for 15 h at 120° C. and the mixture allowed to cool to rt. The solvent was removed by evaporation under reduced pressure, the residue partitioned between ethyl acetate and water and the organic layer dried over sodium sulphate, filtered and evaporated to dryness. Purification of the residue by silica gel column chromatography, eluting with a stepwise gradient of 3-6% methanol in dichloromethane, afforded the desired compound (0.88 g, 63%) as white solid.

R_{f}=0.34 (MeOH/CH₂Cl₂ 6:94). LCMS m/z 200 [M+H-(t-Bu)]⁺, 156 [M+H—BOC]⁺. ¹H NMR (CDCl₃) δ 1.45 (s, 9H, CH₃), 1.61-1.74 (m, 4H, pip-H), 2.34 (s, 2H, H-4), 3.38-3.52 (m, 4H, pip-H), 4.01 (s, 1H, NH), 7.15 (s, 1H, NH).

### Example 282

### 1-(4-Fluorobenzyl)-3-oxo-1,2,8-triaza-spiro[4.5]decane-8-carboxylic acid tert-butyl ester (84AF84-19)

4-Fluorobenzylbromide (0.37 mL, 2.97 mmol) was added dropwise to a solution of 84AF15 (289 mg, 1.13 mmol) in dry DMF (50 mL) at rt. The mixture was stirred at rt for 6 days under argon atmosphere. The solvent was removed by evaporation under reduced pressure and the residue partitioned between chloroform and water. The organic layer was dried over sodium sulphate, filtered and concentrated in vacuo. Purification of the residue by column chromatography on silica gel, eluting with dichloromethane, afforded the desired compound (160 mg, 39%).

R_{f}=0.53 (MeOH/CH₂Cl₂ 6:94). LCMS m/z 308 [M+H—BOC]⁺, 364 [M+H]⁺.¹H NMR (CDCl₃) δ 1.47 (s, 9H, CH₃), 1.71-1.76 (m, 2H, pip-H), 1.87-1.93 (m, 2H, pip-H), 2.45 (s, 2H, H-4), 3.31-3.38 (m, 2H, pip-H), 3.64-3.70 (m, 2H, pip-H), 3.80 (s, 2H, CH₂Ar_{F}), 6.62 (s, 1H, NH), 7.01-7.30 (m, 4H, Ar—H).

### Example 283

### 1-(4-Fluorobenzyl)-2-(4-isobutoxybenzyl)-3-oxo-1,2,8-triaza-spiro[4.5]decane-8-carboxylic acid tert-butyl ester (118AF94-23)

NaH (24 mg, 0.50 mmol) was added slowly to a solution of 84AF84-19 (0.149 g, 0.41 mmol) in dry DMF (5 mL) and the mixture stirred at rt for 30 min. A solution of 4-isobutoxybenzyl bromide 69NLS69 (117 mg, 0.48 mmol) in dry DMF (1 mL) was added dropwise to the mixture. After 1 h stirring at rt the mixture was partitioned between ethyl acetate and water. The organic layer was dried over sodium sulphate, filtered and concentrated in vacuo. Purification of the residue by column chromatography on silica gel, eluting with a stepwise gradient of 3-6% methanol in dichloromethane, followed by preparative reversed phase HPLC (C₁₈) afforded the desired compound (109 mg, 50%).

R_{f}=0.51 (MeOH/CH₂Cl₂ 1:99). LCMS m/z 526 [M+H]⁺, 470 [M+H-(t-Bu)]⁺.¹H NMR (CDCl₃) δ 1.00 (d, 6H, J=6.8, CH(CH₃)₂), 1.35 (m, 4H, pip-H), 1.40 (s, 9H, O(CH₃)₃), 2.06 (m, 1H, CH(CH₃)₂), 2.41 (s, 2H, H-4), 3.08-3.15 (m, 2H, pip-H), 3.22-3.29 (m, 2H, pip-H), 3.67 (d, 2H, J=6.4, OCH₂), 3.92 (m, 4H, CH₂Ar_{F}, CH₂Ar_{OiBu}), 6.77-7.37 (m, 8H, Ar—H).

### Example 284

### 1-(4-Fluorobenzyl)-2-(4-isobutoxybenzyl)-1,2,8-triaza-spiro[4.5]decane-3-one (84AF99-24)

118AF94-23 (100 mg, 0.21 mmol) was N-—BOC deprotected as described in the preparation of 69NLS79-II to give the title compound, which was used without further purification.

R_{f}=0.40 (MeOH/CH₂Cl₂ 1:9). LCMS m/z 426 [M+H]⁺. ¹H NMR (CDCl₃) δ 0.99 (d, 6H, J=6.4, CH(CH₃)₂), 1.35 (m, 4H, pip-H), 1.49 (s, 1H, NH), 2.00-2.05 (m, 1H, CH(CH₃)₂), 2.42 (s, 2H, H-4), 2.50 (m, 2H, pip-H), 2.76 (m, 2H, pip-H), 3.66 (d, 2H, J=6.4, OCH₂), 3.92 (s, 2H, CH₂Ar), 3.95 (s, 2H, CH₂Ar), 6.75-7.34 (m, 8H, Ar—H).

### Example 285

### 1-(4-Fluorobenzyl)-2-(4-isobutoxybenzyl)-8-methyl-1,2,8-triazaspiro[4.5]decane-3-one, hydrochloride (84AF100-25)

The desired spirocycle was obtained from 84AF99-24 (60 mg, 0.14 mmol) following the same procedure described for the preparation of 103NLS35. Formation of the hydrochloride salt was carried out as for 69NLS75 giving the title compound (39 mg, 63%) as a colorless solid.

R_{f}=0.57 (MeOH/CH₂Cl₂ 1:9). LCMS m/z 440 [M+H]⁺. ¹H NMR (CDCl₃) δ 0.94 (d, 6H, J=6.4, CH(CH₃)₂), 1.38-1.68 (m, 4H, pip-H), 1.96-2.02 (m, 1H, CH(CH₃)₂), 2.23-2.46 (m, 9H, pip-H, NCH₃, H-4), 3.61 (d, 2H, J=6.4, OCH₂), 3.85 (m, 4H, CH₂Ar_{OiBu}, CH₂Ar_{F}), 6.70-7.28 (m, 8H, Ar—H).

### Example 286

### 8-(2-[1.3]-Dioxan-2-yl-ethyl)-4-(4-fluorobenzyl)-3-(4-isobutoxy-benzyl)-1-oxa-3,8-diaza-spiro[4.5]decane-3-one, oxalate (118AF02-74)

69NLS77 was N-—BOC deprotected as described in the preparation of 69NLS79-II and the resulting compound (172 mg, 0.40 mmol) alkylated with 2-(2-bromoethyl)-1,3-dioxane following the same procedure as for the preparation of 69NLS85. Conversion of the compound (139 mg, 64%) into its oxalate salt form gave the title compound as a colorless solid.

R_{f}=0.33 (MeOH/CH₂Cl₂ 1:24). LCMS m/z 541 [M+H]⁺. ¹H NMR (CDCl₃) δ 1.00 (d, 6H, J=6.8, CH(CH₃)₂), 1.23-1.36 (m, 2H, pip-H, dioxane-H), 1.54-1.66 (m, 1H, pip-H), 1.67-1.74 (m, 3H, CH₂CH₂N, pip-H), 1.86-1.90 (m, 1H, pip-H), 1.97-2.15 (m, 2H, dioxane-H, CH(CH₃)₂), 2.31-2.43 (m, 4H, CH₂CH₂N, pip-H), 2.55 (m, 1H, pip-H), 2.69 (m, 1H, pip-H), 2.71-2.89 (m, 2H, CH₂Ar_{F}), 3.36 (t, 1H, J=6.8, H-4), 3.56 (d, 1H, J=15.2, CH₂Ar_{OiBu}), 3.67-3.74 (m, 4H, OCH₂CH(CH₃)₂, dioxane-H), 4.03-4.07 (m, 2H, dioxane-H), 4.51 (t, 1H, J=5.2, OCHO), 4.72 (d, 1H, J=15.2, CH₂Ar_{OiBu}), 6.79-7.04 (m, 8H, Ar—H).

### Example 287

### 4-(4-Fluorobenzyl)-3-(4-isobutoxy-benzyl)-8-{3-[(S)-4-isopropyl-2-oxo-oxazolidin-3-yl]-propyl}-1-oxa-3,8-diaza-spiro[4.5]decane-3-one, oxalate (118AF04-75)

69NLS77 was N-—BOC deprotected as described in the preparation of 69NLS79-II and the resulting compound (172 mg, 0.40 mmol) alkylated with (4S)-3-(3-chloropropyl)-4-isopropyl-2-oxazolidin-2-one in DMF (3 mL) in presence of sodium iodide (72 mg, 0.48 mmol), following the same procedure as for the preparation of 69NLS85. Conversion of the compound (130 mg, 55%) into its oxalate salt form gave the title compound as a colorless solid.

R_{f}=0.29 (MeOH/CH₂Cl₂ 1:19). LCMS m/z 596 [M+H]⁺. ¹H NMR (CDCl₃) δ 0.84-0.88 (m, 6H, CH_{3iPr}), 1.01 (d, 6H, J=6.8, CH_{3iBu}), 1.24-1.34 (m, 1H, pip-H), 1.55-1.74 (m, 4H, pip-H, CH_{2chain}), 1.87-1.93 (m, 1H, pip-H), 1.97-2.13 (m, 2H, CH_{OiBu}, CH_{iPr}), 2.27-2.40 (m, 4H, pip-H, NCH_{2chain}), 2.56 (m, 1H, pip-H), 2.69 (m, 1H, pip-H), 2.75-2.80 (m, 1H, CH₂Ar_{F}), 2.87-2.98 (m, 2H, CH₂Ar_{F}, NCH_{2chain}), 3.37 (t, 1H, J=6.8, H-4), 3.48-3.55 (m, 1H, NCH_{2chain}), 3.58-3.64 (m, 1H, CH₂Ar_{OiBu}), 3.65-3.71 (m, 3H, OCH₂CH(CH₃)₂, CHᵢₛₒₓ), 4.00-4.04 (m, 1H, OCHᵢₛₒₓ), 4.11-4.16 (m, 1H, OCHᵢₛₒₓ), 4.75 (d, 1H, J=15.2, CH₂Ar_{OiBu}), 6.80-7.03 (m, 8H, Ar—H).

Pharmacological Data

### Example 288

### Receptor Selection and Amplification (R-SAT) Assays

The functional receptor assay, Receptor Selection and Amplification Technology (R-SAT), was used (with minor modifications from that previously described U.S. Pat. No. 5,707,798, which is incorporated herein by reference in its entirety) to screen compounds for efficacy at the 5-HT2A receptor. The RSAT assay was conducted as described herein. The results obtained for several compounds of the invention are presented in Table 6, below.

TABLE 6
Efficiency and pIC50 of Compounds at the 5-HT2A Receptor Compared to
Ritanserin.

| | 5HT2A | | | 5HT2A % | N % | Std Dev |
|---|---|---|---|---|---|---|
| Compound ID | pIC₅₀ | N IC₅₀ | STD DEV | inhibition | inhibition | % inhibition |
| 69NLS21 | 9.1 | 4 | 0.5 | 83 | 4 | 11 |
| 69NLS35 | 9.6 | 7 | 0.2 | 103 | 7 | 20 |
| 69NLS75 | 9.5 | 15 | 0.3 | 94 | 16 | 22 |
| 69NLS52 | 9.6 | 7 | 0.3 | 87 | 7 | 14 |
| 69NLS79-II | 8.1 | 4 | 0.2 | 101 | 4 | 12 |
| 69NLS81 | 8.3 | 4 | 0.1 | 85 | 4 | 10 |
| 69NLS83 | 8 | 4 | 0.1 | 85 | 4 | 10 |
| 69NLS85 | 9.1 | 4 | 0.3 | 95 | 8 | 9 |
| 38-PH16-HCl | 8.1 | 4 | 0.2 | 95 | 4 | 18 |
| 38-PH17-HCl | 8.8 | 5 | 0.3 | 84 | 8 | 12 |
| 38-PH20 | 9.3 | 5 | 0.3 | 100 | 10 | 9 |
| 78NLS59 | 9.6 | 17 | 0.3 | 98 | 22 | 19 |
| 78NLS62 | 8.2 | 5 | 0.3 | 89 | 4 | 7 |

### Example 289

### Selectivity Profile for 4-(4-Fluorobenzyl)-3-(4-isobutoxybenzyl)-8-methyl-1-oxa-3,8-diaza-spiro[4.5]decan-2-one (69NLS75)

The R-SAT assay (described above in example 289) was used to investigate the selectivity of 4-(4-Fluoro-benzyl)-3-(4-isobutoxy-benzyl)-8-methyl-1-oxa-3,8-diaza-spiro[4.5]decan-2-one. The results from a broad profiling of this compound at a variety of receptors are reported in Table 7 below. NR means No Response, i.e. the compound investigated showed no effect at the receptor studied.

TABLE 7
Selectivity of 4-(4-Fluorobenzyl)-3-(4-isobutoxybenzyl)-8-
methyl-1-oxa-3,8-diaza-
spiro[4.5]decan-2-one.

| Receptor | Assay | pEC50 or pIC50 |
|---|---|---|
| 5HT2A | agonist | NR |
| | inverse | 9.5 (94%) |
| 5HT2B | agonist | NR |
| | inverse | <5.6 |
| | antagonist | NR |
| 5HT2C | agonist | NR |
| | inverse | 7.5 (77%) |
| 5HT1A | agonist | NR |
| | antagonist | 6.4 |
| 5HT1B | agonist | NR |
| | antagonist | <5.5 |
| 5HT1D | agonist | NR |
| | antagonist | <6.25 |
| 5HT1E | agonist | NR |
| | antagonist | <6.2 |
| 5HT1F | agonist | NR |
| | antagonist | NR |
| 5HT6A | inverse | NR |
| 5HT7A | agonist | NR |
| | inverse | NR |
| D1 | agonist | NR |
| | antagonist | NR |
| D2 | agonist | NR |
| | antagonist | NR |
| D3 | agonist | NR |
| | antagonist | NR |
| m1 | agonist | NR |
| m2 | agonist | NR |
| m3 | agonist | NR |
| | inverse | NR |
| | antagonist | 6.4 |
| m4 | agonist | <5.5 |
| m5 | agonist | NR |
| H1 | agonist | NR |
| | antagonist | 6.7 |
| alpha 2A | agonist | NR |
| | antagonist | 6.3 |
| alpha 2B | agonist | NR |
| | antagonist | NR |
| alpha 2C | agonist | NR |
| | antagonist | <5.5 |
| alpha 1B | agonist | NR |
| | antagonist | NR |
| oprk 1 | agonist | NR |
| | antagonist | NR |

### Example 290

### Preparation of N-(4-fluorobenzyl)-N-(1-methylpiperidin-4-yl)-N′-(4-(2-methylpropyloxy)phenylmethyl)carbamide

### N-(1-methylpiperidin-4-yl)-N-(4-fluorophenylmethyl)-N′-(4-(2-methylpropyloxy)phenylmethyl)carbamide

### a) Preparation of

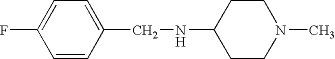

Triacetoxy borohydride (6.5 kg) was added over 1.5 h to a solution of N-methylpiperid-4-one (3.17 kg) and 4-fluorobenzylamine (3.50 kg) in methanol (30 L) maintaining the temperature under 27° C. The reaction mixture was stirred for 15 h at 22° C. The residual amine was checked by gel chromatography (4-fluorobenzylamine: <5%). A solution of 30% sodium hydroxide (12.1 kg) in water (13.6 kg) was added in 75 minutes (min) maintaining the temperature under 20° C. Methanol was distilled off to a residual volume of 26 litres. Ethyl acetate was added (26 L), the solution was stirred for 15 min, the phases were decanted over 15 min and the lower aqueous phase was discarded. Ethyl acetate was distilled under reduced pressure from the organic phase at 73-127° C. At this stage the residue was mixed with a second crude batch prepared according to this method. The combined products were then distilled at 139-140° C./20 mbar to yield 11.2 kg product (>82%).

### b) Preparation of

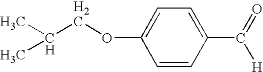

4-Hydroxybenzaldehyde (4.0 kg) and ethanol (20 L) were added to a solution of isobutyl bromide (9.0 kg) in ethanol (15 L). Potassium carbonate (13.6 kg) was added and the suspension was refluxed (74-78° C.) for 5 days. The residual 4-hydroxybenzaldehyde was checked by HPLC (<10%). The suspension was cooled to 20° C. and used in the next step.

### c) Preparation of

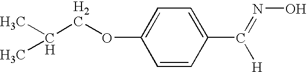

Hydroxylamine (50% in water, 8.7 kg) was added to the product from previous step b) (174 L, 176 kg) and ethanol (54 L). The suspension was refluxed (77° C.) for 3 h. Unreacted residual was checked by HPLC (<5%). The suspension was cooled to 30° C., filtered and the filter was washed with ethanol (54 L). The solution was concentrated by distillation under reduced pressure at 30° C. to a residual volume of 67 litters. The solution was cooled to 25° C. and water (110 L) was added. The suspension was concentrated by distillation under reduced pressure at 30° C. to a residual volume of 102 litters. Petrol ether (60-90 fraction, 96 L) was added and the mixture was heated to reflux (70° C.). The solution was cooled to 40° C. and crystallization was initiated by seeding. The suspension was cooled to 5° C. and stirred for 4 h. The product was centrifuged and the cake was washed with petrol ether (60-90 fraction, 32 L). The wet cake was dried at about 40° C. to yield 16 kg product (63%).

### d) Preparation of

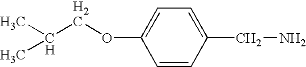

The product from previous step c) (15.7 kg) was dissolved in ethanol (123 L). Acetic acid (8.2 kg) and palladium on charcoal 5% wet (1.1 kg) were added. The oxime was hydrogenated at 22° C. and 1.5 bar for 4 h. Consumption of oxime was checked by HPLC. The catalyst was filtered and the solvent was distilled under reduced pressure at 36° C. to a final volume of 31 L. Ethyl acetate (63 L) was added and the mixture was heated to reflux (75° C.) until dissolution. The solution was cooled to 45° C. and the crystallization was initiated by seeding. The suspension was cooled to 6-10° C. and stirred for 2.5 h. The product was centrifuged and the cake was washed with 2 portions of ethyl acetate (2×0.8 L). The wet cake was dried at a temperature of about 40° C. to yield 8 kg (41%).

### e) Preparation of

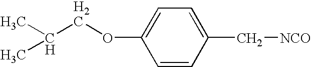

Aqueous sodium hydroxide (30%, 5.0 kg) was added to a suspension of the product from previous step d) (7.9 kg) in heptane (41 L). The solution was heated to 47° C., stirred for 15 min and decanted over 15 min. The pH was checked (pH>12) and the aqueous phase was separated. The solvent was removed by distillation under reduced pressure at 47-65° C. Heptane was added (15 L) and then removed by distillation under reduced pressure at 58-65° C. Heptane was added (7 L), the solution was filtered, and the filter was washed with heptane (7 L). The solvent was removed by distillation under reduced pressure at 28-60° C. Tetrahydrofuran (THF, 107 L) and triethylamine (TEA, 6.8 kg) were added and the temperature was fixed at 22° C. In another reactor, phosgene (5.0 kg) was introduced in tetrahydrofuran (88 L) previously cooled to −3° C. The THF and TEA solution was added to the solution of phosgene in 3 h 50 min, maintaining the temperature at −3° C. The reactor was washed with tetrahydrofuran (22 L). The mixture was stirred for 45 min at 20° C. and then for 90 min at reflux (65° C.). The solvent was distilled under reduced pressure at 25-30° C. to a residual volume of 149 L. The absence of phosgene was controlled. At this stage, phosgene was still present and the suspension was degassed by bubbling nitrogen through it. After this operation, the level of phosgene above the solution was below 0.075 ppm. The suspension was filtered and washed with tetrahydrofuran (30 L). The solvent was distilled under reduced pressure at 20-25° C. to a residual volume of 40 L. Tetrahydrofuran (51 L) was added and the solvent was distilled under reduced pressure at 20-25° C. to a residual volume of 40 L. The final volume was adjusted to about 52 litters by addition of tetrahydrofuran (11 L). The solution was analysed and used in the next step.

### f) Preparation of N-(1-methylpiperidin-4-yl)-N-(4-fluorophenylmethyl)-N′-(4-(2-methylpropyloxy)phenylmethyl)carbamide

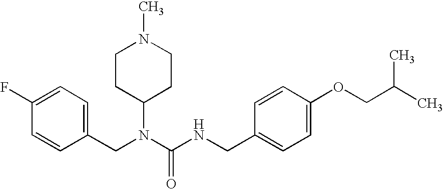

The product from previous step e) (51 L) was added in 1 h to a solution of the product from step a) (7.3 kg) in tetrahydrofuran (132 L) at 17° C. The line was washed with tetrahydrofuran (12 L) and the mixture was stirred for 15 h. Residual product from the first step was checked by HPLC. The solvent was removed by distillation under reduced pressure at 20-38° C. to a residual volume of 165 L. Charcoal (Norit SX1-G, 0.7 kg) was added, the mixture was stirred for 15 min and filtered. The line was washed with tetrahydrofuran (7 L) and the solvent was removed by distillation under reduced pressure at 20-25° C. to a residual volume of 30 L. Isopropyl acetate (96 L) was added to obtain a solution of the title compound, which contains a small amount of impurities (mainly side products from the previous reactions.) Removal of the solvent from a sample yields a substantially amorphous solid.

The solution with the crude product was used for the direct preparation of the hemi-tartrate and simultaneously for the purification of the free base via the hemi-tartrate through crystallization from suitable solvents.

### Example 291

### R-SAT Assay of N-(1-methylpiperidin-4-yl)-N-(4-fluorophenylmethyl)-N′-(4-(2-methylpropyloxy)phenylmethyl)carbamide

The functional receptor assay Receptor Selection and Amplification Technology (R-SAT) was used to investigate the activity of N-(1-methylpiperidin-4-yl)-N-(4-fluorophenylmethyl)-N′-(4-(2-methylpropyloxy)phenylmethyl)carbamide as an inverse agonist at 5HT2A receptors. The compound exhibited high potency (pIC50 of 9.1) and high efficacy (98%) at 5HT2A receptors.

### Example 292

### Effects of N-(1-methylpiperidin-4-yl)-N-(4-fluorophenylmethyl)-N′-(4-(2-methylpropyloxy)phenylmethyl)carbamide on Slow Wave Sleep and Other Sleep Parameters

A double-blind, placebo-controlled study involving 45 healthy volunteers ranging in age from 40 to 64 was conducted. The purpose of the study was to determine the effects of N-(1-methylpiperidin-4-yl)-N-(4-fluorophenylmethyl)-N′-(4-(2-methylpropyloxy)phenylmethyl)carbamide on slow wave sleep and other sleep parameters as measured by polysomnography (PSG) in older adults. The effects of N-(1-methylpiperidin-4-yl)-N-(4-fluorophenylmethyl)-N′-(4-(2-methylpropyloxy)phenylmethyl)carbamide were measured before and after a single dose and at plasma steady state. The subjects were randomized to one of five treatment arms, including placebo and four different doses (1 mg, 2.5 mg, 5 mg, and 20 mg) of N-(1-methylpiperidin-4-yl)-N-(4-fluorophenylmethyl)-N′-(4-(2-methylpropyloxy)phenylmethyl)carbamide. Each group was administered placebo or the specified dose of N-(1-methylpiperidin-4-yl)-N-(4-fluorophenylmethyl)-N′-(4-(2-methylpropyloxy)phenylmethyl)carbamide once-daily each morning for 14 consecutive days. All subjects underwent a two-night screening and baseline PSG evaluation. Additional PSG measurements were performed on the evening of study days 1 and 13. The subjects also completed a Continuous Performance Test (CPT) to assess the potential impact on daytime functioning. Blood samples were collected at the beginning and end of the 14-day period to determine N-(1-methylpiperidin-4-yl)-N-(4-fluorophenylmethyl)-N′-(4-(2-methylpropyloxy)phenylmethyl)carbamide levels in plasma. Of the 45 subjects, 20 of them also underwent a positron emission tomography (PET) study to measure 5-HT_{2A} brain receptor occupancy.

Inclusion criteria included a reported bedtime between 9 pm and midnight, a usual sleep duration>4 hours, and habitual time in bed between 6 and 9 hours per night. Women had to be postmenopausal or previously surgically sterilized in order to participate. Exclusion criteria included a history or current diagnosis of other sleep disorders (such as restless leg syndrome, periodic leg movements with arousals, narcolepsy, rapid eye movement (REM) Behavior Disorder, circadian rhythm sleep disorder, breathing related sleep disorder, or parasomnias), smoking more than 10 cigarettes (or equivalent) per day, a history of alcohol or substance abuse or positive drug or alcohol screen, or drinking more than 5 cups of caffeine containing beverages, more than 1 liter of xanthine containing beverages, or more than 40 g of alcohol per day. Participants with an occupational history that included shift work or recent significant travel across three or more time zones within the prior two weeks or self reported napping of >45 min per day were also excluded. In addition, after a screening PSG, participants with an apnea-hypopnea index>15 or a periodic leg movement arousal index>15 were also excluded.

Twenty-three (23) men and 22 women were included in the study. The mean (±standard deviation) age of the participants was 51.8±6.9 years (range 40 to 64). The mean BMI was 24.05±2.72 kg/m². The mean height was 168.8±10 cm, and the mean weight was 68.7±10.4 kg. All 45 participants completed the study and were included in data analysis.

Among the 45 participants included, 34 were non-smokers and 11 were smokers in whom the mean daily consumption was between 3.8 and 9.2 cigarettes. Eight participants drank alcohol with a mean daily consumption between 4.1 and 28.5 g. Thirty-nine of the 45 drank beverages containing caffeine with daily consumption between 1.1 and 3.5 cups. Lastly, 6 drank beverages containing xanthine with daily consumption was between 0.2 and 0.8 L. These values were not within the range for exclusion and none of these were thought to interfere with study results.

During an assessment period, participants stayed overnight in the clinic for one night from Day −1 to Day 2 and one night from Day 13 to Day 14. Participants underwent a one-night PSG evaluation on the evening of Day 1, following the first dose of treatment and a final one-night PSG evaluation on the evening of Day 13. Between Day 3 and Day 12, participants were at home.

The PSG recordings consisted of an all night recording of sleep parameters including two channels of EEG (C3 and O1 referred to contralateral mastoid), two channels of EOG (with electrode placement at the outer canthi in a superior and inferior position), and a single channel of EMG (with electrode placement in a sub-mental location). Polysomnographic recordings were performed from 23:00 to 07:00 on the following day.

Drug Dosing: A single dose of a solution of placebo or one of the four doses of N-(1-methylpiperidin-4-yl)-N-(4-fluorophenylmethyl)-N′-(4-(2-methylpropyloxy)phenylmethyl)carbamide was administered between 7:30 and 9:00 in the morning every day for 14 consecutive days. The drug was dispensed as a solution (in vials) per os (p.o.), in a double-blind fashion as determined by the randomization schedule. The content of the vials was completely consumed followed by the consumption of one vial of water.

Performance Assessment: A Continuous Performance Test (CPT; Rosvold M E, Mirsky A F, Sarason I, Bransome E R Jr., Beck L H (1956) A continuous performance test for brain damage. J Consult Psychol 20:343-350, which is incorporated herein by reference in its entirety), was used to evaluate attention and vigilance. The test was administered both during the evening and the morning following the baseline PSG on Day 1 and the evaluation PSG on Day 13. Participants were rapidly presented a numerical train with a target number as point of reference. Participants pushed a button rapidly when the target number appeared in the number list. The procedure lasted three minutes. The number of targets detected and the number of false alarms were assessed.

Safety: 3-positional vital signs, ECGs, body temperature, and respiratory rates as well as blood chemistries, hematology, and urinalysis served as safety measures. Safety measures were assessed at screening, baseline (Day −1), Day 1, Day 2, Day 13, and Day 14. Any observed or reported adverse events were recorded. Blood samples were taken on Day −1 (baseline) and on Day 14 (steady state). Two weeks after the termination of treatment administration (Day 28), an end-of-study visit was conducted, and included a clinical safety evaluation.

Plasma Levels: Blood samples were collected into Vacutainer® (dry sodium heparin, green top tube, 6 mL capacity) on Day −1 and again on Day 14 (pre-dose). The time interval between blood collection and freezing of the plasma was no longer than 45 minutes. Within 45 minutes of collection, the plasma fractions were separated by centrifugation at 3,000 rotations per minutes for 10 minutes. The plasma fractions were divided and transferred into two properly labeled storage tubes and frozen at approximately −70° C. Plasma samples were evaluated for N-(1-methylpiperidin-4-yl)-N-(4-fluorophenylmethyl)-N′-(4-(2-methylpropyloxy)phenylmethyl)carbamide using a validated high performance liquid chromatography/tandem mass spectrometric (LC/MS/MS) assay.

Data Analysis: A multivariate analyses of variance (MANOVA) with 5 clusters of sleep parameters as dependent variables (sleep continuity architecture and profile parameters for the visual analysis, and NREM and REM parameters for the spectral power analysis as shown in Table 1) was conducted initially. Change from baseline to the last day of drug administration was assessed by an analysis of covariance with repeated measures (ANCOVA®), with the baseline variables as covariates, to detect group differences (drug at each dose versus placebo) in the PSG sleep findings. For each ANCOVA, two main effects (Treatment, 4 levels, and Study Day, 2 levels) and their interaction were analyzed. Drug to placebo contrasts were calculated as a whole (Day 1 and Day 13 combined) as well as separately for each Study Day. Change from baseline to the last day of drug administration, by group, on the results of the CPT was also analyzed. Additional pharmacodynamic analysis, including the electrographic results obtained on Study Day 1, was performed. SAS® Version 8.2 was used for non-pharmacokinetic parameters and WinNonLin V4.0 was used to compute all pharmacokinetic parameters.

Results

Compliance with the dosing regimen was reported to be high and was confirmed with the determination of drug plasma concentrations on the morning of Day 14. The mean plasma steady-state concentrations increased with increased dose and ranged from 1.6 ng/mL to 35.0 ng/mL (Table 8).

TABLE 8
Pharmacokinetic exposure of N-(1-methylpiperidin-4-yl)-N-(4-
fluorophenylmethyl)-N′-(4-(2-methylpropyloxy)phenylmethyl)
carbamide after 13 days once daily administration.
Concentrations given in ng/mL (n = 9/dose).

| | 1 mg | 2.5 mg | 5 mg | 20 mg |
|---|---|---|---|---|
| Mean | 1.628 | 3.647 | 8.516 | 35.033 |
| Median | 1.750 | 3.360 | 8.720 | 34.100 |
| SEM | 0.153 | 0.355 | 0.711 | 3.395 |
| SD | 0.458 | 1.065 | 2.134 | 10.184 |
| Minimum | 0.92 | 2.07 | 5.91 | 21.90 |
| Maximum | 2.33 | 5.55 | 13.00 | 52.70 |

SEM = Standard error of the mean
SD = Standard deviation

MANOVA on treatment by study day effects resulted in significant treatment effects for all clusters of sleep parameters except sleep continuity variables, suggesting that N-(1-methylpiperidin-4-yl)-N-(4-fluorophenylmethyl)-N′-(4-(2-methylpropyloxy)phenylmethyl)carbamide significantly influenced, as a whole, most of the sleep parameters under study. A Study Day effect was evidenced only for NREM relative parameters for the spectral power density suggesting that, as a whole, NREM spectral power was influenced by time (single versus repeated treatment administration). No Study Day effect was observed for the 4 other clusters suggesting that most of the sleep EEG parameters under study were not influenced by time.

Results of the ANCOVA on the primary endpoint, slow wave sleep or stages 3 and 4 combined, showed a significant (p<0.001) effect of Treatment and no main effect of Study Day nor Treatment by Study Day interaction, suggesting that the treatment effect was similar across the 2 study days and that the 4 treatment conditions were not differently influenced by study day. FIG. 1 is a graph depicting the duration slow wave sleep observed for the various administrations (BL=baseline, D1=Study Day 1, D13=Study Day 13). Table 9 provides the slow sleep duration numbers. The graph shows the baseline duration as well as the absolute change from baseline. Table 10 provides the change in slow wave sleep from baseline in minutes, percentage, and statistical significance between each dose and placebo.

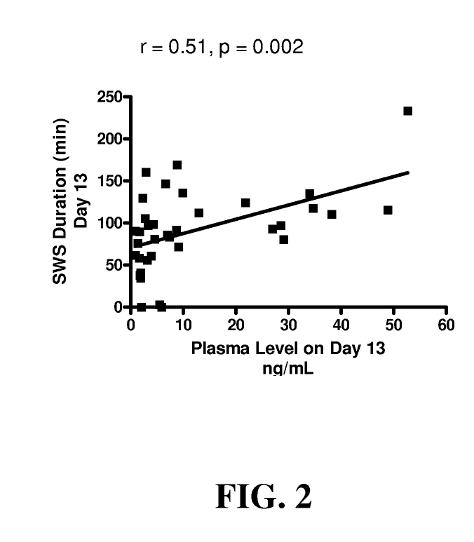

These results show the slow wave sleep increase seen with administration of N-(1-methylpiperidin-4-yl)-N-(4-fluorophenylmethyl)-N′-(4-(2-methylpropyloxy)phenylmethyl)carbamide. The data demonstrated that once-daily administration of 5 mg and 20 mg of N-(1-methylpiperidin-4-yl)-N-(4-fluorophenylmethyl)-N′-(4-(2-methylpropyloxy)phenylmethyl)carbamide, the two highest doses used in this study, induced statistically significant increases in slow wave sleep (p<0.001) as defined by the time spent in Stage 3 and Stage 4 sleep. These stages are commonly referred to as deep, or slow wave, sleep. This robust effect was demonstrated both acutely (on study day 1) and after chronic administration (on study day 13). The two lower doses of N-(1-methylpiperidin-4-yl)-N-(4-fluorophenylmethyl)-N′-(4-(2-methylpropyloxy)phenylmethyl)carbamide were less effective than the two higher doses, demonstrating that the sleep effects of N-(1-methylpiperidin-4-yl)-N-(4-fluorophenylmethyl)-N′-(4-(2-methylpropyloxy)phenylmethyl)carbamide were dose-related. No significant differences between study days were found, suggesting that N-(1-methylpiperidin-4-yl)-N-(4-fluorophenylmethyl)-N′-(4-(2-methylpropyloxy)phenylmethyl)carbamide increased slow wave sleep after a single dose and that the effect was maintained with repeated administration. As depicted by the graph in FIG. 2, a positive correlation was observed (r 0.51, 95% CI=0.22-0.72; r²=0.26, p=0.002) between slow wave sleep duration and drug plasma level.

TABLE 9
Effects of N-(1-methylpiperidin-4-yl)-N-(4-fluorophenylmethyl)-N′-
(4-(2-methylpropyloxy)phenylmethyl) carbamide on slow
wave sleep duration, number of awakenings, and wake after
sleep onset duration.

| | | Placebo | 1 mg | 2.5 mg | 5 mg | 20 mg |
|---|---|---|---|---|---|---|
| SWS | Baseline | 67.39 ± | 63.39 ± | 54.00 ± | 57.61 ± | 83.94 ± |
| duration | | 32.09 | 23.12 | 30.88 | 33.06 | 21.44 |
| (min) | Day 1 | 56.22 ± | 69.78 ± | 75.83 ± | 94.89 ± | 129.94 ± |
| p < 0.001 | | 19.12 | 32.14 | 50.91 | 39.84 | 36.29 |
| | Day 13 | 64.28 ± | 68.78 ± | 73.61 ± | 99.56 ± | 122.94 ± |
| | | 13.96 | 31.01 | 50.82 | 49.64 | 44.69 |
| Number of | Baseline | 20.78 ± | 21.11 ± | 24.11 ± | 26.22 ± | 20.67 ± |
| awaken- | | 9.51 | 6.41 | 7.20 | 7.28 | 7.94 |
| ings | Day 1 | 27.89 ± | 20.22 ± | 21.44 ± | 20.67 ± | 20.89 ± |
| after sleep | | 10.39 | 3.15 | 6.84 | 6.56 | 11.82 |
| onset | Day 13 | 23.11 ± | 20.78 ± | 19.78 ± | 21.78 ± | 18.89 ± |
| p < 0.05 | | 5.80 | 5.70 | 6.00 | 6.46 | 6.81 |
| Duration | Baseline | 23.50 ± | 33.22 ± | 49.06 ± | 56.17 ± | 52.39 ± |
| (min) of | | 14.02 | 31.20 | 27.26 | 28.41 | 24.16 |
| wake after | Day 1 | 43.72 ± | 47.67 ± | 51.00 ± | 64.56 ± | 33.44 ± |
| sleep | | 17.73 | 34.57 | 29.32 | 51.90 | 32.67 |
| onset | Day 13 | 51.61 ± | 40.06 ± | 30.44 ± | 43.89 ± | 29.89 ± |
| p = 0.1 | | 33.47 | 35.24 | 26.16 | 28.64 | 14.85 |

Mean ± standard deviations are shown (n = 9/group)
SWS: slow wave sleep.

TABLE 10
Change in Slow Wave Sleep From Baseline in Minutes, Percentage, and Statistical
Significance Between Each Dose and Placebo

| | | Day 1 | | | Day 13 | | |
|---|---|---|---|---|---|---|---|
| | | Change From Baseline | | | Change From Baseline | | |
| | N | Minutes | Percentage | | Minutes | Percentage | |
| Drug (20 mg) | 9 | 46.0 | 55% | p < 0.001 | 39.0 | 46% | p < 0.001 |
| Drug (5 mg) | 9 | 37.3 | 65% | p < 0.001 | 41.9 | 73% | p < 0.001 |
| Drug (2.5 mg) | 9 | 21.8 | 40% | p < 0.05 | 19.6 | 36% | n.s. |
| Drug (1 mg) | 9 | 6.4 | 10% | n.s. | 5.4 | 9% | n.s. |
| Placebo | 9 | −11.2 | −17% | n/a | −3.1 | −4.6% | n/a |

n.s. = not significant

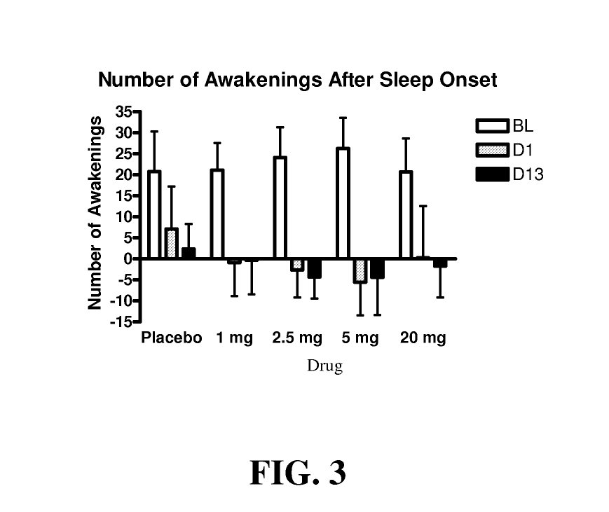

Sleep Continuity Parameters: In agreement with MANOVA results showing no significant effect of treatment, ANCOVAs performed on each sleep continuity parameter taken separately were not significant for Treatment, for Study Day, or for Treatment by Study Day interaction. The sole exception was the number of awakenings after sleep onset for which a significant (p<0.05) effect of treatment was evidenced, consistent with a shift to deeper sleep. FIG. 3 is a graph depicting the number of awakenings after sleep onset. The graph shows the baseline number as well as the absolute change from baseline. This data is also presented in Table 9. Drug to placebo contrasts indicated that the 4 doses of N-(1-methylpiperidin-4-yl)-N-(4-fluorophenylmethyl)-N′-(4-(2-methylpropyloxy)phenylmethyl)carbamide significantly decreased number of awakenings after sleep onset, with the most significant effects being observed in the 2.5 mg and 5 mg groups. The effects of N-(1-methylpiperidin-4-yl)-N-(4-fluorophenylmethyl)-N′-(4-(2-methylpropyloxy)phenylmethyl)carbamide on number of awakenings were more pronounced on Study Day 1 than on Study Day 13. A trend level of minutes awake after sleep onset (WASO), the other hallmark of sleep maintenance insomnia, was observed in the group of older healthy adults (Table 9). Other sleep continuity variables that were measured but not affected by N-(1-methylpiperidin-4-yl)-N-(4-fluorophenylmethyl)-N′-(4-(2-methylpropyloxy)phenylmethyl)carbamide included sleep period time, total sleep time, sleep onset latency, number of stage shifts, total time awake, early morning wake, sleep efficiency index, and microarousal index.

Sleep Architecture Parameters: In agreement with MANOVA results showing a significant Treatment effect (p<0.001), ANCOVAs evidenced significant Treatment effects for most of the sleep architecture parameters: increased non-REM sleep duration (p<0.05), increased non-REM sleep proportion (p<0.01), decreased stage 2 (duration as well as proportion, p<0.05), increased stage 3 (duration and proportion, p<0.05) and stage 4 (duration and proportion, p<0.001), and increased slow wave sleep proportion (stages 3 and 4 combined, p<0.001). The direction and intensity of effects are shown in Table 11. For all sleep architecture parameters, neither significant Study Day effects nor Treatment by Study Day interactions were found. N-(1-methylpiperidin-4-yl)-N-(4-fluorophenylmethyl)-N′-(4-(2-methylpropyloxy)phenylmethyl)carbamide had no effect on REM sleep duration, proportion of REM sleep, REM sleep latency, REM activity or REM density.

TABLE 11
Effects of N-(1-methylpiperidin-4-yl)-N-(4-fluorophenylmethyl)-N′-(4-(2-
methylpropyloxy)phenylmethyl) carbamide on sleep architecture and profile parameters.

| | 1 mg | | | 2.5 mg | | | 5 mg | | | 20 mg | | |
|---|---|---|---|---|---|---|---|---|---|---|---|---|
| Parameter | C | D1 | D13 | C | D1 | D13 | C | D1 | D13 | C | D1 | D13 |
| NREM sleep duration | [symbol] | [symbol] | [symbol] | ↑ | [symbol] | ↑ | [symbol] | [symbol] | [symbol] | ↑ | [symbol] | [symbol] |
| NREM sleep % | [symbol] | [symbol] | [symbol] | ↑ | [symbol] | ↑↑ | [symbol] | [symbol] | [symbol] | ↑↑ | [symbol] | ↑↑ |
| SWS % | [symbol] | [symbol] | [symbol] | ↑↑ | ↑ | [symbol] | ↑↑ | ↑↑ | ↑↑ | ↑↑ | ↑↑ | ↑↑ |
| | | | | | | | ↑ | ↑ | ↑ | ↑ | ↑ | ↑ |
| SWS 1^{st} third | [symbol] | [symbol] | [symbol] | [symbol] | [symbol] | [symbol] | ↑↑ | ↑↑ | ↑ | ↑↑ | [symbol] | [symbol] |
| | | | | | | | ↑ | | | | | |
| SWS 2^{nd} third | [symbol] | [symbol] | [symbol] | ↑↑ | ↑ | [symbol] | ↑↑ | [symbol] | ↑ | ↑↑ | ↑↑ | ↑↑ |
| | | | | | | | | | | ↑ | ↑ | |
| SWS 3^{rd} third | [symbol] | [symbol] | [symbol] | [symbol] | [symbol] | [symbol] | [symbol] | [symbol] | [symbol] | [symbol] | [symbol] | [symbol] |
| Stage 1% | [symbol] | [symbol] | [symbol] | [symbol] | [symbol] | [symbol] | ↓ | ↓ | [symbol] | [symbol] | [symbol] | [symbol] |
| Stage 2 duration | [symbol] | [symbol] | [symbol] | [symbol] | [symbol] | [symbol] | [symbol] | [symbol] | [symbol] | ↓ | ↓ | [symbol] |
| Stage 2% | [symbol] | [symbol] | [symbol] | [symbol] | [symbol] | [symbol] | ↓ | ↓ | [symbol] | [symbol] | [symbol] | [symbol] |
| Stage 3 duration | [symbol] | [symbol] | [symbol] | ↑ | ↑ | [symbol] | ↑ | ↑ | [symbol] | ↑ | [symbol] | [symbol] |
| Stage 3% | [symbol] | [symbol] | [symbol] | ↑ | ↑ | [symbol] | ↑ | ↑ | [symbol] | ↑ | [symbol] | [symbol] |
| Stage 4 duration | [symbol] | [symbol] | [symbol] | ↑ | [symbol] | [symbol] | ↑↑ | ↑↑ | ↑↑ | ↑↑ | ↑↑ | ↑↑ |
| | | | | | | | ↑ | | ↑ | ↑ | ↑ | ↑ |
| Stage 4% | [symbol] | [symbol] | [symbol] | ↑ | [symbol] | [symbol] | ↑↑ | ↑↑ | ↑↑ | ↑↑ | ↑↑ | ↑↑ |
| | | | | | | | ↑ | | ↑ | ↑ | ↑ | ↑ |
| REM sleep | [symbol] | [symbol] | [symbol] | [symbol] | [symbol] | [symbol] | [symbol] | [symbol] | [symbol] | [symbol] | [symbol] | [symbol] |

Drug to placebo contrasts are schematised in the following way: [symbol] : no change; ↓ : decrease [↓↓↓, <.001; ↓↓, <.01; ↓, <.05] and ↑: increase [↑↑↑, <.001; ↑↑, <.01; ↑, <.05].
C: combined visits; D1: Study Day 1; D13: Study Day 13.
REM: rapid eye movement; NREM: non-rapid eye movement; SWS: slow wave sleep.

Sleep Profile Parameters: The MANOVA performed on sleep profile parameters showed a significant treatment effect (p<0.001). Results of the ANCOVAs revealed that this treatment effect mostly related to slow wave sleep parameters since significant treatment effects were observed on duration of slow wave sleep on the first (p<0.01) and second (p<0.001) third of the night. The direction and intensity of effects are shown in Table 11. There were no other treatment effects for the other sleep profile parameters. For all ANCOVAs, neither significant Study Day effect nor a Treatment by Study Day interaction were observed.

Spectral Power Density Parameters: MANOVAs performed on spectral power density parameters evidenced a significant Treatment effect (p<0.01) without Study Day effect during REM sleep and a significant Treatment effect (p<0.001) with a significant (p<0.001) Study Day effect during NREM sleep. For REM sleep parameters, the sole significant ANOVAs treatment effect was for Beta1 (p<0.05). For non-REM sleep parameters, significant (p<0.001) treatment effects were evidenced for slow delta, fast delta, slow wave activity, theta, spindle frequency activity and Beta1 (data not shown).

Performance: On the CPT, ANCOVAs indicated an absence of effect of any dose of N-(1-methylpiperidin-4-yl)-N-(4-fluorophenylmethyl)-N′-(4-(2-methylpropyloxy)phenylmethyl)carbamide on the detected targets and false alarm change scores, regardless evaluation session and group (p>0.10). None of the planned comparisons were significant or approached statistical significance (p>0.10). For the evening evaluation, a p value of 0.67 was determined for accurate detections and a p value of 0.51 was determined for false alarms. For the morning evaluation, a p value of 0.50 was determined for accurate detections and a p value of 0.87 was determined for false alarms.

### CONCLUSION

Morning administration of N-(1-methylpiperidin-4-yl)-N-(4-fluorophenylmethyl)-N′-(4-(2-methylpropyloxy)phenylmethyl)carbamide resulted in a significant dose-related increase of slow wave sleep during the subsequent night. This effect was observed after a single administration and was sustained over time with repeated administration. Results of the clinical trial also showed that treatment with N-(1-methylpiperidin-4-yl)-N-(4-fluorophenylmethyl)-N′-(4-(2-methylpropyloxy)phenylmethyl)carbamide produced trends for improvement on measures for sleep maintenance, including decreases in the number of awakenings (p=0.04) and in time awake after sleep onset (p=0.09). Importantly, in contrast to most currently marketed insomnia drugs, which are sedative, sleep-inducing agents and have the potential to impair daytime functioning, N-(1-methylpiperidin-4-yl)-N-(4-fluorophenylmethyl)-N′-(4-(2-methylpropyloxy)phenylmethyl)carbamide did not alter latency to sleep onset and did not impair daytime functioning.

Doses of 5 mg and 20 mg and associated mean plasma levels ranging from 8.5 to 35.0 ng/mL showed robust and significant increases in different parameters directly related to slow wave sleep including slow wave sleep (stages 3 and 4 combined) duration and proportion, duration and proportion of stage 4 and the slowest components of the NREM sleep EEG activities. Increased plasma levels generally were associated with higher magnitude of effects. The highest dose of N-(1-methylpiperidin-4-yl)-N-(4-fluorophenylmethyl)-N′-(4-(2-methylpropyloxy)phenylmethyl)carbamide (20 mg) also decreased duration and proportion of wake after sleep onset. These dose dependent observations are consistent with a shift to deeper sleep. Other PSG measures such as sleep onset latency, total sleep time, sleep efficiency and REM sleep parameters were unaffected by N-(1-methylpiperidin-4-yl)-N-(4-fluorophenylmethyl)-N′-(4-(2-methylpropyloxy)phenylmethyl)carbamide treatment in this population.

N-(1-methylpiperidin-4-yl)-N-(4-fluorophenylmethyl)-N′-(4-(2-methylpropyloxy)phenylmethyl)carbamide was safe and well tolerated in the study subjects and there were no serious adverse events reported. All adverse events were mild to moderate in nature and were comparable across the placebo and N-(1-methylpiperidin-4-yl)-N-(4-fluorophenylmethyl)-N′-(4-(2-methylpropyloxy)phenylmethyl)carbamide-treated groups.

Although the invention has been described with reference to embodiments and examples, it should be understood that numerous and various modifications can be made without departing from the spirit of the invention. Accordingly, the invention is limited only by the following claims.

What is claimed is:

1. A method of increasing slow-wave sleep comprising administering N-(1-methylpiperidin-4-yl)-N-(4-fluorophenylmethyl)-N′-(4-(2-methylpropyloxy)phenylmethyl)carbamide to a subject in need of increased slow wave sleep in an amount sufficient to increase slow wave sleep.

2. The method of claim 1, wherein the administration of N-(1-methylpiperidin-4-yl)-N-(4-fluorophenylmethyl)-N′-(4-(2-methylpropyloxy)phenylmethyl)carbamide comprises oral administration.

3. The method of claim 1, wherein the amount of N-(1-methylpiperidin-4-yl)-N-(4-fluorophenylmethyl)-N′-(4-(2-methylpropyloxy)phenylmethyl)carbamide is in the range of about 0.5 mg to about 50 mg.

4. The method of claim 1, wherein the amount of N-(1-methylpiperidin-4-yl)-N-(4-fluorophenylmethyl)-N′-(4-(2-methylpropyloxy)phenylmethyl)carbamide is in the range of about 1 mg to about 40 mg.

5. The method of claim 1, wherein the amount of N-(1-methylpiperidin-4-yl)-N-(4-fluorophenylmethyl)-N′-(4-(2-methylpropyloxy)phenylmethyl)carbamide is in the range of about 2.5 mg to about 30 mg.

6. The method of claim 1, wherein the amount of N-(1-methylpiperidin-4-yl)-N-(4-fluorophenylmethyl)-N′-(4-(2-methylpropyloxy)phenylmethyl)carbamide is in the range of about 5 mg to about 20 mg.

7. The method of claim 1, wherein the administration of N-(1-methylpiperidin-4-yl)-N-(4-fluorophenylmethyl)-N′-(4-(2-methylpropyloxy)phenylmethyl)carbamide does not affect a sleep parameter selected from the group consisting of sleep period time, total sleep time, sleep onset latency, number of stage shifts, total time awake, early morning wake, sleep efficiency index, microarousal index, and a REM sleep parameter.

8. The method of claim 7, wherein the REM sleep parameter is selected from the group consisting of REM sleep duration, proportion of REM sleep, REM sleep latency, REM activity and REM density.

9. The method of claim 1, wherein the amount of N-(1-methylpiperidin-4-yl)-N-(4-fluorophenylmethyl)-N′-(4-(2-methylpropyloxy)phenylmethyl)carbamide results in a steady state blood plasma concentration of N-(1-methylpiperidin-4-yl)-N-(4-fluorophenylmethyl)-N′-(4-(2-methylpropyloxy)phenylmethyl)carbamide in the range of about 2 ng/mL to about 60 ng/mL.

10. The method of claim 1, wherein the amount of N-(1-methylpiperidin-4-yl)-N-(4-fluorophenylmethyl)-N′-(4-(2-methylpropyloxy)phenylmethyl)carbamide results in a steady state blood plasma concentration of N-(1-methylpiperidin-4-yl)-N-(4-fluorophenylmethyl)-N′-(4-(2-methylpropyloxy)phenylmethyl)carbamide in the range of about 4 ng/mL to about 50 ng/mL.

11. The method of claim 1, wherein the amount of N-(1-methylpiperidin-4-yl)-N-(4-fluorophenylmethyl)-N′-(4-(2-methylpropyloxy)phenylmethyl)carbamide results in a steady state blood plasma concentration of N-(1-methylpiperidin-4-yl)-N-(4-fluorophenylmethyl)-N′-(4-(2-methylpropyloxy)phenylmethyl)carbamide in the range of about 6 ng/mL to about 40 ng/mL.

12. The method of claim 1, wherein the amount of N-(1-methylpiperidin-4-yl)-N-(4-fluorophenylmethyl)-N′-(4-(2-methylpropyloxy)phenylmethyl)carbamide results in a steady state blood plasma concentration of N-(1-methylpiperidin-4-yl)-N-(4-fluorophenylmethyl)-N′-(4-(2-methylpropyloxy)phenylmethyl)carbamide in the range of about 8.5 ng/mL to about 35 ng/mL.

13. The method of claim 1, wherein the subject is administered N-(1-methylpiperidin-4-yl)-N-(4-fluorophenylmethyl)-N′-(4-(2-methylpropyloxy)phenylmethyl)carbamide once every day.

14. The method of claim 1, wherein the subject is administered a first dosage form of N-(1-methylpiperidin-4-yl)-N-(4-fluorophenylmethyl)-N′-(4-(2-methylpropyloxy)phenylmethyl)carbamide at least 24 hours prior to administration of a second dosage form of N-(1-methylpiperidin-4-yl)-N-(4-fluorophenylmethyl)-N′-(4-(2-methylpropyloxy)phenylmethyl)carbamide.

15. The method of claim 14, wherein the first dosage form comprises a higher dose of N-(1-methylpiperidin-4-yl)-N-(4-fluorophenylmethyl)-N′-(4-(2-methylpropyloxy)phenylmethyl)carbamide than the second dosage form.

16. The method of claim 1, further comprising informing the subject that N-(1-methylpiperidin-4-yl)-N-(4-fluorophenylmethyl)-N′-(4-(2-methylpropyloxy)phenylmethyl)carbamide increases slow wave sleep.

17. The method of claim 16, wherein the informing comprises providing printed matter that advises that N-(1-methylpiperidin-4-yl)-N-(4-fluorophenylmethyl)-N′-(4-(2-methylpropyloxy)phenylmethyl)carbamide increases slow wave sleep.

18. The method of claim 17, wherein the printed matter is a label.

19. The method of claim 1, wherein N-(1-methylpiperidin-4-yl)-N-(4-fluorophenylmethyl)-N′-(4-(2-methylpropyloxy)phenylmethyl)carbamide is administered at a time other than immediately before a sleep period.

20. The method of claim 19, wherein N-(1-methylpiperidin-4-yl)-N-(4-fluorophenylmethyl)-N′-(4-(2-methylpropyloxy)phenylmethyl)carbamide is administered in the morning.

21. A method of treating insomnia comprising administering N-(1-methylpiperidin-4-yl)-N-(4-fluorophenylmethyl)-N′-(4-(2-methylpropyloxy)phenylmethyl)carbamide to a subject suffering from insomnia in an amount sufficient to ameliorate insomnia.

22. The method of claim 21, wherein the amount of N-(1-methylpiperidin-4-yl)-N-(4-fluorophenylmethyl)-N′-(4-(2-methylpropyloxy)phenylmethyl)carbamide is in the range of about 0.5 mg to about 50 mg.

23. The method of claim 21, wherein the amount of N-(1-methylpiperidin-4-yl)-N-(4-fluorophenylmethyl)-N′-(4-(2-methylpropyloxy)phenylmethyl)carbamide is in the range of about 1 mg to about 40 mg.

24. The method of claim 21, wherein the amount of N-(1-methylpiperidin-4-yl)-N-(4-fluorophenylmethyl)-N′-(4-(2-methylpropyloxy)phenylmethyl)carbamide is in the range of about 2.5 mg to about 30 mg.

25. The method of claim 21, wherein the amount of N-(1-methylpiperidin-4-yl)-N-(4-fluorophenylmethyl)-N′-(4-(2-methylpropyloxy)phenylmethyl)carbamide is in the range of about 5 mg to about 20 mg.

26. The method of claim 21, wherein the administration of N-(1-methylpiperidin-4-yl)-N-(4-fluorophenylmethyl)-N′-(4-(2-methylpropyloxy)phenylmethyl)carbamide does not affect a sleep parameter selected from the group consisting of sleep period time, total sleep time, sleep onset latency, number of stage shifts, total time awake, early morning wake, sleep efficiency index, microarousal index, and a REM sleep parameter.

27. The method of claim 26, wherein the REM sleep parameter is selected from the group consisting of REM sleep duration, proportion of REM sleep, REM sleep latency, REM activity and REM density.

28. The method of claim 21, wherein the amount of N-(1-methylpiperidin-4-yl)-N-(4-fluorophenylmethyl)-N′-(4-(2-methylpropyloxy)phenylmethyl)carbamide results in a steady state blood plasma concentration of N-(1-methylpiperidin-4-yl)-N-(4-fluorophenylmethyl)-N′-(4-(2-methylpropyloxy)phenylmethyl)carbamide in the range of about 2 ng/mL to about 60 ng/mL.

29. The method of claim 21, wherein the amount of N-(1-methylpiperidin-4-yl)-N-(4-fluorophenylmethyl)-N′-(4-(2-methylpropyloxy)phenylmethyl)carbamide results in a steady state blood plasma concentration of N-(1-methylpiperidin-4-yl)-N-(4-fluorophenylmethyl)-N′-(4-(2-methylpropyloxy)phenylmethyl)carbamide in the range of about 4 ng/mL to about 50 ng/mL.

30. The method of claim 21, wherein the amount of N-(1-methylpiperidin-4-yl)-N-(4-fluorophenylmethyl)-N′-(4-(2-methylpropyloxy)phenylmethyl)carbamide results in a steady state blood plasma concentration of N-(1-methylpiperidin-4-yl)-N-(4-fluorophenylmethyl)-N′-(4-(2-methylpropyloxy)phenylmethyl)carbamide in the range of about 6 ng/mL to about 40 ng/mL.

31. The method of claim 21, wherein the amount of N-(1-methylpiperidin-4-yl)-N-(4-fluorophenylmethyl)-N′-(4-(2-methylpropyloxy)phenylmethyl)carbamide results in a steady state blood plasma concentration of N-(1-methylpiperidin-4-yl)-N-(4-fluorophenylmethyl)-N′-(4-(2-methylpropyloxy)phenylmethyl)carbamide in the range of about 8.5 ng/mL to about 35 ng/mL.

32. The method of claim 21, wherein the subject is administered N-(1-methylpiperidin-4-yl)-N-(4-fluorophenylmethyl)-N′-(4-(2-methylpropyloxy)phenylmethyl)carbamide once every day.

33. The method of claim 21, wherein the subject is administered a first dosage form of N-(1-methylpiperidin-4-yl)-N-(4-fluorophenylmethyl)-N′-(4-(2-methylpropyloxy)phenylmethyl)carbamide at least 24 hours prior to administration of a second dosage form of N-(1-methylpiperidin-4-yl)-N-(4-fluorophenylmethyl)-N′-(4-(2-methylpropyloxy)phenylmethyl)carbamide.

34. The method of claim 33, wherein the first dosage form comprises a higher dose of N-(1-methylpiperidin-4-yl)-N-(4-fluorophenylmethyl)-N′-(4-(2-methylpropyloxy)phenylmethyl)carbamide than the second dosage form.

35. The method of claim 21, further comprising informing the subject that N-(1-methylpiperidin-4-yl)-N-(4-fluorophenylmethyl)-N′-(4-(2-methylpropyloxy)phenylmethyl)carbamide ameliorates insomnia.

36. The method of claim 35, wherein the informing comprises providing printed matter that advises that N-(1-methylpiperidin-4-yl)-N-(4-fluorophenylmethyl)-N′-(4-(2-methylpropyloxy)phenylmethyl)carbamide ameliorates insomnia.

37. The method of claim 36, wherein the printed matter is a label.

38. The method of claim 21, wherein N-(1-methylpiperidin-4-yl)-N-(4-fluorophenylmethyl)-N′-(4-(2-methylpropyloxy)phenylmethyl)carbamide is administered at a time other than immediately before a sleep period.

39. The method of claim 38, wherein N-(1-methylpiperidin-4-yl)-N-(4-fluorophenylmethyl)-N′-(4-(2-methylpropyloxy)phenylmethyl)carbamide is administered in the morning.

40. A method for decreasing the number of awakenings after sleep onset comprising administering N-(1-methylpiperidin-4-yl)-N-(4-fluorophenylmethyl)-N′-(4-(2-methylpropyloxy)phenylmethyl)carbamide to a subject in need of a decreased number of awakenings after sleep onset in an amount sufficient to decrease the number of awakenings after sleep onset.

41. A method for decreasing the time awake after sleep onset comprising administering N-(1-methylpiperidin-4-yl)-N-(4-fluorophenylmethyl)-N′-(4-(2-methylpropyloxy)phenylmethyl)carbamide to a subject in need of decreased time awake after sleep onset in an amount sufficient to decrease time awake after sleep onset.

42. A method of manufacturing a pharmaceutical composition, said method comprising:
obtaining a first dosage form comprising a first amount of N-(1-methylpiperidin-4-yl)-N-(4-fluorophenylmethyl)-N′-(4-(2-methylpropyloxy)phenylmethyl)carbamide;
obtaining a second dosage form comprising a second amount of N-(1-methylpiperidin-4-yl)-N-(4-fluorophenylmethyl)-N′-(4-(2-methylpropyloxy)phenylmethyl)carbamide; and
packaging together said first dosage form and said second dosage form.

43. The method of claim 42, wherein the first dosage form and the second dosage form each comprise an oral dosage form.

44. The method of claim 42, wherein the first dosage form comprises a higher dose of N-(1-methylpiperidin-4-yl)-N-(4-fluorophenylmethyl)-N′-(4-(2-methylpropyloxy)phenylmethyl)carbamide than the second dosage form.

45. A packaged pharmaceutical composition, comprising N-(1-methylpiperidin-4-yl)-N-(4-fluorophenylmethyl)-N′-(4-(2-methylpropyloxy)phenylmethyl)carbamide in a container and instructions for using N-(1-methylpiperidin-4-yl)-N-(4-fluorophenylmethyl)-N′-(4-(2-methylpropyloxy)phenylmethyl)carbamide to treat insomnia.

46. A packaged pharmaceutical composition, comprising N-(1-methylpiperidin-4-yl)-N-(4-fluorophenylmethyl)-N′-(4-(2-methylpropyloxy)phenylmethyl)carbamide in a container and instructions for using N-(1-methylpiperidin-4-yl)-N-(4-fluorophenylmethyl)-N′-(4-(2-methylpropyloxy)phenylmethyl)carbamide to increase slow wave sleep, decrease the number of awakenings after sleep onset or decrease the time awake after sleep onset.

47. A kit comprising a first dosage form of N-(1-methylpiperidin-4-yl)-N-(4-fluorophenylmethyl)-N′-(4-(2-methylpropyloxy)phenylmethyl)carbamide and a second dosage form of N-(1-methylpiperidin-4-yl)-N-(4-fluorophenylmethyl)-N′-(4-(2-methylpropyloxy)phenylmethyl)carbamide.

48. The kit of claim 47, wherein the first dosage form contains a higher dose of N-(1-methylpiperidin-4-yl)-N-(4-fluorophenylmethyl)-N′-(4-(2-methylpropyloxy)phenylmethyl)carbamide than the second dosage form.

49. The kit of claim 47, wherein the kit further comprises instructions for taking the first dosage form at least 24 hours before taking the second dosage form.

50. The kit of claim 47, wherein the instructions are on a label.

51. The kit of claim 50, wherein the instructions further inform a subject that N-(1-methylpiperidin-4-yl)-N-(4-fluorophenylmethyl)-N′-(4-(2-methylpropyloxy)phenylmethyl)carbamide increases slow wave sleep.

52. The kit of claim 47, wherein the instructions further inform a subject that N-(1-methylpiperidin-4-yl)-N-(4-fluorophenylmethyl)-N′-(4-(2-methylpropyloxy)phenylmethyl)carbamide ameliorates insomnia.

53. The kit of claim 47, wherein the instructions further inform a subject that N-(1-methylpiperidin-4-yl)-N-(4-fluorophenylmethyl)-N′-(4-(2-methylpropyloxy)phenylmethyl)carbamide decreases the number of awakenings after sleep onset.

54. The kit of claim 47, wherein the instructions further inform a subject that N-(1-methylpiperidin-4-yl)-N-(4-fluorophenylmethyl)-N′-(4-(2-methylpropyloxy)phenylmethyl)carbamide decreases the time awake after sleep onset.
